(19) United States
(12) Patent Application Publication
(10) Pub. No.: US 2025/0034581 A1
(43) Pub. Date: Jan. 30, 2025

(54) COMPOSITIONS AND METHODS RELATED TO FUNGAL HYPOXIA RESPONSIVE MORPHOLOGY FACTOR A (HRMA) AND BIOFILM ARCHITECTURE FACTOR (BAF) PROTEINS

(71) Applicant: THE TRUSTEES OF DARTMOUTH COLLEGE, Hanover, NH (US)

(72) Inventors: Robert A. Cramer, JR., Hanover, NH (US); Caitlin Kowalski, Hanover, NH (US)

(73) Assignee: THE TRUSTEES OF DARTMOUTH COLLEGE, Hanover, NH (US)

(21) Appl. No.: 18/440,756

(22) Filed: Feb. 13, 2024

Related U.S. Application Data
(62) Division of application No. 17/642,592, filed on Mar. 11, 2022, now Pat. No. 12,116,579.
(60) Provisional application No. 62/899,660, filed on Sep. 12, 2019.
(60) Provisional application No. 63/006,930, filed on Apr. 8, 2020.

(51) Int. Cl. C12N 15/80; C07K 14/38; C12N 15/52

(52) U.S. Cl. CPC C12N 15/80; C07K 14/38; C12N 15/52

(57) ABSTRACT

Filamentous fungal host cells expressing hypoxia responsive morphology factor A (hrmA) and biofilm architecture factor (baf) proteins are provided. Methods of producing filamentous fungal host cells expressing hrmA and baf proteins are also provided.

### CROSS-REFERENCE TO RELATED APPLICATIONS

This application claims the benefit of priority to U.S. Provisional Patent Application No. 62/899,660 filed on Sep. 12, 2019, and U.S. Provisional Patent Application No. 63/006,930 filed Apr. 8, 2020, the entire contents of which are incorporated herein by reference.

### STATEMENT OF FEDERALLY SPONSORED RESEARCH

This invention was made with government support under Grant Nos. F31 AI138354 and R01 AI130128 awarded by the National Institutes of Health. The Government has certain rights in this invention.

### SEQUENCE LISTING

The instant application contains a Sequence Listing which has been submitted via Patent Center and is hereby incorporated by reference in its entirety. Said .xml copy, created on Jul. 18, 2024 is named DRTC_P2035USD1_00600973, and is 98,239 bytes in size.

### FIELD OF THE INVENTION

This disclosure relates to compositions and methods of using fungal hypoxia responsive morphology factor A (hrmA) proteins and biofilm architecture factor (baf) proteins.

### BACKGROUND

Fungi, from yeasts like Saccharomyces spp. to molds like Aspergillus spp., serve as efficient powerhouses for the mass production of many biological products (Bennett et al. J Biotechnol 66, 101-107, 1998). While many of these organisms have been genetically designed to carry out efficient production of diverse products, there remain important areas where biological improvements could significantly reduce costs of production scale fungal fermentation products. In particular, the dissolved oxygen requirements in many fungal fermentations have a significant impact on fermentation design, product yield, microbial biomass, and ultimately production costs (Show et al. Frontiers in Life Science 8, 271-283, 2015). For example, the production of citric acid through microbial fermentation requires an excess of not only glucose but also oxygen (Show, supra; Max et al. Braz J Microbiol 41, 862-875, 2010). Oxygen is an essential requirement for all fungi currently used in industrial fermentations. With yeast based fermentations, millions of cubic feet of air are introduced daily into fermentations at high cost. Moreover, the capacity to host air supply systems of sufficient size often imposes restrictions on the type of fermentations that can be conducted by a given facility.

While chemical production of citric acid has been around since the 1880's, the production efficiency hardly compares to the microbial fermentation yields (Show, supra). Aspergillus niger, introduced as a citric acid producer in 1916, is considered the microbe of choice in citric acid production due to its high yields and ability to ferment a variety of inexpensive carbon sources (Show, supra). Among molds, A. niger is also relatively tolerant to low oxygen tensions, however citric acid production by the fungus is irreversibly altered in the total absence of oxygen, and thus constant aeration is required at a rate of 0.2-1 vvm to maintain dissolved oxygen at approximately 20% of saturation (Max, supra). A reduction in the amount of oxygen required during citric acid production is expected to have significant cost benefits. The demand for citric acid in estimated to be growing annually at ˜3.5-4%; a large portion of which is accounted for by both the food industry, where citric acid is an acidifier and major ingredient in soft drinks, and industrial applications, such as metal finishing (Show, supra).

Aspergillus fumigatus is also capable of producing abundant amounts of citric acid (Bhattacharjee et al. IOSR Journal of Environmental Science, Toxicology and Food Technology 9, 19-23, 2015); and between reference genomes, A. fumigatus and A. niger share ˜69% genomic sequence similarity across orthologous protein-coding genes (Fedorova et al. PLoS Genet 4, e1000046, 2008). However, unlike A. niger, A. fumigatus is a major cause of pulmonary mycosis in immune compromised patient populations, making it ill-suited for use in biotechnological applications (Sugui et al. Cold Spring Harb Perspect Med 5, a019786, 2014). We propose herein to utilize an in vitro evolved allele of a sub-telomeric gene cluster discovered in A. fumigatus but also present in other Aspergillus species to reduce fungal oxygen consumption in A. niger and other industrial used fungi such as Trichoderma reesei, A. oryzae, and the yeast Saccharomyces cerevisiae among others, with the ultimate objective to reduce dissolved oxygen requirements in industrial scale fermentations and heterologous protein production cultures.

### SUMMARY

Disclosed herein are compositions and methods of using fungal hypoxia responsive morphology factor A (hrmA) proteins and biofilm architecture factor (baf) proteins, including variants, homologs, and orthologs thereof.

In one aspect, the disclosure provides a filamentous fungal host cell, comprising a nucleotide sequence encoding an Aspergillus fumigatus hypoxia responsive morphology factor A (hrmA) protein, or a homolog or ortholog thereof.

In certain embodiments, the filamentous fungal host cell is not Aspergillus fumigatus.

In certain embodiments, the hrmA protein comprises at least 90% identity to the amino acid sequence of SEQ ID NO: 11. In certain embodiments, the hrmA protein comprises a D304G mutation relative to the amino acid sequence of SEQ ID NO: 11. In certain embodiments, the hrmA protein comprises at least 90% identity to the amino acid sequence of SEQ ID NO: 12. In certain embodiments, the hrmA protein comprises the amino acid sequence of SEQ ID NO: 12.

In certain embodiments, the filamentous fungal host cell is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Saccharomyces, Schizophyllum, Talaromyces, Thermoascus, Thielavia, Tolypocladium, Trametes and Trichoderma.

In certain embodiments, the filamentous fungal host cell is an Aspergillus host cell. In certain embodiments, the host cell is selected from the group consisting of: Aspergillus awamori, Aspergillus flavus, Aspergillus foetidus, Aspergillus japonicus, Aspergillus luchensis, Aspergillus nidulans, Aspergillus niger or an Aspergillus oryzae host cell.

In certain embodiments, the production of any one or more of aconitate, malate, isocitrate, and citrate are increased relative to a fungal host cell that does not comprise the nucleotide sequence encoding the Aspergillus fumigatus hypoxia hrmA protein.

In certain embodiments, the fungal host cell is less adherent to plastic and glass surfaces relative to a fungal host cell that does not comprise the nucleotide sequence encoding the Aspergillus fumigatus hypoxia hrmA protein.

In another aspect, the disclosure provides a filamentous fungal host cell, comprising a nucleotide sequence encoding an Aspergillus fumigatus biofilm architecture factor (baf) protein, or a homolog or ortholog thereof.

In certain embodiments, the filamentous fungal host cell is not Aspergillus fumigatus.

In certain embodiments, the baf protein comprises bafA, or a homolog or ortholog thereof. In certain embodiments, the baf protein comprises bafB, or a homolog or ortholog thereof. In certain embodiments, the baf protein comprises bafC, or a homolog or ortholog thereof. In certain embodiments, the bafA protein comprises at least 90% identity to the amino acid sequence of SEQ ID NO: 13. In certain embodiments, the bafA protein comprises the amino acid sequence of SEQ ID NO: 13. In certain embodiments, the bafB protein comprises at least 90% identity to the amino acid sequence of SEQ ID NO: 14. In certain embodiments, the bafB protein comprises the amino acid sequence of SEQ ID NO: 14. In certain embodiments, the bafC protein comprises at least 90% identity to the amino acid sequence of SEQ ID NO: 15. In certain embodiments, the bafC protein comprises the amino acid sequence of SEQ ID NO: 15.

In certain embodiments, the filamentous fungal host cell is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Saccharomyces, Schizophyllum, Talaromyces, Thermoascus, Thielavia, Tolypocladium, Trametes and Trichoderma.

In certain embodiments, the filamentous fungal host cell is an Aspergillus host cell. In certain embodiments, the host cell is selected from the group consisting of: Aspergillus awamori, Aspergillus flavus, Aspergillus foetidus, Aspergillus japonicus, Aspergillus luchensis, Aspergillus nidulans, Aspergillus niger or an Aspergillus oryzae host cell.

In certain embodiments, the fungal host cell is less adherent to plastic and glass surfaces relative to a fungal host cell that does not comprise the nucleotide sequence encoding the Aspergillus fumigatus baf protein, or a homolog or ortholog thereof.

In another aspect, the disclosure provides a filamentous fungal host cell, comprising a nucleotide sequence encoding an Aspergillus niger biofilm architecture factor (baf) protein, or a homolog or ortholog thereof.

In certain embodiments, the filamentous fungal host cell is not Aspergillus niger.

In certain embodiments, the filamentous fungal host cell is a modified Aspergillus niger and the baf protein or a homolog or ortholog thereof is expressed to a higher level than an unmodified Aspergillus niger host cell.

In certain embodiments, the baf protein comprises bafA, or a homolog or ortholog thereof. In certain embodiments, the bafA protein comprises at least 90% identity to the amino acid sequence of SEQ ID NO: 16. In certain embodiments, the bafA protein comprises the amino acid sequence of SEQ ID NO: 16.

In certain embodiments, the fungal host cell is less adherent to plastic and glass surfaces relative to a fungal host cell that does not comprise the nucleotide sequence encoding the Aspergillus niger baf protein, or a homolog or ortholog thereof.

In certain embodiments, the filamentous fungal host cell further comprises a heterologous polynucleotide encoding a secreted polypeptide of interest.

In certain embodiments, the filamentous fungal host cell produces one or more products of interest at a higher level than a filamentous fungal host cell that does not comprise a nucleotide sequence encoding an Aspergillus fumigatus hrmA protein, an Aspergillus fumigatus baf protein, an Aspergillus niger baf protein, or homologs or orthologs thereof.

In certain embodiments, the filamentous fungal host cell secretes one or more products of interest at a higher level than a filamentous fungal host cell that does not comprise a nucleotide sequence encoding an Aspergillus fumigatus hrmA protein, an Aspergillus fumigatus baf protein, an Aspergillus niger baf protein, or homologs or orthologs thereof.

In certain embodiments, the one or more products of interest comprise citric acid, gluconic acid, fumaric acid, kojic acid, lactic acid, itaconic acid, proteins, and secondary metabolites.

In certain embodiments, the secondary metabolites are selected from the group consisting of: β-lactams, compactin, cyclosporines, gibberellins, griseofulvin, lovastatin, mycophenolic acid, pigments, siderophores, and taxol.

In certain embodiments, the filamentous fungal host cell grows at a higher level in the presence of reduced oxygen than a filamentous fungal host cell that does not comprise a nucleotide sequence encoding an Aspergillus fumigatus hrmA protein, an Aspergillus fumigatus baf protein, an Aspergillus niger baf protein, or homologs or orthologs thereof.

In certain embodiments, the filamentous fungal host cell oxygen consumption is reduced compared to a filamentous fungal host cell that does not comprise a nucleotide sequence encoding an Aspergillus fumigatus hrmA protein, an Aspergillus fumigatus baf protein, an Aspergillus niger baf protein, or homologs or orthologs thereof.

In certain embodiments, the oxygen consumption is reduced by about 10% to about 90%. In certain embodiments, the oxygen consumption is reduced by about 10%, about 20%, about 30%, about 40%, or about 50%.

In one aspect, the disclosure provides an Aspergillus niger host cell that is modified to express a biofilm architecture factor (baf) protein at a higher level than an unmodified Aspergillus niger host cell.

In certain embodiments, the baf protein comprises bafA, or a homolog or ortholog thereof. In certain embodiments, the bafA protein comprises at least 90% identity to the amino acid sequence of SEQ ID NO: 16. In certain embodiments, the bafA protein comprises the amino acid sequence of SEQ ID NO: 16.

In one aspect, the disclosure provides a method of increasing fungal secretion of one or more products of interest, comprising introducing into a filamentous fungal host cell one or more polynucleotide sequences encoding one or both of an hrmA protein and a baf protein.

In another aspect, the disclosure provides a method of increasing the production of one or more products of interest, comprising introducing into a filamentous fungal host cell one or more polynucleotide sequences encoding one or both of an hrmA protein and a baf protein.

In another aspect, the disclosure provides a method of reducing oxygen consumption of a filamentous fungal host cell, comprising introducing into a filamentous fungal host cell one or more polynucleotide sequences encoding one or both of an hrmA protein and a baf protein.

In certain embodiments, the hrmA protein comprises the amino acid sequence of SEQ ID NO: 12.

In certain embodiments, the baf protein comprises the amino acid sequence of SEQ ID NO: 13, SEQ ID NO: 14, SEQ ID NO: 15, or SEQ ID NO: 16.

In certain embodiments, oxygen consumption is reduced by about 10% to about 90%. In certain embodiments, oxygen consumption is reduced by about 10%, about 20%, about 30%, about 40%, or about 50%.

In certain embodiments, the polynucleotide sequence is introduced into the filamentous fungal host cell via transformation.

In certain embodiments, the transformation comprises one or more of protoplast-mediated transformation, Agrobacterium-mediated transformation, electroporation, biolistic transformation, and shock-wave-mediated transformation

In certain embodiments, the polynucleotide sequence is introduced into the filamentous fungal host cell transiently.

In certain embodiments, the polynucleotide sequence is stably integrated into the filamentous fungal host cell genome.

In certain embodiments, the polynucleotide sequence is introduced into the filamentous fungal host cell genome with a genetic-editing system.

In certain embodiments, the genetic-editing system comprises one or more of a meganuclease system, a ZFN system, a TALEN system, and a CRISPR system.

In certain embodiments, the polynucleotide sequence further comprises a promoter to express one or both of the hrmA protein and the baf protein. In certain embodiments, the promoter is inducible or constitutive. In certain embodiments, the inducible promoter is selected from the group consisting of: alcA, amyB, bli-3, bphA, catR, cbhI, crc1, exylA, gas, glaA, mir1, niiA, qa-2, Smxyl, tcu-1, thiA, vvd, xyl1, xylP, xyn1, or zeaR. In certain embodiments, the constitutive promoter comprises cDNA1, eno1, gpdA, gpd1, pdc1, pki1, poliC, tef1, or rp2.

In one aspect, the disclosure provides an isolated polynucleotide, comprising a nucleotide sequence encoding an hrmA allele (D304G), or a homolog or ortholog thereof, of a fungi.

In one aspect, the disclosure provides an isolated hrmA polypeptide, encoded by the polynucleotide recited above.

In one aspect, the disclosure provides a vector comprising the isolated polynucleotide recited above.

In one aspect, the disclosure provides a fungus, comprising the isolated polynucleotide recited above.

In one aspect, the disclosure provides a culture comprising the fungus recited above.

In one aspect, the disclosure provides a method for producing a biological product, wherein the method comprises culturing a fungus recited above under oxygen replete conditions, and harvesting the biological product.

In one aspect, the disclosure provides a polynucleotide, polypeptide, vector, fungi, culture, or method recited above, wherein the fungi is an Aspergillus, a Trichoderma, or a Saccharomyces.

In one aspect, the disclosure provides a polynucleotide, polypeptide, vector, fungi, culture, or method recited above, wherein the fungi is an Aspergillus niger, an Aspergillus oryzae. a Trichoderma reesei, or a Saccharomyces cerevisiae.

In one aspect, the disclosure provides a polynucleotide, polypeptide, vector, fungi, culture, or method recited above, wherein the polynucleotide is an hrmA associated gene cluster.

In one aspect, the disclosure provides a polynucleotide, polypeptide, vector, fungi, culture, recited above wherein the polynucleotide comprises a sequence according to any of the hrmA protein-coding regions of the hrmA, or homolog, or ortholog thereof, polynucleotide sequences provided herein.

In one aspect, the disclosure provides a polynucleotide, polypeptide, vector, fungi, culture, or method recited above, wherein the polynucleotide comprises a sequence according to any of the hrmA, or homolog, or ortholog thereof, polynucleotide sequences provided herein.

In one aspect, the disclosure provides a polynucleotide, polypeptide, vector, fungi, culture, recited above, wherein the polynucleotide comprises an hrmA allele (D304G) of Aspergillus, or a homolog, or ortholog, thereof.

In one aspect, the disclosure provides a polynucleotide, polypeptide, vector, fungi, culture, or method recited above, wherein the polynucleotide comprises an hrmA allele (D304G) of Aspergillus fumigatus or Aspergillus Niger, or a homolog, or ortholog thereof.

In one aspect, the disclosure provides a polynucleotide, polypeptide, vector, fungi, culture, or method recited above, wherein the fungi is a recombinant fungus or an evolved fungus.

In one aspect, the disclosure provides a polynucleotide, polypeptide, vector, fungi, culture, or method recited above, wherein the fungus is a recombinant fungus and the polynucleotide is from a different fungal species than the recombinant fungus.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts that the macroscopic morphotypes and biofilm architecture of A. fumigatus are influenced by oxygen tension. FIG. 1a. Environmental (n=29 biologically independent samples) and clinical (n=29 biologically independent samples) isolates of A. fumigatus strains plotted for morphotype characteristics (furrowing and percent vegetative mycelia) when grown at 0.2% O₂ or 21% O₂ (FIG. 1b). Two-way ANOVA shows oxygen tension significantly contributes to the variation of colony furrowing (31.67%, p<0.0001) (FIG. 1c) and percent vegetative mycelia (PVM) (55.77%, p<0.0001) (FIG. 1d) in clinical (n=29 biologically independent samples) and environment (n=29 biologically independent samples) strains. Dashed lines indicated the mean values per condition; error bars indicate standard error with measure of centre at the mean. FIG. 1e. Representative isolates with an increased PVM (white) and furrowing when cultured at 0.2% O₂ vs. 21% O₂. Images representative of three biologically independent experiments. FIG. 1f. Example clinical strains that adopt H-MORPH during growth at 21% O₂ (closed blue circles in b, c, d). Images representative of three biologically independent replicates. FIG. 1g. Representative side-view slices of submerged fungal biofilms from A. fumigatus H-Morph clinical isolates in FIG. 1f. FIG. 1h shows the quantification of vertical alignment of filaments as a function of biofilm depth. FIG. 1i shows representative side-view slices of submerged fungal biofilms of AF293 and the H-MORPH EVOL20 at 21% O₂ (24 hours) and 0.2% O₂ (36 hours) with (FIG. 1j) vertical alignment quantification. For FIG. 1h and FIG. 1j each lane is a representative alignment from a minimum of three independent biological replicates. Biofilm images are sample volumes of approximately 300 μm (h)×500 μm (l)×200 μm (w) and represent a minimum of 3 biologically independent experiments.

FIG. 2 depicts that whole genome sequencing and RNA sequencing of the hypoxia-evolved EVOL20 identified three nonsynonymous mutations. FIG. 2a. Example identification and quantification of the morphological characteristics of interest: furrowing and PVM. This is a representative example of a single analysis that was repeated three times per sample and averaged. FIG. 2b. H-MORPH strains (CDC20.2, F11698, F16311) have significantly increased hypoxia fitness (***: p<0.001 by One-way ANOVA with Dunnett multiple comparison test) compared to AF293. n=3 biologically independent samples. Error bars indicate standard error around the mean (centre). FIG. 2c. Experimental evolution schematic. FIG. 2d. Following 20 passages in 0.2% O₂ heterogeneous population was sorted into individual clonal colonies. Images are representative of three independent experiments. FIG. 2e. Afu5g14900/hrmA, Afu5g08390/ssk1, Afu5g13710 have non-synonymous mutations in EVOL20 v. AF293. RNA-sequencing shows all three to be oxygen responsive. Two-tailed unpaired t tests show hrmA to be elevated in EVOL20 (***: p<0.0001) as well as AFu5g13710 (**:p=0.0020, ***: p<0.0001) and Afu5g08390/ssk1 (**:p=0.0085, NS=0.1350). n=3 independent biological samples. FIG. 2f. The SNP in hrmA (D304G) is only found in the EVOL20 clonal colony.

FIG. 3 depicts the full field of view biofilm images used in alignment analysis. Full volume view of the submerged fungal biofilms (XZ plane) with constitutively fluorescent fungi or biofilms dyed with calcofluor white. In FIG. 3a, FIG. 3b, FIG. 3c biofilms are 1400 pm×1400 pm×−500 μm. In FIG. 3d, biofilms are 665.5 pm×665.5 pm×−300 pm. Experiments were repeated a minimum of 3 times showing similar results.

FIG. 4 depicts the Aspergillus fumigatus phylogeny of publicly available genomes depicting presence and absence of hrmA and cgnA. A. fumigatus species phylogeny indicates the presence of hrmA (red square, 95% ID) and cgnA (purple circle, 95% ID detected within same contig as hrmA) in a genetically diverse set of strains, while the majority of strains encode least one cgnA-like sequence (blue circle). Strains investigated further in this study are indicated with black circles and known H-morph strains are indicated with green circles.

FIG. 5 depicts the phenotypic characterization of H-MORPH clinical strains. FIG. 5a. Co-isolated clinical strains display N-MORPH and H-MORPH (representative of 3 independent experiments) FIG. 5b. IFM 59356-3 biofilm architecture has many horizontal filaments quantified as having greater deviation from the vertical compared to the N-MORPH IFM 59356-1 (FIG. 5c) FIG. 5d. IFM 59356-3 has reduced ECM attachment (representatives from 2 independent experiments) than IFM 59356-1. FIG. 5e. IFM 59356-3 adheres less well (***:p=0.0002 by two-tailed unpaired t test, n=5 biologically independent samples per group) compared to IFM 59356-1. Error bars indicate standard error around the mean (centre). FIG. 5f. IFM 59356-3 (n=10 biologically independent animals) has 40% greater mortality at 14 dpi compared to IFM 59356-1 (n=10 biologically independent animals) (p=0.0651 by Gehan-Breslow-Wilcoxon). FIG. 5g hrmA nor cgnA mRNA are elevated in IFM 59356-3 compared to IFM 59356-1 (n=3 biologically independent samples, error bars indicate standard error around the mean (centre)). FIG. 5h. F11698 shows H-MORPH (representative from 3 independent experiments). FIG. 5i. F11698 (n=7 biologically independent animals) is more virulent than AF293 (n=5 biologically independent animals) (*:p=0.0128 by Gehan-Breslow-Wilcoxon).

FIG. 6 depicts that the hypoxia-evolved allele of the sub-telomeric gene hrmA is sufficient to generate H-MORPH and collapse biofilm architecture. FIG. 6a. A hypoxia evolved allele of hrmA, from the hypoxia evolved strain EVOL20, is sufficient to generate H-MORPH in AF293 (hrmA^{R-EV}) and necessary for H-MORPH in EVOL20. Images representative of three biologically independent experiments. FIG. 6b. Morphotype quantification indicates hrmA^{R-EV} and EVOL20 are above the mean (dashed lines) for furrowing and PVM regardless of oxygen tension but dependent on hrmA/cgnA. FIG. 6c. hrmA^{R-EV} (n=3 biologically independent samples per group) has increased fitness (a: p=0.9942, b: p=0.0033 by One-way ANOVA with Tukey's Multiple Comparison test) in low oxygen as determined by dry weight. Error bars indicate standard error with the center at the mean. FIG. 6d. Representative side-view slices of submerged fungal biofilms reveal altered biofilm architecture in hrmA^{R-EV} is dependent on cgnA and is quantified in FIG. 6e. FIG. 6f. Representative side-view slices of submerged fungal biofilms revealing hrmA and cgnA are necessary for the collapse in biofilm architecture observed with EVOL20 and is quantified in FIG. 6g. Colony and biofilm analysis are representative of three biological replicates. Biofilm images are sample volumes of approximately 300 μm (h)×500 μm (l)×200 μm (w).

FIG. 7 depicts that the transcriptional profiles cluster overexpression of the reference allele of hrmA (hrmA^{OE}) with expression of the hypoxia-evolved allele (hrmA^{R-EV}). All genes were plotted that have differential expression across the genotypes with p<0.05 (two-sided) as determined through DESeq2 which utilizes Wald test for differential expression and adjusted for multiple comparisons using Benjamini and Hochberg procedures. Any genes where total FPKM<1 were excluded. These data indicate that AF293 and ΔhrmA^{AF} cluster closely together at 0.2% O₂ and 21% O₂. The hrmA^{R-EV} and hrmA^{OE} cluster together in normoxia and hypoxia indicating that these strains have similar transcriptional profiles despite the differences in the hrmA allele. EVOL20 has a more complex genetic background and clusters separately.

FIG. 8 depicts that the transcriptional rewiring of the hypoxia response is dependent on the hypoxia-evolved allele of hrmA and primes for improved growth in low oxygen. FIG. 8a. A heat map of collapsed biological replicates showing top genes with p<0.05 (two-sided) and log 2 differential expression>1 with a minimum FPMK of 5. Significance was determined with DESeq2 which utilizes DESeq2 which utilizes the Wald test for differential expression and adjustments for multiple comparisons using Benjamini and Hochberg procedures. Within this subset, hrmA^{R-EV} vs. AF293 reveals large scale changes in both 21% and 0.2% O₂ as a result of the hypoxia-evolved hrmA allele. FIG. 8b. Classification of all genes increased or decreased by 2-fold in hrmA^{R-EV}/AF293 at 21% O₂ or 0.2% O₂ reveals activation of the hypoxia transcriptional response at ambient oxygen and reduction in this response at low oxygen in hrmA^{R-EV}. FIG. 8c. The growth advantage of hrmA^{R-EV} at 0.2% O₂ (p=0.0282) coincides with a significant (p=0.0088) reduction in fungal growth at 21% O₂. Two-tailed unpaired t test performed on n=3 independent biological replicates with error bars showing standard error centered at the mean. FIG. 8d. A transition from 48 hours of growth at 21% O₂ to growth at 0.2% O₂ shows an initial increase (p<0.0001) in growth rate during the first 24 hours at 0.2% O₂ for hrmA^{R-EV} that is not present (ns, p=0.0789) 24-48 hours post shift to 0.2% O₂. One-way ANOVA with Tukey's Multiple Comparison Test performed on n=3 biologically independent samples. Error bars indicate standard error around the mean (centre).

FIG. 9 depicts that HrmA localizes to the nucleus where it facilitates induction of a sub-telomeric gene cluster. FIG. 9a. hrmA transcripts are detected in vivo during murine invasive disease with WT A. fumigatus and increase in abundance from 24 to 72 hours post inoculation (n=8 biologically independent animals, unpaired two-tailed students t-test, p=0.0151). FIG. 9b. Introduction of the evolved allele of hrmA (hrmA^{R-EV}) is sufficient to induce significantly increased mRNA levels of hrmA compared to AF293 (p=0.0290), and similar to EVOL20 (p=0.3066) (n=3 biologically independent samples). Students unpaired two-tailed t test performed, error bars indicated standard error around the mean (centre). FIG. 9c. In the hypoxia-evolved EVOL20, loss of hrmA leads to a significant reduction in mRNA for the gene cluster surrounding the hrmA native locus (HAC: hrmA-associated cluster) (n=3 biologically independent samples). Unpaired students two-tailed t test performed between EVOL20 and ΔhrmA^{EV} with error bars representing standard error around the mean (centre) (Afu5g14880: p<0.0001, Afu5g14890: p<0.0001, hrmA: p<0.0001, cgnA: p=0.0017, Afu5g14920: p=0.0604). FIG. 9d. Ectopic integration of a constitutively over expression WT allele of hrmA in AF293 acts in trans to significantly increase mRNA levels HAC (n=3 biologically independent samples). Unpaired students two-tailed t test performed between AF293 and hrmA^{OE} with error bars representing standard error around the mean (centre) (Afu5g14880: p=0.0002, Afu5g14890: p=0.0184, hrmA: p=0.0371, cgnA: p=0.0009, Afu5g14920: p<0.0001). FIG. 9e. In a minimum of 3 independent experiments, overexpression of hrmA (hrmA^{OE-WT-GFP}) generates H-MORPH independent of oxygen tension but dependent on an N-terminal NLS (FIG. 9f) and nuclear localization of HrmA (FIG. 9g, FIG. 9h). Scale bars=5 μm. FIG. 9i. Localization to the nucleus is necessary for the induction of the HAC gene cgnA (Afu5g14900) (n=3 biologically independent samples). Error bars indicate standard error around the mean (centre).

FIG. 10 depicts that HAC is conserved in A1163, a strain that additionally encodes 2 putative orthologous gene clusters. FIG. 10a. Heat map of co-regulated HAC genes from Afu5g14865 to Afu5g14930. FIG. 10b. Comparisons of HAC average gene size and exon GC content with the published averages for the AF293 genome reveal a slightly increased average gene size and a similar GC content to the genome. Error bars indicate standard error around the mean (centre). FIG. 10c. Closely related strains AF293 and A1163 have assembled genomes and reveal synteny within HAC across these two strains. A1163 also encodes two putative orthologous clusters with putative hrmA orthologs: AFUB_044390/hrmB and AFUB 096600/hrmC.

FIG. 11(SEQ ID NO(s): 28-31) depicts that HrmA has a RNA recognition motif (RRM) with two conserved phenylalanine residues necessary for H-MORPH. FIG. 11a. Model of HrmA with an N-terminal bipartite NLS, a weakly predicted RRM domain (E-VALUE 0.01), EVOL20 SNP locale, and the GFP tag. Green residues are the result of the site directed mutagenesis. FIG. 11b. Two conserved phenylalanine residues with the RRM domain are not necessary for nuclear localization (images: n=10 biologically independent samples from 3 independent experiments), but are necessary for induction of cgnA (n=3 biologically independent samples). Scale bars are 10 pm. Error bars indicate standard error around the mean (centre). FIG. 11c. The RRM mutant no longer adopts the H-MORPH characteristics of elevated PVM and furrowing (representative of 3 independent experiments). FIG. 11d. Quantification of the localization signal (representative of 5 independent experiments). FIG. 11e. iTASSER protein modeling of the RRM domain of HrmA (156-229 residues) maps to the structure of the chromatin remodeling protein Spt16 in Saccharomyces cerevisiae and Ashbya gossypii.

FIG. 12 depicts that HrmA-facilitated induction of the surrounding sub-telomeric gene cluster leads to increased hypoxia fitness and a modified hyphal surface. FIG. 12a. In a minimum of 3 independent experiments, loss of cgnA in hrmA^{R-EV} abolishes H-MORPH generating a AF293-like oxygen-responsive morphotype in regards to furrows and PVM (FIG. 12b). FIG. 12c. Representative HAC gene cgnA is necessary for the elevated hypoxia fitness of hrmA^{R-EV} (n=3 biological independent samples, One-way ANOVA with Dunnett's Multiple Comparison test, *: p=0.0433 and ns: p=0.9847). FIG. 12d. Adherence to plastic is reduced in response to HAC induction (EVOL20, hrmA^{OE}, hrmA^{R-EV}) and is dependent on cgnA. n=6 biologically independent biological samples from 2 independent experimental repetitions. One-way ANOVA with Sidak's Multiple Comparison test performed, ***: p<0.0001. Error bars indicate standard error around the mean (centre). FIG. 12e. SEM of 24 hour submerged fungal biofilms reveal detachment of extracellular matrix from hrmA^{OE} hyphae that is dependent on cgnA. White arrows indicate extracellular matrix. Representative images of 3 replicates. FIG. 12f. Addition of culture supernatants with secreted ECM to the non-adherent strain Δuge3^{AF} significantly (***: p<0.0001) rescues adherence. n=8 biologically independent samples with One-way ANOVA with Tukey's Multiple Comparison Test. Error bars indicate standard error around the mean (centre). FIG. 12g. The cell walls of hrmA^{R-EV} and EVOL20 are significantly (a: p<0.001, b: p>0.9999, c: p=0.0334) thinner than that of AF293 or ΔcgnA^{EV}. n=13 independent biological samples with One-way ANOVA and Tukey's Multiple Comparisons test. Error bars indicate standard error around the mean (centre). FIG. 12h hrmA^{R-EV} has reduced total chitin staining by CFW fluorescence (n=10 independent biological samples, a: p=0.0492, b: p=0.0049, c: p<0.0001) and increased β-glucan staining by Dectin-1 (n=10 independent biological samples, a: p<0.0001, b: p=0.0006, c: p=0.0044) (FIG. 12g). One-way ANOVA with Sidak's Multiple Comparison test for significant; error bars indicate standard error around the mean (centre) (FIG. 12f and FIG. 12g).

FIG. 13 depicts that overexpression of cgnA alone is not sufficient to generate H-MORPH. FIG. 13a. Ectopic over expression of cgnA does not generate H-MORPH in AF293 (representative of 3 independent experiments), does not impact hrmA RNA levels (n=4 biologically independent samples, **: p=0.0014 and NS: p=0.3960 by two-tailed unpaired t test) (FIG. 13b), does not alter fungal growth at 21% O₂ or 0.2% O₂ (n=3 biologically independent samples) (FIG. 13c), and does not impact surface adherence of AF293 (n=6 biologically independent samples, NS: p=0.5347 by unpaired t test) (FIG. 13d). FIG. 13e. Loss of cgnA in the HAC inducing strains EVOL20 and hrmA^{OE} results in a loss of H-MORPH (representative of 3 independent experiments) and a reduction in hypoxia fitness (n=3 biologically independent samples, **: p<0.0001, *: p=0.0134, NS: p=1103, p=0.1579, by One-way ANOVA with Sidak's multiple comparisons test) (FIG. 13f). FIG. 13g. ECM detachment from EVOL20 but not AF293 (representative of 2 independent experiments). Scale bars: 20 pm. All error bars indicate the standard error around the mean (centre).

FIG. 14 depicts that loss of uge3 does not impact H-MORPH or hypoxia fitness. Deletion of uge3 from hrmA^{OE} or EVOL20 does not impact the macroscopic H-MORPH (representative of 3 independent experiments) (FIG. 14a), but as expected reduces adherence of the strains (n=6 independent biological samples, ***: p<0.0001 One-way ANOVA with Dunnett multiple comparisons test) (FIG. 14b). FIG. 14c. Δuge3EVOL does not impact hypoxia fitness of EVOL20 (n=3 biologically independent samples, a: p=0.0294, b: p=0.0370, NS: p=0.9988 by One-way ANOVA with Tukey's Multiple Comparisons test) (FIG. 14d) nor hypoxia fitness of AF293 (n=3 biologically independent samples, NS: p=0.0672 by two-tailed unpaired t test). FIG. 14e. Loss of uge3 in AF293 does not impact biofilm architecture (representative of 3 independent experiments). All error bars indicate standard error around the mean (centre).

FIG. 15 depicts representative TEM images used for cell wall thickness analysis. Black arrows indicate electron-dense material presumed to be the extracellular matrix that is aggregated in the hrmA^{R-EV} and EVOL20 H-MORPH strains. Scale bars for 4000× images are 600 μm, for 10000× images are 200 μm, and for 25000× images are 100 pm. Images are representative of 13 biologically independent samples from 2 biologically independent experiment.

FIG. 16 depicts cell wall staining and cell wall perturbing agents. FIG. 16a. Calcofluor white (CFW) staining of hyphae and processed as SUM projections. FIG. 16b. Soluble Dectin-1 staining for detection of β-glucan and processed as SUM projections. Scale bars are 5 pm. Images representative from 3 biologically independent experiments. FIG. 16c. At 21% or 0.2% O₂, H-MORPH hrmA^{R-EV} (n=7 biologically independent samples) is more sensitive to growth on CFW than hrmA^{R-EV}; ΔcgnA (n=4 biologically independent samples) (a: p=0.0016, b: p=0.0037 by two-tailed unpaired t test). FIG. 16d. In contrast, there is no impact of hrmA/cgnA on fungal sensitivity to the Echinocandin Caspofungin (n=3 biologically independent samples, c: p=0.9313, d: p=0.4550 by two-tailed unpaired t test). All error bars indicate standard error around the mean (centre).

FIG. 17 depicts that H-MORPH contributes to increased virulence through increased inflammation and diffuse lesion morphology. AF293^{tdtomato} (FIG. 17a) or EVOL20^{tdtomato} (FIG. 17b) murine lesions stained with DAPI and FITC-SBA. Scale bars: 4 DPI: 10 m, 5 DPI: 100 μm. Represents 5 independent animals from 2 independent preparations. FIG. 17c. Nearest-neighbor algorithm shows significantly increased distances between intralesion filaments in AF293 and EVOL20 (**:p=0.0052) and hrmA^{R-EV} (a: p=0.0003, b: p=0.0068) (AF293 n=45, EVOL20 n=24, ΔhrmA n=25, hrmA^{R-EV}n=61, hrmA^{R-EV}; ΔcgnA n=22 biologically independent samples). One-way ANOVA with Sidak's Multiple Comparison test. FIG. 17d. hrmA^{R-EV} (n=28 independent animals) is significantly more virulent (p=0.0346 by Gehan-Breslow-Wilcoxon test (GBW)) than AF293 (n=16 independent animals) and hrmA^{R-EV}; ΔcgnA (n=10 independent animals, p=0.0417 by GBW). AF293 and hrmA^{R-EV}; ΔcgnA do not differ in virulence (ns: p=0.2087 by GBW). FIG. 17e. EVOL20 (n=30 independent animals) is significantly more virulent than ΔhrmA^{EV} (n=20 independent animals, p=0.0008 by GBW), ΔcgnA^{EV} (n=10 independent animals, p=0.0353 by GBW), and AF293 (n=10 independent animals, p=0.0465 by GBW). AF293, ΔhrmA^{EV} (p=0.1731 by GBW) nor ΔcgnA^{EV} (p=0.7812 by GBW) differ in virulence. FIG. 17f. hrmA^{R-EV} does not increase fungal burden (a: p=0.4669, b: p=0.1322, c: p=0.7960, One-way ANOVA with Dunnett's Multiple Comparison test, n=5 independent animals each). FIG. 17g Histopathology (n=6 independent animals each from 2 separate preparations) at 4 DPI indicate a cgnA-dependent increase in cellular infiltrate within hrmA^{R-EV} lesions (Scale bar: 20 μm). FIG. 17h-FIG. 17k Mice inoculated with hrmA^{R-EV} (n=8 independent animals per group) at 60 hours post-inoculation show elevated (FIG. 17h) LDH (a: p<0.0001, b: p=0.0013, c: p=0.0009), (FIG. 17i) chemoattractant KC (a: p=0.0013, b: p=0.0052, c: p=0.0001), (FIG. 17j) BALF neutrophils (a: p<0.0001), and (k) spleen weight (a: p=0.0009, b: p=0.0191, c: p=0.0021). One-way ANOVA with Dunnett's Multiple Comparisons test for FIG. 17h-FIG. 17k. All Error bars indicate standard error centered at the mean.

FIG. 18 depicts that FunPact images reflect lesion morphology from histopathology. FIG. 18a. AF293 lesions show complex interconnection between fungal filaments. FIG. 18b. EVOL20 lesions show diffuse fungal lesions at both day 4 and 5 post inoculation. FIG. 18c. AF293 lesions are compact in the airways while EVOL20 lesions are more diffuse. Scale bars—4 DPI: 10 μm, 5 DPI: 100 pm, histopathology: 20 pm. All images are representative from 5 biologically independent animals from 2 independent sample preparations.

FIG. 19 depicts histopathology slides utilized in the analysis of lesion diffuseness. Four representative images of GMS stained fungal lesions within the murine lung per strain group. Airways inoculated with hrmA^{R-EV} have more diffuse lesions that spread across the span of the airway and are not localized in a single foci of infection, as observed with AF293, Δhrm^{AF}, and hrmA^{R-EV}; ΔcgnA. Scale bars: 20 pm. Images are representative of a minimum of 5 biologically independent animals from a 2 independent experiments.

FIG. 20 depicts cellularity from tissue and histopathology. In murine lung tissue total cells (a: p<0.0001, b: p=0.0384 by One-way ANOVA with Dunnett's Multiple Comparison test) (FIG. 20a) and CD45+ leukocytes (a: p<0.0001, c: p=0.0033, d: p=0.0049 One-way ANOVA with Dunnett's Multiple Comparison test) (FIG. 20b) are elevated at 60 hpi with hrmA^{R-EV} (n=7 biologically independent animals) relative to the other strains (n=8 biologically independent animals per group). FIG. 20c. Macrophages are significantly reduced between AF293 (n=8 biologically independent animals) and hrmA^{R-EV} (n=7 biologically independent animals) in the lung tissue (e:p=0.0002 by One-way ANOVA with Dunnett's Multiple Comparison test), but (FIG. 20d) hrmA^{R-EV} neutrophils (n=7 biologically independent animals) are significantly elevated relative to all groups (n=8 biologically independent animals per group) (a: p<0.0001 by One-way ANOVA with Dunnett's Multiple Comparison test). In BAL, total cells (a: p<0.0001 by One-way ANOVA with Dunnett's Multiple Comparison test) (FIG. 20e) and CD45+ leukocytes (a: p<0.0001 One-way ANOVA with Dunnett's Multiple Comparison test) (FIG. 20f) are elevated in hrmA^{R-EV} (n=8 biologically independent animals per group), but not macrophages (b: p=0.2356, c: p=0.0755, d: p=0.9774, by One-way ANOVA with Dunnett's Multiple Comparison test) (FIG. 20g). FIG. 20h. Deletion of cgnA in hrmA^{R-EV} does not impact hrmA mRNA in vitro (n=3 biologically independent samples) (NS: p=0.4508, **: p=0.0002, One-way ANOVA with Dunnett's Multiple Comparison test). All error bars indicate standard error around the mean (centre). FIG. 20i. GMS and H&E histopathology of airway lesions of EVOL20, ΔhrmA^{EV}, and ΔcgnA^{EV} (Scale bars: 20 pm). Images are representative of 5 biologically independent animals from 2 independent experiments.

FIG. 21 depicts a model for the impact of macroscopic morphology of disease progression of A. fumigatus.

FIG. 22 depicts a conceptual overview of oxygen consumption modulation technology in industrial relevant fungi.

FIG. 23 depicts that an hrmA evolved allele facilitates reduced oxygen consumption in the hypoxia-evolved strain EVOL20. FIG. 23A. Agilent Seahorse XFe96 technology for basal oxygen consumption reveals significantly reduced oxygen consumption by the hypoxia-evolved strain of Aspergillus fumigatus EVOL20 (p<0.001 by Students t test). FIG. 23B. Unisense microelectrode technology (OX-25) with 5 mL of 12 hour planktonic cultures reveals reduced oxygen depletion by EVOL20 in fresh aerated media that is dependent on the hypoxia-evolved hrmA allele in EVOL20. FIG. 23C. The fungal biomass used in FIG. 23B to quantify the reduction in dissolved oxygen are not significantly different (p=0.08 by One-Way ANOVA with Tukey multiple comparisons test). Error bars indicate standard error around the mean.

FIG. 24 depicts that the hypoxia evolved strain EVOL20 has increased TCA cycle intermediates and reduces surface adherence. FIG. 24A. 24 hours cultures of EVOL20 and AF293 were shifted from spent media at 21% oxygen to fresh media in 0.2% oxygen for 120 minutes. Following incubation, fungal tissue was collected, washed, quenched, and flash frozen. Unbiased metabolomics was performed at the University of California Davis Metabolomics Center. FIG. 24B. Adherence to polystyrene 96-well plates after 24 hour incubation at normal oxygen is significantly reduced (p<0.001, student's t test) in EVOL20 compared to AF293. Error bars indicate standard error around the mean.

FIG. 25 (SEQ ID NO(s): 16, 32-33) depicts A. niger putative HAC homolog alignments. Clustal omega alignment of A. niger CBS 513.88 An08g12010, A fumigatus A1163 AFUB_044360, and the uncharacterized, unannotated A. fumigatus AF293 HAC gene Afu5g14915 with open reading frame predictions based on RNA-sequencing and alignments with AFUB_044360.

FIG. 26 depicts three example cloning strategies for the engineering of A. niger. FIG. 26A. Example plasmid for ectopic introduction and overexpression of A. fumigatus hrmA from EVOL20 into A. niger using Hygromycin as a dominant marker. FIG. 26B. Example plasmid for the ectopic introduction and overexpression of A. niger putative HAC ortholog An08g12010 with Hygromycin as a dominant marker. FIG. 26C. Example plasmid for the targeted introduction of a larger portion of HAC including the 3′ 4 genes Afu5g14900/hrmA, Afu5g14910, Afu5g14915, and Afu5g14920. This plasmid encodes the dominant marker for pyrithiamine resistance (ptrA) and can be used in conjunction with phrmA_OE (A) to facilitate introduction and high expression of the HAC genes.

FIG. 27 depicts the predicted results from a successfully engineered A. niger strain. The following phenotypes are expected. FIG. 27A. The A. niger strain with the overexpression of hrmA from A. fumigatus (evolved allele: D304G) or the over expression of the putative HAC homolog An08g12010 will result in A. niger strains that consume less oxygen in submerged cultures. FIG. 27B. These genetic changes will also result in increased, or unaltered, production of citrate, and these technologically-relevant phenotypes will not result in a significant reduction in fungal biomass (FIG. 27C).

FIG. 28 depicts that a cryptic gene within the hrmA-associated gene cluster is necessary for the hypoxia-evolved phenotypes of EVOL20. (FIG. 28A) 96-hour colony biofilms in normoxia (21% O2) and hypoxia (0.2% O2). Images are representative of three independent biological samples. (FIG. 28B) Quantification of colony biofilm morphological features from three independent biological samples. One-way ANOVA with Dunnett's posttest for multiple comparisons was performed relative to EVOL20 within each oxygen environment. (FIG. 28C) The ratio of fungal biomass in hypoxia (0.2% O2) relative to fungal biomass in normoxia (21% O2) (H/N) in shaking flask cultures. One-way ANOVA with Dunnett's posttest for multiple comparisons was performed relative to EVOL20. n=3 independent biological samples. (FIG. 28D) Adherence to plastic measured through a crystal violet assay. Dashed line marks the mean value for media alone. One-way ANOVA with Dunnett's posttest for multiple comparisons was performed relative to ΔcgnAEVOL. n=6 independent biological replicates. (FIG. 28E) Schematics of DNA constructs utilized in generating the cgnAOE and cgnARECON strains in the ΔcgnAEVOL strain. The sequence for the gpdA promoter and trpC terminator are from A. nidulans. (FIG. 28F) Gene expression measured by qRT-PCR for cgnA and the cryptic ORF. n=3 independent biological replicates. One-way ANOVA with Tukey's multiple comparison test was performed. (FIG. 28G) Schematic alignment of the hrmA associated gene cluster (HAC) and the putative orthologous gene clusters identified in the strain CEA10. Grey boxes align putative orthologous genes. Not drawn to scale. Error bars indicate standard error around the mean. (ns: p>0.05, not significant).

FIG. 29 depicts that the putative ortholog of the cryptic gene, bafB, is sufficient to complement the loss of the HAC genes cgnA and bafA. (FIG. 29A) 96-hour colony biofilms from 21% O2 where hypoxia-locked (H-MORPH) morphological features, furrows and vegetative mycelia, can be visualized. Images are representative of three independent biological samples. (FIG. 29B) Quantification of the H-MORPH features from colony biofilms of three independent biological samples. Student's two-tailed non-parametric t tests were performed between each isogenic strain set. (FIG. 29C) The ratio of fungal biomass in hypoxia (0.2% O2) relative to fungal biomass in normoxia (21% O2) (H/N) in shaking flask cultures. Student's two-tailed non-parametric t test performed between isogenic strain sets. n=3 independent biological samples. (FIG. 29D) Adherence to plastic measured through a crystal violet assay. Dashed line marks the mean value for media alone. Student's two-tailed non-parametric t test performed between isogenic strain sets. n=7 independent biological samples. (FIG. 29E) Gene expression measured by qRT-PCR for representative HAC genes as a result of bafB over expression at 21% O2. n=3 independent biological samples. (FIG. 29F) Representative images (n=3 biological samples) of submerged biofilms on the orthogonal plane (XZ). Scale bar is 200 μm. Error bars indicate standard error around the mean.

FIG. 30 depicts that the introduction of the HAC cryptic gene bafA is sufficient to generate H-MORPH in AF293 and impacts biofilm architecture in the baf+ strain CEA10. (FIG. 30A) 96-hour colony biofilms in normoxia (21% O2) and hypoxia (0.2% O2) of AF293 and AF293 with the over expression of bafA. Images are representative of three independent biological samples. (FIG. 30B) 96-hour colony biofilms in normoxia (21% O2) and hypoxia (0.2% O2) of CEA10 and CEA10 with the over expression of bafA. Images are representative of three independent biological samples. (FIG. 30C) Quantification of the H-MORPH features from colony biofilms of three independent biological samples. Student's two-tailed non-parametric t tests were performed between each isogenic strain set. (FIG. 30D) Representative images of submerged biofilms (n=3 biological samples) on the orthogonal plane (XZ). Scale bar is 200 μm. (FIG. 30E) Adherence to plastic measured through a crystal violet assay. Dashed line marks the mean value for media alone. Student's two-tailed non-parametric t test performed between isogenic strain sets. n=6 independent biological samples. (FIG. 30F) The ratio of fungal biomass in hypoxia (0.2% O2) relative to fungal biomass in normoxia (21% O2) (H/N) in shaking flask cultures. Student's two-tailed non-parametric t test performed between isogenic strain sets. n=4 independent biological samples. (FIG. 30G) Gene expression measured by qRT-PCR for bafA, bafB, and bafC in AF293 and CEA10. n=3 independent biological samples. Error bars indicate standard error around the mean.

FIG. 31 depicts that the introduction of bafB and bafC impact colony and submerged biofilm morphology in independent strain backgrounds. (FIG. 31A) 96-hour colony biofilms in normoxia (21% O2) and hypoxia (0.2% O2) of AF293 and AF293 with the over expression of bafB or bafC. Images are representative of three independent biological samples. (FIG. 31B) 96-hour colony biofilms in normoxia (21% O2) and hypoxia (0.2% O2) of CEA10 and CEA10 with the over expression of bafB or bafC. Images are representative of three independent biological samples. (FIG. 31C) Quantification of the H-MORPH features from colony biofilms of AF293, AF293 bafBOE, and AF293 bafCOE with three independent biological samples. One-way ANOVA with Dunnett's posttest for multiple comparisons relative to AF293 were performed. (FIG. 31D) Quantification of the H-MORPH features from colony biofilms of CEA10, CEA10 bafBOE, and CEA10 bafCOE with three independent biological samples. One-way ANOVA with Dunnett's posttest for multiple comparisons relative to CEA10 were performed. (FIG. 31E) Quantification of conidiation from three independent biological samples of CEA10 and CEA10 bafBOE in normoxia (21% O2) or hypoxia (0.2% O2). Student's two-tailed non parametric t tests were performed between CEA10 and CEA10 bafBOE for each time point. (a: p=0.0004, b: p=0.0006, c: p<0.0001). (FIG. 31F) Adherence to plastic measured through a crystal violet assay. Dashed line marks the mean value for media alone. One-way ANOVA with Dunnett's posttest for multiple comparisons was performed between isogenic strain sets relative to AF293 or CEA10. n=6 independent biological samples for AF293 strains and n=8 independent biological samples for CEA10 strains. (FIG. 31G) Representative images of submerged biofilms (n=3 biological samples) on the orthogonal plane (XZ) of AF293, AF293 bafBOE, and AF293 bafCOE. Scale bar is 200 μm. (FIG. 31H) Representative images of submerged biofilms (n=3 biological samples) on the orthogonal plane (XZ) of CEA10, CEA10 bafBOE, and CEA10 bafCOE. Scale bar is 200 μm.

FIG. 32 depicts that the introduction of A. fumigatus bafA is sufficient to generate H-MORPH in Aspergillus niger. (FIG. 32A) 96-hour colony biofilms in normoxia (21% O2) and hypoxia (0.2% O2) of A. niger reference strain A1144 and two independent strains of A1144 with the over expression of A. fumigatus bafA (AfbafAOE). Images are representative of three independent biological samples. (FIG. 32B) Quantification of the H-MORPH features from colony biofilms of three independent biological samples in normoxia (21% O2). One-way ANOVA with Dunnett's posttest for multiple comparisons relative to A1144. (FIG. 32C) Representative images of submerged biofilms (n=3 biological samples) on the orthogonal plane (XZ). Scale bar is 200 m. (FIG. 32D) The ratio of fungal biomass in hypoxia (0.2% O2) relative to fungal biomass in normoxia (21% O2) (H/N) in shaking flask cultures. Student's two-tailed non-parametric t test performed between isogenic strain sets. n=3 independent biological samples. (FIG. 32E) Adherence to plastic measured through a crystal violet assay. Student's two-tailed non-parametric t test performed between samples within each media type. n=6 independent biological samples for minimal media and n=7 independent biological samples for complex media (minimal media with yeast extract).

### DETAILED DESCRIPTION

Filamentous fungal host cells engineered to express and/or overexpress an hrmA protein, or homolog, or ortholog thereof, are provided. Also provided are filamentous fungal host cells engineered to express and/or overexpress a baf protein, or homolog, or ortholog thereof.

Generally, nomenclature used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “filamentous fungal host cell” refers to a fungal host cell that produces elongated and thread-like (filamentous) structures called hyphae. Filamentous fungal host cells are capable of secreting proteins and various metabolites, including many commercially relevant products, such as industrial enzymes. Non-limiting examples of filamentous fungal host cells include filamentous fungal host cells belonging to a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Saccharomyces, Schizophyllum, Talaromyces, Thermoascus, Thielavia, Tolypocladium, Trametes and Trichoderma. In certain embodiments, the filamentous fungal host cell is an Aspergillus host cell. In certain embodiments, the filamentous fungal host cell is an Aspergillus host cell other than Aspergillus fumigatus.

In certain embodiments, the filamentous fungal host cell is a fungal species useful in industrial production of products of interest. In certain embodiments, the host cell is selected from the group consisting of: Aspergillus awamori, Aspergillus flavus, Aspergillus foetidus, Aspergillus japonicus, Aspergillus luchensis, Aspergillus nidulans, Aspergillus niger or an Aspergillus oryzae host cell.

Hypoxia Responsive Morphology Factor A (hrmA)

As used herein, the term “Hypoxia Responsive Morphology Factor A” or “hrmA” or “Afu5g14900” refers to a protein encoded by an Aspergillus fumigatus hrmA gene. In certain embodiments, the hrmA protein can be a homolog or ortholog of the Aspergillus fumigatus hrmA. In certain embodiments, the hrmA protein comprises at least 80%, at least 85%, at least 90%, or at least 95% identity to the amino acid sequence of SEQ ID NO: 11. In certain embodiments, the hrmA protein comprises or consists of the amino acid sequence of SEQ ID NO: 11. In certain embodiments, the hrmA protein comprises a D304G mutation relative to the amino acid sequence of SEQ ID NO: 11. In certain embodiments, the hrmA protein comprises at least 80%, at least 85%, at least 90%, or at least 95% identity to the amino acid sequence of SEQ ID NO: 12. In certain embodiments, the hrmA protein comprises or consists of the amino acid sequence of SEQ ID NO: 12.

It has been surprisingly discovered that expression (including overexpression) of hrmA in a filamentous fungal host cell promotes a hypoxia-specific morphology (H-MORPH) that is characterized, in part, by increased colony furrowing and high vegetative mycelia (white, non-conidiating mycelia) (PVM). In certain embodiments, a PVM of greater than about 30%, 35%, 40%, 45%, or 50% is considered indicative of a hypoxia-specific morphology. This morphology is associated with increased low oxygen fitness, increased production of products of interest, and increased secretion of products of interest.

In one aspect, the disclosure provides a filamentous fungal host cell, comprising a nucleotide sequence encoding an Aspergillus fumigatus hypoxia responsive morphology factor A (hrmA) protein, or a homolog or ortholog thereof.

In certain embodiments, the filamentous fungal host cell is not Aspergillus fumigatus. In certain embodiments, the filamentous fungal host cell is engineered to overexpress an hrmA protein, or homolog, or ortholog thereof. In certain embodiments, the filamentous fungal host cell is engineered to overexpress an hrmA protein that comprises at least 90% identity to the amino acid sequence of SEQ ID NO: 11. In certain embodiments, the filamentous fungal host cell is engineered to overexpress an hrmA protein that comprises at least 90% identity to the amino acid sequence of SEQ ID NO: 12. In certain embodiments, the filamentous fungal host cell is an engineered Aspergillus fumigatus host cell that overexpresses an hrmA protein, or homolog, or ortholog thereof. Overexpression of the hrmA protein, or homolog, or ortholog thereof, is relative to a wild-type, un-engineered Aspergillus fumigatus host cell. The engineered Aspergillus fumigatus host cell may overexpress the hrmA protein, or homolog, or ortholog thereof, by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 500-fold, or 1000-fold, relative to a wild-type Aspergillus fumigatus host cell.

The filamentous fungal host cell that can express (including overexpress) the hrmA protein can be any filamentous fungal host cell known in the art. In certain embodiments, the filamentous fungal host cell belongs to a fungal genus useful in industrial production of products of interest. In certain embodiments, the filamentous fungal host cell is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Saccharomyces, Schizophyllum, Talaromyces, Thermoascus, Thielavia, Tolypocladium, Trametes and Trichoderma. In certain embodiments, the filamentous fungal host cell is an Aspergillus host cell. In certain embodiments, the filamentous fungal host cell is an Aspergillus host cell other than Aspergillus fumigatus.

In certain embodiments, the filamentous fungal host cell is a fungal species useful in industrial production of products of interest. In certain embodiments, the host cell is selected from the group consisting of: Aspergillus awamori, Aspergillus flavus, Aspergillus foetidus, Aspergillus japonicus, Aspergillus luchensis, Aspergillus nidulans, Aspergillus niger or an Aspergillus oryzae host cell.

In certain embodiments of the filamentous fungal host cell, the production of any one or more of aconitate, malate, isocitrate, and citrate are increased relative to a fungal host cell that does not comprise a nucleotide sequence encoding the Aspergillus fumigatus hrmA protein, or homolog, or ortholog thereof.

The filamentous fungal host cell expressing hrmA may comprise a hypoxia-specific morphology. In certain embodiments, the fungal host cell (e.g., the fungal host cell engineered to express or overexpress the hrmA protein, or homolog, or ortholog thereof) is less adherent to plastic and glass surfaces relative to a fungal host cell that does not comprise a nucleotide sequence encoding the Aspergillus fumigatus hrmA protein, or homolog, or ortholog thereof. Adherence may be reduced by about 10%, 20%, 30%, 40%, 50%, 60%, 70%, or 80%.

Biofilm Architecture Factor (baf)

As used herein, the term “Biofilm Architecture Factor” or “baf” refers to a class of proteins found in select fungal species that play a role in generating a hypoxia-specific morphology (H-MORPH) and promote biofilm architecture reorganization. In certain embodiments, the baf protein can be a homolog or ortholog of an Aspergillus fumigatus baf protein. In certain embodiments, the Aspergillus fumigatus baf protein is bafA, or homolog or ortholog thereof. In certain embodiments, the Aspergillus fumigatus baf protein is bafB, or homolog or ortholog thereof. In certain embodiments, the Aspergillus fumigatus baf protein is bafC, or homolog or ortholog thereof.

In certain embodiments, the bafA protein comprises at least 80%, at least 85%, at least 90%, or at least 95% identity to the amino acid sequence of SEQ ID NO: 13. In certain embodiments, the bafA protein comprises or consists of the amino acid sequence of SEQ ID NO: 13. In certain embodiments, the bafB protein comprises at least 80%, at least 85%, at least 90%, or at least 95% identity to the amino acid sequence of SEQ ID NO: 14. In certain embodiments, the bafB protein comprises or consists of the amino acid sequence of SEQ ID NO: 14. In certain embodiments, the bafC protein comprises at least 80%, at least 85%, at least 90%, or at least 95% identity to the amino acid sequence of SEQ ID NO: 15. In certain embodiments, the bafC protein comprises or consists of the amino acid sequence of SEQ ID NO: 15.

It has been surprisingly discovered that expression (including overexpression) of a baf protein in a filamentous fungal host cell promotes a hypoxia-specific morphology (H-MORPH) that is characterized, in part, by increased colony furrowing and high vegetative mycelia (white, non-conidiating mycelia) (PVM). In certain embodiments, a PVM of greater than about 30%, 35%, 40%, 45%, or 50% is considered indicative of a hypoxia-specific morphology. This morphology is associated with increased low oxygen fitness, increased production of products of interest, and increased secretion of products of interest.

In one aspect, the disclosure provides a filamentous fungal host cell, comprising a nucleotide sequence encoding an Aspergillus fumigatus biofilm architecture factor (baf) protein, or a homolog or ortholog thereof. In certain embodiments, the filamentous fungal host cell is not Aspergillus fumigatus. In certain embodiments, the filamentous fungal host cell is engineered to overexpress a baf protein, or homolog, or ortholog thereof. In certain embodiments, the filamentous fungal host cell is engineered to overexpress an baf protein that comprises at least 90% identity to the amino acid sequence of SEQ ID NO: 13. In certain embodiments, the filamentous fungal host cell is engineered to overexpress an baf protein that comprises at least 90% identity to the amino acid sequence of SEQ ID NO: 14. In certain embodiments, the filamentous fungal host cell is engineered to overexpress an baf protein that comprises at least 90% identity to the amino acid sequence of SEQ ID NO: 15. In certain embodiments, the filamentous fungal host cell is an engineered Aspergillus fumigatus host cell that overexpresses a baf protein, or homolog, or ortholog thereof. Overexpression of the baf protein, or homolog, or ortholog thereof, is relative to a wild-type, un-engineered Aspergillus fumigatus host cell. The engineered Aspergillus fumigatus host cell may overexpress the baf protein, or homolog, or ortholog thereof, by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 500-fold, or 1000-fold, relative to a wild-type Aspergillus fumigatus host cell.

In certain embodiments, the baf protein comprises bafA, or a homolog or ortholog thereof. In certain embodiments, the baf protein comprises bafB, or a homolog or ortholog thereof. In certain embodiments, the baf protein comprises bafC, or a homolog or ortholog thereof.

The filamentous fungal host cell that can express (including overexpress) a baf protein (such as bafA, bafB, and bafC) can be any filamentous fungal host cell known in the art. In certain embodiments, the filamentous fungal host cell belongs to a fungal genus useful in industrial production of products of interest. In certain embodiments, the filamentous fungal host cell is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Saccharomyces, Schizophyllum, Talaromyces, Thermoascus, Thielavia, Tolypocladium, Trametes and Trichoderma. In certain embodiments, the filamentous fungal host cell is an Aspergillus host cell. In certain embodiments, the filamentous fungal host cell is an Aspergillus host cell other than Aspergillus fumigatus.

In certain embodiments, the filamentous fungal host cell is a fungal species useful in industrial production of products of interest. In certain embodiments, the host cell is selected from the group consisting of: Aspergillus awamori, Aspergillus flavus, Aspergillus foetidus, Aspergillus japonicus, Aspergillus luchensis, Aspergillus nidulans, Aspergillus niger or an Aspergillus oryzae host cell.

In certain embodiments, of the filamentous fungal host cell, the production of any one or more of aconitate, malate, isocitrate, and citrate are increased relative to a fungal host cell that does not comprise the nucleotide sequence encoding an Aspergillus fumigatus baf protein (such as bafA, bafB, and bafC).

The filamentous fungal host cell expressing a baf protein may comprise a hypoxia-specific morphology. In certain embodiments, the fungal host cell is less adherent to plastic and glass surfaces relative to a fungal host cell that does not comprise the nucleotide sequence encoding an Aspergillus fumigatus baf protein (such as bafA, bafB, and bafC).

In another aspect, the disclosure provides a filamentous fungal host cell, comprising a nucleotide sequence encoding an Aspergillus niger biofilm architecture factor (baf) protein, or a homolog or ortholog thereof. In certain embodiments, the filamentous fungal host cell is not Aspergillus niger.

In certain embodiments, the baf protein comprises bafA, or a homolog or ortholog thereof. In certain embodiments, the bafA protein comprises at least 90% identity to the amino acid sequence of SEQ ID NO: 16. In certain embodiments, the bafA protein comprises or consists of the amino acid sequence of SEQ ID NO: 16. In certain embodiments, the fungal host cell is less adherent to plastic and glass surfaces relative to a fungal host cell that does not comprise the nucleotide sequence encoding the Aspergillus niger baf protein, or a homolog or ortholog thereof.

In one aspect, the disclosure provides a modified Aspergillus niger host cell, wherein the Aspergillus niger host cell is modified to express a biofilm architecture factor (baf) protein at a higher level than an unmodified Aspergillus niger host cell. In certain embodiments, the modified Aspergillus niger host cell is engineered to overexpress a baf protein, or homolog, or ortholog thereof, by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 500-fold, or 1000-fold, relative to a unmodified Aspergillus niger host cell.

In certain embodiments, the baf protein comprises bafA, or a homolog or ortholog thereof. In certain embodiments, the bafA protein comprises at least 90% identity to the amino acid sequence of SEQ ID NO: 16. In certain embodiments, the bafA protein comprises or consists of the amino acid sequence of SEQ ID NO: 16.

### Additional Filamentous Fungal Host Cell Features

In certain embodiments, the filamentous fungal host cell expressing one or more of an hrmA protein and baf protein further comprises a heterologous polynucleotide encoding a secreted polypeptide of interest. In certain embodiments, the polypeptide of interest is an enzyme.

In certain embodiments, the filamentous fungal host cell produces one or more products of interest at a higher level than a filamentous fungal host cell that does not comprise a nucleotide sequence encoding an Aspergillus fumigatus hrmA protein, an Aspergillus fumigatus baf protein, an Aspergillus niger baf protein, or homologs or orthologs thereof.

In certain embodiments, the filamentous fungal host cell secretes one or more products of interest at a higher level than a filamentous fungal host cell that does not comprise a nucleotide sequence encoding an Aspergillus fumigatus hrmA protein, an Aspergillus fumigatus baf protein, an Aspergillus niger baf protein, or homologs or orthologs thereof.

In certain embodiments, the one or more products of interest comprise citric acid, gluconic acid, fumaric acid, kojic acid, lactic acid, itaconic acid, proteins, and secondary metabolites.

As used herein, the term “secondary metabolites” refers a group of fungal-produced low-molecular weight compounds. The secondary metabolites generally are not directly involved in fundamental metabolic processes of growth and energy generation; however, they display varied biologic activities that contribute to the survival of the producing fungus under particular conditions. Secondary metabolites can belong to three broad classes, polyketides, non-ribosomal peptides, and terpenes. Non-limiting examples of secondary metabolites include, β-lactams (such as cephalosporins and penicillin), compactin, cyclosporines (such as cyclosporine A), gibberellins (such as gibberelic acid), griseofulvin, lovastatin, mycophenolic acid, pigments (such as astaxanthin, β-carotene, monascin, ankaflavin, monascorubrin, and rubropunctatin), siderophores, and taxol. In certain embodiments, the secondary metabolites are selected from the group consisting of: β-lactams, compactin, cyclosporines, gibberellins, griseofulvin, lovastatin, mycophenolic acid, pigments, siderophores, and taxol. Additional description and examples of secondary metabolites may be found in Boruta (Bioengineered, 9(1): 12-16, 2018) and Hoffmeister et al. (Nat Prod Rep. 24(2):393-416, 2007).

In certain embodiments, the filamentous fungal host cell grows at a higher level in the presence of reduced oxygen than a filamentous fungal host cell that does not comprise a nucleotide sequence encoding an Aspergillus fumigatus hrmA protein, an Aspergillus fumigatus baf protein, an Aspergillus niger baf protein, or homologs or orthologs thereof.

In certain embodiments, filamentous fungal host cell oxygen consumption is reduced compared to a filamentous fungal host cell that does not comprise a nucleotide sequence encoding an Aspergillus fumigatus hrmA protein, an Aspergillus fumigatus baf protein, an Aspergillus niger baf protein, or homologs or orthologs thereof.

In certain embodiments, the oxygen consumption is reduced by about 10% to about 90%. In certain embodiments, the oxygen consumption is reduced by about 10%, about 15%, about 20%, about 25%, about 30%, about 35%, about 40%, about 45%, about 50%, about 55%, about 60%, about 65%, about 70%, about 75%, about 80%, about 85%, or about 90%.

In certain embodiments, the present disclosure provides a plasmid harboring a polynucleotide sequence encoding a hrmA protein or baf protein (such as bafA, bafB, or bafC). In certain embodiments, the plasmid is an expression vector harboring a polynucleotide sequence encoding a hrmA protein or baf protein (such as bafA, bafB, or bafC). In certain embodiments, the polynucleotide sequence further comprises a promoter to express one or both of the hrmA protein and the baf protein. In certain embodiments, the expression vector harboring the polynucleotide sequence further comprises a promoter to express one or both of the hrmA protein and the baf protein.

The promoter can be one chosen based on the filamentous fungal host cell being employed. For example, but in no way limiting, the promoter can be a naturally-occurring promoter in the filamentous fungal host cell being employed. In another non-limiting example, the promoter can be a heterologous promoter not found in the filamentous fungal host cell being employed.

In certain embodiments, the promoter is inducible or constitutive. In certain embodiments, the inducible promoter is selected from the group consisting of: alcA, amyB, bli-3, bphA, catR, cbhI, cre1, exylA, gas, glaA, mir1, niiA, qa-2, Smxyl, tcu-1, thiA, vvd, xyl1, xylP, xyn1, or zeaR. In certain embodiments, the constitutive promoter comprises cDNA1, eno1, gpdA, gpd1, pdc1, pki1, poliC, tef1, or rp2. Further details on fungal host cell promoters useful for the expression of genes of interest are described in Fitz et al, (Front. Bioeng. Biotechnol. 6: 135, 2018) and Kluge et al. (Appl Microbiol Biotechnol. 102(15):6357-6372, 2018).

Polynucleotides encoding one or both of an hrmA protein and a baf protein (such as bafA, bafB, or bafC) of the disclosure may be introduced into the filamentous fungal host cells by any means known in the art, including via transformation.

As used herein, the term “transformation” refers to a non-viral method of DNA transfer in bacteria and non-animal eukaryotic cells, such as fungal cells. Numerous methods of fungal cell transformation are known in the art. Examples include, but are not limited to, protoplast-mediated transformation, Agrobacterium-mediated transformation, electroporation, biolistic transformation (i.e., particle bombardment), and shock-wave-mediated transformation. Methods of fungal host cell transformation are described in greater detail in Li et al. (Microb Cell Fact. 16: 168, 2017).

The polynucleotides encoding one or both of an hrmA protein and a baf protein may be introduced into the filamentous fungal host cells transiently or stably integrated into the host cell genome. If stable integration is employed, the polynucleotides can have homology arms at the 5′ and 3′ ends to facilitate integration.

Genomic modification of the filamentous fungal host cells may be performed with any known genetic editing technology. Non-limiting examples of genetic editing technologies include, meganucleases, zinc finger nucleases (ZFN), TALENs, and CRISPR.

The use of CRISPR genetic editing can be performed with CRISPR/Cas9-based systems or CRISPR/Cas12-based systems. The CRISPR system is composed of a CRISPR nuclease (such as Cas9 or Cas12) and a site-specific genome-targeting guide RNA (gRNA). The CRISPR system can be introduced via one or more expression cassettes that expresses the CRISPR nuclease and gRNA, such as a vector. The CRISPR nuclease and gRNA can be expressed off of a single expression cassette or separate expression cassettes. The CRISPR system can be introduced as a ribonucleoprotein (RNP) complex, where the CRISPR nuclease and gRNA form a complex in vitro (the CRISPR RNP), and the RNP is introduced into the filamentous fungal host cell. The filamentous fungal host cells can be transformed with a CRISPR system with any of the above recited transformation methods. The use of CRISPR genetic editing of fungal cells is described in greater detail in Dong et al. (J Microbiol Methods 163, 105655, 2019), Leynaud-Kieffer et al. (PLoS One 14, e0210243, 2019), and Song et al. (Appl Microbiol Biotechnol. 2019; 103(17): 6919-6932.).

The hrmA and baf proteins of the disclosure, and the polynucleotides that encode the same, are recited below in Table 1 and Table 2.

TABLE 1
Polynucleotide Sequences of the Disclosure. Lower case italicized
sequences represent introns.

| Sequence Notes | Sequence |
|---|---|
| A. fumigatus_hrmA/ | ATGGCATCCACAAAGCCCGCTTCGAGTCTCATTTACC |
| Afu5gl4900 hypoxia | AGGCATGGAACAAACTCAGTATCAACCAAACCATCC |
| evolved genomic sequence | CTAGTGACTCCCTTGAATTACTTGGGGAGCGTTTGGC |
| Bold, underlined, and | TATTGCCTTCGCACCCAAACTCAAGGAGCAACGAAG |
| italicized “G” corresponds to | GAATGGCCGGCGTCGGAATCTGGAATATGTGGCACA |
| the sequence change as a | ACATCGACGGAAGATTGCTCGAAAAATCTACTTGGA |
| result of in vitro evolution in | GATTCTGGAGAAAGACCCAAATATCTTTCTTCCTTTT |
| hypoxia to produce the hrmA | ATCCTGGCTGTTTCCCCTAGAGCATGCTTATCCTTTGA |
| D304G amino acid variant. | TATCTCGAGCTTTCTTGAACAGCACCAAAGCCAAGGA |
| (SEQ ID NO: 1) | AGACATTTCCTCCGCAACAATGCCGAAGCGATCCTCT |
| | GGGGTCTCGCAAAGAAACATGACATTGATGGCTCCCT |
| | CCATTTCAGGAAGCTGATGCGTGAGATTTTCCAACTG |
| | TCTCCTCCAGCGACAGAAGCCGAAGGCAAGGAGCAT |
| | TATTCATTGCATTTAAGCACTCTCCCCGCAATCCGCA |
| | ATGCCTTCGGTGATGTTATCTTTGACGCAATTGAACG |
| | TTCCCCTACACAGGTGACAGCGAGAGCTAAAGGTTAT |
| | TTCTCTGAGAAAACCGAAAGTGTTTGGACAAAAGTTC |
| | CCTACAGAAGTTCTCAAGACGCAATCATATCTCTTGA |
| | AGTAGGGTCGGCAATCGAGCTTGCGAATGTGTTGTTC |
| | CCAATCGCAACCCAAAAAATTGTCTCTATCCTTTCCG |
| | CATGTTCTCCCACTGTGCGCCAGAAGAACTTTTCTGA |
| | GGCTATTCTCGGCCCAGACCCTCAGGATACACCGGCA |
| | ACATCATCAGAAATCGgtatgaagtttaaggtacacatgactgcagttg |
| | ctaattccaccctgtgctagATGTGGCGTATTTTACTCTGCGAGG |
| | AGCAACGGTCTCGGCAATTGAATCAGTCTTTCGCGCT |
| | GATATTTGCGAAGGTATTAAGGGCAGCGAACTGAGA |
| | AACTGGGAAAAGGAGCAGCTGCTCATCGACACGACA |
| | GATTGTGTCACGATGCAGATATGGCGGGCACAACCTC |
| | AACATGGAACCATCAAGTTGCGTATTGGATTCTATGC |
| | AGCGGTGAATTTGGCAAATCGGCTGTATGCAGAAAC |
| | ACCCCAAGATCACATATAG |
| A. fumigatus 3′ HAC region | CGCCGTAACGTAACAAAGCGGGGTTGGTAGTGTTTGC |
| (Afu5gl4900-Afu5gl4920) | AAATGCATTCACATGGACCGATCACTTTTCTTTCCAG |
| Bold, underlined, and | TCTGTCCATTCTGTCCAATCTGTCCGATCTGACCTGCC |
| italicized “G” corresponds to | CAGTCTGTCCAGTCTGTCCCTTGTGTCGTCCGATCCA |
| the sequence change as a | AGCTGGTTATCATGGCATCCACAAAGCCCGCTTCGAG |
| result of in vitro evolution in | TCTCATTTACCAGGCATGGAACAAACTCAGTATCAAC |
| hypoxia to produce the hrmK | CAAACCATCCCTAGTGACTCCCTTGAATTACTTGGGG |
| D304G amino acid variant. | AGCGTTTGGCTATTGCCTTCGCACCCAAACTCAAGGA |
| (SEQ ID NO: 2) | GCAACGAAGGAATGGCCGGCGTCGGAATCTGGAATA |
| | TGTGGCACAACATCGACGGAAGATTGCTCGAAAAAT |
| | CTACTTGGAGATTCTGGAGAAAGACCCAAATATCTTT |
| | CTTCCTTTTATCCTGGCTGTTTCCCCTAGAGCATGCTT |
| | ATCCTTTGATATCTCGAGCTTTCTTGAACAGCACCAA |
| | AGCCAAGGAAGACATTTCCTCCGCAACAATGCCGAA |
| | GCGATCCTCTGGGGTCTCGCAAAGAAACATGACATTG |
| | ATGGCTCCCTCCATTTCAGGAAGCTGATGCGTGAGAT |
| | TTTCCAACTGTCTCCTCCAGCGACAGAAGCCGAAGGC |
| | AAGGAGCATTATTCATTGCATTTAAGCACTCTCCCCG |
| | CAATCCGCAATGCCTTCGGTGATGTTATCTTTGACGC |
| | AATTGAACGTTCCCCTACACAGGTGACAGCGAGAGC |
| | TAAAGGTTATTTCTCTGAGAAAACCGAAAGTGTTTGG |
| | ACAAAAGTTCCCTACAGAAGTTCTCAAGACGCAATC |
| | ATATCTCTTGAAGTAGGGTCGGCAATCGAGCTTGCGA |
| | ATGTGTTGTTCCCAATCGCAACCCAAAAAATTGTCTC |
| | TATCCTTTCCGCATGTTCTCCCACTGTGCGCCAGAAG |
| | AACTTTTCTGAGGCTATTCTCGGCCCAGACCCTCAGG |
| | ATACACCGGCAACATCATCAGAAATCGGTATGAAGT |
| | TTAAGGTACACATGACTGCAGTTGCTAATTCCACCCT |
| | GTGCTAGATGTGGCGTATTTTACTCTGCGAGGAGCAA |
| | CGGTCTCGGCAATTGAATCAGTCTTTCGCGCTGATAT |
| | TTGCGAAGGTATTAAGGGCAGCGAACTGAGAAACTG |
| | GGAAAAGGAGCAGCTGCTCATCGACACGACAGATTG |
| | TGTCACGATGCAGATATGGCGGGCACAACCTCAACA |
| | TGGAACCATCAAGTTGCGTATTGGATTCTATGCAGCG |
| | GTGAATTTGGCAAATCGGCTGTATGCAGAAACACCCC |
| | AAGATCACATATAGTAACCTTTCATCTTTTCGGCCTTC |
| | TTAAATCATTGCCTTTCTGTGAGTCGCGACTTTCCACC |
| | CTTTATGAATACACCAATACCAGGGGGAAGAACGAT |
| | TTCACCGCTTCCCTTGGCAATCCATATAGTTCCCTTCT |
| | CATTCTGGAACCTGATTTCATCGCAGTTGAAGCAATA |
| | TAAATTCCTTTCGAGGTTTTCTGCATTGTAGGGATGG |
| | AATGGGTGCGTGAAATATTTGTCGAAAATAGAAGGG |
| | TCCGAAAGTTCTTTCCAAGCTGCGCGCTGAAAGTTGT |
| | TAGCCCATCTTTCGAGCATATGGTTTGGCCCACATAC |
| | GAGAGATTCTTTTGTGATCGGTTGAGATTCTTCCGTG |
| | ATCGGTTCTGTCATTTTCATTAAGTCAGAGAGCCCTC |
| | TTGTATGCCGGCTTTTGCTGTCGGATCCGGCGAGATA |
| | ATCGCTCCTAAGCCAGTCAGTCAGGGAAAAGCAAAG |
| | ATAAATAAAATATAGGCGAGGAGTACAACCAGGCAC |
| | GCGTCGTAGACTATTTTTCTGAAGAGTTTGTCACGTA |
| | ACCTACCTCATATGGATGGGTAGTTCGAATACTTGAT |
| | TGACTTGACCCGAGGTTCTGAAGGCGGCGGAGGAAA |
| | TTGCCCAACCCCACCATTGCATTTTCAGGTATCAATC |
| | TCTGCCACACTGTGGCTAAATTCGTCTTTATCGACAC |
| | GTGATCACGTTCCCTCTTCCAGCCCTGGTATCAGAGA |
| | ATCATCGAGTTATCGCTTGTTTCAATTTCGTCTTGCAA |
| | TTAGCTTAGGGAATAAGCATGTGGTCACATCAACCTA |
| | CAGAGCGCTACCGGTCTTTGCGCTGAGACTCTCAGTG |
| | ATCCGCCCAACAGACAACTAGACTTTGAGGTTGTCGA |
| | TATAACCACAACAAATGGCCTGTATATCAACGATGTC |
| | CACGCAATTGTCTCAAGCCTCTTCACCCGACTTCCAG |
| | CACTAGCATCCAAGCGGCCTCTCCTCTTCTCCCATGTT |
| | TCTCGTAGCGCGCCTGCATATACTTATATCTGGAGAT |
| | ATGTTAAAGGAGCTGGAAGCCTGGAGCATACGCTGG |
| | AAGCCTGGAGCATACGCTTCAAGTGCTGCCATATTCA |
| | GATAGCTGAGTAGGCACAATTAGGTCTAAGTTCAGG |
| | GAATTGCACCTCTCGCTTCATTGTCCGTCGATTCGTAT |
| | CGGTCTCTAGTTCTCCCCGTTTATCACTCTCACTCGGT |
| | GGACAGTCCGTCCAGTCCGTCCAGTCCGTCGAGCCTG |
| | TCCAATCTGTCCAATCTGTCCAATCTGTCCAATCTGTC |
| | CAATCTGTCCAATCTGTCCAATCTGTCCAATCTGTCC |
| | AATCTGTCCAATCTGTCCAATCTGTCCAATCTGTCCA |
| | ATCTGTCCAATCTGTCCAATCTGTCCAATCTGTCCACT |
| | CTGTCCACTCTGTCCACTCTGTCCAATCTGTCCACTCT |
| | GTCCCCTCTGTCCACTCTGTCCCCTCTGTCCACTCTGT |
| | CCCCTCTGTCCAATCGGTCCAATCTGTCCAATCTTGAT |
| | GATCTCGATGATCAATACCATTTAGCGAGCGCTAGTG |
| | ACGCCTTACAGCGTTGCGGCGTCTTATGGCTTATACA |
| | TCTTCCGAATATCAGTCGTTCGATCTCCAGATCACAC |
| | CTCGGGGTGAAACAAGCGCCATAGTTCTTGGTGCGCC |
| | TGAGCTTTTCCCTGCCCGGCCACTCAGTCGTGATGGC |
| | TTCCCAGAGTATTCCATAAGTCGAAACCAGAGATAG |
| | GCCAGCGGACGGCAAATCTCCTCTGCTCCGTTCTCAA |
| | CCAATACTGCCAAAGTGAGCAATGGAAAGTGTTCCA |
| | GGAGCACCGTGGCCTTTAAAAGCGCCAGCCTTGTCCC |
| | AGATCCTCACCGCAATTCGGCACAGACCAACGCAGA |
| | CTTTCATTGACCATTCTTCATTTCCAGATCGCTGCATA |
| | GTTTCCGGTATGGCTTGGTATAGAGCCTTACTCCCGT |
| | GCATACCGTTGTGGCGGAAGGTTCTGGGGCGTAACA |
| | GCACCGATGAGGACGGACGGAGTGAAGACGACCTTA |
| | CTTCATTGAGCGATAAAATGCCTACTTTTGAAGAAAC |
| | GACAACTGTGAGTACTGTTCGTGAAATTGCCTCCAGC |
| | TGTCTAATGTCTCCGTCGGTCAGATCACTTCTGCAAA |
| | GTACATCAACGGTGAAAAAATCATGGAGCATACCGT |
| | TGTGGAGACCAAGCACATTGACGAACGTGGAGACAC |
| | CAGCGTCAGTAACGGTGATTCGAACAGCACTGCGGT |
| | AACCAGACATTCTGGGCTTAGCTCTGTCAGCCTTTCA |
| | GATCAAAGTACAATTGTCGAGGACGCGAATGCTCTG |
| | GAAGAACCTGAACTCTTTGCTGTTCACTCTCCATATG |
| | TTGACGACTCAACCGGCGAGCAGATGGTTAGACTCTA |
| | CTACGAGCTCCCAGTAAGCCTCGATGATCTTGAGATC |
| | ATAGGCCTTGAATCTCGTATTCCAGAGTCTGACGATG |
| | ATTCAATTGAGGCCCGCTTTCGTTATCGAGGAGAGGA |
| | TTTTTGGCTACCTGTTCGTTATTCTTATGCCAAAGCTC |
| | GAATGGTTTTAACGGGTGTATGCTGAATGGTCTGACT |
| | TCTTCACTGTTGTTATTTTCTATTTCCCGGCTGCTGGC |
| | CACTCAATATTATCGCAAGCACTATACAAAATAAATA |
| | GTCCTATCTCTATAACAGAGTACGTCACAAACAGCGT |
| | TCGTCCTTGGCAAGAATATAAATAGTGTCAATCTGCT |
| | GAGAAAAGGAGGTATGAAATCCACTTCATTCAAGCA |
| | ATGGTTCCCTTCGTATACATTATTATTTGTATATGGAT |
| | GGGAACTTTTCTTGTCTTAGTTGCCCTGAACGCCCGC |
| | TTTAAGAAGAAGCGCATTGCTGATCCTGAGTCTTCTG |
| | CTGCCTTCACTGATCATCCCCATCAAGAGTAACATGG |
| | ATTCTGTATGTCCCTTTGGGCATGTTTATGTGGCAGTT |
| | ACTAATATATTAGATGGAAACTAGGAAGCGGCAAAA |
| | CACAGGGGCAGGAAGGGAAGGACTCATTCCAAGAAA |
| | GAAGGACAAGGAAGAACACAAGAGTGTAAGGGGTA |
| | GCAATCCTCATTGGCGTCTACTAGCTGATGGGTTAAA |
| | GCAAGATACAGGTGTTCATACGCTCCAAGACAACGA |
| | TCCTGTTGTCGAGAAGCCGCCATTAATTTACAGGAGA |
| | GCTGGAGAAGCGCCGAAACATGATTGGGACAAGAAG |
| | TCACCCGGTTTGCGCCTTAGACGGTCCTGCTGTAGCT |
| | GCGGGAAGGAAGTACCGGTAGGCCTGGTCTGTCGTA |
| | TTTGTCACCATGAGTCTTGTCCTGAGTGCTTGAACAT |
| | GCAAAAGCGAGATTACGGATGTTGATCAGATACACG |
| | GGCCCCTATACTAGGACTAGTTACCACTAGGATTGTA |
| | CTCAACCTTAACAGTGGCGTTTGGTACTTCGTTACAG |
| | CTAAGGGGAGAGGACAGTTCCTACTTTCATGTGCTTC |
| | AAGGGAGAGGCTCCTCCAACTACTGTTGCTAGGGAG |
| | GAAAGACACTCTATTCTTAGCTGAGGGCTGCAGGAAT |
| | TCGCATCCATGCAGTCATACCATCCATGGCATGCAAT |
| | CTATGTTGCATTAGATGCAAGAAGTAGGATAGAGAA |
| | CCCATGTACTTGATTCACGCTAGCAGGACAGAGAGA |
| | GATACCATACCCGACGGGAGCCCCTGCATGACTTCCT |
| | GTCTGCAGCTTGTCGTGCGTGTATCATTCCCATGCGC |
| | CACGAACTCATAGGCAGTGGTAGTTCAGAACACTCTT |
| | TTTTTTTAAAAAAAAAAAAGATAGGAAAATAATAAT |
| | TTAGGGGAAGAAAAGTAAAAATTAAAAAGAAAAAGT |
| | TCCAGATGGCGCTTCTACTTCTATATTCGATCGTTATT |
| | CAATACCCCAGAGGCACAGGCATTCCGATCTCTCATC |
| | GCCAACACTGAAAAGCAGCCATTTTCCCCCGTCTTAA |
| | AGCTCCAATCCTCCTTCTTCTCATCTACTTCCTCGCTC |
| | CTTCAGGACCTTGAGTGTTCCGTTGAGCTATTGGGTA |
| | ACTTCTCACCTGTCAATCATCGATTGTCCTTTCTCTTG |
| | ACTTGACTTCGTGTCGCCATTCTCATTTACGATACATA |
| | TCCCTGGAGCAGAAAACAAAGAAAAGGGCCAATTAC |
| | TCTTGATCTAGTTCCAACTCTGTTGCTGCTTGGAACAT |
| | CCGCCCATCTGTGTGGTGAAATCAGATGCCAGCATCC |
| | ATCTTGCAGCTTCTCCCACTTCCTGGGCCGATCTTGA |
| | ATGGGAGTCAATCTGCCTCGAAATGGCTCGTCTGCCT |
| | TTCTCATCTGGGTACATCCTGTGAGTAGCATGTCGTC |
| | ACTTGTCACACATACTACCCGCTCTCAAATCTGTTTG |
| | ATGGGAGTCAATCTGCCTCGAAATGGCTCGTCTGCCT |
| | TCACAAGCAAACTACAGCAGATGGCGGGGGCATGGA |
| | CTCGAGCCACAGTGCTGGCTCTCGCTTGCATCTGGAC |
| | CTTCTTATTCTTTCTCATTGCTGTATCTTTTTCCCCCTT |
| | GAGGCTTCTGGCGCGCTGCACCTTTCCAAGTATCAAA |
| | CCAAAGCTAATCAGGGGCGTTTGGCGTCCTGCCATGG |
| | CTTCACTAGACCTGGATCTCTGCAGCCTCATCACCAT |
| | CTCGGATCACCTGGTTCTGATCACCTTGGAAGAAAGC |
| | ACAAAGACCTTGGAGACAATACATATTGCCGCCATC |
| | GCAGCTCCCTCCAATCTCGACAGCATTTTCATGTGTC |
| | GGGCACTATCTACCTCTCGGCAATTCAGTAACCGTAC |
| | TGCCTGAGAAACATCAACCTCTCAAATTACACAATGG |
| | TGTTCAGCGCACCTGCTCCTGGTGTGGGCTCCAGTAA |
| | AAGGCCAGCGTCATGCATGCAGGACGATGTTGATGA |
| | GCGGGATAATGTCCCAGTAAGTGATACCAGCATTGG |
| | AAAGGCAGATGGAGCTGACTCATCTCCTATATAGCCC |
| | ATGGGTCTATGCGTGCGTTCATCGATCAACATCCCTT |
| | ACTTCCACTACGCCATGATCTATCTCGACAACATGGG |
| | CAGACTTAAGGTGATGGAATCTCCGTCTATCCAGGAG |
| | CAAAATGAGACTGTTTTCACAACCGAAGTACGTGAA |
| | AGATTTTTGGAAATCCTTGGTGCCAAGGTAGGATATC |
| | AACCGCCCATGGTTCGAAGTATGTAAACACTCCGCGC |
| | ACAAGTACAATATTCTTGCTGATCTCAATTGAACAGG |
| | GTTGTCAGCTGCCGGTGCTACACCATACAGCTATGAT |
| | CCTCAACAACCGCTTGGTTGCTTGTCTTACCGTCAAA |
| | CTAAGCGGGACAGAAATTCCCCAGCCCACTCTATGTA |
| | CGGTGTGCCGCCATCCGTCCAGTTCTCAGCCCCGGTT |
| | GAGGAATCGCCCTCTTGTGGATCAGTGGACATGGTCG |
| | GGCTCGAGATTGGTGATACTCCTAATGTCCTTGACTA |
| | CTATGAGAGATCCTTAAAGCACTTTCGGCAGGTCAAC |
| | TGTCGCCAGATCCTAAAGACATTCATTAAGTTCATTG |
| | AGCCACGAAAGCAAGCCAAGCACCCCTATAATGGAG |
| | GTAAACCCCCTGCAGGAGCCCCTCCTGGTAAGAAGG |
| | GCGACCCAGAGAAGACAAAGCCTGAATGGTGGCCCG |
| | CCAATGTGGTCCACAAGGAGCCTGACCATCTTCGAAA |
| | GGATCGTACGTGTAACCCTTCAGAAAATCTTCAGTGT |
| | CAAGTAACTTTGCTGACAGACTTAGAACGCCTGTCTC |
| | TGTTAATTCATATCATCCGCAGGCTTGGAAGATTTGG |
| | TATCACCACGGATCAATTGCAGGAAATTGCCCACGAC |
| | TGCAAGCGGCGGCTCAGCGACCCCCACAAACTCCAA |
| | ATCTTGGACGAGGTCTTCAGAGTGAGAAGGATTGAA |
| | GAACGCTACGAAAGAGGAGAAGTTGGTAAGCGGCAT |
| | CATCTTTCCATGAAATTCATTTTGACAGCTGTTGACG |
| | AGCCTCAGATGCCAACAAGATCGTATATGTTGTCAAC |
| | CGAGAGTCGAATCAGAAAGAGAAGGATGGCGACTCC |
| | AACGTGGATCCGGACCAGAAGCATGAGCAAGAAGAC |
| | GATAATGCGCGGGAGGCACTTCCCATTCTCCACTCCG |
| | AGAAGAACTCAACCAGCCCGATGTCGAACTCAGCCG |
| | AGCACACGGGCATGGCGGCACCAAGTCGTCCAATGA |
| | ATATGGGAGGTGACAGAAACCAGTTGTTTCCTTTACC |
| | GGAGTGGCCGAGCTTCGGTGAGACACCCCAGGATGA |
| | TCGAATTTTCTTTCCCACGACCTCTAAGTATACCGAA |
| | GATTATGCATCGCAGCAGATGCCTAGAACACCTGCA |
| | ACAACAGCACTTGTCAGCACTAATGAGACACATGCG |
| | GCCTTTGATTATATGACACAGGAGTCCATCACCTCCT |
| | CCTCCCCAGAGCAGACTTCCCACCACCGCCAAGCACC |
| | CCTGCCCATGCAGCACTCGGCCAGCCTCGACCCTTGG |
| | ACCCCTACGTTCCGACATAATTTCTTCAACCCAATGG |
| | TGTATAGTACTGCACCCCGTCACGCCATGTCCCAGGC |
| | TACTATGTTATCTCAGTTTCCCAGGTCCACGACGTCTC |
| | ATGGCCAGGAAATGCCTCACATGGCTCACGGCCTGCC |
| | GAACCTGCCTCAAGACAGACCTTCAAGCATGGATGG |
| | CATGAGCATGAGAGGCCCTTCTTTCCGCACAGGATTT |
| | TTGAGTCATCCCTGTGACCCATCACAGCAGGCTCCTC |
| | ATTCTAGCGGATGCGGCCATCCTGACAGTTGGACTCA |
| | AAATAGACCACATGTATAATCTTAACTGATTGATCCT |
| | TGACCACTGTTTTGACCCTCCTGCAGCCTTGAAGCTT |
| | CGTTTCACTGATGATTGTTCTTCGACTTTGTTTCTGTC |
| | CCTGACTTTGTTGTCAATGCGGACTTATCCATGCGGC |
| | TTGTTCCACGTCAAGTGACTACCAGGACACTCCGTGG |
| | TTTTATATGGCAGGTACTGGCGATGACTTTCCAATTC |
| | TTCTTCGTTTAGTATATATACTCGTTTCTTGTTCTATG |
| | TTCGATCATGTCTTTTTCCTTATACATACCTCCAAAAA |
| | TCCTGTTGGAGATGGCGCCAGATGGCATGAGATGCA |
| | AATATGGATGATGTTCTTGTGTTTGTTCATTTCAATTT |
| | CTTTCTCTTAATCATGATTTGAACAATTGGCAGCGAG |
| | GTATGGCGGAGCTCGTTCTCTTTGGATGCCGATCAGC |
| | TGAATAGGAGGTAACGAGGCATGAGGGTGTTTCATT |
| | ATGACTCTCTCCGGTGTTTGTCATTTAAGGGTGCGAG |
| | GGGGAAGTGTCCGTTTCGATGTCCTAGGATATCGAAA |
| | ATCTGAGTAGTAGCCACGTGACCCTATGCTGACGGCT |
| | GGGCTGGAAGACAAGCAGGTTGCTGCTTACGAGAAT |
| | ATGTTGAGGTATTCTCGTTATCTTCGTGAAGAATGCC |
| | GTCTCCTTGGCCCTCTAGCCAAAGTCTGGGTTGCTGA |
| | AAGGCTAGCTGGAATTGAGAATCGACTGTCTGCGTCC |
| | GAGTCGCCTAGAGGTGGGAAGGCCCCCTCTTTCTCAT |
| | ACATATGCTGACTCTGCAGACCATACCAATTCGCTGC |
| | CCGAA |
| A. fumigatus_AF293_bafA | ATGGCTTGGTATAGAGCCTTACTCCCGTGCATACCGT |
| (SEQ ID NO: 3) | TGTGGCGGAAGGTTCTGGGGCGTAACAGCACCGATG |
| | AGGACGGACGGAGTGAAGACGACCTTACTTCATTGA |
| | GCGATAAAATGCCTACTTTTGAAGAAACGACAACTgtg |
| | agtactgttcgtgaaattgcctccagctgtctaatgtctccgtcggtcagatcACTTC |
| | TGCAAAGTACATCAACGGTGAAAAAATCATGGAGCA |
| | TACCGTTGTGGAGACCAAGCACATTGACGAACGTGG |
| | AGACACCAGCGTCAGTAACGGTGATTCGAACAGCAC |
| | TGCGGTAACCAGACATTCTGGGCTTAGCTCTGTCAGC |
| | CTTTCAGATCAAAGTACAATTGTCGAGGACGCGAATG |
| | CTCTGGAAGAACCTGAACTCTTTGCTGTTCACTCTCC |
| | ATATGTTGACGACTCAACCGGCGAGCAGATGGTTAG |
| | ACTCTACTACGAGCTCCCAGTAAGCCTCGATGATCTT |
| | GAGATCATAGGCCTTGAATCTCGTATTCCAGAGTCTG |
| | ACGATGATTCAATTGAGGCCCGCTTTCGTTATCGAGG |
| | AGAGGATTTTTGGCTACCTGTTCGTTATTCTTATGCCA |
| | AAGCTCGAATGGTTTTAACGGGTGTATGCTGA |
| A. fumigatus_CEA10_bafB_ | ATGGTGTGGTATAGGGCCATACTCGTTTGCATGCCGT |
| AFUB_044360 | GGTGGCTCATGGGGCGTAACAGCACCAATGAGGGCA |
| (SEQ ID NO: 4) | AACGGAGTGAAGGCGAACGGGCTCCAATGATTGATA |
| | AGGTGCCCACTTTCGAAGAAATGACAATTgtgagtactgtttt |
| | gtggggttgcctccagctgtctaatgcttccttgcgcagACCACCTCTGCAA |
| | AGTATGTCAACGGTGAAAAAATCATGGAGCATACCG |
| | TTGTGGAGACCAAGCAAATTGACAACCGAGGAGACA |
| | CCAGCGTCAGTAACAATGATTCAAACAGCACTGCGG |
| | AAACCAGACATTCTGGGCTTAGCTCCGTCAGTCATTC |
| | AGATCAAAGTAAAGTTGTTGAGGACGCGAATGCCCT |
| | GGAAAAACCTGAACTCTTTGCTGTTCACTCTCCATAT |
| | GTTGACGCCTCAACCGGCAAGCAGATGTTAAGACTCT |
| | ACTACGAGCTCCCAGTAAGCCTCGATGATCTTGAGAT |
| | CACAGGCCTTGAATCTCGTATTCCAGAGTCTGATGAT |
| | GATTCAATTGAGGCCTGCTTTTGTTATCGGGGGGAGA |
| | AATTTTGGCTACATGTTCCTTATTCTTATGCCAAAGCT |
| | CGAATGGTTTTAATGGGTGTATACTGA |
| A. fumigatus_CEA10_bafC_ | ATGGCTTGGTATGAAGTCTTCGAGCAATGGGTGTACT |
| AFUB_096610 | GGTGCTGGCAGCGCATATGGCCCTTCGACGACAGCA |
| (SEQ ID NO: 5) | GGAGGGACGGACGAAACGAAGACGACCTAACTTCGT |
| | TAACCGATAAAATGCCTGTTTTTGAAGATAAGATCAT |
| | CgtgagtactgtccatgaggaggcttccatctccctaaccttggcagAACACCT |
| | CTGTCAGATATGTCAATGGAGAGATCGCGGCATATGT |
| | CGTCCAGACCCAGTATCTCGATACCCAAGAGGTCTCC |
| | TCTGCTAGGGACTCTTATTGGAAAAGCGTTGCGGATA |
| | TCAAACCGGGTGACTTCTGCTCCCATAGTATTTCGGA |
| | TCAGAGCACAATTGTCGAAGAAAACGAAGCGAAGGC |
| | GCTGGAAGGACCTGAACCCTTTGCTGTTCGCCCTTCG |
| | TATATTGGATCCACTGGCAAACGCACGGTCGACTTCT |
| | TCTACAAGGTCTCTCTACCACTGGATGATCTTGAGAT |
| | GAGAGACAAGGAATCGCGTGTTCCGGAGTCTAGCGA |
| | AGATCTGATTGAGGCTCTCTTCCACTATCAAGGGGCC |
| | GATATTTGGGTATATGTTCCTTATTCGTACGCCAATG |
| | CTCGAATGGTTTCAGGCGGTCCCACTGAATGA |
| A. niger_CBS 513.88_ | ATGGCTTGGTACAGTGCTTTACTCCCGTGCATGCTAT |
| An08gl2010 | GGTGGCGGAACCTCCTGTGGCGTAACAGCACCAATA |
| (SEQ ID NO: 6) | GGTATAGACAGAGTACAGACGACCTTACTTCACTGAC |
| | TGATAAGATACCTAATCTTGGAGAAAGGgtaagtgtaaatac |
| | tttttgaggtcgcttctaggagtctaatcactttggaaagacaactcccacagataacat |
| | cgatggaaagagcttcgtggcacatactgtcgtgcagaccaggcggattggtgaccg |
| | agaacgttgctgcttcaatgactcggatttgaacagcggcacagTTACCAAAA |
| | TTATTGAGCTTTGTTAATAGCATTCCAGATCAGAGTA |
| | CTACTGCCGGAAAAATTAATCACAAGGCCCTGCGAG |
| | ATCCTGAGCTCTTTGCTATCCGCTCGTCATGCATCGA |
| | CAAATCAGCCAGCAAGTGGATGGTTAGTCTCTACTAC |
| | GAACCCCCACCCAGCCTTGATGACCTCGAGATTAAGA |
| | ACTTCGGATCTCGTATTCCAGAGTCGGAGGATGATCC |
| | AATTGAGGCTATCTTTCACTATGAGGGAGAGAACATT |
| | TGGGTATCTGTTCCTTATTTGTATGCTAGAACTAGAA |
| | GCCTTTCAAGCGGTCTGTTCTGA |
| A. fumigatus_AF293_bafA-cDNA | ATGGCTTGGTATAGAGCCTTACTCCCGTGCATACCGT |
| (SEQ ID NO: 7) | TGTGGCGGAAGGTTCTGGGGCGTAACAGCACCGATG |
| | AGGACGGACGGAGTGAAGACGACCTTACTTCATTGA |
| | GCGATAAAATGCCTACTTTTGAAGAAACGACAACTTC |
| | TGCAAAGTACATCAACGGTGAAAAAATCATGGAGCA |
| | TACCGTTGTGGAGACCAAGCACATTGACGAACGTGG |
| | AGACACCAGCGTCAGTAACGGTGATTCGAACAGCAC |
| | TGCGGTAACCAGACATTCTGGGCTTAGCTCTGTCAGC |
| | CTTTCAGATCAAAGTACAATTGTCGAGGACGCGAATG |
| | CTCTGGAAGAACCTGAACTCTTTGCTGTTCACTCTCC |
| | ATATGTTGACGACTCAACCGGCGAGCAGATGGTTAG |
| | ACTCTACTACGAGCTCCCAGTAAGCCTCGATGATCTT |
| | GAGATCATAGGCCTTGAATCTCGTATTCCAGAGTCTG |
| | ACGATGATTCAATTGAGGCCCGCTTTCGTTATCGAGG |
| | AGAGGATTTTTGGCTACCTGTTCGTTATTCTTATGCCA |
| | AAGCTCGAATGGTTTTAACGGGTGTATGCTGA |
| A. fumigatus_CEA10_bafB_ | ATGGTGTGGTATAGGGCCATACTCGTTTGCATGCCGT |
| AFUB_044360-cDNA | GGTGGCTCATGGGGCGTAACAGCACCAATGAGGGCA |
| (SEQ ID NO: 8) | AACGGAGTGAAGGCGAACGGGCTCCAATGATTGATA |
| | AGGTGCCCACTTTCGAAGAAATGACAATTACCACCTC |
| | TGCAAAGTATGTCAACGGTGAAAAAATCATGGAGCA |
| | TACCGTTGTGGAGACCAAGCAAATTGACAACCGAGG |
| | AGACACCAGCGTCAGTAACAATGATTCAAACAGCAC |
| | TGCGGAAACCAGACATTCTGGGCTTAGCTCCGTCAGT |
| | CATTCAGATCAAAGTAAAGTTGTTGAGGACGCGAAT |
| | GCCCTGGAAAAACCTGAACTCTTTGCTGTTCACTCTC |
| | CATATGTTGACGCCTCAACCGGCAAGCAGATGTTAAG |
| | ACTCTACTACGAGCTCCCAGTAAGCCTCGATGATCTT |
| | GAGATCACAGGCCTTGAATCTCGTATTCCAGAGTCTG |
| | ATGATGATTCAATTGAGGCCTGCTTTTGTTATCGGGG |
| | GGAGAAATTTTGGCTACATGTTCCTTATTCTTATGCC |
| | AAAGCTCGAATGGTTTTAATGGGTGTATACTGA |
| A. fumigatus_CEA10_bafC_ | ATGGCTTGGTATGAAGTCTTCGAGCAATGGGTGTACT |
| AFUB_096610-cDNA | GGTGCTGGCAGCGCATATGGCCCTTCGACGACAGCA |
| (SEQ ID NO: 9) | GGAGGGACGGACGAAACGAAGACGACCTAACTTCGT |
| | TAACCGATAAAATGCCTGTTTTTGAAGATAAGATCAT |
| | CAACACCTCTGTCAGATATGTCAATGGAGAGATCGCG |
| | GCATATGTCGTCCAGACCCAGTATCTCGATACCCAAG |
| | AGGTCTCCTCTGCTAGGGACTCTTATTGGAAAAGCGT |
| | TGCGGATATCAAACCGGGTGACTTCTGCTCCCATAGT |
| | ATTTCGGATCAGAGCACAATTGTCGAAGAAAACGAA |
| | GCGAAGGCGCTGGAAGGACCTGAACCCTTTGCTGTTC |
| | GCCCTTCGTATATTGGATCCACTGGCAAACGCACGGT |
| | CGACTTCTTCTACAAGGTCTCTCTACCACTGGATGAT |
| | CTTGAGATGAGAGACAAGGAATCGCGTGTTCCGGAG |
| | TCTAGCGAAGATCTGATTGAGGCTCTCTTCCACTATC |
| | AAGGGGCCGATATTTGGGTATATGTTCCTTATTCGTA |
| | CGCCAATGCTCGAATGGTTTCAGGCGGTCCCACTGAA |
| | TGA |
| A. niger_CBS_513.88_ | ATGGCTTGGTACAGTGCTTTACTCCCGTGCATGCTAT |
| An08gl2010-cDNA | GGTGGCGGAACCTCCTGTGGCGTAACAGCACCAATA |
| (SEQ ID NO: 10) | GGTATAGACAGAGTACAGACGACCTTACTTCACTGAC |
| | TGATAAGATACCTAATCTTGGAGAAAGGTTACCAAAT |
| | TTATTGAGCTTTGTTAATAGCATTCCAGATCAGAGTA |
| | CTACTGCCGGAAAAATTAATCACAAGGCCCTGCGAG |
| | ATCCTGAGCTCTTTGCTATCCGCTCGTCATGCATCGA |
| | CAAATCAGCCAGCAAGTGGATGGTTAGTCTCTACTAC |
| | GAACCCCCACCCAGCCTTGATGACCTCGAGATTAAGA |
| | ACTTCGGATCTCGTATTCCAGAGTCGGAGGATGATCC |
| | AATTGAGGCTATCTTTCACTATGAGGGAGAGAACATT |
| | TGGGTATCTGTTCCTTATTTGTATGCTAGAACTAGAA |
| | GCCTTTCAAGCGGTCTGTTCTGA |

In certain embodiments, the disclosure provides a filamentous fungal host cell that is modified through the introduction of any one or more of SEQ ID NOs 1-10 recited above. In certain embodiments, any one or more of SEQ ID NOs 1-10 can be integrated into the filamentous fungal host cell genome. In certain embodiments, any one or more of SEQ ID NOs 1-10 are incorporated into an expression vector comprising a promoter for expressing the one or more of SEQ ID NOs 1-10 in the filamentous fungal host cell.

TABLE 2
Amino Acid Sequences of the Disclosure.

| Sequence | Sequence |
|---|---|
| A. fumigatus_hrmA/ | MASTKPASSLIYQAWNKLSINQTIPSDSLELLGERLAIAF |
| Afu5g14900 | APKLKEQRRNGRRRNLEYVAQHRRKIARKIYLEILEKDP |
| (SEQ ID NO: 11) | NIFLPFILAVSPRACLSFDISSFLEQHQSQGRHFLRNNAE |
| | AILWGLAKKHDIDGSLHFRKLMREIFQLSPPATEAEGKE |
| | HYSLHLSTLPAIRNAFGDVIFDAIERSPTQVTARAKGYFS |
| | EKTESVWTKVPYRSSQDAIISLEVGSAIELANVLFPIATQ |
| | KIVSILSACSPTVRQKNFSEAILGPDPQDTPATSSEIDVAY |
| | FTLRGATVSAIESVFRADICEGIKDSELRNWEKEQLLIDT |
| | TDCVTMQIWRAQPQHGTIKLRIGFYAAVNLANRLYAET |
| | PQDHI |
| A. fumigatus_hrmA/ | MASTKPASSLIYQAWNKLSINQTIPSDSLELLGERLAIAF |
| Afu5g14900 D304G amino | APKLKEQRRNGRRRNLEYVAQHRRKIARKIYLEILEKDP |
| acid variant | NIFLPFILAVSPRACLSFDISSFLEQHQSQGRHFLRNNAE |
| (SEQ ID NO: 12) | AILWGLAKKHDIDGSLHFRKLMREIFQLSPPATEAEGKE |
| | HYSLHLSTLPAIRNAFGDVIFDAIERSPTQVTARAKGYFS |
| | EKTESVWTKVPYRSSQDAIISLEVGSAIELANVLFPIATQ |
| | KIVSILSACSPTVRQKNFSEAILGPDPQDTPATSSEIDVAY |
| | FTLRGATVSAIESVFRADICEGIKGSELRNWEKEQLLIDT |
| | TDCVTMQIWRAQPQHGTIKLRIGFYAAVNLANRLYAET |
| | PQDHI |
| A. fumigatus_AF293_BafA | MAWYRALLPCIPLWRKVLGRNSTDEDGRSEDDLTSLSD |
| (SEQ ID NO: 13) | KMPTFEETTTSAKYINGEKIMEHTVVETKHIDERGDTSV |
| | SNGDSNSTAVTRHSGLSSVSLSDQSTIVEDANALEEPEL |
| | FAVHSPYVDDSTGEMVRLYYELPVSLDDLEIIGLESRIPE |
| | SDDDSIEARFRYRGEDFWLPVRYSYAKARMVLTGVC |
| A. fumigatus_CEA10_BafB_ | MVWYRAILVCMPWWLMGRNSTNEGKRSEGERAPMID |
| AFUB_044360 | KVPTFEEMTITTSAKYVNGEKIMEHTVETKQIDNRGDTS |
| (SEQ ID NO: 14) | VSNNDSNSTAETRHSGLSSVSHSDQSKVVEDANALEKP |
| | ELFAVHSPYVDASTGKQMLRLYYELPVSLDDLEITGLES |
| | RIPESDDDSIEACFCYRGEKFWLHVPYSYAKARMVLMG |
| | VY |
| A. fumigatus_CEA10_BafC_ | MAWYEVFEQWVYWCWQRIWPFDDSRRDGRNEDDLTS |
| AFUB_096610 | LTDKMPVFEDKIINTSVRYVNGEIAAYVVQTQYLDTQE |
| (SEQ ID NO: 15) | VSSARDSYWKSVADIKPGDFCSHSISDQSTIVEENEAKA |
| | LEGPEPFAVRPSYIGSTGKRTVDFFYKVSLPLDDLEMRD |
| | KESRVPESSEDLIEALFHYQGADIWVYVPYSYANARMV |
| | SGGPTE |
| A. niger_CBS_513.88_ | MAWYSALLPCMLWWRNLLWRNSTNRYRQSTDDLTSL |
| An08g12010 | TDKIPNLGERLPNLLSFVNSIPDQSTTAGKINHKALRDPE |
| (SEQ ID NO: 16) | LFAIRSSCIDKSASKWMVSLYYEPPPSLDDLEIKNFGSRI |
| | PESEDDPIEAIFHYEGENIWVSVPYLYARTRSLSSGLF |

### Methods of Use

In one aspect, the disclosure provides a method of increasing fungal secretion of one or more products of interest, comprising introducing into a filamentous fungal host cell one or more polynucleotide sequences encoding one or both of an hrmA protein and a baf protein.

In another aspect, the disclosure provides a method of increasing the production of one or more products of interest, comprising introducing into a filamentous fungal host cell one or more polynucleotide sequences encoding one or both of an hrmA protein and a baf protein.

In yet another aspect, the disclosure provides a method of reducing oxygen consumption of a filamentous fungal host cell, comprising introducing into a filamentous fungal host cell one or more polynucleotide sequences encoding one or both of an hrmA protein and a baf protein. In certain embodiments, a first polynucleotide sequence (e.g., a vector) encodes an hrmA protein and a second polynucleotide sequence (e.g., a vector) encodes a baf protein. In certain embodiments, more than one polynucleotide sequence is introduced into the filamentous fungal host cell, each polynucleotide sequence encoding for a different baf protein (e.g., a first polynucleotide sequence encoding the baf protein amino acid sequence of SEQ ID NO: 13 and a second polynucleotide sequence encoding the baf protein amino acid sequence of SEQ ID NO: 14).

In certain embodiments, the hrmA protein comprises or consists of the amino acid sequence of SEQ ID NO: 12. In certain embodiments, the baf protein comprises or consists of the amino acid sequence of SEQ ID NO: 13, SEQ ID NO: 14, SEQ ID NO: 15, or SEQ ID NO: 16.

In certain embodiments, oxygen consumption is reduced by about 10% to about 90%. In certain embodiments, oxygen consumption is reduced by about 10%, about 15%, about 20%, about 25%, about 30%, about 35%, about 40%, about 45%, about 50%, about 55%, about 60%, about 65%, about 70%, about 75%, about 80%, about 85%, or about 90%.

In certain embodiments, the polynucleotide sequence is introduced into the filamentous fungal host cell via transformation. In certain embodiments, the transformation comprises one or more of protoplast-mediated transformation, Agrobacterium-mediated transformation, electroporation, biolistic transformation, and shock-wave-mediated transformation.

In certain embodiments, the polynucleotide sequence is introduced into the filamentous fungal host cell transiently.

In certain embodiments, the polynucleotide sequence is stably integrated into the filamentous fungal host cell genome.

In certain embodiments, the polynucleotide sequence is introduced into the filamentous fungal host cell genome with a genetic-editing system.

In certain embodiments, the genetic-editing system comprises one or more of a meganuclease system, a ZFN system, a TALEN system, and a CRISPR system.

In certain embodiments, the polynucleotide sequence further comprises a promoter to express one or both of the hrmA protein and the baf protein. In certain embodiments, the promoter is inducible or constitutive. In certain embodiments, the inducible promoter is selected from the group consisting of: alcA, amyB, bli-3, bphA, catR, cbhI, cre1, exylA, gas, glaA, mir1, niiA, qa-2, Smxyl, tcu-1, thiA, vvd, xyl1, xylP, xyn1, or zeaR. In certain embodiments, the constitutive promoter comprises cDNA1, eno1, gpdA, gpd1, pdc1, pki1, poliC, tef1, or rp2.

The following non-limiting examples are provided to further illustrate the present disclosure.

### EXAMPLES

### Example 1—Fungal Biofilm Morphology Impacts Hypoxia Fitness and Disease Progression

Surface-dwelling microorganisms organize into macroscopic colonies of intricately structured populations. For bacteria and yeast, the inter- and intra-species heterogeneity of these macroscopic morphologies in vitro have been studied (Kuthan et al. Mol Microbiol 47, 745-754, 2003; Workentine et al. PLoS One 8, e60225, 2013); and microbial colony morphology (CM) variants are observed in clinical samples (Haussler et al. J Med Microbiol 52, 295-301, 2003; Hagiwara et al. J Clin Microbiol 52, 4202-4209, 2014). The challenge remains to determine how CM diversity reflects physiological variation and contributes to environmental fitness. CM is associated with changes in extracellular matrix (ECM) (Fong et al. J Bacteriol 189, 2319-2330, 2007), stress resistance (Drenkard et al. Nature 416, 740-743, 2002), reproduction (Miller et al. Cell 110, 293-302, 2002), and metabolism (Workentine et al. Environ Microbiol 12, 1565-1577, 2010).

Intraspecies CM variation can arise through accumulated genetic changes or through transcriptional rewiring resulting in phenotypic switching (Jain et al. FEMS Yeast Res 6, 480-488, 2006; Jain et al. Curr Fungal Infect Rep 2, 180-188, 2008). The human pathogenic mold Aspergillus fumigatus exhibits phenotypic plasticity at 0.2% O₂, where CM differs compared to 21% O₂ growth and is variable across strains (Kowalski et al. MBio 7, pii: e01515-16, 2016). Physiological changes and genetic mechanisms facilitating stable morphotype variants in A. fumigatus and other human pathogenic filamentous fungi are not well characterized, nor is their impact on pathogenesis and disease progression. Progress on understanding fungal CM and phenotypic variability has been limited in part by the underlying genetic complexity. Given the intraspecies CM variation found in A. fumigatus isolates and the impact of oxygen on CM, we sought to assess how a low oxygen CM variant impacts A. fumigatus pathogenesis and invasive aspergillosis (IA) disease progression and identify genetic factors involved in CM variation.

### Example 1 Materials and Methods

Strains and growth conditions: A. fumigatus AF293 was used in the published experimental evolution approach that generated EVOL20 (Kowalski 2016, supra). Mutant strains were generated in AF293, the uracil/uridine auxotroph AF293.1, or EVOL20. IFM 59356-1 and IFM 59356-3 were kindly provided by Dr. D. Hagiwara (Hagiwara 2014, supra). Strains were cultured on 1% glucose minimal media (GMM) and collected for experimentation as previously described (Beattie et al. PLoS Pathog 13, e1006340, 2017).

Strain construction: Strain genotypes are provided in Table 3 below. Gene replacement mutants were generated as previously described using overlap extension PCR (Szewczyk et al. Nat Protoc 1, 3111-3120, 2006). The hrmA-GFP alleles were constructed through overlap extension PCR to tag HrmA at the C-terminus. Site-directed mutation of hrmA was carried out using QuikChange Site-Directed Mutagenesis (Agilent). Overexpression strains utilized the A. nidulans gpdA promoter for constitutive expression and was introduced ectopically. Fluorescent strains expressing tdtomato were transformed with linear constructs of gpdA-driven tdtomato. Protoplasting was done with Trichoderma harzianum (Sigma) lysing enzyme and strains were confirmed by Southern blotting as described previously (Grahl et al. PLoS Pathog 7, e1002145, 2011; Willger et al. PLoS Pathog 4, e1000200, 2008).

TABLE 3
Aspergillus fumigatus strains utilized in this disclosure,
their genotypes, and their origination.

| | Back- | | |
|---|---|---|---|
| | ground | | |
| Strain | strain | Genotype | Origin |
| AF293 | reference/ | n/a | Nierman |
| | Parent | | et al. |
| | strain | | 2005 |
| EVOL20 | serially | n/a | Kowalski |
| | passed | | et |
| | AF293 | | al. 2016 |
| | (27) | | |
| AF293.1 | AF293 | pyrG− | Xue et al. |
| | | | 2004 |
| ΔhrmA^{AF} | AF293.1 | hrmA−; pyrG+ | This |
| | | | study |
| ΔhrmA^{EV} | EVOL20 | hrmA−; ptrA+ | This |
| | | | study |
| hrmA^{R-EV} | ΔhrmA^{AF} | hrmAEV+; | This |
| | | pyrG+; ptrA+ | study |
| hrmA^{R-EV}; | hrmA^{R-EV} | hrmAEV+; | This |
| ΔcgnA | | cgnA−; pyrG+; | study |
| | | ptrA+; hyg+ | |
| hrmA^{OE} | AF293.1 | gpdA-hrmA+; | This |
| | | pyrG+ | study |
| hrmA^{OE}-GFP | AF293.1 | gpdA-hrmA- | This |
| | | GFP+; pyrG+ | study |
| hrmA^{OE/NLS}- | AF293.1 | gpdA-hrmA-NLS/ | This |
| GFP | | GFP+; pyrG+ | study |
| hrmA^{OE}; | hrmA^{OE} | gpdA-hrmA+; | This |
| ΔcgnA | | pyrG+; ΔcgnA; | study |
| | | ptrA+ | |
| ΔcgnA^{EV} | EVOL20 | cgnA−; ptrA+ | This |
| | | | study |
| Δuge3^{AF} | AF293 | uge3−; ptrA+ | This |
| | | | study |
| Δuge3^{EV} | EVOL20 | uge3−; ptrA+ | This |
| | | | study |
| hrmA^{OE}; Δuge3 | AF293.1 | gpdA-hrmA+; | This |
| | | pyrG+ | study |
| cgnA^{OE} | AF293.1 | gpda-cgnA; | This |
| | | pyrG+ | study |
| EVOL20^{tdtomato} | EVOL20 | gpdA-tdtomato; | This |
| | | ptrA+ | study |
| AF293^{tdtomato} | AF293 | gpdA-tdtomato; | This |
| | | ptrA+ | study |
| IFM 59356-1 | clinical | n/a | Hagiwara |
| | isolate | | et al. |
| | | | 2017 |
| IFM 59356-3 | clinical | n/a | Hagiwara |
| | isolate | | et al. |
| | | | 2017 |
| ΔhrmA^{AF-tdtomato} | ΔhrmA^{AF} | hrmA−; pyrG+; | This |
| | | gpda-tdtomato; | study |
| | | hyg+ | |
| ΔhrmA^{EV-tdtomato} | ΔhrmA^{EV} | hrmA−; ptrA+; | This |
| | | gpda-tdtomato; | study |
| | | hyg+ | |
| hrmA^{R-EV-tdtomato} | hrmA^{R-EV} | hrmAEV+; pyrG+; | This |
| | | ptrA+; gpda- | study |
| | | tdtomato; hyg+ | |
| hrmA^{R-EV-} | hrmA^{R-EV}; | hrmAEV+; | This |
| | | cgnA−; pyrG+; | study |
| ΔcgnA^{tdtomato} | ΔcgnA | ptrA+; hyg+; | |
| | | phleo+; gpda- | |
| | | tdtomato | |
| ΔcgnA^{EV-tdtomato} | ΔcgnA^{EV} | cgnA−; ptrA+; | This |
| | | gpda-tdtomato; | study |
| | | hyg+ | |
| Δuge3^{AF-GFP} | AF293 | gpdA-gfp; ptrA+; | This |
| | | uge3−; hyg+ | study |
| AF293^{GFP} | AF293 | gpdA-gfp; ptrA+ | This |
| | | | study |
| F16311 | clinical | n/a | Howard |
| | isolate | | et al. |
| | | | 2009 |
| CDC20.2 | clinical | n/a | This |
| | isolate | | study |
| 204NB-8 | clinical | n/a | This |
| | isolate | | study |
| F11698/ | clinical | n/a | Howard |
| NCPF-7816 | isolate | | et al. |
| | | | 2009 |
| F1631^{tdtomato} | F16311 | gpdA-tdtomato; | This |
| | | hyg+ | study |
| CDC20.2^{tdtomato} | CDC20.2 | gpdA-tdtomato; | This |
| | | hyg+ | study |
| F11698/NCPF- | F11698/ | gpdA-tdtomato; | This |
| 7816^{tdtomato} | NCPF- | hyg+ | study |
| | 7816 | | |

Growth and colony morphology assays: Growth assays were performed as previously described (Kowalski 2016, supra). Macroscopic morphology was quantified on GMM. 1000 spores were spotted at the center of the plates and grown for 72-96 hours at 21% O₂ or 0.2% 02. Representative images are of 3 biological replicates. Statistics were performed with One-Way ANOVA with Tukey Post Test for multiple comparisons or two-tailed Students t-test. Error bars indicate standard error of the mean (StEM) centered at the mean. For shift experiments, cultures were started as described at 21% O₂ for 48 hours then shifted to 0.2% O₂ for 48 hours.

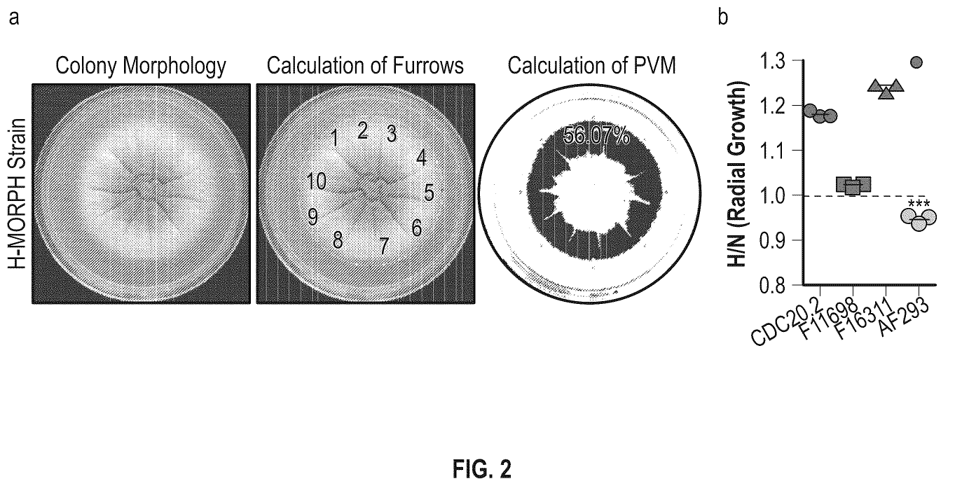

Macroscopic Morphology Quantification: Colonies were imaged with a Canon PowerShot SX40 HS. In Fiji (ImageJ) images were converted to 8-bit. Colony perimeter was selected and a Color Threshold was set to quantify percent of the colony that was ‘white’. Furrows were counted by selecting only those that radiated away from the point of inoculation. A ‘branched’ furrow counted as a single furrow (FIG. 2). The influence of oxygen on morphology was measured with a Two-Way ANOVA (GraphPad Prism).

RNA extraction and qRT-PCR: Mycelia from liquid shaking cultures was flash frozen (˜50 mg) and bead beat for 1 minute with 2.3 mm beads in 200 μl of Trisure (Bioline Reagents). Homogenate was brought to a total volume of 1 mL Trisure and RNA was extracted as previously described (Beattie 2017, supra). For RNA-sequencing and qRT-PCR, 50 mL cultures of 10⁶ spores/mL were grown in normoxia (21% O₂) at 37° C. at 200 rpm for 18 hours before being shifted to low oxygen (0.2% O₂). When necessary, 25 mL of the culture was collected at 18 hours for the normoxia samples. For qRT-PCR and RNA-sequencing, 5 g of RNA was DNAse treated with Ambion Turbo DNAse (Life Technologies) according to the manufacturer's instruction. For qRT-PCR DNase treated-RNA was processed as previously described (Beattie 2017, supra). mRNA levels were normalized to actA and tub2 for all qRT-PCR analyses. Statistical analysis for n>2 was performed with One-Way ANOVA with Dunnet Post Test for multiple comparisons. Error bards indicate StEM. qRT-PCR data was collected on a CFX Connect Real-Time PCR Detection System (Bio-Rad) with CFX Maestro Software (Bio-Rad).

RNA-sequencing and analysis: RNA-sequencing and RNA library preparation was carried out by SeqMatic LLC (Fremont, CA). Briefly, DNAse treated RNA (400-600 ng/L) were sent for QC using RNA Screen Tape Analysis (Agilent) and RNA library preparation using an Illumina TruSeq Standard mRNA library preparation kit with Poly A mRNA enrichment. RNA-sequencing was performed as Illumina NextSeq High Output Run with single end reads at 1×75 bp. Analysis of RNA-Seq was performed by aligning sequence reads to the annotated genome of A. fumigatus strain Af293 obtained from FungiDB (release 35) with GSNAP (2018 Feb. 12) with splice-aware, single-ended mode. The alignments were processed with Picard (v2.14.1) to clean, sort, and assign read groups (tools CleanSam, AddOrReplaceReadGroups) (http://broadinstitute.github.io/picard/). Sequence read counts overlapping genes were computed with featureCount tool in the Subread package (v1.6.2). The read count table was processed in R using the DESeq2 (3.8) to identify differentially regulated genes and generate heat maps. Pipeline BASH scripts for the alignment, read count pipeline, and R analysis is available in github repository (https://github.com/stajichlab/Afum_RNASeq_hrmA; BioProject PRJNA551460). Heatmaps were drawn using collapsed replicates showing top DESeq2 with a P-value<0.05 and log of differential expression>1 and a minimum FPKM of 5.

Surface attachment assays: Briefly, 10⁴ spores seeded per well in a round-bottom 96-well polystyrene plate were incubated for 24 hours at 37° C. at ambient oxygen in 1% GMM. Wells were washed 2× with water and stained for 10 min. with 0.1% (wt/vol) crystal violet. Following 2× washes with water, remaining crystal violet was dissolved in 100% ethanol and absorbance was quantified at 600 nm. For matrix complementation experiments, matrix donating strains were cultured in RPMI 1640 (Gibco) at 5×10⁷ spores/mL in 100 mL for 24 hours at 37° C. at ambient oxygen. Cultures were filtered through Miracloth to remove fungus, and supernatants were further filtered through a 0.22 μm PVDF sterile filter syringe. Filtered supernatants containing secreted GAG were diluted to 40% in fresh RPMI 1640 and used to perform the adherence assay with the attachment-deficient strain Δuge3.

### Murine Virulence Assays

Survival: Female CD-1 outbred mice (Charles River Laboratory, Raleigh, NC), 20-24 grams were immune-suppressed with a single dose of triamcinolone acetonide (Kenalong-10, Bristol-Myer Squibb) at 40 mg/kg 24 hours prior to inoculation. Mice were inoculated with 10⁵ spores/40 μL sterile PBS, as previously described (Kowalski 2016, supra) and monitored for end-point criteria. Kaplan-Meier curves were generated and Log-rank Mantel-Cox tests and Gehan-Breslow-Wilcoxon tests performed.

Histopathology, fungal burden, and nearest neighbor calculation: Lungs from mice immune-suppressed as described were harvested on 4 days post-inoculation (dpi). Lungs were prepared for Gömöri methenamine silver (GMS) and hematoxylin and eosin (H&E) staining or fungal burden quantification as described (Beattie 2017, supra). A nearest neighbor calculation was applied to GMS images. In Matlab (MathWorks Inc.), binary images were generated and filaments defined as objects. Lesions within airways were analyzed blindly. Mean distances between each object in a lesion and its 30 nearest neighbors was calculated. For nearest neighbor calculations four murine lungs were processed per experimental group with two histopathology slides prepared per animal. For fungal burden 4-5 animals were used per group.

FunPACT sample preparation: Lungs from mice immune-suppressed as described above were harvested on day 4 and day 5 post-inoculation. Lungs were harvested and perfused with 1% paraformaldehyde and fixed for 24 hours at room temperature. Following fixation, lobes of fixed lungs were separated with 1 lobe per 1.75 mL microcentrifuge tube. Lobes were washed with PBS and embedded in 4% (vol/vol) 29:1 acrylamide:bis-acrylamide (Bio-Rad) and 0.25% (wt/vol) VA-044 (Wako) in PBS. To facilitate polymerization, tubes were left open at 0.2% O₂ at 37° C. for 1 hour, and then closed and incubated at 37° C. in a water bath for 4 hours. Embedded lobes were maintained at 4° C. or were processed for PACT tissue clearing. To clear the lobes, embedded lobes were trimmed of excess polymer and cut into 1 mm cubes using a stereomicroscope. Cubes were incubated in 20 mL of 8% (wt/vol) sodium dodecyl sulfate (SDS) in PBS shaking at 150 rpm at 37° C. for 6-8 weeks in the dark. When cubes became transparent, they were processed for staining and imaging.

After clearing, the cubes were washed 3× with PBS for 1 hour each. A subset of cubes was then transferred to a 1.75 mL microcentrifuge tube and stained for 48 hours with FITC-Soy Bean Agglutinin at 20 g/mL (SBA) (Vector Labs). Lectin labeled cubes were washed in PBS for 24 hours to remove excess lectin, and cubes were placed in a refractive index matching solution (RIMS) (40 g HistoDenz: Sigma, in 30 mL PBS) with DAPI (10 g/mL). Stained cubes in RIMS+DAPI were mounted on standard 24×40×1.5 glass slides with a Press-to-Seal™ Silicone Isolator (Invitrogen: P24744).

Cellularity and Immunological Studies: Mice were immune suppressed and inoculated as described above with 8 mice per group. After 60 hpi, animals were sacrificed using a lethal dose of pentobarbital and bronchoalveolar lavage (BAL) was performed and BAL fluid (BALF) and cells, lungs and spleens were collected. Cells from BAL and lungs were prepared for staining. Lung tissue was minced and digested with 2.2 mg/mL Collagenase IV (Worthington), 1 U/mL DNase1 (Zymo Research) and 5% FBS at 37° C. for 45 minutes. BALF was centrifuged to isolate cells and suspended in red blood cell (RBC) lysis buffer. Re-suspended cells from lung homogenate were also treated for RBC lysis. Cell numbers were enumerated with Trypan Blue staining. For cellularity analysis, the cells were stained with Fixable Viability Dye (eFluor™ 780, eBioscience), anti-CD45 (Pacific orange, Invitrogen), anti-CD11b (PECy5, BioLegend), anti-Ly6G (FITC, BioLegend) anti-SiglecF (BV421, BD bioscience) and analyzed on a MacsQuant VYB cytometer. The neutrophils were identified as CD45⁺SiglecF⁻Ly6G⁺CD11b⁺ cells and alveolar macrophages as CD45⁺SigletF⁺CD11b^{dim} cells. Samples were run on a MacsQuant VYB cytometer and analyzed with FlowJo version 9.9.6. BALF was used to quantify host cell damage and KC through the use of LDH-Cytotoxicity Colorimetric assay (BioVision #K311) and Mouse CXCL1/KC DuoSet ELISA (R&D Systems #DY453), respectively.

Fungal Biofilm Sample Preparation: Biofilms for imaging were cultured in MatTek dishes (MatTek #P35G-1.0-14-C) by seeding 10⁵ spores/mL of GMM with 2 mL per dish for 24 hours at 37° C. with 5% CO₂ at 21% O₂ or 0.2% O₂. Calcofluor white stain (CFW) (Sigma) was used to visualize the hyphae at a final concentration of 25 g/mL for 15 minutes.

Fluorescent Microscopy: Fluorescent confocal microscopy was performed on an Andor W1 Spinning Disk Confocal with a Nikon Eclipse Ti inverted microscope stand with Perfect Focus, a Zeiss LSM880 with two multi-alkali photomultiplier tubes, GaAsP detector, and a transmitted light detector, or a Zeiss LSM800 AxioObserver.

HrmA Localization Studies: Fungi were cultured on coverslips in GMM at 30° C. for 18 hours until short hyphae, were washed, UV fixed, stained with 5 g/mL DAPI (Life Technologies), and mounted on slides. Images were acquired with a 100× oil immersion objective at 488 nm (GFP) and 405 nm (DAPI) on the Andor W1 Spinning Disk Confocal. Z-stacks were assembled in Fiji (ImageJ) with sum intensity projections. Images are representative of at least 10 images. Quantification was performed as previously described (Danhof et al. Infect Immun 83, 4416-4426, 2015).

Fungal Biofilm Imaging and Quantification: Biofilms were imaged in MatTek dishes with a 20× multi-immersion objective (Nikon) or 10× multi-immersion objective (Zeiss, C-Apochromat 10×/0.45 W M27) using water. CFW biofilms were imaged at 405 nm and tdtomato biofilms were imaged at 561 nm at depths from 300-500 nm. 3D projections were generated in Nikon NIS-Elements Viewer (Nikon) or Zeiss Blue (Zeiss). For quantification of biofilm architecture strains expressed tdtomato and were imaged on the Zeiss LSM880 AxioObserver with the exception of IFM 59356-1 and IFM 59356-3 which were stained with CFW (25 g/mL). For quantification see supplemental methods. To quantify the branch length and branch density distribution of the hyphae network image stacks were processed in BiofilmQ (https://drescherlab.org/data/biofilmQ/) as follows: First, noise and background fluorescence where removed by local averaging, i.e. Tophat-filtering, respectively. Second, the hyphae structure was binarized by thresholding using Otsu's method (Liao et al. J Inf Sci Eng 17, 713-727, 2001). Third, the obtained data was skeletonized with a custom BiofilmQ analysis module and all branches above a threshold length were considered for further investigation. Visualization of branch features was performed in BiofilmQ.

FunPACT Imaging: Mounted samples for funPACT were imaged on the Andor W1 Spinning Disk Confocal with a 20× multi-immersion objective lens used with oil or a 40× oil-immersion objective. Areas of fungal growth were identified by manual scanning at 561 nm. Lesions were imaged at 405 nm, 488, and 561 nm at various depths. Images were processed in Nikon NIS-Elements Viewer for deconvolution and 3D rendering.

Cell wall staining: Hyphae were generated as described for localization studies. Filaments were stained with 25 g/mL calcofluor white (Fluorescent Brightener 28—Sigma) for 15 min. or soluble Dectin-1 as described previously (Shepardson et al. Microbes Infect 15, 259-269, 2013). 10 hyphae images were processed per strain.

Scanning and transmission electron microscopy: Fungal biofilms for scanning and transmission electron microscopy were grown on 12 mm sterile glass coverslips in 6-well plates for 24 hours at 37° C. at 21% O₂ with 10⁶ spores/mL in RPMI 1640 (Gibco). Two coverslips were generated per sample. Samples were processed for SEM though a critical point drying method. Briefly, media was removed and replaced with fixative (2% GTA/2% PF in 0.05M NaCacodylate pH 7.4) for 15 minutes at room temperature. Fresh fixative was then added for 24 hours. Coverslips were then washed 3× (0.05M NaCacodylate pH 7.4 for 5 min) and then incubated for 1 hour at room temperature in 1% OsO₄ in 0.05M NaCacodylate before 3× washings as before. Samples were then ethanol dehydrated for 10 min. in each 30%, 50%, 70%, and 85% ethanol, and were then washed 3× in 100% ethanol. Coverslips were then transferred to a CPD holder and incubated in 100% hexamethyldisilazane 2× for 10 min. each. Samples were then mounted on AI SEM stubs and coated with osmium plasma coater (4 nm) and were stored in a desiccator prior to imaging. SEM images were acquired on an FEI (Thermo Fisher Scientific) Scios2 LoVac dual bean FEG/FIB Scanning Electron Microscope with a Schottky emitter source. Images were acquired at 15.0 kV with 3 nm spot size.

Transmission electron microscopy and cell wall measurements: For transmission electron microscopy fungal biofilms were fixed in 5 mL 2× fixative (2% GTA/2% PF in 0.05M NaCacodylate pH 7.4) for 1 hour and then replaced with fresh fixative. Biofilms were scraped from coverslips and hyphae were pelleted and excess fixative removed. Hyphae were transferred to 100 μl 2% molten agar and solidified. Agar drops were trimmed to removed excess agar and transferred to 1 mL fresh fixative and rotated for 3 hours at room temperature then 48 hours at 4° C. Pellet was rinsed in 0.1M NaCac/0.1M Sucrose to remove GTA and then post-fix treated with 2% OsO₄ in 0.1M NaCac/0.07M Sucrose for 2 hours. Soft agar pellet was then rinsed twice with dH₂O and then transferred to En-bloc stain with 1% Uranyl Acetate for 2 hours at room temperature in the dark. Pellet was then dehydrated through ethanol series at room temperature with 30%, 50%, 70% for 30 minutes each, then on a rotator for two days, followed by further dehydration with 85% then 95% ethanol for 30 minutes and then 100% ethanol for 6 rinses over 6 hours. Samples were then left at 4° C. for 48 hours. Samples were then incubated 2× in propylene oxide for 30 minutes each, then immersed in 1:1 LX112 (LADD, Inc. Burlington, VT):PO for 1 hour at room temperature and then in 1.5:1 LX112:PO for 18 hours. LX112 from LADD epoxy solution used in 6A:4B for medium hard block. Excess fluid was removed and samples were placed in vacuum desiccator for 24 hours before being transferred to BEEM capsules with fresh LX112, centrifuged for 30 min at 1500 rpm and returned to vacuum desiccator for 12 hours. Samples were polymerized at 45° C. for 24 hours, 60° C. for 24 hours, and then cooled and thin sectioned and places in 2% UA_{MeOH} for 10 minutes followed by 3% Reynolds lead citrate for 2-3 minutes. Protocol was based on Burghardt & Droleskey (Burghardt et al. Curr Protoc Microbiol Chapter 2, Unit 2B 1, 2006). Samples were imaged on JEOL JEM 1010 transmission electron microscope at 100.0 kV. To determine cell wall size, ImageJ was used to open images files and for each cross-section of a filament 10 measurements of cell wall thickness, disregarding the electron-dense ECM, were averaged per filament.

Statistics and Reproducibility: All statistical analysis was performed in GraphPad Prism 5, GraphPad Prism 8, and R. Unless otherwise noted, all statistical analyses were performed with a minimum of three biologically independent samples. All images are representative of a minimum of three biologically independent samples that represent a minimum of three independent experimentations unless otherwise noted. funPact images are representative of five independent animals, but to reduce the use of animals, samples for funPact images were generated from two independent sample preparations. For comparisons between two groups two-tailed unpaired t-tests were performed. For comparisons between greater than two groups One-Way ANOVA with Tukey, Sidak, or Dunnett post tests for multiple comparisons were performed. All error bars indicate standard error and are centered around the mean.

### Oxygen Tension Significantly Influences Fungal Colony Morphology and Biofilm Architecture

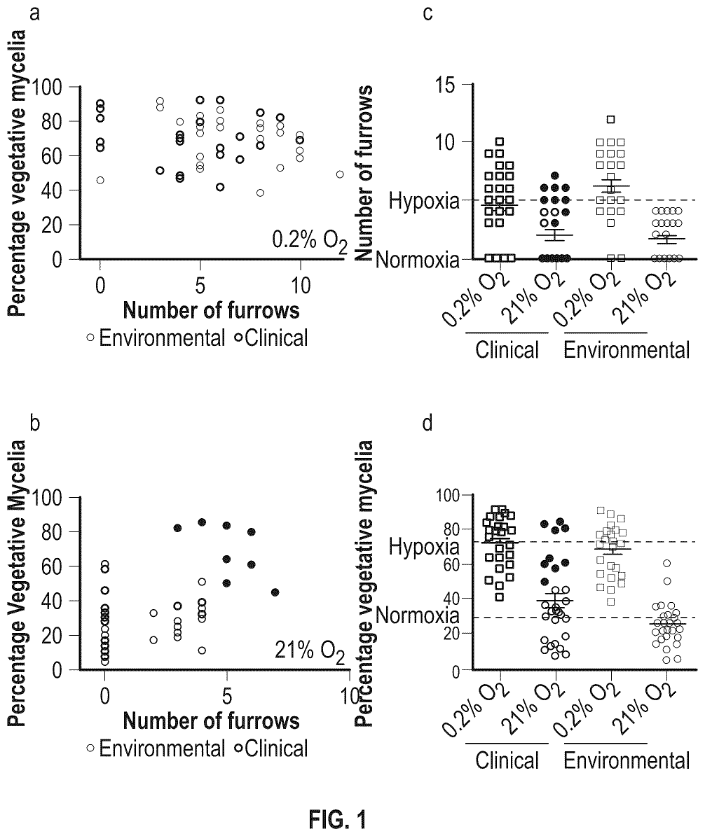

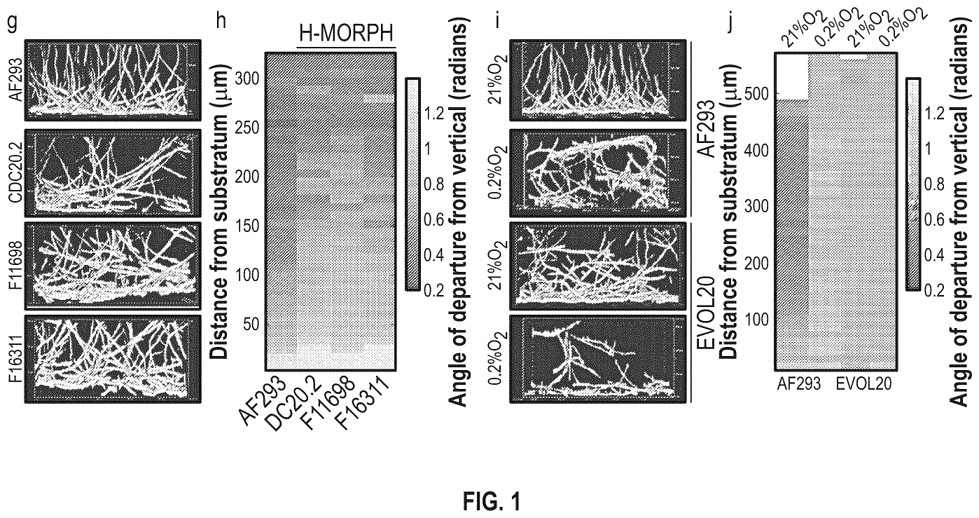

A. fumigatus CM is heterogeneous in response to oxygen tension (Kowalski 2016, supra). A screen of 58 isolates at 0.2% O₂ for two morphological features (1) colony furrowing and (2) percent vegetative mycelia (white, non-conidiating mycelia) (PVM) revealed abundant furrowing (mean: 5.30) and a high PVM (mean: 70.4%) (FIG. 1a, FIG. 2a). Colonies at 21% O₂ have significantly fewer furrows (mean: 1.85, p<0.0001) and significantly reduced PVM (mean: 32%, p<0.0001) (FIG. 1b). Oxygen tension is a significant source of variation for both colony furrowing (31.67%, p<0.0001) and PVM (55.77%, p<0.0001) (FIG. 1c, FIG. 1d). Most isolates screened have low furrowing and low PVM at normal oxygen (N-MORPH) and elevated furrowing and PVM at low oxygen (hypoxia) (H-MORPH) (FIG. 1e). A strain was considered to be H-MORPH if furrows are greater than 3 and PVM is greater than 40% when grown in the above described culture conditions. A subset of clinical strains adopt H-MORPH even at 21% O2 (filled circles FIG. 1b, FIG. 1c, FIG. 1d, FIG. 1f). Three such strains, CDC20.2, F11698, and F16311, have significantly increased low oxygen fitness (H/N) relative to the N-MORPH reference AF293 (FIG. 1f, FIG. 2b).

H-MORPH submerged fungal biofilms have altered biofilm architecture compared to AF293 (FIG. 1g). AF293 biofilms have a mat of filaments at the base perpendicular to the vertical axis. Above ˜50 m, filaments grow polarized toward the air liquid interface with little deviation from the vertical axis (FIG. 1h). The N-MORPH strain AF293 cultured at 21% O₂ formed an organized biofilm with straight filaments growing toward the air-liquid interface. Clinical H-MORPH strains are similar within the first ˜50 μm, but the remaining volume contains filaments that deviate from the vertical axis (FIG. 1h, FIG. 3). This pattern of altered architecture is similar to AF293 cultured at 0.2% O₂, which forms a highly disorganized biofilm with many filaments that divert from the vertical, and in the AF293 hypoxia-evolved H-MORPH strain EVOL20 independent of oxygen tension, which forms a highly disorganized biofilm with many filaments that divert from the vertical at both 21% O₂ and 0.2% O₂ (FIG. 1i, FIG. 1j, FIG. 2c, FIG. 3). These data suggest that CM is an indicator of microscopic biofilm architecture impacted by oxygen.

H-MORPH Occurs Throughout Genetically Diverse Strains of A. fumigatus

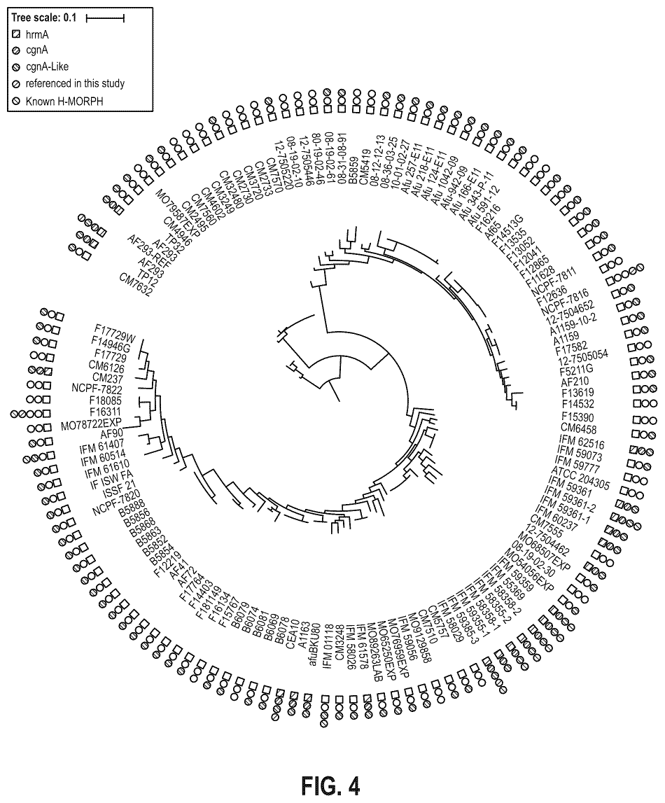

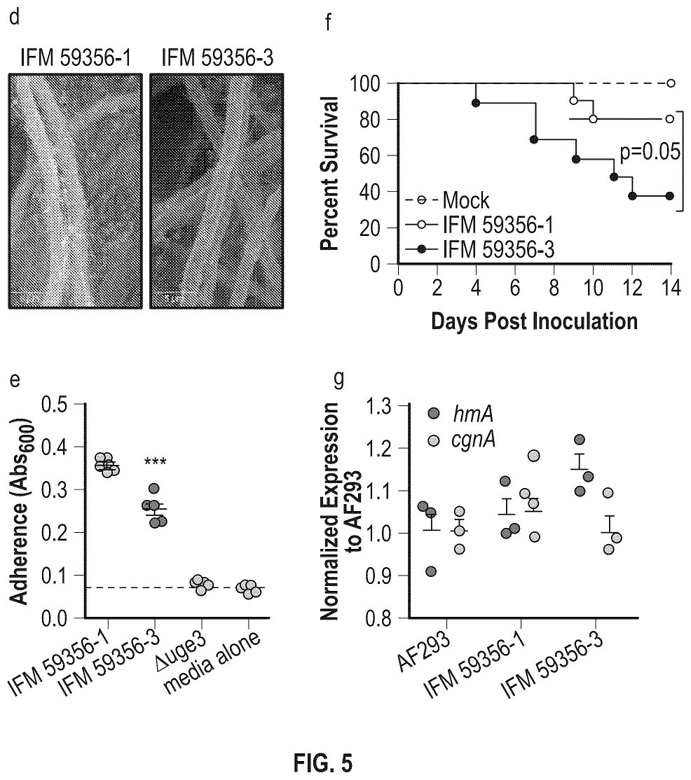

H-MORPH is not segregated by clade within the A. fumigatus phylogeny (FIG. 4). Two H-MORPH clinical strains, F11698/NCPF-7816 and F13611, represent the abundant A. fumigatus genetic diversity with one present in each of the two major clades (FIG. 4). Genetically similar, co-isolated clinical strains, IFM 59356-3 and IFM 59356-1, have H-MORPH and N-MORPH respectively (FIG. 5a). Consistent with H-MORPH (FIG. 1g, FIG. 1h), IFM 59356-3 has a biofilm with greater filament deviation from the vertical relative to its N-MORPH counterpart IFM 59356-1 (FIG. 5b, FIG. 5c). The lack of clustering of H-MORPH within the phylogeny and the ability to generate this CM suggest multiple genetic mechanisms likely exist through which A. fumigatus evolves these morphological features.

A Sub-Telomeric Gene hrmA Allele is Sufficient to Generate H-MORPH

An in vitro experimental evolution approach with AF293 in 0.2% O₂ generated the strain EVOL20 that adopts H-MORPH independent of oxygen tension (FIG. 2c, FIG. 2d). Whole genome sequence analysis of EVOL20 revealed three non-synonymous mutations compared to AF293, including a missense mutation in an uncharacterized hypothetical protein Afu5g14900. This single nucleotide polymorphism (SNP) (D304G) was only identified in H-MORPH EVOL20 from the passaged population (FIG. 2f). RNA sequencing indicates that Afu5g14900 transcript is significantly elevated in EVOL20 relative to AF293 in both normal (p=0.0002) and low oxygen conditions (p<0.0001) (FIG. 2e). Due to the generation of H-MORPH in EVOL20 the gene Afu5g14900 is named hypoxia responsive morphology factor A, hrmA.

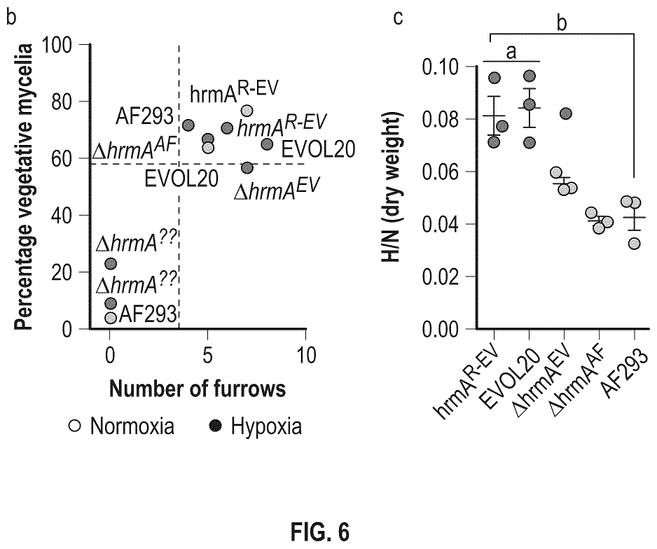

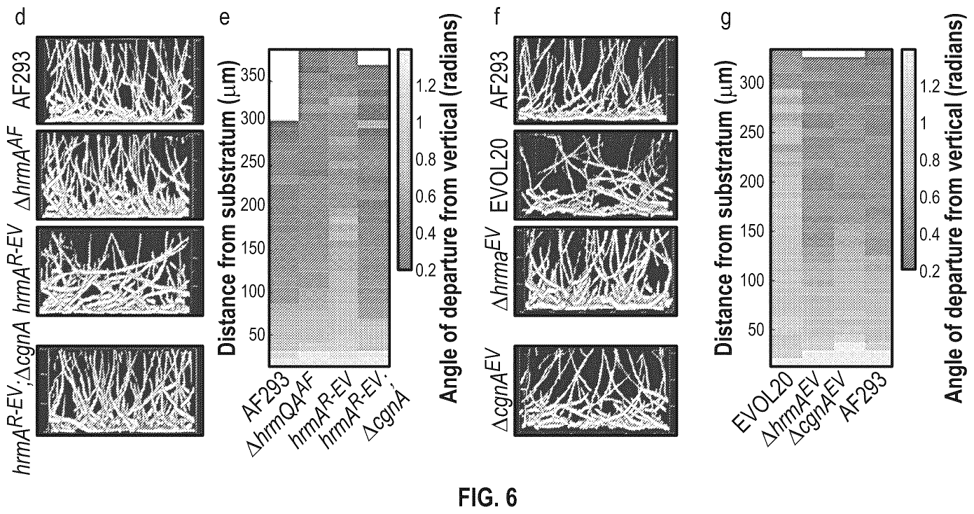

In AF293, hrmA loss (ΔhrmA^{AF}) did not alter in vitro CM in terms of furrowing and PVM, however, reconstitution of ΔhrmA^{AF} with the EVOL20 allele of hrmA (hrmA^{R-EV}) was sufficient to generate H-MORPH independent of oxygen tension (FIG. 6a, FIG. 6b). hrmA^{R-EV} has hypoxia fitness equivalent to EVOL20 (FIG. 6c). Conversely, hrmA loss in EVOL20 (ΔhrmA^{EV}) resulted in a loss of H-MORPH during growth at 21% O₂ (FIG. 6a, FIG. 6b), and a reduction in hypoxia fitness (FIG. 6c). Similar to H-MORPH locked clinical isolates (FIG. 1g) and EVOL20, hrmA^{R-EV} generated a biofilm with vertically misaligned filaments above the first ˜50 μm (FIG. 6d, FIG. 6e). Loss of hrmA in EVOL20 restored AF293-like biofilm architecture (FIG. 2f, FIG. 2g). Thus, the hypoxia-evolved allele of hrmA is shown herein to be sufficient and necessary to generate H-MORPH in AF293 and EVOL20, respectively.

H-MORPH Coincides with the Initiation of the Hypoxia Transcriptional Response at Ambient Oxygen Tensions

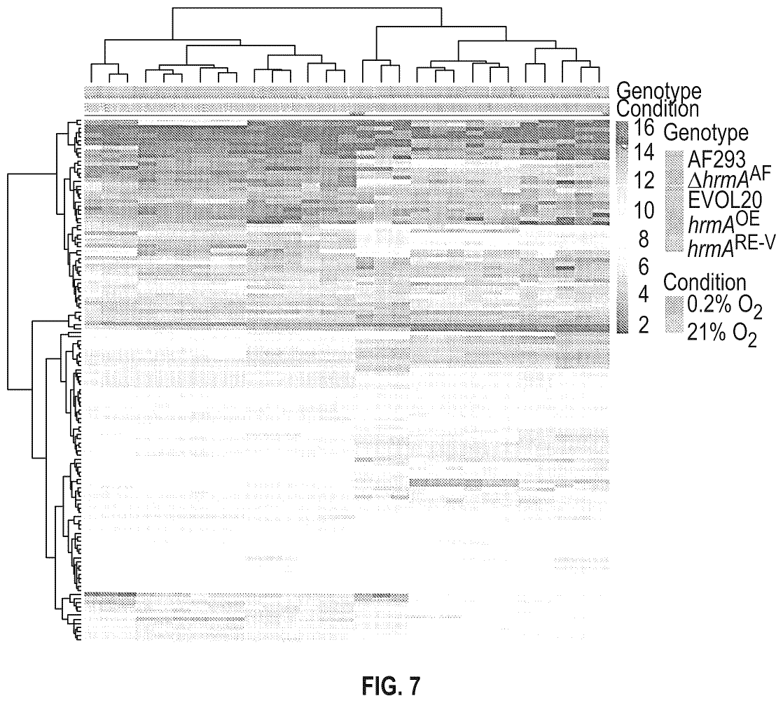

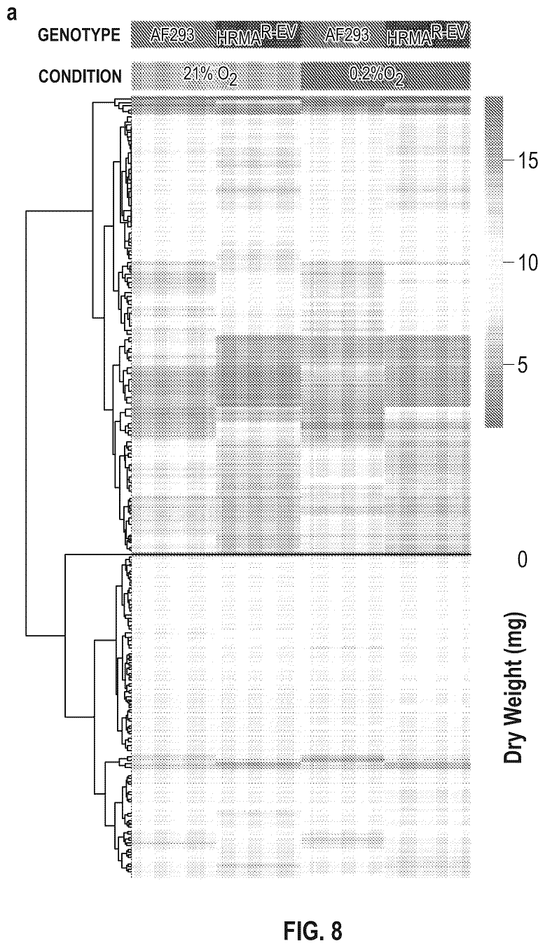

RNA sequencing was utilized to visualize broad consequences of H-MORPH at normal and low oxygen tensions. Hierarchical clustering of the transcriptomes reveals H-MORPHs hrmA^{R-EV} and hrmA^{OE} (over expression of the AF293 allele in AF293) cluster independently from N-MORPHs AF293 and ΔhrmA^{AF} (FIG. 7). Of the differentially expressed transcripts between hrmA^{R-EV} and AF293 in 21% and 0.2% O₂, 58% are oxygen-response genes in AF293 (FIG. 8a). The Gene Ontology Functional Categories GO:0016491 Oxidoreductase Activity (32/904) and GO:0005506 Iron Ion Binding (7/142) are significantly enriched in the differentially expressed genes between hrmA^{R-EV} and AF293; two categories shown previously to be enriched during the hypoxia response (Barker et al. BMC Genomics 13, 62, 2012).

Transcripts with an increase or decrease of at least 4-fold between AF293 and hrmA^{R-EV} were categorized as “Hypoxia Induced Genes” (H/N>4), “Hypoxia Reduced Genes” (H/N<−4), or “Hypoxia Non-Responsive Genes” (4>H/N<−4). At 21% O₂, 51% of the transcripts increased in hrmA^{R-EV} compared to AF293 are “Hypoxia Induced Genes'”; conversely, 45% of the transcripts reduced in hrmA^{R-EV} compared to AF293 are “Hypoxia Reduced Genes” (FIG. 8b). Thus, H-MORPH strains, mediated by hrmA, activate the transcriptional hypoxic response despite oxygen replete conditions. At 0.2% O₂ where hrmA^{R-EV} is more fit than AF293, 71.8% of increased transcripts are “Hypoxia Reduced Transcripts” further supporting an altered physiological response to hypoxic stress in H-MORPH strains (FIG. 8b). The inverted hypoxia response of hrmA^{R-EV} coincides with reduced fungal biomass at 21% O₂ and increased biomass at 0.2% O₂ (FIG. 8c). However, following a shift from ambient oxygen to low oxygen the H-MORPH hrmA^{R-EV} has increased growth rate compared to the N-MORPH AF293 (FIG. 8d).

### HrmA is Induced During Murine Pulmonary Aspergillosis and Facilitates the Expression of a Sub-Telomeric Gene Cluster

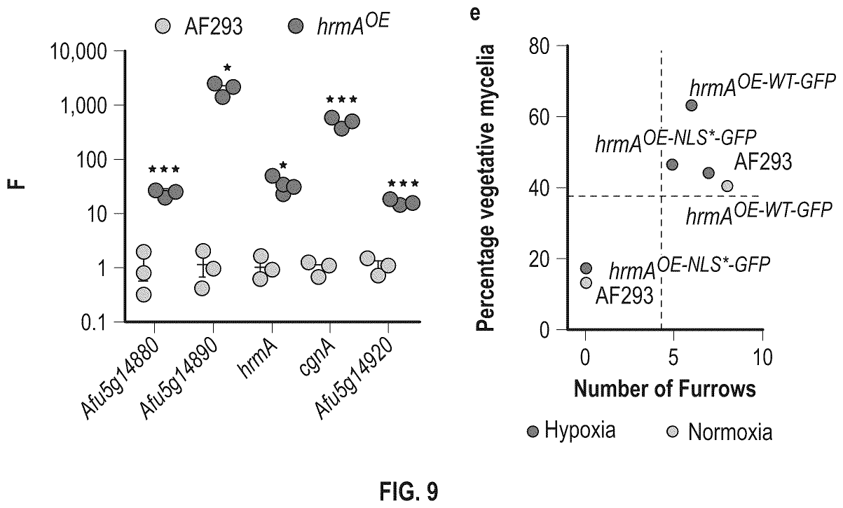

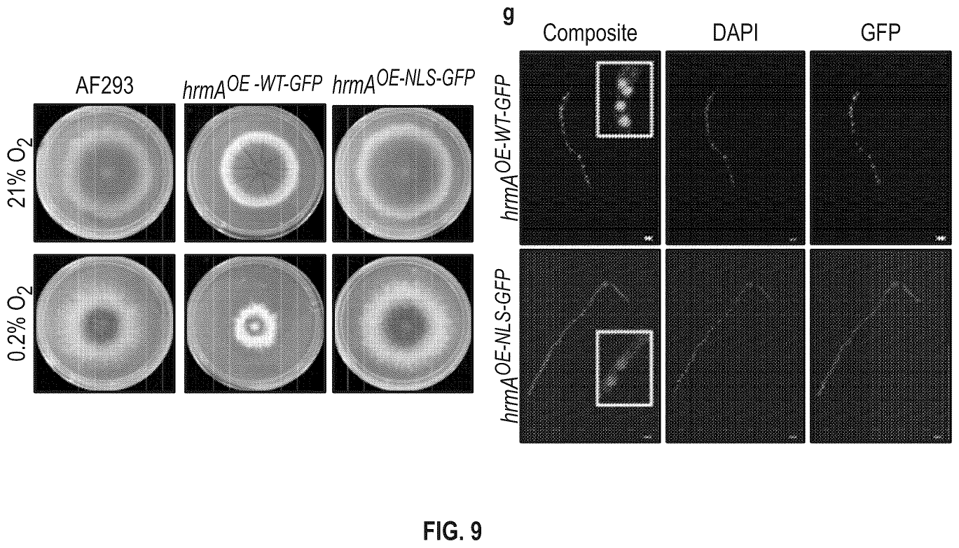

Previous reports suggest increased hrmA expression in vivo in a triamcinolone murine model of IA (Kale et al. Sci Rep 7, 17096, (2017). In that model, hrmA transcript levels significantly increase from 24 to 72 hours post fungal inoculation (hpi) (FIG. 9a). An increase in hrmA transcript in hrmA^{R-EV} (at the native locus) is also observed (FIG. 9b). HrmA is a member of a sub-telomeric gene cluster that responds to nitrogen starvation, a laboratory condition that transcriptionally correlates with a host-adaptation transcriptional response (McDonagh et al. PLoS Pathog 4, e1000154, 2008). Consistent with the assignment of hrmA to a sub-telomeric gene cluster, an influence of hrmA on transcript levels of genes surrounding its native locus was observed, termed here the hrmA associated cluster (HAC). In ΔhrmA^{EV}, the mRNA levels of three surrounding genes (Afu5g14880, Afu5g14890, Afu5g14910) are significantly reduced compared to EVOL20 (FIG. 9c). Ectopic overexpression of the AF293 allele of hrmA (hrmA^{OE}) acts in trans to facilitate an increase in transcripts of four HAC genes (Afu5g14880, Afu5g14890, Afu4g14910, Afu5g14920) (FIG. 9d).

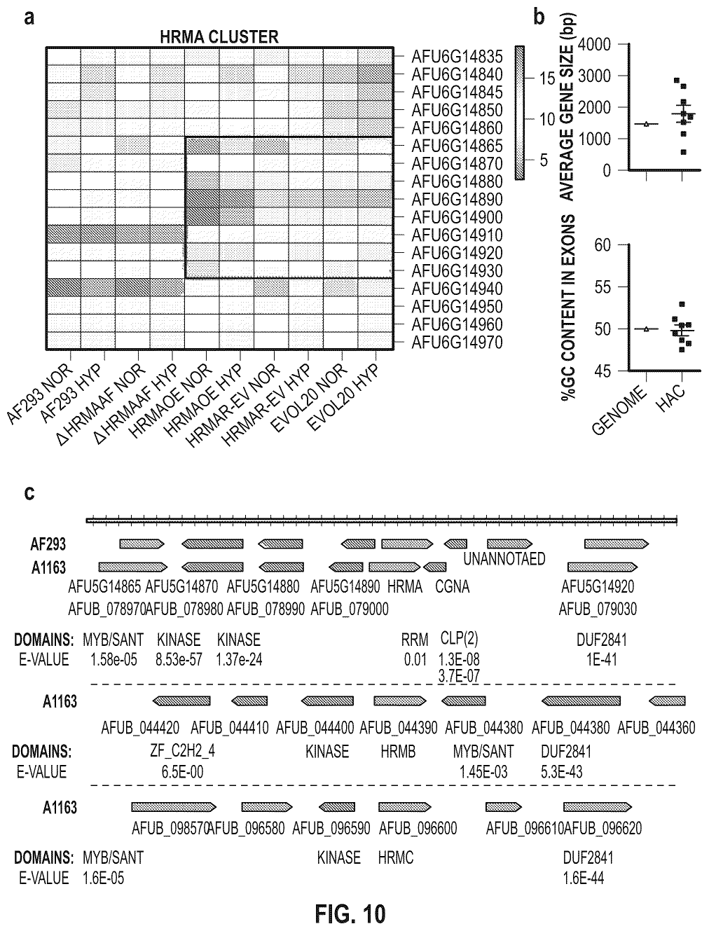

Analysis of co-regulated transcripts from RNA-sequencing predicts that HAC extends from Afu5g14865 to Afu5g14920, and includes a putative unannotated ORF 3′ to Afu5g14910 (Supplementary FIG. 10a, FIG. 10c). The average gene size and % GC content of HAC is not different from the AF293 genomic average (FIG. 10b) (Fedorova et al. PLoS Genet 4, e1000046, 2008); but in the hypoxia-fit strain A1163 (Kowalski 2016, supra), there is a sub-telomeric HAC that is syntenic to AF293 HAC and two additional putative homologous clusters that are not present in AF293 (FIG. 10c). The presence of these potential homologous clusters in a distantly related A. fumigatus strain suggests intragenomic movement of this genomic region. The clusters share certain genic components including genes encoding a MyB/SANT domain, a kinase domain, a DUF2841 domain, and putative hrmA paralogs (hrmB: AFUB_044390, hrmC: AFUB_096600). Analysis of HAC across sequenced strains indicates heterogeneous abundance of the original and homologous gene clusters (FIG. 4, alignment: https://github.com/stajichlab/Afum_hrmA_cluster_evolution; DOI: 10.5281/zenodo.3257606), potentially highlighting a role for these homologous clusters in H-MORPH generation where HAC is absent. Other Ascomycetes encode genes similar to hrmA, including the human fungal pathogens Histoplasma capsulatum and Coccidioides immitis (https://github.com/stajichlab/Afum_hrmA_cluster_evolution).

### HrmA Nuclear Localization is Necessary for the Induction of HAC

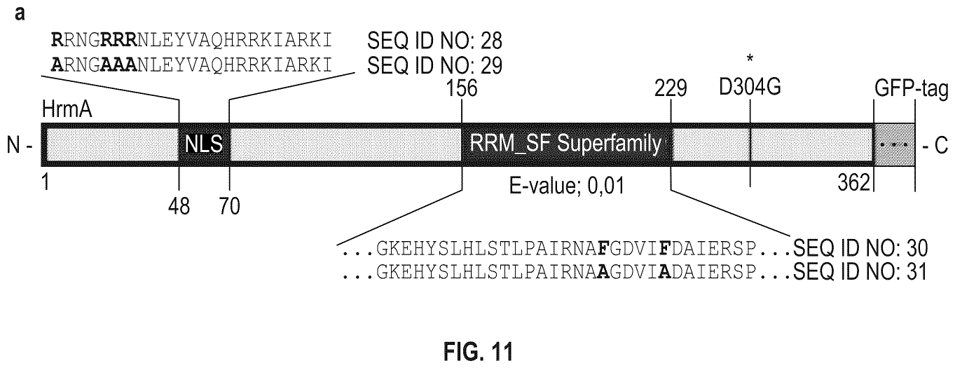

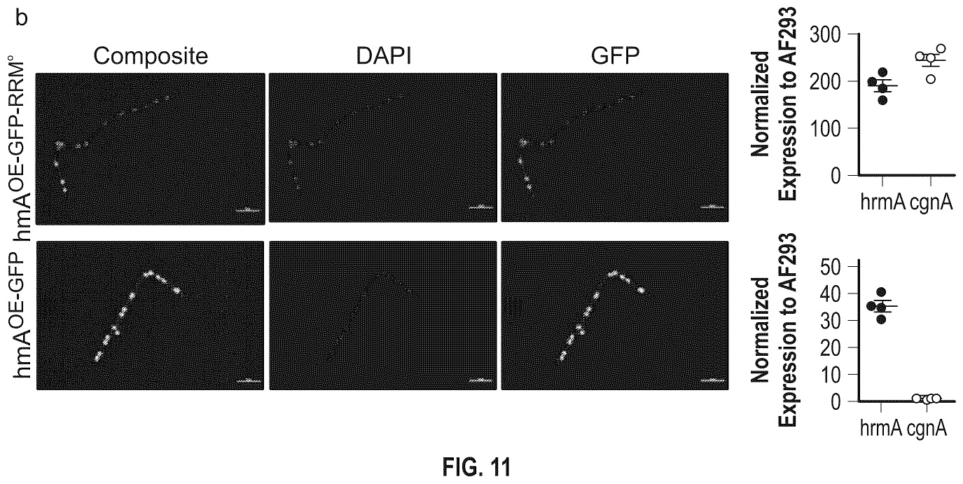

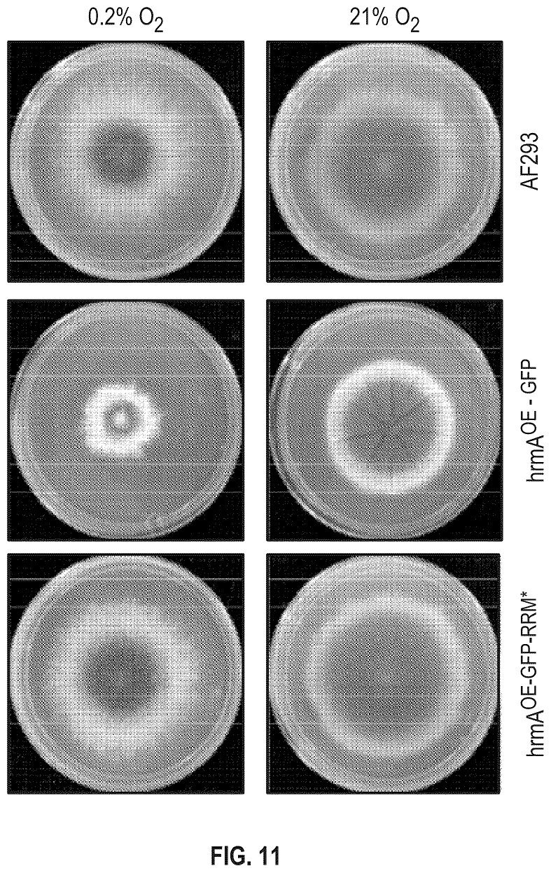

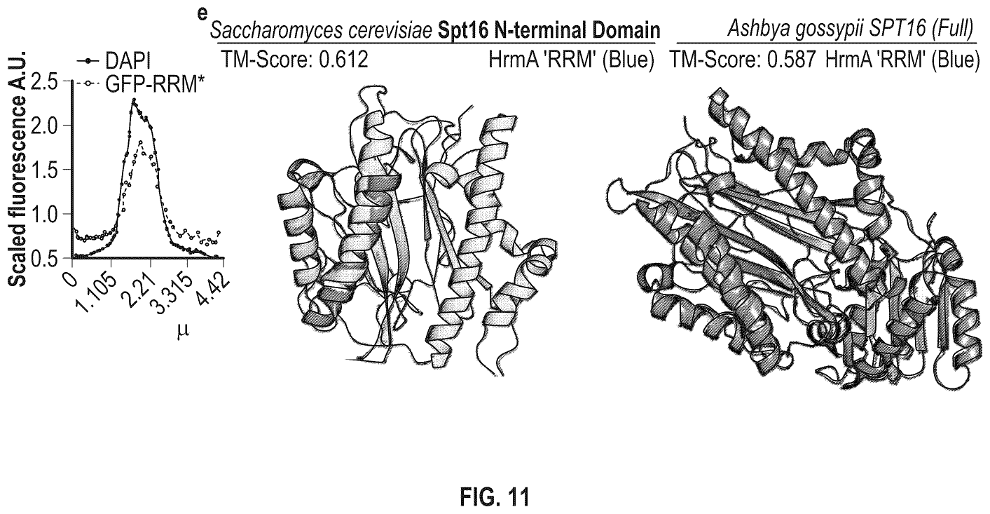

The HrmA protein sequence reveals a predicted N-terminal bipartite nuclear localization signal (NLS) (http://nls-mapper.iab.keio.ac.jp/) and a weakly predicted RNA Recognition Motif (RRM) domain (E-value: 0.01) (FIG. 11a). Overexpression of the parental allele of hrmA with a C-terminal GFP tag in AF293 generates oxygen-independent H-MORPH (FIG. 9e, FIG. 9f). In contrast, over expression of hrmA with a disrupted NLS is unable to generate H-MORPH (FIG. 4e, FIG. 4f) despite elevated levels of hrmA transcript (FIG. 9i). Confocal imaging reveals GFP signal enriched in the same location as the nuclear DAPI stain for the WT allele but a lack of this enrichment for the NLS mutant (FIG. 9g, FIG. 9h). Without localization to the nucleus or nuclear region, HrmA is unable to facilitate HAC induction as shown by the cluster gene cgnA (Afu5g14910) (FIG. 9i).

Despite low sequence similarity in the alignment to the RRM domain in HrmA, there are two conserved phenylalanine residues within this domain that are also present within hrmB and hrmC in strain A1163. When these conserved phenylalanine residues are each mutated to alanine, overexpression of this allele cannot generate H-MORPH despite observing hrmA nuclear region localization (FIG. 11b, FIG. 11c, FIG. 11d). Aromatic residues are critical in many RRM protein structures for direct interaction with nucleic acids (Law et al. Nucleic Acids Res 33, 2917-2928, 2005).

### H-MORPH is Generated Through HrmA-Mediated Induction of HAC

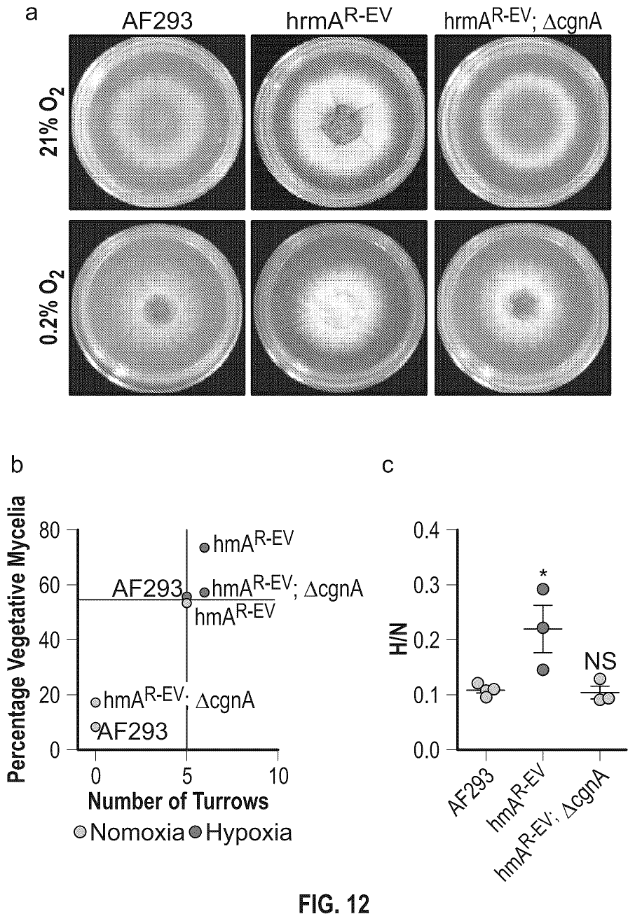

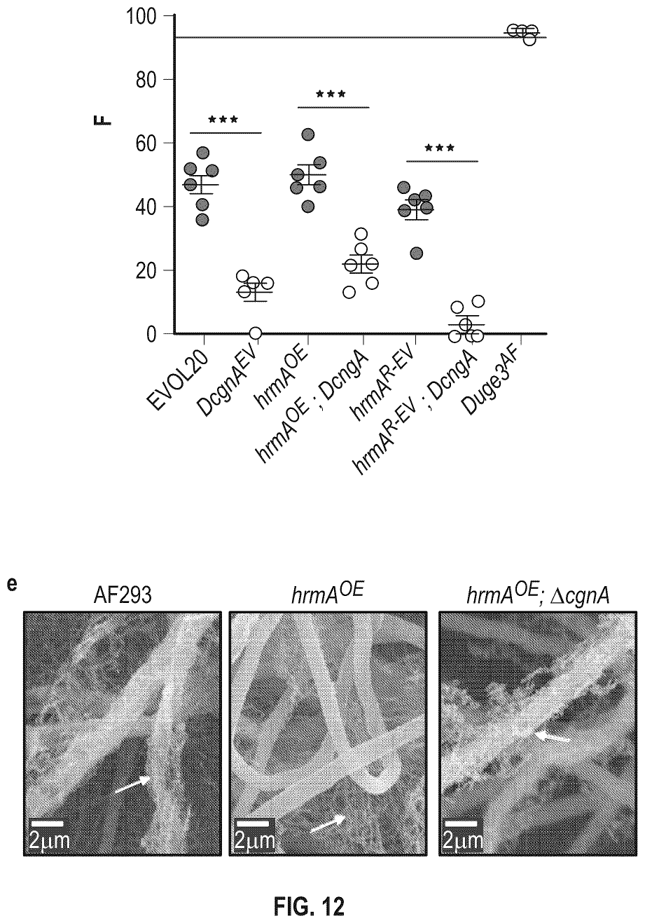

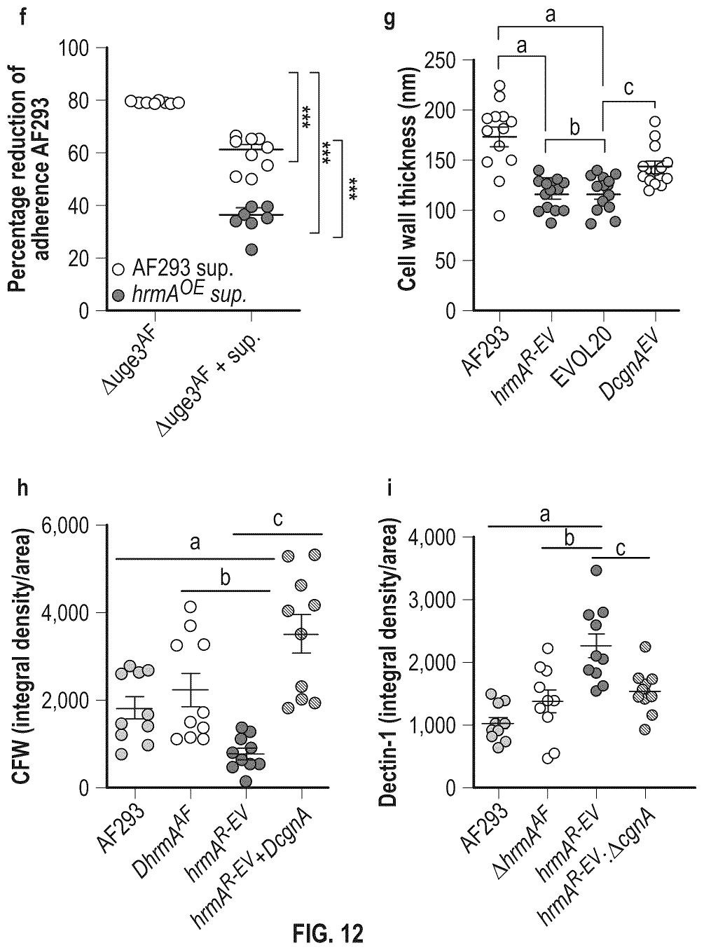

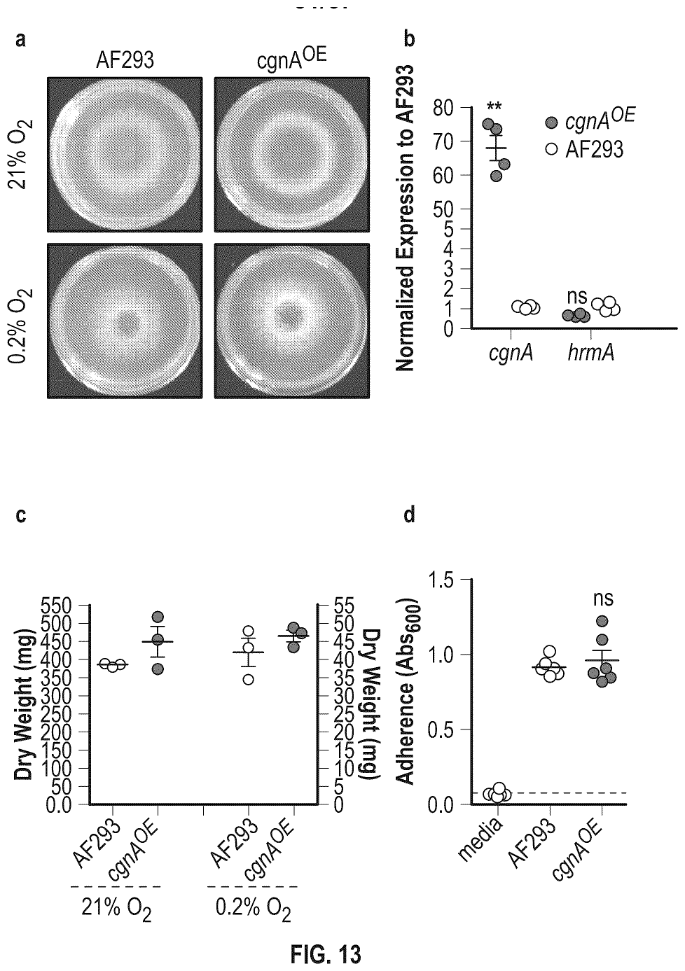

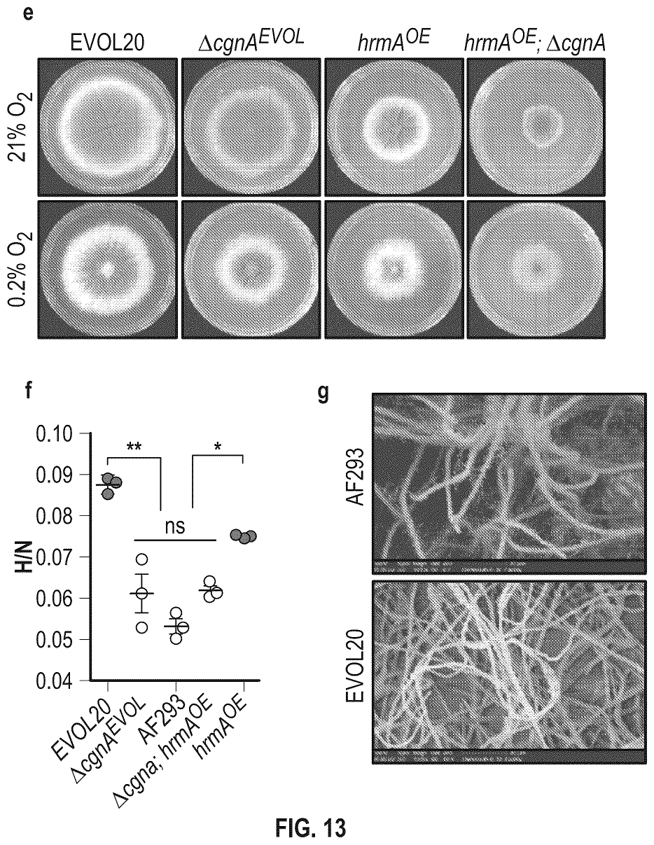

Loss of HAC induction abolishes H-MORPH indicating HAC is necessary for this morphotype and increased hypoxia fitness (FIG. 12, FIG. 11). Expression of the HAC gene Afu5g14910, cgnA, is an indicator of HrmA downstream effects and encodes a predicted collagen-like protein (CLP), a class of proteins present but unstudied in other fungi. In A. fumigatus, CgnA has a tripeptide G-X-Y repeat of G-Q-I and G-Q-S, and lacks a canonical secretion signal. Despite induction of cgnA greater than 100-fold relative to AF293 in hrmA^{OE} (FIG. 9d; morphology FIG. 13e), comparative levels of cgnA over expression in the absence of elevated hrmA (cgnA^{OE}) does not induce H-MORPH nor alter the hypoxic growth of AF293 (FIG. 13). Loss of cgnA in the context of elevated HAC abolishes H-MORPH, indicating a role for cgnA, and possibly other HAC genes, in the generation of H-MORPH (FIG. 12a, FIG. 12b; FIG. 13e). Loss of cgnA in HAC-induced strains EVOL20, and hrmA^{R-EV} reduces the hypoxia fitness of these strains (FIG. 12c, FIG. 13f), and restores the N-MORPH biofilm architecture and filament alignment to that of AF293 (FIG. 6).

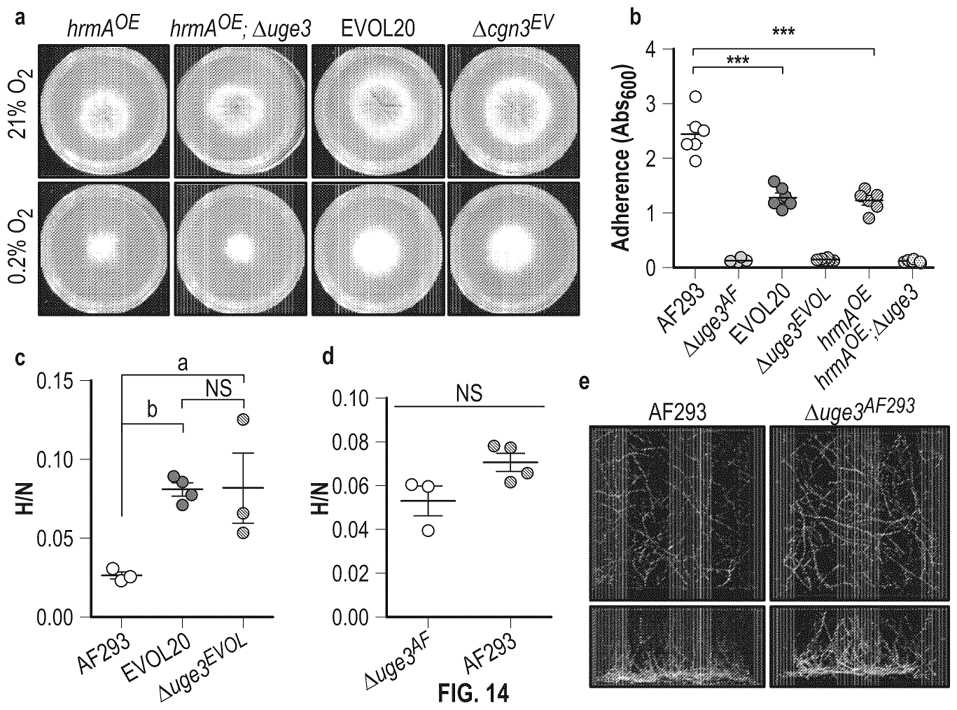

To further characterize the role of cgnA and HAC in the generation of H-MORPH, the features of the hyphal surface were assessed, as surface alteration and adhesion are associated with other microbial CLPs (Abdel-Nour et al. Appl Environ Microbiol 80, 1441-1454, 2014; Chen et al. BMC Microbiol 10, 320, 2010; Wang et al. Proc Natl Acad Sci USA 103, 6647-6652, 2006). Loss of cgnA and regeneration of N-MORPH increases surface adherence of H-MORPH strains (FIG. 12d), likely the consequence of ECM detachment from the H-MORPH strains (Supplementary FIG. 13g, FIG. 12e) that is dependent on cgnA. In the clinical strains IFM 59356-1 (N-MORPH) and IFM 59356-3 (H-MORPH), matrix detachment and reduced surface adherence is observed in H-MORPH (FIG. 5d, FIG. 5e). Matrix detachment from the H-MORPH filaments is not a defect in ECM production as it is still visibly secreted into the biofilms (FIG. 12e). A significant component of the ECM is galactosaminogalactan (GAG), and loss of GAG through deletion of the UDP-Glucose-4-epimerase uge3 abolishes surface adherence. Chemical modifications of GAG also prevents attachment of matrix to the hyphae, so the ability of secreted GAG from hrmA^{OE} to complement the adherence defect of the GAG deficient strain Δuge3^{AF} was investigated. Culture supernatants containing secreted GAG from AF293 and hrmA^{OE} were both able to significantly increase adherence of Δuge3^{AF} (FIG. 12f). These data suggest that HAC/cgnA modifies the hyphal surface mediating matrix/GAG detachment. To determine if GAG secretion was necessary for H-MORPH, uge3 deletions in hrmA^{OE} and EVOL20 were generated; as a result, CM did not change but surface adherence was abolished (FIG. 14 a, FIG. 14b). Loss of GAG production in AF293 does not impact hypoxia fitness nor the biofilm architecture (Supplementary FIG. 14d, FIG. 14e).

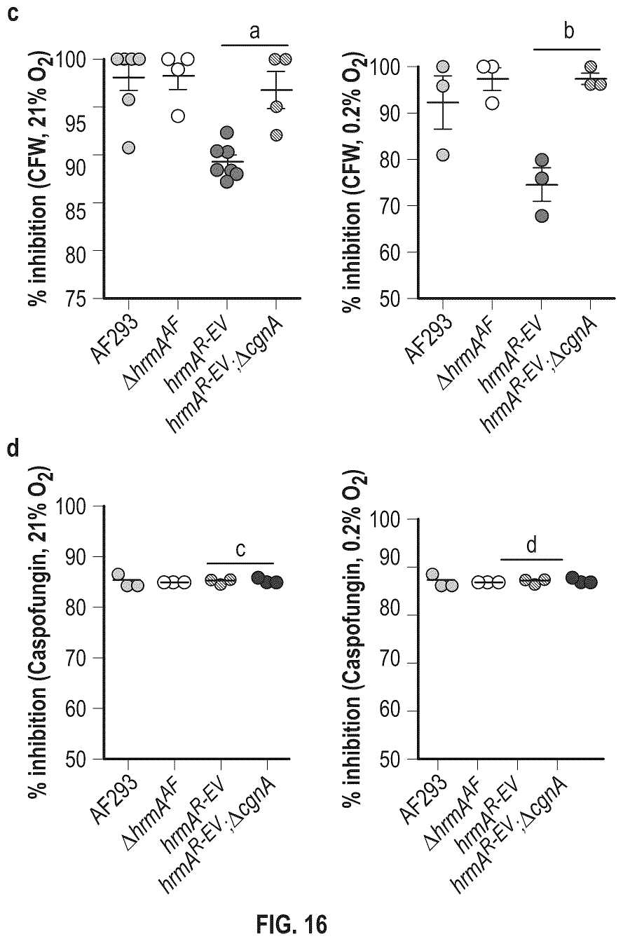

H-MORPHs hrmA^{R-EV} and EVOL20 have significantly thinner cell walls than the N-MORPH AF293, and in EVOL20 this is dependent on cgnA (FIG. 12g, FIG. 15). To determine if the cell wall architecture is altered, we imaged cell wall components through the use of calcofluor white (CFW) for chitin detection and soluble Dectin-1 for β-glucan detection. H-MORPH hrmA^{R-EV} has reduced total chitin that is dependent on the induction of cgnA (FIG. 12h, FIG. 16a). In contrast, hrmA^{R-EV} has significantly increased cgnA-dependent β-glucan exposure (FIG. 12i, FIG. 16b). hrmA^{R-EV} is also more sensitive to growth on CFW in both normal and low oxygen compared to AF293, ΔhrmA^{AF}, and hrmA^{R-EV}; ΔcgnA (FIG. 16c). No difference in sensitivity to the β-glucan synthase inhibitor caspofungin was observed (FIG. 16d). These surface changes appear to alter matrix attachment and inter-hyphal interactions within the developing biofilms resulting in a loss of vertically aligned polarized growing filaments.

### H-MORPH Altered Biofilm Architecture Occurs In Vivo.

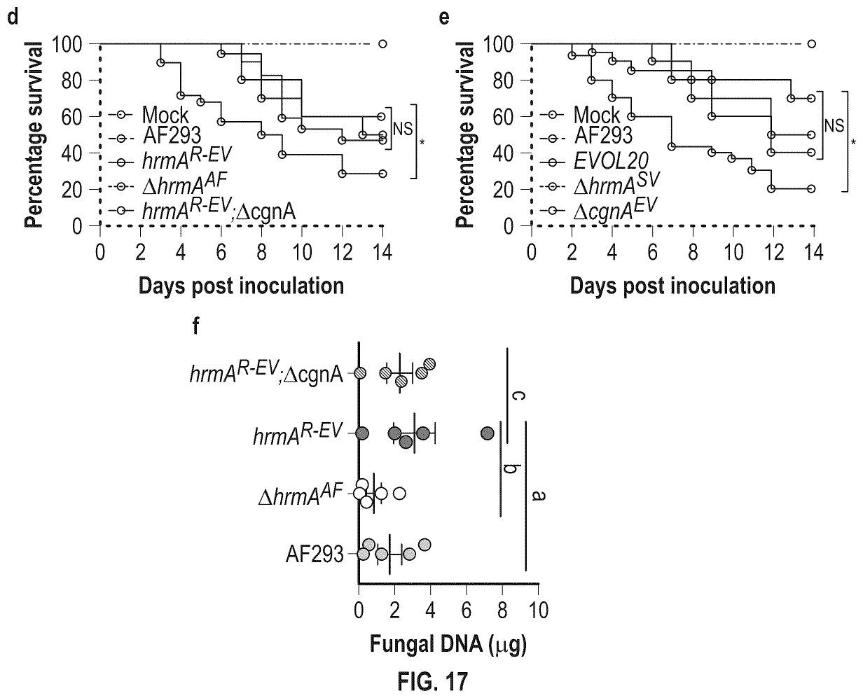

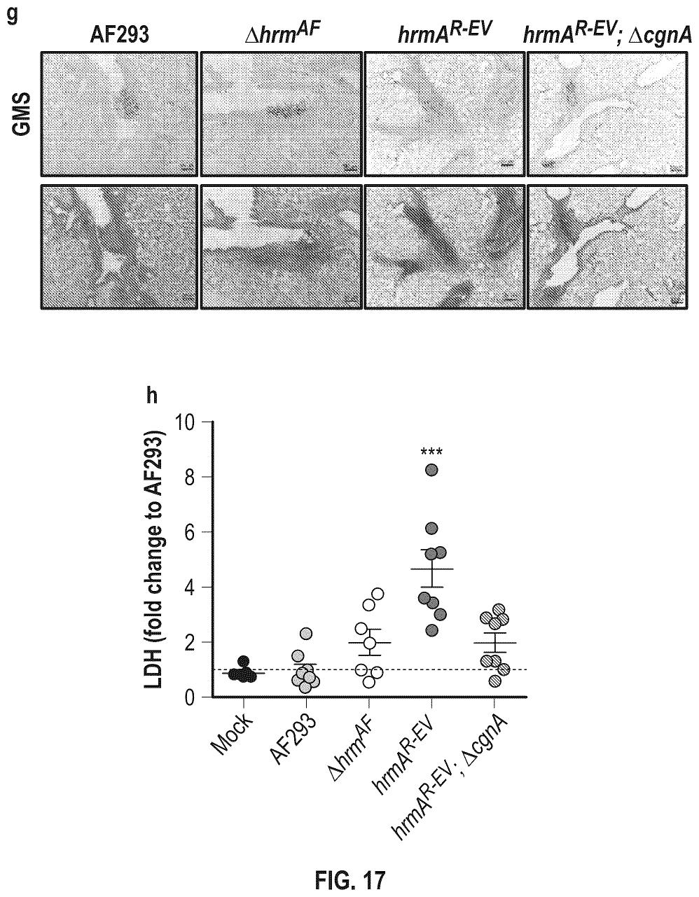

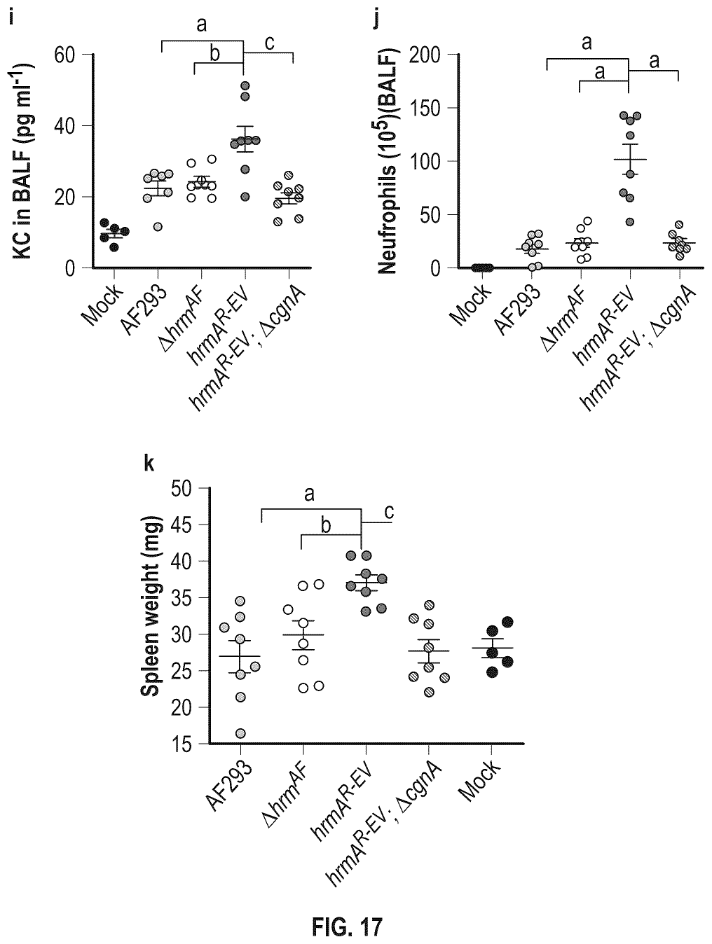

It was next determined if the altered filament surface influences the inter-filament interactions in vivo. The miPACT/PACT tissue clearing methods were adopted (microbial identification after passive clarity technique) to visualize in vivo fungal lesions in three dimensions using fluorescently labeled fungi (the technique is termed: funPACT:fungal imaging after passive clarity technique) (DePas et al. MBio 7, 2016; Yang et al. Cell 158, 945-958, 2014; Chung et al. Nature 497, 332-337, 2013). At 4 dpi and 5 dpi large inflammatory foci with fungal elements are observed within the airways of animals challenged with AF293 or EVOL20. At both time points, AF293 lesions are dense at the center with filaments radiating from the foci of infection, becoming less dense away from the center (FIG. 17a). There is a high degree of connectivity between filaments in AF293 lesions but not in EVOL20 lesions. At 4 dpi and 5 dpi the EVOL20 lesions are visibly more diffuse than those of AF293 (FIG. 17a, FIG. 17b; FIG. 18). There are no dense foci within the EVOL20 lesions, and single filaments can be observed dispersed in distinct locations within the mass of host immune infiltrate (FIG. 17b).

To quantify differences in lesion architecture, we performed Gomori's methenamine silver (GMS) stain and applied a nearest-neighbor algorithm to quantify the “compactness” of fungal lesions within the large airways. The more compact a fungal lesion is, the shorter the distance between each filament and its nearest neighbors; while more diffuse lesions have larger average distances between filaments. Qualitative analysis of the histopathology between N-MORPH AF293 and H-MORPH EVOL20 supported the hypothesis that H-MORPH fungal lesions are more diffuse, and quantification reveals significantly less compact lesions with EVOL20 than AF293 (FIG. 17c). Expansion of this algorithm to lesions of N-MORPHs ΔhrmA^{AF} and hrmA^{R-EV}; ΔcgnA and H-MORPH hrmA^{R-EV} reveal significantly reduced compactness of hrmA^{R-EV} compared to the N-MORPH strains (FIG. 17c, FIG. 19, FIG. 18c). The diffuse nature of the hrmA^{R-EV} lesion is dependent on cgnA and only coincides with H-MORPH.

### H-MORPH Facilitate Disease Progression

H-MORPH F11698 (n=7) is significantly increased in murine virulence relative to AF293 (n=5) (p=0.0096) (Supplementary FIG. 5h, FIG. 5i). However, these are non-isogenic strains with an estimated 35759 SNPs between them that could contribute to differences in virulence and morphology. A second comparison between closely related clinical isolates N-MORPH IFM 59356-1 and H-MORPH IFM 59356-3 reveals a 40% increase in survival at 14 dpi, and a 5-day delay before the first mortality event in N-MORPH inoculated animals. By quantitative real-time PCR (qRT-PCR) no significant difference in mRNA levels of hrmA or the HAC gene cgnA is observed between these two strains that contain 51 nonsynonymous SNPs between them. (FIG. 5g).

Loss of hrmA in AF293 does not impact murine mortality, however introduction of the hypoxia-evolved allele of hrmA (hrmA^{R-EV}) and generation of H-MORPH significantly augments virulence in a cgnA-dependent manner (FIG. 17d). Loss of hrmA or cgnA in the H-MORPH EVOL20 significantly attenuates EVOL20 virulence (FIG. 17e). Despite the H-MORPH strains increased virulence, there is no significant difference in fungal burden between AF293, hrmA^{R-EV}, ΔhrmA^{AF}, and hrmA^{R-EV}; ΔcgnA at 4 dpi (FIG. 17f). Increased β-glucan exposure in the cell wall of H-MOPRH strains is consistent with observed increases in inflammation at 4 dpi (FIG. 17g, FIG. 2h, FIG. 2i). The airways where H-MORPH hrmA^{R-EV} is growing are full of immune cell infiltrate that is reduced around lesions of N-MORPH strains (FIG. 17g).

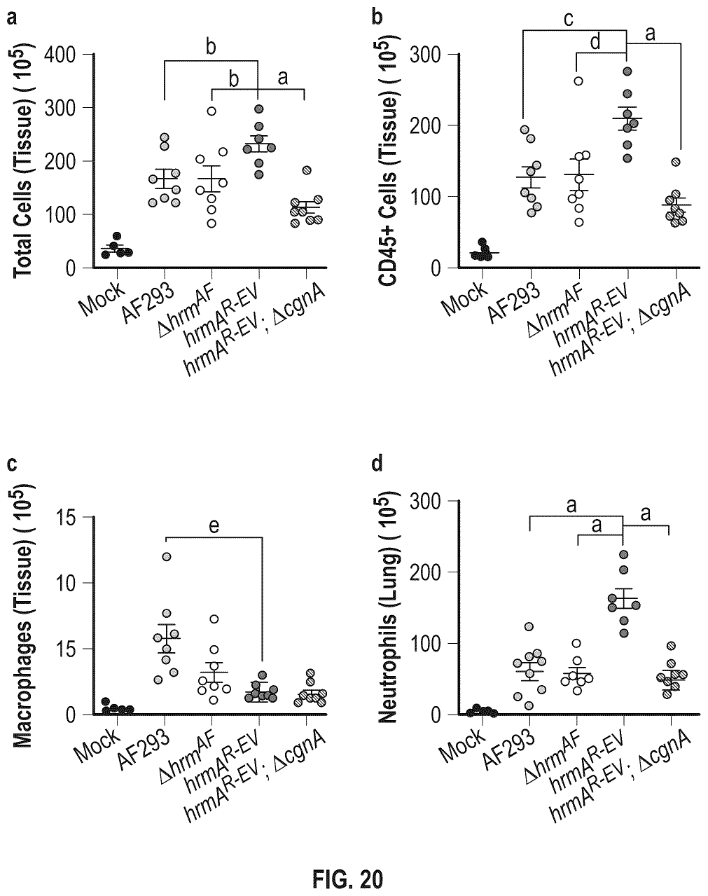

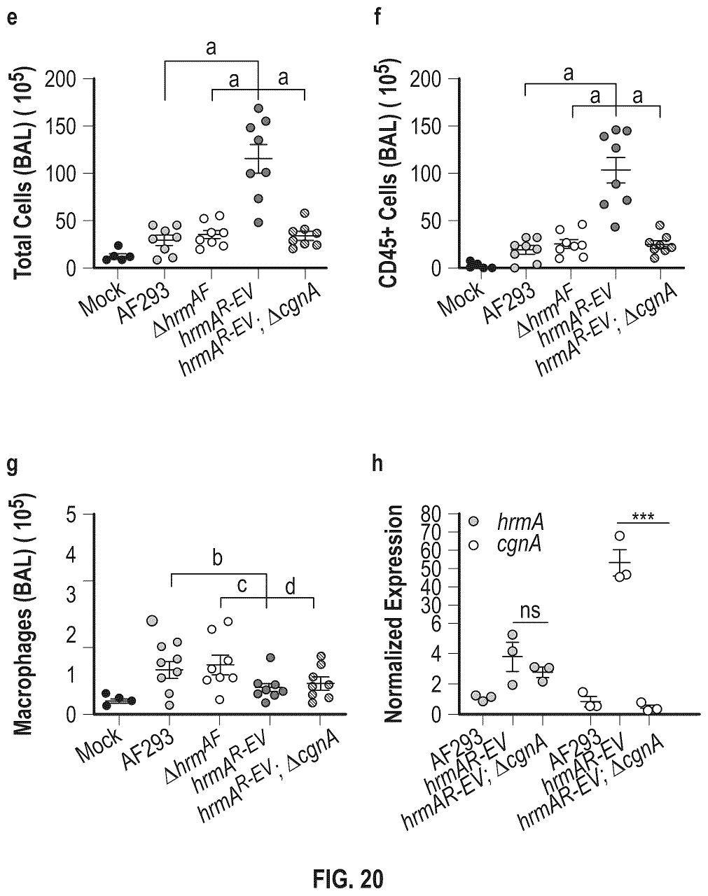

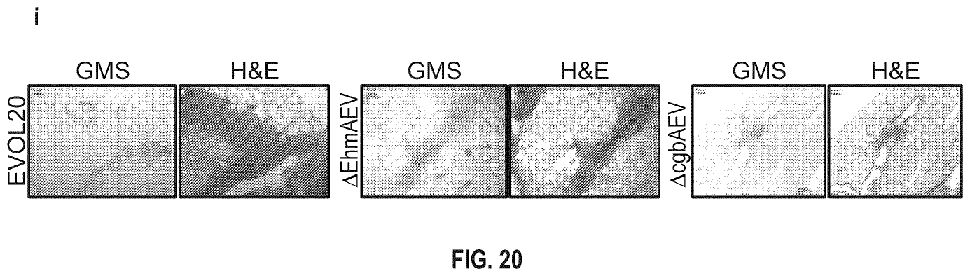

Host cell damage measured through lactate dehydrogenase (LDH) release in BALF after inoculation with hrmA^{R-EV} indicates a significant increase in host cell damage (FIG. 17h). In both the airways and lung tissue, H-MORPH inoculum is associated with a significant increase in total cells (Supplementary FIG. 20a, FIG. 20e) and CD45+ leukocytes (Supplementary FIG. 20b, FIG. 20f). A significant increase in the neutrophil chemoattractant KC from BALF is detected (FIG. 6i) and corresponds with an increase in airway neutrophils (FIG. 17j). The elevated host response to inoculation with H-MORPH hrmA^{R-EV} is dependent on HAC/cgnA, as loss of cgnA does not reduce hrmA transcripts (Supplementary FIG. 20h). These data indicate that localized pulmonary inflammation is elevated following inoculation with H-MORPH; but in addition systemic inflammation, as measured by spleen weight, is significantly increased 60 hpi with hrmA^{R-EV} compared to AF293, Δhrm^{AF}, and hrmA^{R-EV}; ΔcgnA. (FIG. 17k). Together, these data suggest H-MORPH occurs in vivo and significantly impacts disease progression in part through an increase in immunopathogenesis.

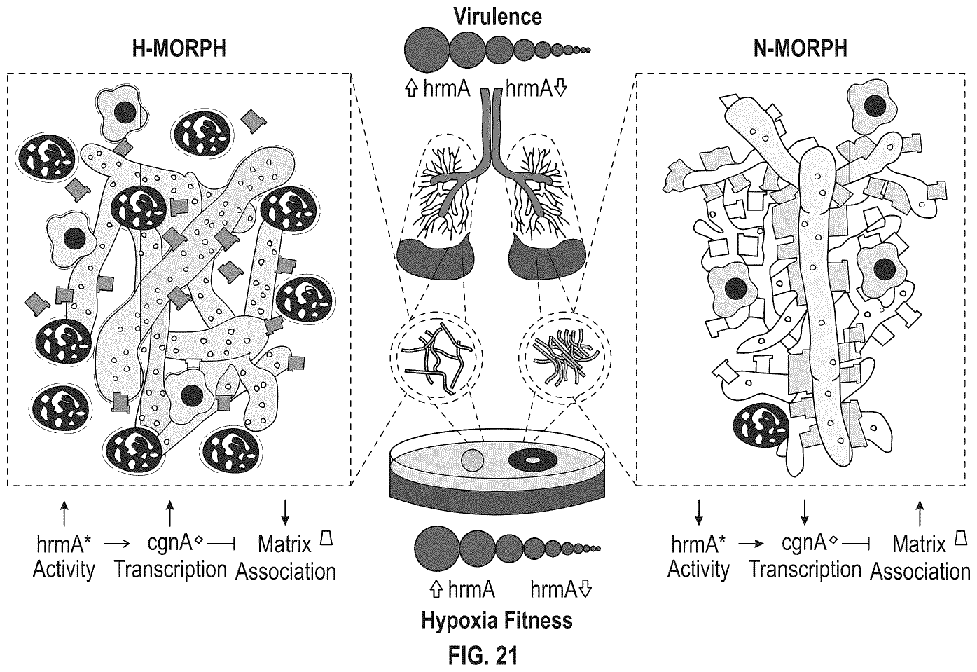

As shown in the model of FIG. 21, hypoxic environments, such as the infected host lung, provide an adaptive pressure to the obligate aerobic mold Aspergillus fumigatus. This selective pressure can lead to the adoption of certain hypoxia-typic morphological features—furrowing, elevated PVM—and that strains where these morphological traits occur regardless of oxygen tensions have a fitness advantage in low oxygen and simultaneously an increase in virulence. The model disclosed herein shows that the macroscopic morphology (N-MORPH v. H-MORPH) is an indicator of biofilm architecture and hyphal surface characteristics that provide a loose disorganized biofilm and highly inflammatory, diffuse fungal lesions. This disclosure describes a sub-telomeric gene cluster regulator HrmA that promotes expression of CLP cgnA and other genes that alter the fungal surface preventing matrix attachment as one mechanism for the generation of H-MORPH characteristics.

As demonstrated in the above example, forcing an H-MORPH phenotype in a fungal host cell can provide said fungal host cell with growth advantages, such as a reduced oxygen demand. As shown herein, the H-MORPH phenotype in a fungal host cell can be created through the expression (including overexpression) of the sub-telomeric gene cluster regulator HrmA in said fungal host cell. This is a useful mechanism that can be exploited in fungal host cells used in industry for the production of useful products, such as enzymes, secondary metabolites, and citric acid.

### Example 2—Reducing Fungal Oxygen Consumption Through Heterologous Expression of a Novel Sub-Telomeric Gene Cluster from Aspergillus Species

Example 1 above described the discovery and isolation of a novel and useful gene in Aspergillus fumigatus for enhancing hypoxia tolerance in fungal host cell.

Identification of A. fumigatus Gene Cluster Implicated in Fungal Oxygen Consumption

Just as A. niger is relatively tolerant to low oxygen concentration (Show 2015, supra), A. fumigatus is also able to grow at oxygen tensions as low as 0.2% O₂ in both solid-surface and submerged cultures (Kowalski et al. MBio 7, 2016). An in vitro evolution experiment with A. fumigatus at 0.2% O₂, performed to identify mechanisms of low oxygen adaptation, generated a strain, EVOL20, with improved growth yields at 0.2% O₂ compared to the parental reference strain AF293 (Kowalski 2016, supra). Whole genome sequencing of the EVOL20 strain led to the identification of an allele (D304G) in a previously uncharacterized nuclear-localized protein hrmA (Afu5g14900) that was responsible for the increase low-oxygen growth of the strain (Example 1 above). The increased hypoxia fitness conferred by the D304G allele of Afu5g14900/hrmA coincides with a transcriptional profile that is consistent with a hypoxia response being activated in oxygen replete conditions, and this appears to prime the fungus for growth in low oxygen during oxygen fluctuations such as those found in fungal fermentations. It has also been shown that during exposure to low-oxygen conditions, A. fumigatus reduces its oxygen consumption, though this usually corresponds with a reduction of growth rate as well (Grahl et al. Mol Microbiol 84, 383-399, 2012). Intriguingly, the strain EVOL20 displays reduced oxygen consumption in oxygen replete conditions both when adhered to a submerged surface (FIG. 23a, analyzed with Agilent Seahorse XFe96) or in a planktonic batch culture (FIG. 23b, analyzed with Unisense oxygen microelectrode OX-25) at equivalent biomass compared to a wild-type strain (FIG. 23c). This is consistent with the strain being constitutively primed for growth in low oxygen. Astoundingly, these data suggest expression of the hrmA evolved allele can reduce oxygen consumption without dramatically impacting fungal biomass yield, by ˜40%.

The D304G allele of Afu5g14900/hrmA was shown as necessary and sufficient for a number of the phenotypes observed in the EVOL20 strain relative to the parent strain AF293 (Example 1 described above). Similarly, during planktonic batch growth, the reduction in 02 consumption by EVOL20 was dependent on the evolved allele of hrmA (FIG. 23b). Intriguingly, the reduction in 02 consumption in EVOL20 co-occurred with an accumulation of the TCA cycle intermediates aconitate, malate, isocitrate, and citrate following a shift from 21% O₂ to 0.2% O₂ for 120 minutes (FIG. 24a). Additionally, this low-oxygen consuming strain was poorly adherent to plastic and glass surfaces (FIG. 24b), another biotic factor implicated in efficient bioproduction with fungi (Colin et al. AMB Express 3, 27, 2013).

It is proposed herein to utilize the novel hypoxia-evolved hrmA allele (D304G) and associated gene cluster to generate strains of A. niger, S. cerevisiae, and other industrial relevant fungi that consume less 02 in production scale fermentations without a detrimental impact on biomass and product yield. Homologous recombination and CRISPR technology will be used to introduce the hrmA evolved allele OR hrmA-associated gene cluster (HAC) in entirety (Afu5g14865-Afu5g14920) into two citric acid producing strains of A. niger: ATCC 1015 and ATCC 11414. In addition, haploid S. cerevisiae strains will be generated expressing HAC and the hrmA evolved allele. Additional introductions of the evolved allele and associated gene cluster HAC will be introduced into other industrial relevant fungi. It is predicted that this technology being applicable in a diverse group of industrial relevant fungi.

Genome Editing of A. niger Strains

Using the sequenced reference strain A. niger CBS 513.88 we have identified a site within the sub-terminal chromosomal region of chromosome 8 with homologous gene content to the region 3′ of HAC in A. fumigatus (Table 4, FungiDB). The HAC gene content will be introduced within this region of the A. niger genome using CRISPR technology (Dong et al. J Microbiol Methods 163, 105655, 2019; Leynaud-Kieffer et al. PLoS One 14, e0210243, 2019). The entire HAC loci contains seven ORFs and spans ˜18 kb. The cluster will therefore be introduced in segments, beginning with the cluster regulator hrmA evolved allele (D304G) that was evolved experimentally and is important for the reduced oxygen consumption of EVOL20 (FIG. 23b). If the introduction of hrmA evolved allele in the absence of the other cluster genes is not sufficient to reduce 02 consumption in A. niger, larger portions of HAC within this region of the A. niger genome will be introduced and expressed. To facilitate the transformation of such large portions of DNA, XL10-GOLD® Ultracompetent cells (Agilent) will be used to generate and clone the desired constructs.

TABLE 4
Amino acid identities for HAC adjacent proteins in A. fumigatus with their
best hits in A. niger. Multiple loci 3′ to HAC in A. fumigatus map with high
identity to a group of adjacent proteins (putative orthologs) in the sub-
terminal region of A. niger CBS 513.88 chromosome 8.

| A. fumigatus | A. niger | Amino |
|---|---|---|
| AF293 | CBS 513.88 | acid identity |
| Afu5g14940 | An08g12090 | 60.48% |
| Afu5g14950 | An08g12080 | 38.43% |
| Afu5g14960 | Pseudogene | Not applicable |
| Afu5g14980 | An08g12070 | 50.20% |
| Afu5g14990 | An08g12060 | 48.96% |
| Afu5g14915 | An08g12010 | 41.03% |

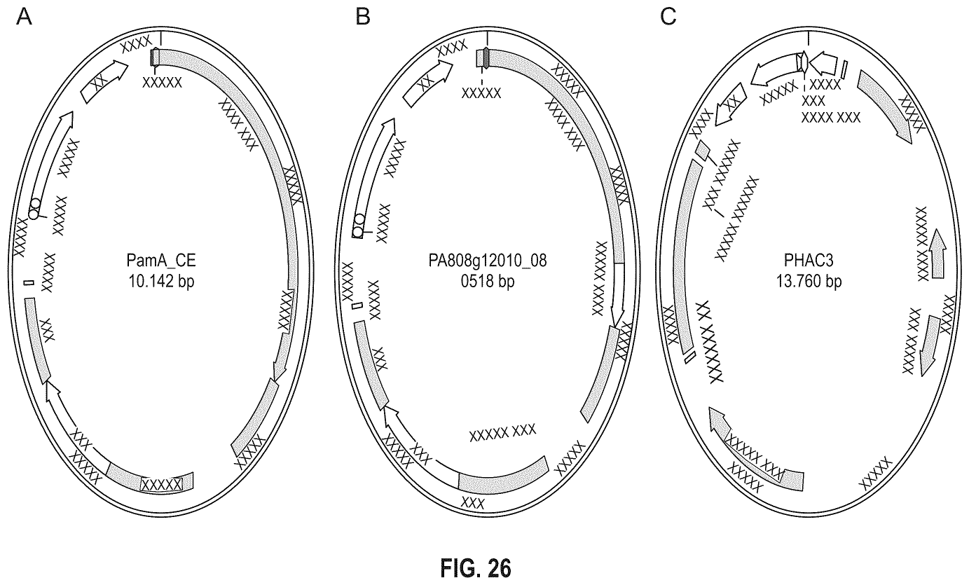

There is also a predicted HAC gene homolog within this region (An08g12010) that shares 41% identify with the predicted protein product of an unannotated ORF within A. fumigatus AF293 HAC (proposed A. fumigatus gene ID: Afu5g14915) and 38% identify with a protein (AFUB_044360) encoded in an orthologous HAC cluster in A. fumigatus strain A1163 (FIG. 25). In both cases higher identity occurs at the c-terminal region (FIG. 25). Afu5g14915 and AFUB_044360 share 78% amino acid identity, and overexpression of AFUB_044360 in a strain where Afu5g14915 has been deleted was sufficient to complement this loss in terms of colony morphology. Therefore, the A. niger (An08g12010) and A. fumigatus (A1163: AFUB_044360; AF293: Afu5g14915) homologs of this gene will be overexpressed using the well-characterized Aspergillus nidulans constitutive promoter gpdA and terminator trpC with the dominant Hygromycin resistance marker to determine if this gene alone, when highly expressed, is sufficient to generate an A. niger strain with reduced O₂ consumption (sample vector: FIG. 26). All strains will be confirmed through Sanger sequencing, Southern analyses, and qRT-PCR. Finalized strain(s) that show phenotypes of interest (high biomass yield, reduced oxygen consumption, citric acid production) will be sent for whole genome sequencing. Example cloning strategies for A. niger are shown in FIG. 26. Sequences and vectors are shown below.

### Sequences and Vectors Utilized in Example 2:

Strategy I: Over expression of A. fumigatus hrmA hypoxia evolved allele in A. niger: the below genomic sequence of hrmA (Insert I) will be amplified with Primer 1 and Primer 2, digested with restriction enzymes Not1-HF and Asc1 and ligated into the below over expression vector sequence with the dominant Hygromycin marker (Vector I) for selection in A. niger. The same insert sequence will also be introduced into Saccharomyces cerevisiae.
Insert I: A. fumigatus hrmA/Afu5g14900 hypoxia evolved genomic sequence

(SEQ ID NO: 1)
ATGGCATCCACAAAGCCCGCTTCGAGTCTCATTTACCAGGCATGGAACAAACTCA
GTATCAACCAAACCATCCCTAGTGACTCCCTTGAATTACTTGGGGAGCGTTTGGC
TATTGCCTTCGCACCCAAACTCAAGGAGCAACGAAGGAATGGCCGGCGTCGGAA
TCTGGAATATGTGGCACAACATCGACGGAAGATTGCTCGAAAAATCTACTTGGA
GATTCTGGAGAAAGACCCAAATATCTTTCTTCCTTTTATCCTGGCTGTTTCCCCTA
GAGCATGCTTATCCTTTGATATCTCGAGCTTTCTTGAACAGCACCAAAGCCAAGG
AAGACATTTCCTCCGCAACAATGCCGAAGCGATCCTCTGGGGTCTCGCAAAGAA
ACATGACATTGATGGCTCCCTCCATTTCAGGAAGCTGATGCGTGAGATTTTCCAA
CTGTCTCCTCCAGCGACAGAAGCCGAAGGCAAGGAGCATTATTCATTGCATTTAA
GCACTCTCCCCGCAATCCGCAATGCCTTCGGTGATGTTATCTTTGACGCAATTGA
ACGTTCCCCTACACAGGTGACAGCGAGAGCTAAAGGTTATTTCTCTGAGAAAAC
CGAAAGTGTTTGGACAAAAGTTCCCTACAGAAGTTCTCAAGACGCAATCATATCT
CTTGAAGTAGGGTCGGCAATCGAGCTTGCGAATGTGTTGTTCCCAATCGCAACCC
AAAAAATTGTCTCTATCCTTTCCGCATGTTCTCCCACTGTGCGCCAGAAGAACTTT
TCTGAGGCTATTCTCGGCCCAGACCCTCAGGATACACCGGCAACATCATCAGAA
ATCGgtatgaagtttaaggtacacatgactgcagttgctaattccaccctgtgctagATGTGGCGTATTTTACTCTG
CGAGGAGCAACGGTCTCGGCAATTGAATCAGTCTTTCGCGCTGATATTTGCGAAG
GTATTAAGGgCAGCGAACTGAGAAACTGGGAAAAGGAGCAGCTGCTCATCGACA
CGACAGATTGTGTCACGATGCAGATATGGCGGGCACAACCTCAACATGGAACCA
TCAAGTTGCGTATTGGATTCTATGCAGCGGTGAATTTGGCAAATCGGCTGTATGC
AGAAACACCCCAAGATCACATATAG

g: the sequence change as a result of in vitro evolution in hypoxia

Primer 1:
(SEQ ID NO: 34)
AAAAAAAAGGCGCGCCATGGCATCCACAAAGCCCGCTT
Primer 2:
(SEQ ID NO: 35)
AAAAAAAAGCGGCCGCCTATATGTGATCTTGGGGTGTTTCTGCAT

Vector I: Overexpression vector with Hygromycin marker

(SEQ ID NO: 36)
ggccgcgaAGCTTGAGATCCACTTAACGTTACTGAAATCATCAAACAGCTTGACG
AATCTGGATATAAGATCGTTGGTGTCGATGTCAGCTCCGGAGTTGAGACAA
ATGGTGTTCAGGATCTCGATAAGATACGTTCATTTGTCCAAGCAGCAAAGAG
TGCCTTCTAGTGATTTAATAGCTCCATGTCAACAAGAATAAAACGCGTTTTC
GGGTTTACCTCTTCCAGATACAGCTCATCTGCAATGCATTAATGCATTGACT
Gcaacctagtaacgccttncaggctccggcgaagagaagaatagcttagcagagctattttcattttcgggagacgagatcaagcag
atcaacggtcgtcaagagacctacgagactgaggaatccgctcttggctccacgcgactatatatttgtctctaattgtactttgacatgct
cctcttctttactctgatagcttgactatgaaaattccgtcaccagcncctgggttcgcaaagataattgcatgtttcttccttgaactctcaa
gcctacaggacacacattcatcgtaggtataaacctcgaaatcanttcctactaagatggtatacaatagtaaccatgcatggttgcctag
tgaatgctccgtaacacccaatacgccggccgaaacttttttacaactctcctatgagtcgtttacccagaatgcacaggtacacttgttta
gaggtaatccttctttggggatctgacagacgggcaattgattacgggatcccattggtaacgaaatgtaaaagctaggagatcgtccg
ccgatgtcaggatgatttcacttgtttcttgtccggctcaccggtcaaagctaaagaggagcaaaaggaacggatagaatcgggtgcc
gctgatctatacggtatagtgcccttatcacgttgactcaacccatgctatttaactcaacccctccttctgaaccccaccatcttcttcctttt
cctctcatcccacacaattctctatctcagatttgaattccaaaagtcctcggacgaaactgaacaagtcttcctcccttcgataaacctttg
gtgattggaataactgaccatcttctatagttcccaaaccaaccgacaatgtaaatacactcctcgattagccctCTAGTatccttgaa
gctgtccctgatggtcgtcatctacctgcctggacagcatggcctgcaacgcgggcatcccgatgccgccggaagcgagaagaatca
taatggggaaggccatccagcctcgcgtcgagctttgaagttgctgcaagctggcttcaagccatcccatccgaatgtgatgga
tgcgttctttctgggccgttgcgactttggggatcgtctttcccgcgcccttggttggaggccctgtctccggtgtcccttgtccctt
ccaggcaagcgagcgaggtccattcagatggtgctccatcagcgttggctttccgtctccattggctcttggcaattcggtcagc
ggggctgactgcctcaggtggggcagtgctagtgtgtgtaccgacccgcaggattggtgctttgcccagagctctacagaatagcgc
gcgcatccatatgttagttctgcaattttcttgtatcggtgctgtgactcatacttccccctttggctggccttgcggcaaccaataagaacg
cacagtgaaatcttgcgggtggggagtggatccatggcgcctgcattggcttggggacgcgcactgtcgcacacttccatctgaccttt
cagaagggtttcgtggtgggcaaggaccaaccggttgcgcggccgtgcgtgggtgcctcgcccggcactgccagggccactgcag
tggcagtttgctgcctgatacaaaatccttccctccgcccagttttccctctttgaccttcctttctcttctctgcaaccaaatccaccctatca
aaccaaaacagtatctcgaccgaggtatcaacctgaatcagcaacatcgtagccagcatttgtctccgtctctgcagaaccagcgagtt
gcaaacattatccaggcaacagggcaccaactcacttcttcggctttcaccaatcggtacagctcttctcagaactcgcgtccgcaaca
gttctacgcttcctcagcaccttcttcagcttcaatcctgaacactcagaaccgcgcacagcagcgccctcctgttcccttgtttcccaaaa
gtaccggtagtatttcgcacggaaagcagggcaacaagatgttctcaggtacccatatgaaaaagcctgaactcaccgcgacgtctgt
cgagaagtttctgatcgaaaagttcgacagcgtctccgacctgatgcagctctcggagggcgaagaatctcgtgctttcagcttcgatgt
aggagggcgtggatatgtcctgcgggtaaatagctgcgccgatggtttctacaaagatcgttatgtttatcggcactttgcatcggccgc
gctcccgattccggaagtgcttgacattggggaattcagcgagagcctgacctattgcatctcccgccgtgcacagggtgtcacgttgc
aagacctgcctgaaaccgaactgcccgctgttctgcagccggtcgcggaggccatggatgcgatcgctgcggccgatcttagccag
acgagcgggttcggcccattcggaccgcaaggaatcggtcaatacactacatggcgtgatttcatatgcgcgattgctgatccccatgt
gtatcactggcaaactgtgatggacgacaccgtcagtgcgtccgtcgcgcaggctctcgatgagctgatgctttgggccgaggactgc
cccgaagtccggcacctcgtgcacgcggatttcggctccaacaatgtcctgacggacaatggccgcataacagcggtcattgactgg
agcgaggcgatgttcggggattcccaatacgaggtcgccaacatcttcttctggaggccgtggttggcttgtatggagcagcagacgc
gctacttcgagcggaggcatccggagcttgcaggatcgccgcggctccgggcgtatatgctccgcattggtcttgaccaactctatca
gagcttggttgacggcaatttcgatgatgcagcttgggcgcagggtcgatgcgacgcaatcgtccgatccggagccgggactgtcgg
gcgtacacaaatcgcccgcagaagcgcggccgtctggaccgatggctgtgtagaagtactcgccgatagtggaaaccgacgcccc
agcactcgtccgagggcaaaggaatagagtagatgccgaccgggatccacttaacgttactgaaatcatcaaacagcttgacgaatct
ggatataagatcgttggtgtcgatgtcagctccggagttgagacaaatggtgttcaggatctcgataagatacgttcatttgtccaagcag
caaagagtgccttctagtgatttaatagctccatgtcaacaagaataaaacgcgtttcgggtttacctcttccagatacagctcatctgcaa
tgcattaatgcattggacctcgcaaccctagtacgcccttcaggctccggcgaagcagaagaatagcttagcagagtctattttcattttc
gggagacgagatcaagcagatcaacggtcgtcaagagacctacgagactgaggaatccgctcttggctccacgcgactatatatttgt
ctctaattgtactttgacatgctcctcttctttactctgatagcttgactatgaaaattccgtcaccagcccctgggttcgcaaagataattgc
actgtttcttccttgaactctcaagcctacaggacacacattcatcgtaggtataaacctcgaaaatcattcctactaagatgggtatacaat
agtaaccatggttgcctagtgaatgctccgtaacacccaatacgccggccgaaacttttttacaactctcctatgagtcgtttacccagaat
gcacaggtacacttgtttagaggtaatccttctttctagctagaggatcctctacgccggacgcatcgtggccggcatcaccggcgcca
caggtgcggttgctgActagaataattatgtgtaacaagaaagacagtataatacaaacaaagatgcaagagcggctcatcgtcaccc
catgatagctagagcttggcactggccgtcgttttacaacgtcgtgactgggaaaaccctggcgttacccaacttaatcgccttgcagca
catccccctttcgccagctggcgtaatagcgaagaggcccgcaccgatcgcccttcccaacagttgcgcagcctgaatggcgaatgg
cgcctgatgcggtattttctccttacgcatctgtgcggtatttcacaccgcatatggtgcactctcagtacaatctgctctgatgccgcatag
ttaagccagccccgacacccgccaacacccgctgacgcgccctgacgggcttgtctgctcccggcatccgcttacagacaagctgtg
accgtctccgggagctgcatgtgtcagaggttttcaccgtcatcaccgaaacgcgcgagacgaaagggcctcgtgatacgcctattttt
ataggttaatgtcatgataataatggtttcttagacgtcaggtggcacttttcggggaaatgtgcgcggaacccctatttgtttatttttctaaa
tacattcaaatatgtatccgctcatgagacaataaccctgataaatgcttcaataatattgaaaaaggaagagtatgagtattcaacatttcc
gtgtcgcccttattcccttttttgcggcattttgccttcctgtttttgctcacccagaaacgctggtgaaagtaaaagatgctgaagatcagtt
gggtgcacgagtgggttacatcgaactggatctcaacagcggtaagatccttgagagttttcgccccgaagaacgttttccaatgatga
gcacttttaaagttctgctatgtggcgcggtattatcccgtattgacgccgggcaagagcaactcggtcgccgcatacactattctcaga
atgacttggttgagtactcaccagtcacagaaaagcatcttacggatggcatgacagtaagagaattatgcagtgctgccataaccatg
agtgataacactgcggccaacttacttctgacaacgatcggaggaccgaaggagctaaccgcttttttgcacaacatgggggatcatgt
aactcgccttgatcgttgggaaccggagctgaatgaagccataccaaacgacgagcgtgacaccacgatgcctgtagcaatggcaac
aacgttgcgcaaactattaactggcgaactacttactctagcttcccggcaacaattaatagactggatggaggcggataaagttgcag
gaccacttctgcgctcggcccttccggctggctggtttattgctgataaatctggagccggtgagcgtgggtctcgcggtatcattgcag
cactggggccagatggtaagccctcccgtatcgtagttatctacacgacggggagtcaggcaactatggatgaacgaaatagacaga
tcgctgagataggtgcctcactgattaagcattggtaactgtcagaccaagtttactcatatatactttagattgatttaaaacttcatttttaat
ttaaaaggatctaggtgaagatcctttttgataatctcatgaccaaaatcccttaacgtgagttttcgttccactgagcgtcagaccccgta
gaaaagatcaaaggatcttcttgagatcctttttttctgcgcgtaatctgctgcttgcaaacaaaaaaaccaccgctaccagcggtggtttg
tttgccggatcaagagctaccaactctttttccgaaggtaactggcttcagcagagcgcagataccaaatactgtccttctagtgtagccg
tagttaggccaccacttcaagaactctgtagcaccgcctacatacctcgctctgctaatcctgttaccagtggctgctgccagtggcgat
aagtcgtgtcttaccgggttggactcaagacgatagttaccggataaggcgcagcggtcgggctgaacggggggttcgtgcacacag
cccagcttggagcgaacgacctacaccgaactgagatacctacagcgtgagctatgagaaagcgccacgcttcccgaagggagaa
aggcggacaggtatccggtaagcggcagggtcggaacaggagagcgcacgagggagcttccagggggaaacgcctggtatcttt
atagtcctgtcgggtttcgccacctctgacttgagcgtcgatttttgtgatgctcgtcaggggggcggagcctatggaaaaacgccagc
aacgcggcctttttacggttcctggccttttgctggccttttgctcacatgttctttcctgcgttatcccctgattctgtggataaccgtattacc
gcctttgagtgagctgataccgctcgccgcagccgaacgaccgagcgcagcgagtcagtgagcgaggaagcggaagagcgccca
atacgcaaaccgcctctccccgcgcgttggccgattcattaatgcagctggcacgacaggtttcccgactggaaagcgggcagtgag
cgcaacgcaattaatgtgagttagctcactcattaggcaccccaggctttacactttatgcttccggctcgtatgttgtgtggaattgtgag
cggataacaatttcacacaggaaacagctatgaccatgattacgaattcccttgtatctctacacacaggctcaaatcaataagaaga
acggttcgtctttttcgtttatatcttgcatcgtcccaaagctattggcgggatattctgtttgcagttggctgacttgaagtaatctctgca
gatctttcgacactgaaatacgtcgagcctgctccgcttggaagcggcgaggagcctcgtcctgtcacaactaccaacatggagta
cgataagggccagttccgccagctcattaagagccagttcatgggcgttggcatgatggccgtcatgcatctgtacttcaagtacacc
aacgctcttctgatccagtcgatcatccgctgaaggcgctttcgaatctggttaagatccacgtcttcgggaagccagcgactggtgacc
tccagcgtccctttaaggctgccaacagctttctcagccagggccagcccaagaccgacaaggcctccctccagaacgccgagaag
aactggaggggtggtgtcaaggaggagtaagctccttattgaagtcggaggacggagcggtgtcaagaggatattcttcgactctgta
ttatagataagatgatgaggaattggaggtagcatagcttcatttggatttgctttccaggctgagactctagcttggagcatagagggtc
ctttggctttcaatattctcaagtatctcgagtttgaacttattccctgtgaaccttttattcaccaatgagcattggaatgaacatgaatctga
ggactgcaatcgccatgaggttttcgaaatacatccggatgtcgaaggcttggggcacctgcgttggttgaatttagaacgtggcactat
tgatcatccgatagctctgcaaagggcgttgcacaatgcaagtcaaacgttgctagcagttccaggtggaatgttatgatgagcattgtat
taaatcaggagatatagcatgatctctagttagctcaccacaaaagtcagacggcgtaaccaaaagtcacacaacacaagctgtaagg
atttcggcacggctacggaagacggagaagccaccttcagtggactcgagtaccatttaattctatttgtgtttgatcgagacctaataca
gcccctacaacgaccatcaaagtcgtatagctaccagtgaggaagtggactcaaatcgacttcagcaacatctcctggataaactttaa
gcctaaactatacagaataagataggtggagagcttataccgagctcccaaatctgtccagatcatggttgaccggtgcctggatcttcc
tatagaatcatccttattcgttgacctagctgattctggagtgacccagagggtcatgacttgagcctaaaatccgccgcctccaccatttg
tagaaaaatgtgacgaactcgtgagctctgtacagtgaccggtgactctttctggcatgcggagagacggacggacgcagagagaag
ggctgagtaataagccactggccagacagctctggcggctctgaggtgcagtggatgattattaatccgggaccggccgcccctccg
ccccgaagtggaaaggctggtgtgcccctcgttgaccaagaatctattgcatcatcggagaatatggagcttcatcgaatcaccggca
gtaagcgaaggagaatgtgaagccaggggtgtatagccgtcggcgaaatagcatgccattaacctaggtacagaagtccaattgcttc
cgatctggtaaaagattcacgagatagtaccttctccgaagtaggtagagcgagtacccggcgcgtaagctccctaattggcccatcc
ggcatctgtagggcgtccaaatatcgtgcctctcctgctttgcccggtgtatgaaaccggaaaggccgctcaggagctggccagcgg
cgcagaccgggaacacaagctggcagtcgacccatccggtgctctgcactcgacctgctgaggtccctcagtccctggtaggcagct
ttgccccgtctgtccgcccggtgtgtcggcggggttgacaaggtcgttgcgtcagtccaacatttgttgccatattttcctgctctccccac
cagctgctcttttcttttctctttcttttcccatcttcagtatattcatcttcccatccaagaacctttatttcccctaagtaagtactttgcta
catccatactccatccttcccatcccttattcctttgaacctttcagttcgagctttcccacttcatcgcagcttgactaacagctaccccgctt
gagcagacatcaccatgggg

### Key:

gpdA Promoter

### TRPC Terminator

### Hygromycin Marker

g: the sequence change as a result of in vitro evolution in hypoxia
Product I: Over expression of A. fumigatus hrmA evolved allele with Hygromycin

(SEQ ID NO: 37)
gaattcccttgtatctctacacacaggctcaaatcaataagaagaacggttcgtctttttcgtttatatcttgcatcgtcccaaagctattggc
gggatattctgtttgcagttggctgacttgaagtaatctctgcagatctttcgacactgaaatacgtcgagcctgctccgcttggaagcgg
cgaggagcctcgtcctgtcacaactaccaacatggagtacgataagggccagttccgccagctcattaagagccagttcatgggcgtt
ggcatgatggccgtcatgcatctgtacttcaagtacaccaacgctcttctgatccagtcgatcatccgctgaaggcgctttcgaatctggt
taagatccacgtcttcgggaagccagcgactggtgacctccagcgtccctttaaggctgccaacagctttctcagccagggccagccc
aagaccgacaaggcctccctccagaacgccgagaagaactggaggggtggtgtcaaggaggagtaagctccttattgaagtcgga
ggacggagcggtgtcaagaggatattcttcgactctgtattatagataagatgatgaggaattggaggtagcatagcttcatttggatttg
ctttccaggctgagactctagcttggagcatagagggtcctttggctttcaatattctcaagtatctcgagtttgaacttattccctgtgaacc
ttttattcaccaatgagcattggaatgaacatgaatctgaggactgcaatcgccatgaggttttcgaaatacatccggatgtcgaaggctt
ggggcacctgcgttggttgaatttagaacgtggcactattgatcatccgatagctctgcaaagggcgttgcacaatgcaagtcaaacgtt
gctagcagttccaggtggaatgttatgatgagcattgtattaaatcaggagatatagcatgatctctagttagctcaccacaaaagtcaga
cggcgtaaccaaaagtcacacaacacaagctgtaaggatttcggcacggctacggaagacggagaagccaccttcagtggactcga
gtaccatttaattctatttgtgtttgatcgagacctaatacagcccctacaacgaccatcaaagtcgtatagctaccagtgaggaagtgga
ctcaaatcgacttcagcaacatctcctggataaactttaagcctaaactatacagaataagataggtggagagcttataccgagctccca
aatctgtccagatcatggttgaccggtgcctggatcttcctatagaatcatccttattcgttgacctagctgattctggagtgacccagagg
gtcatgacttgagcctaaaatccgccgcctccaccatttgtagaaaaatgtgacgaactcgtgagctctgtacagtgaccggtgactcttt
ctggcatgcggagagacggacggacgcagagagaagggctgagtaataagccactggccagacagctctggcggctctgaggtg
cagtggatgattattaatccgggaccggccgcccctccgccccgaagtggaaaggctggtgtgcccctcgttgaccaagaatctattg
catcatcggagaatatggagcttcatcgaatcaccggcagtaagcgaaggagaatgtgaagccaggggtgtatagccgtcggcgaa
atagcatgccattaacctaggtacagaagtccaattgcttccgatctggtaaaagattcacgagatagtaccttctccgaagtaggtaga
gcgagtacccggcgcgtaagctccctaattggcccatccggcatctgtagggcgtccaaatatcgtgcctctcctgctttgcccggtgt
atgaaaccggaaaggccgctcaggagctggccagcggcgcagaccgggaacacaagctggcagtcgacccatccggtgctctgc
actcgacctgctgaggtccctcagtccctggtaggcagctttgccccgtctgtccgcccggtgtgtcggcggggttgacaaggtcgttg
cgtcagtccaacatttgttgccatattttcctgctctccccaccagctgctcttttcttttctctttcttttcccatcttcagtatattcatctt
cccatccaagaacctttatttcccctaagtaagtactttgctacatccatactccatccttcccatcccttattcctttgaacctttcagttcgag
ctttcccacttcatcgcagcttgactaacagctaccccgcttgagcagacatcaccatggggcgcgccAAAAAAAAGGCGCGC
CATGGCATCCACAAAGCCCGCTTCGAGTCTCATTTACCAGGCATGGAACAAACTC
AGTATCAACCAAACCATCCCTAGTGACTCCCTTGAATTACTTGGGGAGCGTTTGG
CTATTGCCTTCGCACCCAAACTCAAGGAGCAACGAAGGAATGGCCGGCGTCGGA
ATCTGGAATATGTGGCACAACATCGACGGAAGATTGCTCGAAAAATCTACTTGG
AGATTCTGGAGAAAGACCCAAATATCTTTCTTCCTTTTATCCTGGCTGTTTCCCCT
AGAGCATGCTTATCCTTTGATATCTCGAGCTTTCTTGAACAGCACCAAAGCCAAG
GAAGACATTTCCTCCGCAACAATGCCGAAGCGATCCTCTGGGGTCTCGCAAAGA
AACATGACATTGATGGCTCCCTCCATTTCAGGAAGCTGATGCGTGAGATTTTCCA
ACTGTCTCCTCCAGCGACAGAAGCCGAAGGCAAGGAGCATTATTCATTGCATTTA
AGCACTCTCCCCGCAATCCGCAATGCCTTCGGTGATGTTATCTTTGACGCAATTG
AACGTTCCCCTACACAGGTGACAGCGAGAGCTAAAGGTTATTTCTCTGAGAAAA
CCGAAAGTGTTTGGACAAAAGTTCCCTACAGAAGTTCTCAAGACGCAATCATATC
TCTTGAAGTAGGGTCGGCAATCGAGCTTGCGAATGTGTTGTTCCCAATCGCAACC
CAAAAAATTGTCTCTATCCTTTCCGCATGTTCTCCCACTGTGCGCCAGAAGAACT
TTTCTGAGGCTATTCTCGGCCCAGACCCTCAGGATACACCGGCAACATCATCAGA
AATCGGTATGAAGTTTAAGGTACACATGACTGCAGTTGCTAATTCCACCCTGTGC
TAGATGTGGCGTATTTTACTCTGCGAGGAGCAACGGTCTCGGCAATTGAATCAGT
CTTTCGCGCTGATATTTGCGAAGGTATTAAGGgCAGCGAACTGAGAAACTGGGAA
AAGGAGCAGCTGCTCATCGACACGACAGATTGTGTCACGATGCAGATATGGCGG
GCACAACCTCAACATGGAACCATCAAGTTGCGTATTGGATTCTATGCAGCGGTGA
ATTTGGCAAATCGGCTGTATGCAGAAACACCCCAAGATCACATATAGgcggccgcgaa
gcttgagatccacttaacgttactgaaatcatcaaacagcttgacgaatctggatataagatcgttggtgtcgatgtcagctccggagttg
agacaaatggtgttcaggatctcgataagatacgttcatttgtccaagcagcaaagagtgccttctagtgatttaatagctccatgtcaaca
agaataaaacgcgttttcgggtttacctcttccagatacagctcatctgcaatgcattaatgcattgactgcaacctagtaacgccttncag
gctccggcgaagagaagaatagcttagcagagctattttcattttcgggagacgagatcaagcagatcaacggtcgtcaagagaccta
cgagactgaggaatccgctcttggctccacgcgactatatatttgtctctaattgtactttgacatgctcctcttctttactctgatagcttgac
tatgaaaattccgtcaccagcncctgggttcgcaaagataattgcatgtttcttccttgaactctcaagcctacaggacacacattcatcgt
aggtataaacctcgaaatcanttcctactaagatggtatacaatagtaaccatgcatggttgcctagtgaatgctccgtaacacccaatac
gccggccgaaacttttttacaactctcctatgagtcgtttacccagaatgcacaggtacacttgtttagaggtaatccttctttctagaggcc
tcaaacaatgctcttcaccctcttcgcgggtctgaaataccctcacctggcaacagcaattggcgcttcatggctgtttttccgatctctcta
cttgtacggctatgtgtactcgggtaagccacaaggcaagggcagattgctgggaggtttcttctggttttctcaaggcgctctgtgggct
ctgagtgtgtttggtgttgccaaagacatgatctcttactgagagttattctgtgtctgacgaaatatgttgtgtatatatatatatgtacgttaa
aagttccgtggagttaccagtgattgaccaatgttttatcttctacagttctgcctgtctaccccattctagctgtacctgactacagaatagt
ttaattgtggttgaccccacagtcggaggcggaggaatacagcaccgatgtggcctgtctccatccagattggcacgcaatttttacac
gcggaaaagatcgagatagagtacgactttaaatttagtccccggcggcttctattttagaatatttgagatttgattctcaagcaattgattt
ggttgggtcaccctcaattggataatatacctcattgctcggctacttcaactcatcaatcaccgtcataccccgcatataaccctccattc
ccacgatgtcgtccaagtcgcaattgacttacggtgctcgagccagcaagcaccccaatcctctggcaaagagactttttgagattgcc
gaagcaaagaagacaaacgttaccgtctctgctgatgtgacgacaacccgagaactcctggacctcgctgaccgtacggaagctgtt
ggatccaatacatatgccgtctagcaatggactaatcaacttttgatgatacaggtctcggtccctacatcgccgtcatcaagacacacat
cgacatcctcaccgatttcagcgtcgacactatcaatggcctgaatgtgctggctcaaaagcacaactttttgatcttcgaggaccgcaa
attcatcgacatcggcaataccgtccagaagcaataccacggcggtgctctgaggatctccgaatgggcccacattatcaactgcagc
gttctccctggcgagggcatcgtcgaggctctggcccagaccgcatctgcgcaagacttcccctatggtcctgagagaggactgttgg
tcctggcagagatgacctccaaaggatcgctggctacgggcgagtataccaaggcatcggttgactacgctcgcaaatacaagaactt
cgttatgggtttcgtgtcgacgcgggccctgacggaagtgcagtcggatgtgtcttcagcctcggaggatgaagatttcgtggtcttcac
gacgggtgtgaacctctcttccaaaggagataagcttggacagcaataccagactcctgcatcggctattggacgcggtgccgacttta
tcatcgccggtcgaggcatctacgctgctcccgacccggttgaagctgcacagcggtaccagaaagaaggctgggaagcttatatgg
ccagagtatgcggcaagtcatgatttcctcttggagcaaaagtgtagtgccagtacgagtgttgtggaggaaggctgcatacattgtgc
ctgtcattaaacgatgagctcgtccgtattggcccctgtaatgccatgttttccgcccccaatcgtcaaggttttccctttgttagattcctac
cagtcatctagcaagtgaggtaagctttgccagaaacgccaaggctttatctatgtagtcgataagcaaagtggactgatagcttaatat
ggaaggtccctcagggacaagtcgacctgtgcagaagagataacagcttggcatcacgcatcagtgcctcctctcagacagaatcta
gagcttggcactggccgtcgttttacaacgtcgtgactgggaaaaccctggcgttacccaacttaatcgccttgcagcacatcccccttt
cgccagctggcgtaatagcgaagaggcccgcaccgatcgcccttcccaacagttgcgcagcctgaatggcgaatggcgcctgatgc
ggtattttctccttacgcatctgtgcggtatttcacaccgcatatggtgcactctcagtacaatctgctctgatgccgcatagttaagccagc
cccgacacccgccaacacccgctgacgcgccctgacgggcttgtctgctcccggcatccgcttacagacaagctgtgaccgtctccg
ggagctgcatgtgtcagaggttttcaccgtcatcaccgaaacgcgcgagacgaaagggcctcgtgatacgcctatttttataggttaatg
tcatgataataatggtttcttagacgtcaggtggcacttttcggggaaatgtgcgcggaacccctatttgtttatttttctaaatacattcaaat
atgtatccgctcatgagacaataaccctgataaatgcttcaataatattgaaaaaggaagagtatgagtattcaacatttccgtgtcgccct
tattcccttttttgcggcattttgccttcctgtttttgctcacccagaaacgctggtgaaagtaaaagatgctgaagatcagttgggtgcacg
agtgggttacatcgaactggatctcaacagcggtaagatccttgagagttttcgccccgaagaacgttttccaatgatgagcacttttaaa
gttctgctatgtggcgcggtattatcccgtattgacgccgggcaagagcaactcggtcgccgcatacactattctcagaatgacttggtt
gagtactcaccagtcacagaaaagcatcttacggatggcatgacagtaagagaattatgcagtgctgccataaccatgagtgataaca
ctgcggccaacttacttctgacaacgatcggaggaccgaaggagctaaccgcttttttgcacaacatgggggatcatgtaactcgcctt
gatcgttgggaaccggagctgaatgaagccataccaaacgacgagcgtgacaccacgatgcctgtagcaatggcaacaacgttgcg
caaactattaactggcgaactacttactctagcttcccggcaacaattaatagactggatggaggcggataaagttgcaggaccacttct
gcgctcggcccttccggctggctggtttattgctgataaatctggagccggtgagcgtgggtctcgcggtatcattgcagcactggggc
cagatggtaagccctcccgtatcgtagttatctacacgacggggagtcaggcaactatggatgaacgaaatagacagatcgctgagat
aggtgcctcactgattaagcattggtaactgtcagaccaagtttactcatatatactttagattgatttaaaacttcatttttaatttaaaaggat
ctaggtgaagatcctttttgataatctcatgaccaaaatcccttaacgtgagttttcgttccactgagcgtcagaccccgtagaaaagatca
aaggatcttcttgagatcctttttttctgcgcgtaatctgctgcttgcaaacaaaaaaaccaccgctaccagcggtggtttgtttgccggatc
aagagctaccaactctttttccgaaggtaactggcttcagcagagcgcagataccaaatactgtccttctagtgtagccgtagttaggcc
accacttcaagaactctgtagcaccgcctacatacctcgctctgctaatcctgttaccagtggctgctgccagtggcgataagtcgtgtct
taccgggttggactcaagacgatagttaccggataaggcgcagcggtcgggctgaacggggggttcgtgcacacagcccagcttgg
agcgaacgacctacaccgaactgagatacctacagcgtgagctatgagaaagcgccacgcttcccgaagggagaaaggcggaca
ggtatccggtaagcggcagggtcggaacaggagagcgcacgagggagcttccagggggaaacgcctggtatctttatagtcctgtc
gggtttcgccacctctgacttgagcgtcgatttttgtgatgctcgtcaggggggcggagcctatggaaaaacgccagcaacgcggcct
ttttacggttcctggccttttgctggccttttgctcacatgttctttcctgcgttatcccctgattctgtggataaccgtattaccgcctttgagt
gagctgataccgctcgccgcagccgaacgaccgagcgcagcgagtcagtgagcgaggaagcggaagagcgcccaatacgcaaac
cgcctctccccgcgcgttggccgattcattaatgcagctggcacgacaggtttcccgactggaaagcgggcagtgagcgcaacgcaa
ttaatgtgagttagctcactcattaggcaccccaggctttacactttatgcttccggctcgtatgttgtgtggaattgtgagcggataacaat
ttcacacaggaaacagctatgaccatgattac

Strategy II: Over expression of A. niger putative HAC ortholog An08g12010 in A. niger: the below genomic sequence of Ano8g12010(Insert II) will be amplified with Primer 3 and Primer 4, digested with restriction enzymes Not1-HF and Asc1 and ligated into over expression vector sequence with the dominant Hygromycin marker (Vector I) for selection in A. niger.
Insert II: A. niger An08g12010 genomic sequence

(SEQ ID NO: 6)
ATGGCTTGGTACAGTGCTTTACTCCCGTGCATGCTATGGTGGCGGAACCT
CCTGTGGCGTAACAGCACCAATAGGTATAGACAGAGTACAGACGACCTTA
CTTCACTGACTGATAAGATACCTAATCTTGGAGAAAGGgtaagtgtaaat
actttttgaggtcgcttctaggagtctaatcactttggaaagacaactcc
cacagataacatcgatggaaagagcttcgtggcacatactgtcgtgcaga
ccaggcggattggtgaccgagaacgttgctgcttcaatgactcggatttg
aacagcggcacagTTACCAAATTTATTGAGCTTTGTTAATAGCATTCCAG
ATCAGAGTACTACTGCCGGAAAAATTAATCACAAGGCCCTGCGAGATCCT
GAGCTCTTTGCTATCCGCTCGTCATGCATCGACAAATCAGCCAGCAAGTG
GATGGTTAGTCTCTACTACGAACCCCCACCCAGCCTTGATGACCTCGAGA
TTAAGAACTTCGGATCTCGTATTCCAGAGTCGGAGGATGATCCAATTGAG
GCTATCTTTCACTATGAGGGAGAGAACATTTGGGTATCTGTTCCTTATTT
GTATGCTAGAACTAGAAGCCTTTCAAGCGGTCTGTTCTGA
Primer 3:
(SEQ ID NO: 38)
AAAAGGCGCGCCATGGCTTGGTACAGTGCTTTACTC
Primer 4:
(SEQ ID NO: 39)
AAAAGCGGCCGCTCAGAACAGACCGCTTGAAAGGC

Product II: Over expression of A. niger An08g12010 with Hygromycin

(SEQ ID NO: 40)
gaattcccttgtatctctacacacaggctcaaatcaataagaagaacggttcgtctttttcgtttatatcttgcatcgtcccaaagctattggc
gggatattctgtttgcagttggctgacttgaagtaatctctgcagatctttcgacactgaaatacgtcgagcctgctccgcttggaagcgg
cgaggagcctcgtcctgtcacaactaccaacatggagtacgataagggccagttccgccagctcattaagagccagttcatgggcgtt
ggcatgatggccgtcatgcatctgtacttcaagtacaccaacgctcttctgatccagtcgatcatccgctgaaggcgctttcgaatctggt
taagatccacgtcttcgggaagccagcgactggtgacctccagcgtccctttaaggctgccaacagctttctcagccagggccagccc
aagaccgacaaggcctccctccagaacgccgagaagaactggaggggtggtgtcaaggaggagtaagctccttattgaagtcgga
ggacggagcggtgtcaagaggatattcttcgactctgtattatagataagatgatgaggaattggaggtagcatagcttcatttggatttg
ctttccaggctgagactctagcttggagcatagagggtcctttggctttcaatattctcaagtatctcgagtttgaacttattccctgtgaacc
ttttattcaccaatgagcattggaatgaacatgaatctgaggactgcaatcgccatgaggttttcgaaatacatccggatgtcgaaggctt
ggggcacctgcgttggttgaatttagaacgtggcactattgatcatccgatagctctgcaaagggcgttgcacaatgcaagtcaaacgtt
gctagcagttccaggtggaatgttatgatgagcattgtattaaatcaggagatatagcatgatctctagttagctcaccacaaaagtcaga
cggcgtaaccaaaagtcacacaacacaagctgtaaggatttcggcacggctacggaagacggagaagccaccttcagtggactcga
gtaccatttaattctatttgtgtttgatcgagacctaatacagcccctacaacgaccatcaaagtcgtatagctaccagtgaggaagtgga
ctcaaatcgacttcagcaacatctcctggataaactttaagcctaaactatacagaataagataggtggagagcttataccgagctccca
aatctgtccagatcatggttgaccggtgcctggatcttcctatagaatcatccttattcgttgacctagctgattctggagtgacccagagg
gtcatgacttgagcctaaaatccgccgcctccaccatttgtagaaaaatgtgacgaactcgtgagctctgtacagtgaccggtgactcttt
ctggcatgcggagagacggacggacgcagagagaagggctgagtaataagccactggccagacagctctggcggctctgaggtg
cagtggatgattattaatccgggaccggccgcccctccgccccgaagtggaaaggctggtgtgcccctcgttgaccaagaatctattg
catcatcggagaatatggagcttcatcgaatcaccggcagtaagcgaaggagaatgtgaagccaggggtgtatagccgtcggcgaa
atagcatgccattaacctaggtacagaagtccaattgcttccgatctggtaaaagattcacgagatagtaccttctccgaagtaggtaga
gcgagtacccggcgcgtaagctccctaattggcccatccggcatctgtagggcgtccaaatatcgtgcctctcctgctttgcccggtgt
atgaaaccggaaaggccgctcaggagctggccagcggcgcagaccgggaacacaagctggcagtcgacccatccggtgctctgc
actcgacctgctgaggtccctcagtccctggtaggcagctttgccccgtctgtccgcccggtgtgtcggcggggttgacaaggtcgttg
cgtcagtccaacatttgttgccatattttcctgctctccccaccagctgctcttttcttttctctttcttttcccatcttcagtatattcatcttcc
catccaagaacctttatttcccctaagtaagtactttgctacatccatactccatccttcccatcccttattcctttgaacctttcagttcgagcttt
cccacttcatcgcagcttgactaacagctaccccgcttgagcagacatcaccatggggCGCGCCATGGCTTGGTACA
GTGCTTTACTCCCGTGCATGCTATGGTGGCGGAACCTCCTGTGGCGTAACAGCAC
CAATAGGTATAGACAGAGTACAGACGACCTTACTTCACTGACTGATAAGATACC
TAATCTTGGAGAAAGGgtaagtgtaaatactttttgaggtcgcttctaggagtctaatcactttggaaagacaactcccac
agataacatcgatggaaagagcttcgtggcacatactgtcgtgcagaccaggcggattggtgaccgagaacgttgctgcttcaatgac
tcggatttgaacagcggcacagTTACCAAATTTATTGAGCTTTGTTAATAGCATTCCAGATCA
GAGTACTACTGCCGGAAAAATTAATCACAAGGCCCTGCGAGATCCTGAGCTCTTT
GCTATCCGCTCGTCATGCATCGACAAATCAGCCAGCAAGTGGATGGTTAGTCTCT
ACTACGAACCCCCACCCAGCCTTGATGACCTCGAGATTAAGAACTTCGGATCTCG
TATTCCAGAGTCGGAGGATGATCCAATTGAGGCTATCTTTCACTATGAGGGAGAG
AACATTTGGGTATCTGTTCCTTATTTGTATGCTAGAACTAGAAGCCTTTCAAGCG
GTCTGTTCTGAGCggccgcgaagcttgagatccacttaacgttactgaaatcatcaaacagcttgacgaatctggatataag
atcgttggtgtcgatgtcagctccggagttgagacaaatggtgttcaggatctcgataagatacgttcatttgtccaagcagcaaagagt
gccttctagtgatttaatagctccatgtcaacaagaataaaacgcgttttcgggtttacctcttccagatacagctcatctgcaatgcattaat
gcattgactgcaacctagtaacgccttncaggctccggcgaagagaagaatagcttagcagagctattttcattttcgggagacgagat
caagcagatcaacggtcgtcaagagacctacgagactgaggaatccgctcttggctccacgcgactatatatttgtctctaattgtacttt
gacatgctcctcttctttactctgatagcttgactatgaaaattccgtcaccagcncctgggttcgcaaagataattgcatgtttcttccttga
actctcaagcctacaggacacacattcatcgtaggtataaacctcgaaatcanttcctactaagatggtatacaatagtaaccatgcatgg
ttgcctagtgaatgctccgtaacacccaatacgccggccgaaacttttttacaactctcctatgagtcgtttacccagaatgcacaggtac
acttgtttagaggtaatccttctttggggatctgacagacgggcaattgattacgggatcccattggtaacgaaatgtaaaagctaggag
atcgtccgccgatgtcaggatgatttcacttgtttcttgtccggctcaccggtcaaagctaaagaggagcaaaaggaacggatagaatc
gggtgccgctgatctatacggtatagtgcccttatcacgttgactcaacccatgctatttaactcaacccctccttctgaaccccaccatctt
cttccttttcctctcatcccacacaattctctatctcagatttgaattccaaaagtcctcggacgaaactgaacaagtcttcctcccttcgata
aacctttggtgattggaataactgaccatcttctatagttcccaaaccaaccgacaatgtaaatacactcctcgattagccctCTAGTa
tccttgaagctgtccctgatggtcgtcatctacctgcctggacagcatggcctgcaacgcgggcatcccgatgccgccggaagcgag
aagaatcataatggggaaggccatccagcctcgcgtcgagctttgaagttgctgcaagctggcttcaagccatcccatccgaatgtgat
ggatgcgttctttctgggccgttgcgactttggggatcgtctttcccgcgcccttggttggaggccctgtctccggtgtcccttgtcccttc
caggcaagcgagcgaggtccattcagatggtgctccatcagcgttggctttccgtctccattggctcttggcaattcggtcagcggggc
tgactgcctcaggtggggcagtgctagtgtgtgtaccgacccgcaggattggtgctttgcccagagctctacagaatagcgcgcgcat
ccatatgttagttctgcaattttcttgtatcggtgctgtgactcatacttccccctttggctggccttgcggcaaccaataagaacgcacagt
gaaatcttgcgggtggggagtggatccatggcgcctgcattggcttggggacgcgcactgtcgcacacttccatctgacctttcagaa
gggtttcgtggtgggcaaggaccaaccggttgcgcggccgtgcgtgggtgcctcgcccggcactgccagggccactgcagtggca
gtttgctgcctgatacaaaatccttccctccgcccagttttccctctttgaccttcctttctcttctctgcaaccaaatccaccctatcaaacca
aaacagtatctcgaccgaggtatcaacctgaatcagcaacatcgtagccagcatttgtctccgtctctgcagaaccagcgagttgcaaa
cattatccaggcaacagggcaccaactcacttcttcggctttcaccaatcggtacagctcttctcagaactcgcgtccgcaacagttctac
gcttcctcagcaccttcttcagcttcaatcctgaacactcagaaccgcgcacagcagcgccctcctgttcccttgtttcccaaaagtaccg
gtagtatttcgcacggaaagcagggcaacaagatgttctcaggtacccatatgaaaaagcctgaactcaccgcgacgtctgtcgagaa
gtttctgatcgaaaagttcgacagcgtctccgacctgatgcagctctcggagggcgaagaatctcgtgctttcagcttcgatgtaggag
ggcgtggatatgtcctgcgggtaaatagctgcgccgatggtttctacaaagatcgttatgtttatcggcactttgcatcggccgcgctccc
gattccggaagtgcttgacattggggaattcagcgagagcctgacctattgcatctcccgccgtgcacagggtgtcacgttgcaagac
ctgcctgaaaccgaactgcccgctgttctgcagccggtcgcggaggccatggatgcgatcgctgcggccgatcttagccagacgag
cgggttcggcccattcggaccgcaaggaatcggtcaatacactacatggcgtgatttcatatgcgcgattgctgatccccatgtgtatca
ctggcaaactgtgatggacgacaccgtcagtgcgtccgtcgcgcaggctctcgatgagctgatgctttgggccgaggactgccccga
agtccggcacctcgtgcacgcggatttcggctccaacaatgtcctgacggacaatggccgcataacagcggtcattgactggagcga
ggcgatgttcggggattcccaatacgaggtcgccaacatcttcttctggaggccgtggttggcttgtatggagcagcagacgcgctact
tcgagcggaggcatccggagcttgcaggatcgccgcggctccgggcgtatatgctccgcattggtcttgaccaactctatcagagctt
ggttgacggcaatttcgatgatgcagcttgggcgcagggtcgatgcgacgcaatcgtccgatccggagccgggactgtcgggcgta
cacaaatcgcccgcagaagcgcggccgtctggaccgatggctgtgtagaagtactcgccgatagtggaaaccgacgccccagcact
cgtccgagggcaaaggaatagagtagatgccgaccgggatccacttaacgttactgaaatcatcaaacagcttgacgaatctggatat
aagatcgttggtgtcgatgtcagctccggagttgagacaaatggtgttcaggatctcgataagatacgttcatttgtccaagcagcaaag
agtgccttctagtgatttaatagctccatgtcaacaagaataaaacgcgtttcgggtttacctcttccagatacagctcatctgcaatgcatt
aatgcattggacctcgcaaccctagtacgcccttcaggctccggcgaagcagaagaatagcttagcagagtctattttcattttcgggag
acgagatcaagcagatcaacggtcgtcaagagacctacgagactgaggaatccgctcttggctccacgcgactatatatttgtctctaat
tgtactttgacatgctcctcttctttactctgatagcttgactatgaaaattccgtcaccagcccctgggttcgcaaagataattgcactgtttc
ttccttgaactctcaagcctacaggacacacattcatcgtaggtataaacctcgaaaatcattcctactaagatgggtatacaatagtaacc
atggttgcctagtgaatgctccgtaacacccaatacgccggccgaaacttttttacaactctcctatgagtcgtttacccagaatgcacag
gtacacttgtttagaggtaatccttctttctagctagaggatcctctacgccggacgcatcgtggccggcatcaccggcgccacaggtg
cggttgctgActagaataattatgtgtaacaagaaagacagtataatacaaacaaagatgcaagagcggctcatcgtcaccccatgat
agctagagcttggcactggccgtcgttttacaacgtcgtgactgggaaaaccctggcgttacccaacttaatcgccttgcagcacatcc
ccctttcgccagctggcgtaatagcgaagaggcccgcaccgatcgcccttcccaacagttgcgcagcctgaatggcgaatggcgcct
gatgcggtattttctccttacgcatctgtgcggtatttcacaccgcatatggtgcactctcagtacaatctgctctgatgccgcatagttaag
ccagccccgacacccgccaacacccgctgacgcgccctgacgggcttgtctgctcccggcatccgcttacagacaagctgtgaccg
tctccgggagctgcatgtgtcagaggttttcaccgtcatcaccgaaacgcgcgagacgaaagggcctcgtgatacgcctatttttatag
gttaatgtcatgataataatggtttcttagacgtcaggtggcacttttcggggaaatgtgcgcggaacccctatttgtttatttttctaaataca
ttcaaatatgtatccgctcatgagacaataaccctgataaatgcttcaataatattgaaaaaggaagagtatgagtattcaacatttccgtgt
cgcccttattcccttttttgcggcattttgccttcctgtttttgctcacccagaaacgctggtgaaagtaaaagatgctgaagatcagttgggt
gcacgagtgggttacatcgaactggatctcaacagcggtaagatccttgagagttttcgccccgaagaacgttttccaatgatgagcact
tttaaagttctgctatgtggcgcggtattatcccgtattgacgccgggcaagagcaactcggtcgccgcatacactattctcagaatgact
tggttgagtactcaccagtcacagaaaagcatcttacggatggcatgacagtaagagaattatgcagtgctgccataaccatgagtgat
aacactgcggccaacttacttctgacaacgatcggaggaccgaaggagctaaccgcttttttgcacaacatgggggatcatgtaactcg
ccttgatcgttgggaaccggagctgaatgaagccataccaaacgacgagcgtgacaccacgatgcctgtagcaatggcaacaacgtt
gcgcaaactattaactggcgaactacttactctagcttcccggcaacaattaatagactggatggaggcggataaagttgcaggaccac
ttctgcgctcggcccttccggctggctggtttattgctgataaatctggagccggtgagcgtgggtctcgcggtatcattgcagcactgg
ggccagatggtaagccctcccgtatcgtagttatctacacgacggggagtcaggcaactatggatgaacgaaatagacagatcgctg
agataggtgcctcactgattaagcattggtaactgtcagaccaagtttactcatatatactttagattgatttaaaacttcatttttaatttaaaa
ggatctaggtgaagatcctttttgataatctcatgaccaaaatcccttaacgtgagttttcgttccactgagcgtcagaccccgtagaaaag
atcaaaggatcttcttgagatcctttttttctgcgcgtaatctgctgcttgcaaacaaaaaaaccaccgctaccagcggtggtttgtttgccg
gatcaagagctaccaactctttttccgaaggtaactggcttcagcagagcgcagataccaaatactgtccttctagtgtagccgtagttag
gccaccacttcaagaactctgtagcaccgcctacatacctcgctctgctaatcctgttaccagtggctgctgccagtggcgataagtcgt
gtcttaccgggttggactcaagacgatagttaccggataaggcgcagcggtcgggctgaacggggggttcgtgcacacagcccagc
ttggagcgaacgacctacaccgaactgagatacctacagcgtgagctatgagaaagcgccacgcttcccgaagggagaaaggcgg
acaggtatccggtaagcggcagggtcggaacaggagagcgcacgagggagcttccagggggaaacgcctggtatctttatagtcct
gtcgggtttcgccacctctgacttgagcgtcgatttttgtgatgctcgtcaggggggcggagcctatggaaaaacgccagcaacgcgg
cctttttacggttcctggccttttgctggccttttgctcacatgttctttcctgcgttatcccctgattctgtggataaccgtattaccgcctttga
gtgagctgataccgctcgccgcagccgaacgaccgagcgcagcgagtcagtgagcgaggaagcggaagagcgcccaatacgca
aaccgcctctccccgcgcgttggccgattcattaatgcagctggcacgacaggtttcccgactggaaagcgggcagtgagcgcaacg
caattaatgtgagttagctcactcattaggcaccccaggctttacactttatgcttccggctcgtatgttgtgtggaattgtgagcggataac
aatttcacacaggaaacagctatgaccatgattac

Strategy III: Over expression of A. fumigatus 3′ HAC region (Afu5g14900-Afu5g14920) in A. niger: the below genomic sequence of HAC (Insert III) will be amplified with Primer 5 and Primer 6, digested with restriction enzymes BglII and Not1-HF and ligated into the below over expression vector sequence with the dominant pyrithiamine marker (Vector II) for selection in A. niger.
Insert III: A. fumigatus hrmA/Afu5g14900 genomic sequence from hypoxia-evolved strain

(SEQ ID NO: 2)
cgccgtaacgtaacaaagcggggttggtagtgtttgcaaatgcattcacatggaccgatcacttttctttccagtctgtccattctgtccaat
ctgtccgatctgacctgcccagtctgtccagtctgtcccttgtgtcgtccgatccaagctggttatcatggcatccacaaagcccgcttcg
agtctcatttaccaggcatggaacaaactcagtatcaaccaaaccatccctagtgactcccttgaattacttggggagcgtttggctattg
ccttcgcacccaaactcaaggagcaacgaaggaatggccggcgtcggaatctggaatatgtggcacaacatcgacggaagattgct
cgaaaaatctacttggagattctggagaaagacccaaatatctttcttccttttatcctggctgtttcccctagagcatgcttatcctttgatat
ctcgagctttcttgaacagcaccaaagccaaggaagacatttcctccgcaacaatgccgaagcgatcctctggggtctcgcaaagaaa
catgacattgatggctccctccatttcaggaagctgatgcgtgagattttccaactgtctcctccagcgacagaagccgaaggcaagga
gcattattcattgcatttaagcactctccccgcaatccgcaatgccttcggtgatgttatctttgacgcaattgaacgttcccctacacaggt
gacagcgagagctaaaggttatttctctgagaaaaccgaaagtgtttggacaaaagttccctacagaagttctcaagacgcaatcatatc
tcttgaagtagggtcggcaatcgagcttgcgaatgtgttgttcccaatcgcaacccaaaaaattgtctctatcctttccgcatgttctccca
ctgtgcgccagaagaacttttctgaggctattctcggcccagaccctcaggatacaccggcaacatcatcagaaatcggtatgaagttta
aggtacacatgactgcagttgctaattccaccctgtgctagatgtggcgtattttactctgcgaggagcaacggtctcggcaattgaatca
gtctttcgcgctgatatttgcgaaggtattaagggcagcgaactgagaaactgggaaaaggagcagctgctcatcgacacgacagatt
gtgtcacgatgcagatatggcgggcacaacctcaacatggaaccatcaagttgcgtattggattctatgcagcggtgaatttggcaaat
cggctgtatgcagaaacaccccaagatcacatatagtaacctttcatcttttcggccttcttaaatcattgcctttctgtgagtcgcgactttc
caccctttatgaatacaccaataccagggggaagaacgatttcaccgcttcccttggcaatccatatagttcccttctcattctggaacctg
atttcatcgcagttgaagcaatataaattcctttcgaggttttctgcattgtagggatggaatgggtgcgtgaaatatttgtcgaaaatagaa
gggtccgaaagttctttccaagctgcgcgctgaaagttgttagcccatctttcgagcatatggtttggcccacatacgagagattcttttgt
gatcggttgagattcttccgtgatcggttctgtcattttcattaagtcagagagccctcttgtatgccggcttttgctgtcggatccggcgag
ataatcgctcctaagccagtcagtcagggaaaagcaaagataaataaaatataggcgaggagtacaaccaggcacgcgtcgtagact
atttttctgaagagtttgtcacgtaacctacctcatatggatgggtagttcgaatacttgattgacttgacccgaggttctgaaggcggcgg
aggaaattgcccaaccccaccattgcattttcaggtatcaatctctgccacactgtggctaaattcgtctttatcgacacgtgatcacgttc
cctcttccagccctggtatcagagaatcatcgagttatcgcttgtttcaatttcgtcttgcaattagcttagggaataagcatgtggtcacat
caacctacagagcgctaccggtctttgcgctgagactctcagtgatccgcccaacagacaactagactttgaggttgtcgatataacca
caacaaatggcctgtatatcaacgatgtccacgcaattgtctcaagcctcttcacccgacttccagcactagcatccaagcggcctctcc
tcttctcccatgtttctcgtagcgcgcctgcatatacttatatctggagatatgttaaaggagctggaagcctggagcatacgctggaagc
ctggagcatacgcttcaagtgctgccatattcagatagctgagtaggcacaattaggtctaagttcagggaattgcacctctcgcttcatt
gtccgtcgattcgtatcggtctctagttctccccgtttatcactctcactcggtggacagtccgtccagtccgtccagtccgtcgagcctgt
ccaatctgtccaatctgtccaatctgtccaatctgtccaatctgtccaatctgtccaatctgtccaatctgtccaatctgtccaatctgtccaat
ctgtccaatctgtccaatctgtccaatctgtccaatctgtccaatctgtccactctgtccactctgtccactctgtccaatctgtccactctgtc
ccctctgtccactctgtcccctctgtccactctgtcccctctgtccaatcggtccaatctgtccaatcttgatgatctcgatgatcaataccat
ttagcgagcgctagtgacgccttacagcgttgcggcgtcttatggcttatacatcttccgaatatcagtcgttcgatctccagatcacacct
cggggtgaaacaagcgccatagttcttggtgcgcctgagcttttccctgcccggccactcagtcgtgatggcttcccagagtattccata
agtcgaaaccagagataggccagcggacggcaaatctcctctgctccgttctcaaccaatactgccaaagtgagcaatggaaagtgtt
ccaggagcaccgtggcctttaaaagcgccagccttgtcccagatcctcaccgcaattcggcacagaccaacgcagactttcattgacc
attcttcatttccagatcgctgcatagtttccggtatggcttggtatagagccttactcccgtgcataccgttgtggcggaaggttctgggg
cgtaacagcaccgatgaggacggacggagtgaagacgaccttacttcattgagcgataaaatgcctacttttgaagaaacgacaactg
tgagtactgttcgtgaaattgcctccagctgtctaatgtctccgtcggtcagatcacttctgcaaagtacatcaacggtgaaaaaatcatg
gagcataccgttgtggagaccaagcacattgacgaacgtggagacaccagcgtcagtaacggtgattcgaacagcactgcggtaac
cagacattctgggcttagctctgtcagcctttcagatcaaagtacaattgtcgaggacgcgaatgctctggaagaacctgaactctttgct
gttcactctccatatgttgacgactcaaccggcgagcagatggttagactctactacgagctcccagtaagcctcgatgatcttgagatc
ataggccttgaatctcgtattccagagtctgacgatgattcaattgaggcccgctttcgttatcgaggagaggatttttggctacctgttcgt
tattcttatgccaaagctcgaatggttttaacgggtgtatgctgaatggtctgacttcttcactgttgttattttctatttcccggctgctggcca
ctcaatattatcgcaagcactatacaaaataaatagtcctatctctataacagagtacgtcacaaacagcgttcgtccttggcaagaatata
aatagtgtcaatctgctgagaaaaggaggtatgaaatccacttcattcaagcaatggttcccttcgtatacattattatttgtatatggatgg
gaacttttcttgtcttagttgccctgaacgcccgctttaagaagaagcgcattgctgatcctgagtcttctgctgccttcactgatcatcccc
atcaagagtaacatggattctgtatgtccctttgggcatgtttatgtggcagttactaatatattagatggaaactaggaagcggcaaaaca
caggggcaggaagggaaggactcattccaagaaagaaggacaaggaagaacacaagagtgtaaggggtagcaatcctcattggc
gtctactagctgatgggttaaagcaagatacaggtgttcatacgctccaagacaacgatcctgttgtcgagaagccgccattaatttaca
ggagagctggagaagcgccgaaacatgattgggacaagaagtcacccggtttgcgccttagacggtcctgctgtagctgcgggaag
gaagtaccggtaggcctggtctgtcgtatttgtcaccatgagtcttgtcctgagtgcttgaacatgcaaaagcgagattacggatgttgat
cagatacacgggcccctatactaggactagttaccactaggattgtactcaaccttaacagtggcgtttggtacttcgttacagctaaggg
gagaggacagttcctactttcatgtgcttcaagggagaggctcctccaactactgttgctagggaggaaagacactctattcttagctga
gggctgcaggaattcgcatccatgcagtcataccatccatggcatgcaatctatgttgcattagatgcaagaagtaggatagagaaccc
atgtacttgattcacgctagcaggacagagagagataccatacccgacgggagcccctgcatgacttcctgtctgcagcttgtcgtgcg
tgtatcattcccatgcgccacgaactcataggcagtggtagttcagaacactctttttttttaaaaaaaaaaaagataggaaaataataattt
aggggaagaaaagtaaaaattaaaaagaaaaagttccagatggcgcttctacttctatattcgatcgttattcaataccccagaggcaca
ggcattccgatctctcatcgccaacactgaaaagcagccattttcccccgtcttaaagctccaatcctccttcttctcatctacttcctcgct
ccttcaggaccttgagtgttccgttgagctattgggtaacttctcacctgtcaatcatcgattgtcctttctcttgacttgacttcgtgtcgcca
ttctcatttacgatacatatccctggagcagaaaacaaagaaaagggccaattactcttgatctagttccaactctgttgctgcttggaaca
tccgcccatctgtgtggtgaaatcagatgccagcatccatcttgcagcttctcccacttcctgggccgatcttgaatgtttgctctcgaacc
tcgcttagtatttgatctccattctcatctgggtacatcctgtgagtagcatgtcgtcacttgtcacacatactacccgctctcaaatctgtttg
atgggagtcaatctgcctcgaaatggctcgtctgccttcacaagcaaactacagcagatggcgggggcatggactcgagccacagtg
ctggctctcgcttgcatctggaccttcttattctttctcattgctgtatctttttcccccttgaggcttctggcgcgctgcacctttccaagtatc
aaaccaaagctaatcaggggcgtttggcgtcctgccatggcttcactagacctggatctctgcagcctcatcaccatctcggatcacctg
gttctgatcaccttggaagaaagcacaaagaccttggagacaatacatattgccgccatcgcagctccctccaatctcgacagcattttc
atgtgtcgggcactatctacctctcggcaattcagtaaccgtactgcctgagaaacatcaacctctcaaattacacaatggtgttcagcgc
acctgctcctggtgtgggctccagtaaaaggccagcgtcatgcatgcaggacgatgttgatgagcgggataatgtcccagtaagtgat
accagcattggaaaggcagatggagctgactcatctcctatatagcccatgggtctatgcgtgcgttcatcgatcaacatcccttacttcc
actacgccatgatctatctcgacaacatgggcagacttaaggtgatggaatctccgtctatccaggagcaaaatgagactgttttcacaa
ccgaagtacgtgaaagatttttggaaatccttggtgccaaggtaggatatcaaccgcccatggttcgaagtatgtaaacactccgcgca
caagtacaatattcttgctgatctcaattgaacagggttgtcagctgccggtgctacaccatacagctatgatcctcaacaaccgcttggtt
gcttgtcttaccgtcaaactaagcgggacagaaattccccagcccactctatgtacggtgtgccgccatccgtccagttctcagccccg
gttgaggaatcgccctcttgtggatcagtggacatggtcgggctcgagattggtgatactcctaatgtccttgactactatgagagatcct
taaagcactttcggcaggtcaactgtcgccagatcctaaagacattcattaagttcattgagccacgaaagcaagccaagcacccctat
aatggaggtaaaccccctgcaggagcccctcctggtaagaagggcgacccagagaagacaaagcctgaatggtggcccgccaatg
tggtccacaaggagcctgaccatcttcgaaaggatcgtacgtgtaacccttcagaaaatcttcagtgtcaagtaactttgctgacagactt
agaacgcctgtctctgttaattcatatcatccgcaggcttggaagatttggtatcaccacggatcaattgcaggaaattgcccacgactgc
aagcggcggctcagcgacccccacaaactccaaatcttggacgaggtcttcagagtgagaaggattgaagaacgctacgaaagagg
agaagttggtaagcggcatcatctttccatgaaattcattttgacagctgttgacgagcctcagatgccaacaagatcgtatatgttgtcaa
ccgagagtcgaatcagaaagagaaggatggcgactccaacgtggatccggaccagaagcatgagcaagaagacgataatgcgcg
ggaggcacttcccattctccactccgagaagaactcaaccagcccgatgtcgaactcagccgagcacacgggcatggcggcaccaa
gtcgtccaatgaatatgggaggtgacagaaaccagttgtttcctttaccggagtggccgagcttcggtgagacaccccaggatgatcg
aattttctttcccacgacctctaagtataccgaagattatgcatcgcagcagatgcctagaacacctgcaacaacagcacttgtcagcact
aatgagacacatgcggcctttgattatatgacacaggagtccatcacctcctcctccccagagcagacttcccaccaccgccaagcac
ccctgcccatgcagcactcggccagcctcgacccttggacccctacgttccgacataatttcttcaacccaatggtgtatagtactgcac
cccgtcacgccatgtcccaggctactatgttatctcagtttcccaggtccacgacgtctcatggccaggaaatgcctcacatggctcacg
gcctgccgaacctgcctcaagacagaccttcaagcatggatggcatgagcatgagaggcccttctttccgcacaggatttttgagtcat
ccctgtgacccatcacagcaggctcctcattctagcggatgcggccatcctgacagttggactcaaaatagaccacatgtataatcttaa
ctgattgatccttgaccactgttttgaccctcctgcagccttgaagcttcgtttcactgatgattgttcttcgactttgtttctgtccctgactttg
ttgtcaatgcggacttatccatgcggcttgttccacgtcaagtgactaccaggacactccgtggttttatatggcaggtactggcgatgac
tttccaattcttcttcgtttagtatatatactcgtttcttgttctatgttcgatcatgtctttttccttatacatacctccaaaaatcctgttggag
atggcgccagatggcatgagatgcaaatatggatgatgttcttgtgtttgttcatttcaatttctttctcttaatcatgatttgaacaattggcagcg
aggtatggcggagctcgttctctttggatgccgatcagctgaataggaggtaacgaggcatgagggtgtttcattatgactctctccggt
gtttgtcatttaagggtgcgagggggaagtgtccgtttcgatgtcctaggatatcgaaaatctgagtagtagccacgtgaccctatgctg
acggctgggctggaagacaagcaggttgctgcttacgagaatatgttgaggtattctcgttatcttcgtgaagaatgccgtctccttggcc
ctctagccaaagtctgggttgctgaaaggctagctggaattgagaatcgactgtctgcgtccgagtcgcctagaggtgggaaggcccc
ctctttctcatacatatgctgactctgcagaccataccaattcgctgcccgaa

g: the sequence change as a result of in vitro evolution in hypoxia

Primer 5:
(SEQ ID NO: 41)
AAAAAGATCTgccgtaacgtaacaaagcggg
Primer 6:
(SEQ ID NO: 42)
AAAAGCGGCCGCttcgggcagcgaattggtatggtc

Vector II: Vector for insertion of DNA with pyrithiamine marker

(SEQ ID NO: 43)
CTAAATTGTAAGCGTTAATATTTTGTTAAAATTCGCGTTAAATTTTTGTTAAATCA
GCTCATTTTTTAACCAATAGGCCGAAATCGGCAAAATCCCTTATAAATCAAAAGA
ATAGACCGAGATAGGGTTGAGTGTTGTTCCAGTTTGGAACAAGAGTCCACTATTA
AAGAACGTGGACTCCAACGTCAAAGGGCGAAAAACCGTCTATCAGGGCGATGGC
CCACTACGTGAACCATCACCCTAATCAAGTTTTTTGGGGTCGAGGTGCCGTAAAG
CACTAAATCGGAACCCTAAAGGGAGCCCCCGATTTAGAGCTTGACGGGGAAAGC
CGGCGAACGTGGCGAGAAAGGAAGGGAAGAAAGCGAAAGGAGCGGGCGCTAG
GGCGCTGGCAAGTGTAGCGGTCACGCTGCGCGTAACCACCACACCCGCCGCGCT
TAATGCGCCGCTACAGGGCGCGTCCCATTCGCCATTCAGGCTGCGCAACTGTTGG
GAAGGGCGATCGGTGCGGGCCTCTTCGCTATTACGCCAGCTGGCGAAAGGGGGA
TGTGCTGCAAGGCGATTAAGTTGGGTAACGCCAGGGTTTTCCCAGTCACGACGTT
GTAAAACGACGGCCAGTGAGCGCGCGTAATACGACTCACTATAGGGCGAATTGG
AGCTCggcgcgccagatctacgcgtttaattaaCCGCGGTGGCGGCCGCTCTAGAACTAGTGGAT
CCCCCGGGCTGCAGGAATTCGATATCAAGCTTATCGATACCGTCGAGgtctgagaggag
gcactgatgcgctatcatggggtgacgatgagccgctcttgcatctttgtttgtattatactgtctttcttgttacacataattattct
agaatgccccaccgttacatacgggacacagccatttacatatgcatgtggattacgagctaacgagttcattcaaatctcaga
actatcacataatcatcattcccctatcgtcaaagaccgtaagacaaatccggttcatgcactgaacccattcgggtagtgagt
catttactcagcacactcgcgctgacgctcgtcgaacaccttcaatgcctcctcggcagccttgacaccactgagaaccatggcaccg
aaggtagggcccatgcggttaaagccatcaatttcagacagctccataccgccgattatcaagcccttagtaacctcgcgggtgttcttg
acgatggcatcctcggccgagttcatgtcgagaccacgcatgccacctagcttgtcgacgctgcccatggacaccaagcgcttcgca
cagaaggcgccgaatggcccatcgtgaccagtggtactgatgatgacaggagcgttgatagtgttggggtccatgcaggagtgatca
tcgtggtgaagggtgaccagcgtccagttgacgacaacaccagcaatctgggggttgccgttctcggtcggacgggtgatcaagtcct
caacagcggtagcattgaagagcttgacattggggaaggagagaaccttcgacatgagtgtcgaggtaaacagggaggcgtgcttg
acgacaacgtagttggggtttgcgtcctcttcgtaaggaacacccagctcgttcaggaagacttccgcgggacggcgcatgaccatag
cagaaaagagttggccacccaaccaggcaccgccacctattgcacgttagttccggaaagctgagtgcaaggcaatccatcatggac
tactgaccaggagagacgctggcctcgacgatagcaatcttcaggtccggacgagccttggccaagacgtacgcagtgctcagacc
gcaggaaccagcaccaacaatgacaacgtcactttcagcgtacttgtccaggtcctcaaagtaacgtctggtcatggcacgagagacc
tggctttcgcggataggggcgaacttgaactcgtcccacttgccaccgaaatggtccaacagcttggtctgagaagctccctcaacgg
ggacggtctcagaaaccacgaccttacccttgaggccggtagcggccacagtgggttcgtagatggcagctggaggagacatgtttc
aagttgcaatgactatcatctgttagccattccatcaacaggaagaacgagagaaggcatgacccttttcgctggtattatccagatcaa
gttttagccgtataatctcagaacgaacccagtccatcgatgccatgtccttctagactaggatcctagagtctagggcccagcttaggg
agggcatgtgaatgcatcgatgactgggaacgaacaccggcccacgccaaagacgttacctaagataccttgatcattgtgagagtcc
agccaaaagtattccatgacttccatcgtatgccctctagagggctaatcgaggagtgtatttacattgtcggttggtttgggaactataga
agatggtcagttattccaatcaccaaaggtttatcgaagggaggaagacttgttcagtttcgtccgaggacttttggaattcaaatctgag
atagagaattgtgtgggatgagaggaaaaggaagaagatggtggggttcagaaggaggggttgagttaaatagcatgggttgagtca
acgtgataagggcactataccgtatagatcagcggcacccgattctatccgttccttttgctcctctttagctttgaccggtgagccggac
aagaaacaagtgaaatcatcctgacatcggcggacgatctcctagcttttacatttcgttaccaatgggatcccgtaatcaattgcccgtc
tgtcagatccccagagcattgtttgaggcgaccggtCTCGACCTCGAGGGGGGGCCCGGTACCCAGCT
TTTGTTCCCTTTAGTGAGGGTTAATTGCGCGCTTGGCGTAATCATGGTCATAGCTG
TTTCCTGTGTGAAATTGTTATCCGCTCACAATTCCACACAACATACGAGCCGGAA
GCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTA
ATGAATCGGCCAACGCGCGGGGAGAGGCGGTTTGCGTATTGGGCGCTCTTCCGC
TTCCTCGCTCACTGACTCGCTGCGCTCGGTCGTTCGGCTGCGGCGAGCGGTATCA
GCTCACTCAAAGGCGGTAATACGGTTATCCACAGAATCAGGGGATAACGCAGGA
AAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGA
CGCTCAAGTCAGAGGTGGCGAAACCCGACAGGACTATAAAGATACCAGGCGTTT
CCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAG
CAGAGCGAGGTATGTAGGCGGTGCTACAGAGTTCTTGAAGTGGTGGCCTAACTA
CGGCTACACTAGAAGGACAGTATTTGGTATCTGCGCTCTGCTGAAGCCAGTTACC
TTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTCACG
TTAAGGGATTTTGGTCATGAGATTATCAAAAAGGATCTTCACCTAGATCCTTTTA
AATTAAAAATGAAGTTTTAAATCAATCTAAAGTATATATGAGTAAACTTGGTCTG
ACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTC
CAGATTTATCAGCAATAAACCAGCCAGCCGGAAGGGCCGAGCGCAGAAGTGGTC
CTGCAACTTTATCCGCCTCCATCCAGTCTATTAATTGTTGCCGGGAAGCTAGAGT
AAGTAGTTCGCCAGTTAATAGTTTGCGCAACGTTGTTGCCATTGCTACAGGCATC
GTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGT
CCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCT
CTTGCCCGGCGTCAATACGGGATAATACCGCGCCACATAGCAGAACTTTAAAAG
TGCTCATCATTGGAAAACGTTCTTCGGGGCGAAAACTCTCAAGGATCTTACCGCT
GTTGAGATCCAGTTCGATGTAACCCACTCGTGCACCCAACTGATCTTCAGCATCT
TTTACTTTCACCAGCGTTTCTGGGTGAGCAAAAACAGGAAGGCAAAATGCCGCA
AAAAAGGGAATAAGGGCGACACGGAAATGTTGAATACTCATACTCTTCCTTTTTC
AATATTATTGAAGCATTTATCAGGGTTATTGTCTCATGAGCGGATACATATTTGA
ATGTATTTAGAAAAATAAACAAATAGGGGTTCCGCGCACATTTCCCCGAAAAGT
GCCAC

### Key:

### Pyrithiamine Marker

Product III: Introduction of 3′ HAC genes into A. niger with pyrithiamine

(SEQ ID NO: 44)
CTAAATTGTAAGCGTTAATATTTTGTTAAAATTCGCGTTAAATTTTTGTTAAATCA
GCTCATTTTTTAACCAATAGGCCGAAATCGGCAAAATCCCTTATAAATCAAAAGA
ATAGACCGAGATAGGGTTGAGTGTTGTTCCAGTTTGGAACAAGAGTCCACTATTA
AAGAACGTGGACTCCAACGTCAAAGGGCGAAAAACCGTCTATCAGGGCGATGGC
CCACTACGTGAACCATCACCCTAATCAAGTTTTTTGGGGTCGAGGTGCCGTAAAG
CACTAAATCGGAACCCTAAAGGGAGCCCCCGATTTAGAGCTTGACGGGGAAAGC
CGGCGAACGTGGCGAGAAAGGAAGGGAAGAAAGCGAAAGGAGCGGGCGCTAG
GGCGCTGGCAAGTGTAGCGGTCACGCTGCGCGTAACCACCACACCCGCCGCGCT
TAATGCGCCGCTACAGGGCGCGTCCCATTCGCCATTCAGGCTGCGCAACTGTTGG
GAAGGGCGATCGGTGCGGGCCTCTTCGCTATTACGCCAGCTGGCGAAAGGGGGA
TGTGCTGCAAGGCGATTAAGTTGGGTAACGCCAGGGTTTTCCCAGTCACGACGTT
GTAAAACGACGGCCAGTGAGCGCGCGTAATACGACTCACTATAGGGCGAATTGG
AGCTCggcgcgccaGATCTgccgtaacgtaacaaagcggggttggtagtgtttgcaaatgcattcacatggaccgatcacttt
tctttccagtctgtccattctgtccaatctgtccgatctgacctgcccagtctgtccagtctgtcccttgtgtcgtccgatccaagctggttat
catggcatccacaaagcccgcttcgagtctcatttaccaggcatggaacaaactcagtatcaaccaaaccatccctagtgactcccttga
attacttggggagcgtttggctattgccttcgcacccaaactcaaggagcaacgaaggaatggccggcgtcggaatctggaatatgtg
gcacaacatcgacggaagattgctcgaaaaatctacttggagattctggagaaagacccaaatatctttcttccttttatcctggctgtttcc
cctagagcatgcttatcctttgatatctcgagctttcttgaacagcaccaaagccaaggaagacatttcctccgcaacaatgccgaagcg
atcctctggggtctcgcaaagaaacatgacattgatggctccctccatttcaggaagctgatgcgtgagattttccaactgtctcctccag
cgacagaagccgaaggcaaggagcattattcattgcatttaagcactctccccgcaatccgcaatgccttcggtgatgttatctttgacg
caattgaacgttcccctacacaggtgacagcgagagctaaaggttatttctctgagaaaaccgaaagtgtttggacaaaagttccctaca
gaagttctcaagacgcaatcatatctcttgaagtagggtcggcaatcgagcttgcgaatgtgttgttcccaatcgcaacccaaaaaattgt
ctctatcctttccgcatgttctcccactgtgcgccagaagaacttttctgaggctattctcggcccagaccctcaggatacaccggcaaca
tcatcagaaatcggtatgaagtttaaggtacacatgactgcagttgctaattccaccctgtgctagatgtggcgtattttactctgcgagga
gcaacggtctcggcaattgaatcagtctttcgcgctgatatttgcgaaggtattaagggcagcgaactgagaaactgggaaaaggagc
agctgctcatcgacacgacagattgtgtcacgatgcagatatggcgggcacaacctcaacatggaaccatcaagttgcgtattggattc
tatgcagcggtgaatttggcaaatcggctgtatgcagaaacaccccaagatcacatatagtaacctttcatcttttcggccttcttaaatcat
tgcctttctgtgagtcgcgactttccaccctttatgaatacaccaataccagggggaagaacgatttcaccgcttcccttggcaatccatat
agttcccttctcattctggaacctgatttcatcgcagttgaagcaatataaattcctttcgaggttttctgcattgtagggatggaatgggtgc
gtgaaatatttgtcgaaaatagaagggtccgaaagttctttccaagctgcgcgctgaaagttgttagcccatctttcgagcatatggtttgg
cccacatacgagagattcttttgtgatcggttgagattcttccgtgatcggttctgtcattttcattaagtcagagagccctcttgtatgccgg
cttttgctgtcggatccggcgagataatcgctcctaagccagtcagtcagggaaaagcaaagataaataaaatataggcgaggagtac
aaccaggcacgcgtcgtagactatttttctgaagagtttgtcacgtaacctacctcatatggatgggtagttcgaatacttgattgacttga
cccgaggttctgaaggcggcggaggaaattgcccaaccccaccattgcattttcaggtatcaatctctgccacactgtggctaaattcgt
ctttatcgacacgtgatcacgttccctcttccagccctggtatcagagaatcatcgagttatcgcttgtttcaatttcgtcttgcaattagctta
gggaataagcatgtggtcacatcaacctacagagcgctaccggtctttgcgctgagactctcagtgatccgcccaacagacaactaga
ctttgaggttgtcgatataaccacaacaaatggcctgtatatcaacgatgtccacgcaattgtctcaagcctcttcacccgacttccagca
ctagcatccaagcggcctctcctcttctcccatgtttctcgtagcgcgcctgcatatacttatatctggagatatgttaaaggagctggaag
cctggagcatacgctggaagcctggagcatacgcttcaagtgctgccatattcagatagctgagtaggcacaattaggtctaagttcag
ggaattgcacctctcgcttcattgtccgtcgattcgtatcggtctctagttctccccgtttatcactctcactcggtggacagtccgtccagt
ccgtccagtccgtcgagcctgtccaatctgtccaatctgtccaatctgtccaatctgtccaatctgtccaatctgtccaatctgtccaatctg
tccaatctgtccaatctgtccaatctgtccaatctgtccaatctgtccaatctgtccaatctgtccaatctgtccactctgtccactctgtccac
tctgtccaatctgtccactctgtcccctctgtccactctgtcccctctgtccactctgtcccctctgtccaatcggtccaatctgtccaatcttg
atgatctcgatgatcaataccatttagcgagcgctagtgacgccttacagcgttgcggcgtcttatggcttatacatcttccgaatatcagt
cgttcgatctccagatcacacctcggggtgaaacaagcgccatagttcttggtgcgcctgagcttttccctgcccggccactcagtcgtg
atggcttcccagagtattccataagtcgaaaccagagataggccagcggacggcaaatctcctctgctccgttctcaaccaatactgcc
aaagtgagcaatggaaagtgttccaggagcaccgtggcctttaaaagcgccagccttgtcccagatcctcaccgcaattcggcacag
accaacgcagactttcattgaccattcttcatttccagatcgctgcatagtttccggtatggcttggtatagagccttactcccgtgcatacc
gttgtggcggaaggttctggggcgtaacagcaccgatgaggacggacggagtgaagacgaccttacttcattgagcgataaaatgcc
tacttttgaagaaacgacaactgtgagtactgttcgtgaaattgcctccagctgtctaatgtctccgtcggtcagatcacttctgcaaagta
catcaacggtgaaaaaatcatggagcataccgttgtggagaccaagcacattgacgaacgtggagacaccagcgtcagtaacggtg
attcgaacagcactgcggtaaccagacattctgggcttagctctgtcagcctttcagatcaaagtacaattgtcgaggacgcgaatgctc
tggaagaacctgaactctttgctgttcactctccatatgttgacgactcaaccggcgagcagatggttagactctactacgagctcccagt
aagcctcgatgatcttgagatcataggccttgaatctcgtattccagagtctgacgatgattcaattgaggcccgctttcgttatcgagga
gaggatttttggctacctgttcgttattcttatgccaaagctcgaatggttttaacgggtgtatgctgaatggtctgacttcttcactgttgttat
tttctatttcccggctgctggccactcaatattatcgcaagcactatacaaaataaatagtcctatctctataacagagtacgtcacaaaca
gcgttcgtccttggcaagaatataaatagtgtcaatctgctgagaaaaggaggtatgaaatccacttcattcaagcaatggttcccttcgt
atacattattatttgtatatggatgggaacttttcttgtcttagttgccctgaacgcccgctttaagaagaagcgcattgctgatcctgagtctt
ctgctgccttcactgatcatccccatcaagagtaacatggattctgtatgtccctttgggcatgtttatgtggcagttactaatatattagatg
gaaactaggaagcggcaaaacacaggggcaggaagggaaggactcattccaagaaagaaggacaaggaagaacacaagagtgt
aaggggtagcaatcctcattggcgtctactagctgatgggttaaagcaagatacaggtgttcatacgctccaagacaacgatcctgttgt
cgagaagccgccattaatttacaggagagctggagaagcgccgaaacatgattgggacaagaagtcacccggtttgcgccttagacg
gtcctgctgtagctgcgggaaggaagtaccggtaggcctggtctgtcgtatttgtcaccatgagtcttgtcctgagtgcttgaacatgca
aaagcgagattacggatgttgatcagatacacgggcccctatactaggactagttaccactaggattgtactcaaccttaacagtggcgt
ttggtacttcgttacagctaaggggagaggacagttcctactttcatgtgcttcaagggagaggctcctccaactactgttgctagggag
gaaagacactctattcttagctgagggctgcaggaattcgcatccatgcagtcataccatccatggcatgcaatctatgttgcattagatg
caagaagtaggatagagaacccatgtacttgattcacgctagcaggacagagagagataccatacccgacgggagcccctgcatga
cttcctgtctgcagcttgtcgtgcgtgtatcattcccatgcgccacgaactcataggcagtggtagttcagaacactctttttttttaaaaaaa
aaaaagataggaaaataataatttaggggaagaaaagtaaaaattaaaaagaaaaagttccagatggcgcttctacttctatattcgatc
gttattcaataccccagaggcacaggcattccgatctctcatcgccaacactgaaaagcagccattttcccccgtcttaaagctccaatc
ctccttcttctcatctacttcctcgctccttcaggaccttgagtgttccgttgagctattgggtaacttctcacctgtcaatcatcgattgtccttt
ctcttgacttgacttcgtgtcgccattctcatttacgatacatatccctggagcagaaaacaaagaaaagggccaattactcttgatctagtt
ccaactctgttgctgcttggaacatccgcccatctgtgtggtgaaatcagatgccagcatccatcttgcagcttctcccacttcctgggcc
gatcttgaatgtttgctctcgaacctcgcttagtatttgatctccattctcatctgggtacatcctgtgagtagcatgtcgtcacttgtcacaca
tactacccgctctcaaatctgtttgatgggagtcaatctgcctcgaaatggctcgtctgccttcacaagcaaactacagcagatggcggg
ggcatggactcgagccacagtgctggctctcgcttgcatctggaccttcttattctttctcattgctgtatctttttcccccttgaggcttctgg
cgcgctgcacctttccaagtatcaaaccaaagctaatcaggggcgtttggcgtcctgccatggcttcactagacctggatctctgcagc
ctcatcaccatctcggatcacctggttctgatcaccttggaagaaagcacaaagaccttggagacaatacatattgccgccatcgcagct
ccctccaatctcgacagcattttcatgtgtcgggcactatctacctctcggcaattcagtaaccgtactgcctgagaaacatcaacctctc
aaattacacaatggtgttcagcgcacctgctcctggtgtgggctccagtaaaaggccagcgtcatgcatgcaggacgatgttgatgag
cgggataatgtcccagtaagtgataccagcattggaaaggcagatggagctgactcatctcctatatagcccatgggtctatgcgtgcg
ttcatcgatcaacatcccttacttccactacgccatgatctatctcgacaacatgggcagacttaaggtgatggaatctccgtctatccagg
agcaaaatgagactgttttcacaaccgaagtacgtgaaagatttttggaaatccttggtgccaaggtaggatatcaaccgcccatggttc
gaagtatgtaaacactccgcgcacaagtacaatattcttgctgatctcaattgaacagggttgtcagctgccggtgctacaccatacagc
tatgatcctcaacaaccgcttggttgcttgtcttaccgtcaaactaagcgggacagaaattccccagcccactctatgtacggtgtgccg
ccatccgtccagttctcagccccggttgaggaatcgccctcttgtggatcagtggacatggtcgggctcgagattggtgatactcctaat
gtccttgactactatgagagatccttaaagcactttcggcaggtcaactgtcgccagatcctaaagacattcattaagttcattgagccac
gaaagcaagccaagcacccctataatggaggtaaaccccctgcaggagcccctcctggtaagaagggcgacccagagaagacaa
agcctgaatggtggcccgccaatgtggtccacaaggagcctgaccatcttcgaaaggatcgtacgtgtaacccttcagaaaatcttcag
tgtcaagtaactttgctgacagacttagaacgcctgtctctgttaattcatatcatccgcaggcttggaagatttggtatcaccacggatca
attgcaggaaattgcccacgactgcaagcggcggctcagcgacccccacaaactccaaatcttggacgaggtcttcagagtgagaag
gattgaagaacgctacgaaagaggagaagttggtaagcggcatcatctttccatgaaattcattttgacagctgttgacgagcctcagat
gccaacaagatcgtatatgttgtcaaccgagagtcgaatcagaaagagaaggatggcgactccaacgtggatccggaccagaagca
tgagcaagaagacgataatgcgcgggaggcacttcccattctccactccgagaagaactcaaccagcccgatgtcgaactcagccg
agcacacgggcatggcggcaccaagtcgtccaatgaatatgggaggtgacagaaaccagttgtttcctttaccggagtggccgagctt
cggtgagacaccccaggatgatcgaattttctttcccacgacctctaagtataccgaagattatgcatcgcagcagatgcctagaacac
ctgcaacaacagcacttgtcagcactaatgagacacatgcggcctttgattatatgacacaggagtccatcacctcctcctccccagag
cagacttcccaccaccgccaagcacccctgcccatgcagcactcggccagcctcgacccttggacccctacgttccgacataatttctt
caacccaatggtgtatagtactgcaccccgtcacgccatgtcccaggctactatgttatctcagtttcccaggtccacgacgtctcatggc
caggaaatgcctcacatggctcacggcctgccgaacctgcctcaagacagaccttcaagcatggatggcatgagcatgagaggccct
tctttccgcacaggatttttgagtcatccctgtgacccatcacagcaggctcctcattctagcggatgcggccatcctgacagttggactc
aaaatagaccacatgtataatcttaactgattgatccttgaccactgttttgaccctcctgcagccttgaagcttcgtttcactgatgattgttc
ttcgactttgtttctgtccctgactttgttgtcaatgcggacttatccatgcggcttgttccacgtcaagtgactaccaggacactccgtggtt
ttatatggcaggtactggcgatgactttccaattcttcttcgtttagtatatatactcgtttcttgttctatgttcgatcatgtctttttccttata
catacctccaaaaatcctgttggagatggcgccagatggcatgagatgcaaatatggatgatgttcttgtgtttgttcatttcaatttctttctctta
atcatgatttgaacaattggcagcgaggtatggcggagctcgttctctttggatgccgatcagctgaataggaggtaacgaggcatgag
ggtgtttcattatgactctctccggtgtttgtcatttaagggtgcgagggggaagtgtccgtttcgatgtcctaggatatcgaaaatctgagt
agtagccacgtgaccctatgctgacggctgggctggaagacaagcaggttgctgcttacgagaatatgttgaggtattctcgttatcttc
gtgaagaatgccgtctccttggccctctagccaaagtctgggttgctgaaaggctagctggaattgagaatcgactgtctgcgtccgag
tcgcctagaggtgggaaggccccctctttctcatacatatgctgactctgcagaccataccaattcgctgcccgaaGCGGCCGC
TCTAGAACTAGTGGATCCCCCGGGCTGCAGGAATTCGATATCAAGCTTATCGATA
CCGTCGAGgtctgagaggaggcactgatgcgctatcatggggtgacgatgagccgctcttgcatctttgtttgtattatactgtctt
tcttgttacacataattattctagaatgccccaccgttacatacgggacacagccatttacatatgcatgtggattacgagctaacgagttc
attcaaatctcagaactatcacataatcatcattcccctatcgtcaaagaccgtaagacaaatccggttcatgcactgaacccattcgggt
agtgagtcatttactcagcacactcgcgctgacgctcgtcgaacaccttcaatgcctcctcggcagccttgacaccactgagaaccatg
gcaccgaaggtagggcccatgcggttaaagccatcaatttcagacagctccataccgccgattatcaagcccttagtaacctcgcggg
tgttcttgacgatggcatcctcggccgagttcatgtcgagaccacgcatgccacctagcttgtcgacgctgcccatggacaccaagcg
cttcgcacagaaggcgccgaatggcccatcgtgaccagtggtactgatgatgacaggagcgttgatagtgttggggtccatgcagga
gtgatcatcgtggtgaagggtgaccagcgtccagttgacgacaacaccagcaatctgggggttgccgttctcggtcggacgggtgat
caagtcctcaacagcggtagcattgaagagcttgacattggggaaggagagaaccttcgacatgagtgtcgaggtaaacagggagg
cgtgcttgacgacaacgtagttggggtttgcgtcctcttcgtaaggaacacccagctcgttcaggaagacttccgcgggacggcgcat
gaccatagcagaaaagagttggccacccaaccaggcaccgccacctattgcacgttagttccggaaagctgagtgcaaggcaatcc
atcatggactactgaccaggagagacgctggcctcgacgatagcaatcttcaggtccggacgagccttggccaagacgtacgcagtg
ctcagaccgcaggaaccagcaccaacaatgacaacgtcactttcagcgtacttgtccaggtcctcaaagtaacgtctggtcatggcac
gagagacctggctttcgcggataggggcgaacttgaactcgtcccacttgccaccgaaatggtccaacagcttggtctgagaagctcc
ctcaacggggacggtctcagaaaccacgaccttacccttgaggccggtagcggccacagtgggttcgtagatggcagctggaggag
acatgtttcaagttgcaatgactatcatctgttagccattccatcaacaggaagaacgagagaaggcatgacccttttcgctggtattatcc
agatcaagttttagccgtataatctcagaacgaacccagtccatcgatgccatgtccttctagactaggatcctagagtctagggcccag
cttagggagggcatgtgaatgcatcgatgactgggaacgaacaccggcccacgccaaagacgttacctaagataccttgatcattgtg
agagtccagccaaaagtattccatgacttccatcgtatgccctctagagggctaatcgaggagtgtatttacattgtcggttggtttggga
actatagaagatggtcagttattccaatcaccaaaggtttatcgaagggaggaagacttgttcagtttcgtccgaggacttttggaattcaa
atctgagatagagaattgtgtgggatgagaggaaaaggaagaagatggtggggttcagaaggaggggttgagttaaatagcatgggt
tgagtcaacgtgataagggcactataccgtatagatcagcggcacccgattctatccgttccttttgctcctctttagctttgaccggtgag
ccggacaagaaacaagtgaaatcatcctgacatcggcggacgatctcctagcttttacatttcgttaccaatgggatcccgtaatcaattg
cccgtctgtcagatccccagagcattgtttgaggcgaccggtCTCGACCTCGAGGGGGGGCCCGGTACCC
AGCTTTTGTTCCCTTTAGTGAGGGTTAATTGCGCGCTTGGCGTAATCATGGTCATA
GCTGTTTCCTGTGTGAAATTGTTATCCGCTCACAATTCCACACAACATACGAGCC
GGAAGCATAAAGTGTAAAGCCTGGGGTGCCTAATGAGTGAGCTAACTCACATTA
ATTGCGTTGCGCTCACTGCCCGCTTTCCAGTCGGGAAACCTGTCGTGCCAGCTGC
ATTAATGAATCGGCCAACGCGCGGGGAGAGGCGGTTTGCGTATTGGGCGCTCTT
CCGCTTCCTCGCTCACTGACTCGCTGCGCTCGGTCGTTCGGCTGCGGCGAGCGGT
ATCAGCTCACTCAAAGGCGGTAATACGGTTATCCACAGAATCAGGGGATAACGC
AGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGC
GTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGC
TGTAGGTATCTCAGTTCGGTGTAGGTCGTTCGCTCCAAGCTGGGCTGTGTGCACG
AACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGAT
TAGCAGAGCGAGGTATGTAGGCGGTGCTACAGAGTTCTTGAAGTGGTGGCCTAA
CTACGGCTACACTAGAAGGACAGTATTTGGTATCTGCGCTCTGCTGAAGCCAGTT
ACCTTCGGAAAAAGAGTTGGTAGCTCTTGATCCGGCAAACAAACCACCGCTGGT
AGCGGTGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAAAACTC
ACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATT
TCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGG
CTCCAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTACAGGC
ATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTC
GGTCCTCCGATCGTTGTCAGAAGTAAGTTGGCCGCAGTGTTATCACTCATGGTTA
TGGCAGCACTGCATAATTCTCTTACTGTCATGCCATCCGTAAGATGCTTTTCTGTG
ACTGGTGAGTACTCAACCAAGTCATTCTGAGAATAGTGTATGCGGCGACCGAGTT
GCTCTTGCCCGGCGTCAATACGGGATAATACCGCGCCACATAGCAGAACTTTAA
AAGTGCTCATCATTGGAAAACGTTCTTCGGGGCGAAAACTCTCAAGGATCTTACC
GCTGTTGAGATCCAGTTCGATGTAACCCACTCGTGCACCCAACTGATCTTCAGCA
tcttttactttcaccagcgtttctgggtgagcaaaaacaggaaggcaaaatgccg
CAAAAAAGGGAATAAGGGCGACACGGAAATGTTGAATACTCATACTCTTCCTTT
TTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAA
GTGCCAC

Genome Editing of S. cerevisiae Strains

Integration of heterologous genes of interest into the chromosome is one method for engineering S. cerevisiae for use in industrial scale fermentations and heterologous protein production. Consequently, to demonstrate that the technology disclosed herein can reduce oxygen consumption and requirements in S. cerevisiae based fermentations and protein production, the HAC and the evolved allele of hrmA will be integrated into rDNA sites in the S. cerevisiae genome using a traditional yeast genetic engineering approach based on homologous recombination. Standard laboratory haploid strains of S. cerevisiae will be used for these experiments, BY4741 and BY4742. rDNA sequence will be used as the homologous recombination site flanking HAC or the evolved hrmA allele sequences described above. This will target the heterologous genes for integration at rDNA sites which are commonly used for integration and expression of genes in S. cerevisiae. For selection of transformants, the bacterial kanamycin resistance gene, kan, can be utilized. All strains will be confirmed through initial PCR based confirmation of target integration, Southern analyses, and expression levels of the respective integrated genes using qRT-PCR. Finalized strain(s) that show phenotypes of interest (high biomass yield, reduced oxygen consumption, ethanol production) will be sent for whole genome sequencing. Following successful proof of concept in laboratory strains, introduction of HAC and/or associated genes into industrial strains of S. cerevisiae will be demonstrated.

### Quantification of Fungal Biomass and Morphology

The biomass and morphology of fungi critically impacts the production of fermentation and heterologous protein products in batch cultures (Colin 2013, supra). Genetically-modified A. niger and S. cerevisiae strains generated as described above will be assayed for: 1) spore germination rate (A. niger only), 2) submerged fungal morphology, 3) and relative fungal biomass at a range of oxygen concentrations. A spectrophotometric assay to quantify fungal germination and hyphal extension over 24-36 hours of fungal growth was previously utilized (Beattie 2017, supra). This assay will be used with potato dextrose broth (PDB) to compare differences in early growth rates in normal oxygen (˜21% O₂) across strains of A. niger. To assess morphological changes as a result of the aforementioned genetic modifications, the A. niger and S. cerevisiae strains will be grown in liquid shaking cultures of 100 mL PDB (A. niger) or YPD (yeast) and compare the ability of the strains to form pellets, flocs, or loose filamentous hyphae (A. niger). Using this same assay, the mycelia will be collected, flash freeze, and lyophilize the tissue for A. niger. This will allow for comparisons of total dry weight of filamentous fungal biomass following, 18, 24, 36, and 48 hours of growth. For S. cerevisiae, optical density (O.D.) measurements at O.D. 600 will be used to monitor growth rate and total biomass over a 48 hour time period, sampling every hour. Other media conditions relevant to specific fermentations or protein production will be tested as indicated.

Given that the induction of HAC in A. fumigatus generates a strain, EVOL20, that is better able to grow in low oxygen environments, the ability of HAC (portions or in entirely) to influence the ability of A. niger and S. cerevisiae to grow in low oxygen environments (10%, 5%, 2%, and 0.2% O₂) will be assayed. These assays will be performed in liquid shaking cultures as described above for biomass quantification using our INVIVO₂ 400 Hypoxia Workstation (Ruskinn Technology Limited, Bridgend, UK) equipped with a gas regulator (Kowalski 2016, supra).

### Quantification of Fungal Oxygen Consumption

Fungal oxygen consumption will be quantified using a Unisense Clark-type microsensor (https://www.unisense.com/O2/) as described above (see FIG. 23b). Currently, the majority of citric acid production by A. niger is performed through submerged fermentation, however surface fermentation remains utilized to a lesser extent (Show 2015, supra). Therefore, oxygen consumption of the generated A. niger strains in both planktonic (submerged fermentation) and surface-adhered cultures will be assayed. For S. cerevisiae batch production cultures will be utilized to monitor oxygen consumption of strains engineered to express HAC in comparison to the parental strains.

For A. niger submerged fermentation cultures, strains will be grown to equivalent biomass in 100 mL cultures in PDB. Mycelia will then be collected through vacuum filtration with sterile Miracloth and be resuspended in 20 mL of fresh PDB in a 50 mL plastic conical tubes. Immediately, the Unisense OX-25 electrode will be placed at a depth of 10 mL into the freshly inoculated PDB and will monitor the dissolved oxygen every 120 seconds for 30 minutes. The electrode does not consume oxygen providing fast and accurate readings.

The same protocol will be used for S. cerevisiae fermentations, except strains will be grown in YPD or other appropriate media until mid-log phase, collected, washed with sterile PBS, and resuspended in fresh YPD media prior to monitoring of oxygen consumption. For surface-adhered cultures, A. niger will be grown in static cultures of 4 mL PDB in sterile 6-well polystyrene plates for 36-48 hours. The spent media will be removed and replaced with fresh PDB before immediately being analyzed for dissolved oxygen using the Unisense OX-25 system at a depth of 3 mL. Oxygen readings will be recorded as described above. Surface adhered mycelia of genetically modified strains will be collected to compare biomass to the parental strains.

Quantification of A. niger Citric Acid Production

Genetically engineered A. niger strains that show a reduction in oxygen consumption and a maintenance or increase in fungal biomass yield will be utilized in a colorimetric assay for preliminary quantification of citrate within culture supernatants and within lysed mycelia (BioVision #K655). For supernatant samples, A. niger cultures in PDB or fermentation media (Bhattacharjee 2015, supra) will be cultured for 60 hours and mycelia will be removed through sterile Miracloth. For lysed mycelia, collected mycelia through the Miracloth will be flash frozen in liquid nitrogen and ground using a sterile and cold mortar and pestle. Ground tissue will be suspended in PBS and used in the colorimetric assay. Parental strains will be included as references. Strains with reduced oxygen consumption that do not have significant reduction in citric acid production relative to the parental strain will be considered a strain of interest for larger scale assays.

### Conclusion

Successful engineering of A. niger and S. cerevisiae strains with the technology described herein will be identified by 1) reduced oxygen consumption in planktonic/submerged culture conditions, 2) an increase in or equivalent production of citric acid in inducing conditions for A. niger, and 3) no reduction in fungal biomass compared to the reference A. niger or S. cerevisiae strains (FIG. 27). Additional engineering of other industrial relevant fungi with our technology is a planned future direction.

### Example 3—Introduction of the Cryptic Gene Ortholog is Sufficient to Complement the Loss of cgnA and hrmA in EVOL20

The work described above identified the HAC region as important for hypoxia tolerance. A cryptic subtelomeric gene was identified next. This gene was sufficient to induce the hypoxia-locked colony and biofilm morphology in A. fumigatus, and increase low oxygen growth. It is one three putative orthologs present across A. fumigatus strains, all of which have the capacity to impact hyphal architecture and biofilm development and are herein named biofilm architecture factors (baf). Introduction of the A. fumigatus cryptic gene bafA into A. niger generated the hypoxia-locked colony and biofilm morphotypes indicating the potential broad impacts of these previously uncharacterized genes on biofilm architecture and development both naturally and through synthetic introduction.

The Native 5′ Sequence to cgnA is Required to Complement the Loss of cgnA in EVOL20

Through an experimental evolution approach, where the reference strain AF293 was serially passaged in a low oxygen (0.2% O2) environment, the strain EVOL20 was generated. As a result of the low oxygen passaging, the EVOL20 strain acquired a hypoxia-locked colony morphology (H-MORPH) characterized by colony furrows and increased vegetative mycelia during normal oxygen growth. Genes were identified the responsible for this morphological transition within a subtelomeric gene cluster. The apparent regulator of this gene cluster, hrmA, induces expression of the surrounding genes in the hrmA-associated cluster (HAC) including the adjacent collagen-like protein encoding gene cgnA. Disruption of the gene cluster by deleting cgnA in EVOL20 reverts the colony morphology from H-MORPH to that of the parent strain AF293 which we termed N-MORPH. However, over expression of cgnA in AF293, where basal HAC expression is low, is unable to generated H-MORPH or the elevated hypoxic growth characteristic of the EVOL20 strain.

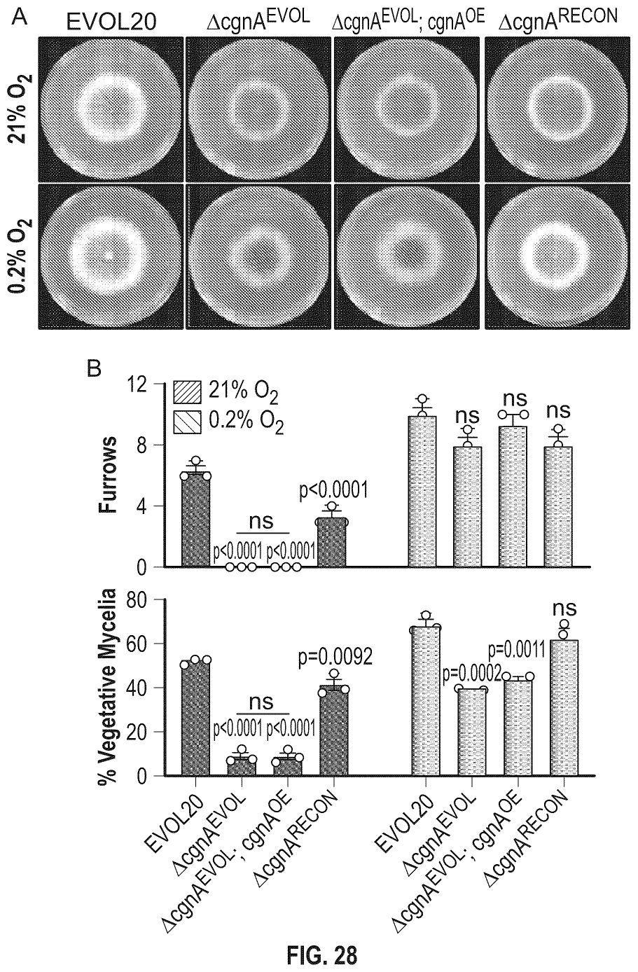

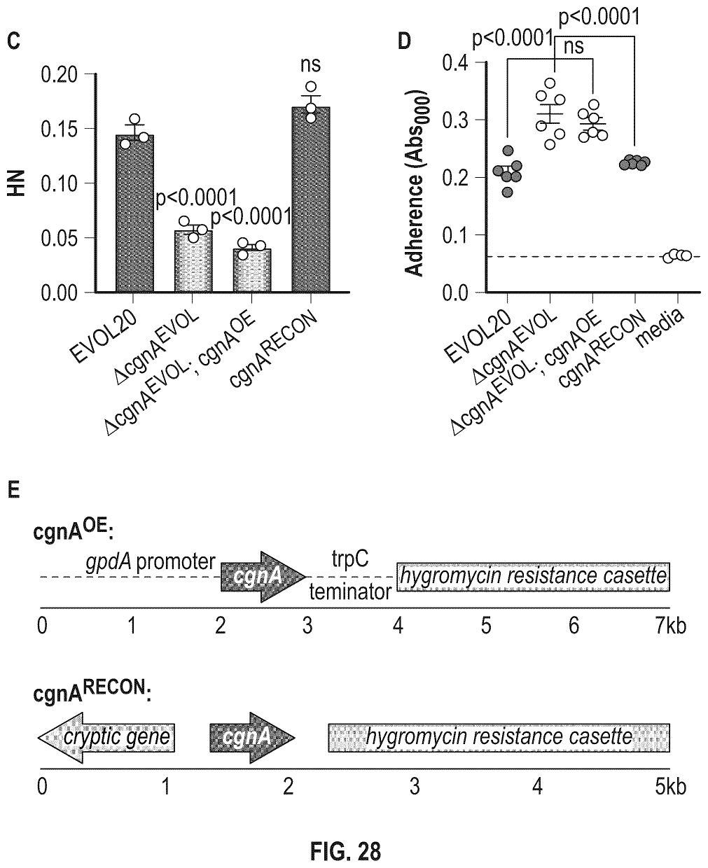

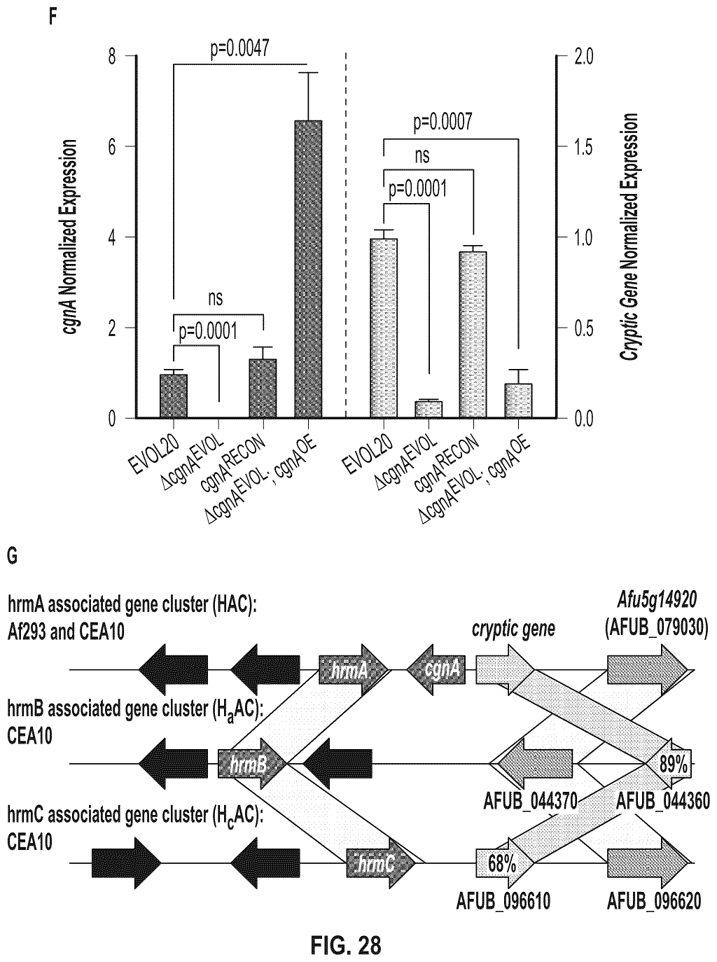

Simultaneous elevated expression of HAC genes may be additionally required for H-MORPH. As the majority of annotated HAC genes, with the exception of Afu5g14920, remain unaltered following the deletion of cgnA in EVOL20, cgnA was overexpressed in this background (ΔcgnA^{EVOL}; cgnA^{OE}) (FIG. 28A). This method did not complement the loss of cgnA in EVOL20 and regenerate H-MORPH, where colony furrows and the percent vegetative mycelia were not significantly altered relative to ΔcgnA^{EVOL} (FIG. 28B). We next hypothesized that the ectopic integration of the cgnA allele may prevent complementation. However, ectopic integration of cgnA with its native promoter and 5′ sequence is able to complement the loss of cgnA in EVOL20 and restore H-MORPH with elevated colony furrows and an increased percentage of vegetative mycelia (FIG. 28A, 28B). In addition to a transition from H-MORPH to N-MORPH colony morphology, the loss of cgnA in EVOL20 (ΔcgnAEVOL) also significantly reduces the ratio of hypoxic to normoxia growth (hypoxia fitness, H/N) of EVOL20 (FIG. 28C) and significantly increases hyphal adherence (FIG. 28D). Where ΔcgnA^{EVOL}; cgnA^{OE} does not complement either of these phenotypes, cgnA^{RECO}N restores both hypoxia fitness and adherence of ΔcgnA^{EVOL} to the levels of EVOL20 (FIG. 28C, 28D). The necessity of the native sequence 5′ of cgnA to complement ΔcgnA^{EVOL} prompted the investigation of this region more closely.

A Cryptic Gene is Encoded 5′ of cgnA within HAC and is Required for H-MORPH and HAC Related Phenotypes

By utilizing previously published RNA-sequencing data, a substantial region of mapped reads 5′ to cgnA in EVOL20 that were absent in AF293 were identified. Neither the AF293 assembled reference genome, nor the partially assembled genome of A1163, annotate a gene within this region. It is unlikely that these reads belong to the same transcript as cgnA as they map to the opposite strand. Therefore, we hypothesize that these reads map to an independent cryptic gene within HAC, and that this gene may be important for H-MORPH and other EVOL20-related phenotypes (i.e. hypoxia fitness, adherence, and biofilm architecture). To determine if our strategies to delete cgnA interrupt the expression of this cryptic gene, we designed primers within the predicted open reading frame (ORF) to quantify relative expression in two isogenic strain sets. Both in EVOL20/ΔcgnAEVOL and in hrmAR-EV/hrmAR-EV; ΔcgnA. In both cases deletion of the cgnA coding sequence reduces cgnA mRNA levels and mRNA levels corresponding to the cryptic gene.

A two exon ORF of 579 base pairs (bp) from the region corresponding to the cryptic gene was predicted. In the DNA construct used to generate ΔcgnAEVOL; cgnAOE, cgnA expression was driven by the constitutive gpdA promoter from A. nidulans and therefore the native 5′ sequence containing the cryptic gene ORF was not re-introduced (FIG. 28E). In contrast, the DNA construct used to generate the cgnARECON strain utilized the native sequence 5′ to cgnA to drive expression. This region included the entire predicted coding sequence of the cryptic gene (FIG. 28E). Gene expression analysis confirms that both ΔcgnAEVOL; cgnAOE and cgnARECON have cgnA mRNA levels equivalent to or greater than those of EVOL20, but only cgnARECON restores mRNA levels of the cryptic gene similarly to EVOL20 (FIG. 28F). Only with the strain cgnARECON, where both cgnA and the cryptic gene are expressed, is H-MORPH restored (FIG. 28A, 28B), hypoxic fitness increased (FIG. 28C), and adherence reduced (FIG. 28D) in ΔcgnAEVOL. Thus, the cgnA sequence alone is not sufficient to generate the EVOL20 phenotypes but requires the 5′ cryptic gene.

Introduction of the Cryptic Gene Ortholog is Sufficient to Complement the Loss of cgnA and hrmA in EVOL20

Although there has been controversy as to whether a colony grown on a semi-solid surface is in fact a biofilm, there is abundant evidence linking colony morphologies with subsequent biofilm formation and structure phenotypes (Haussler et al. J Bacteriol 195(13):2947-2958, 2013). It was demonstrated in Example 1 with A. fumigatus that H-MORPH colony morphology corresponds with architectural changes within submerged biofilms relative to N-MORPH strains. Therefore, the cryptic gene within HAC that is necessary for H-MORPH in EVOL20 with the gene ID Afu5g14915 has been designated biofilm architecture factor A (bafA). Similarly, the uncharacterized genes with high nucleotide and amino acid identity to bafA within HBAC and HCAC will be referred to as bafB (AFUB_044360) and bafC (AFUB_096610), respectively.

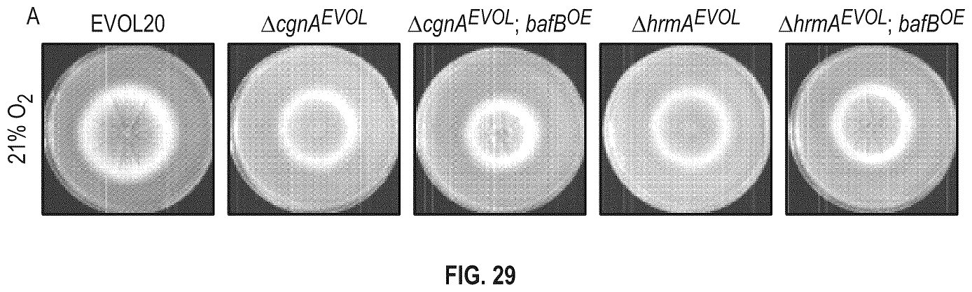

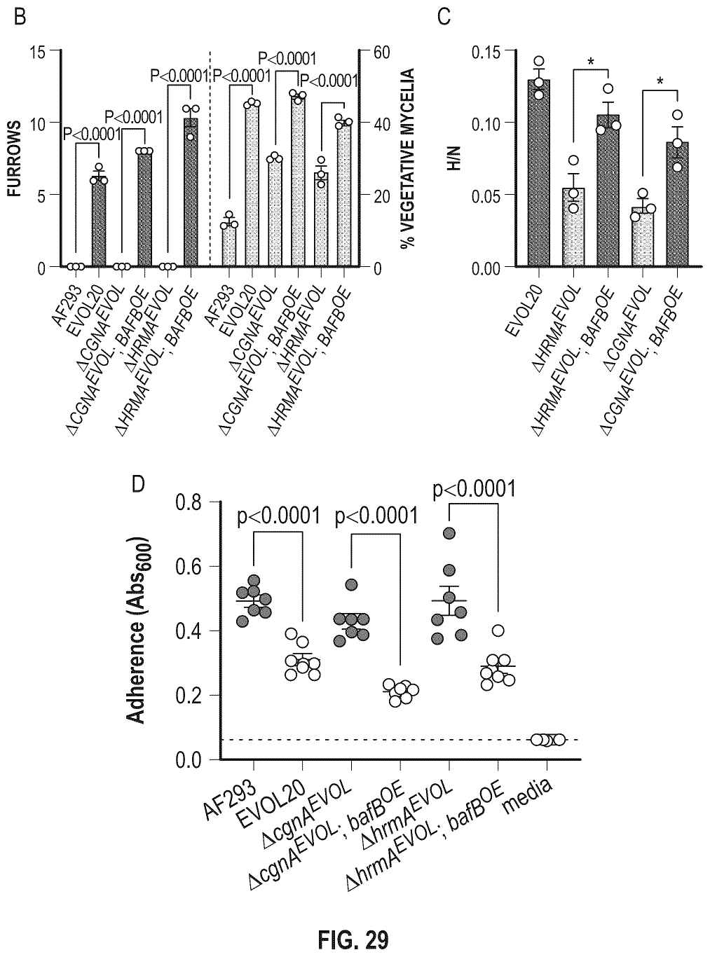

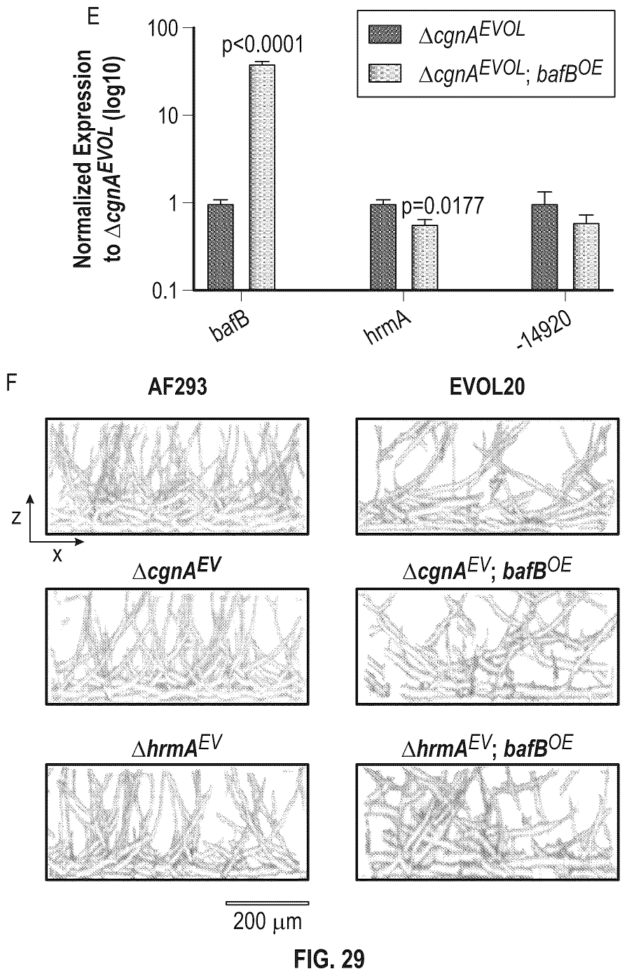

To determine if bafB from CEA10, whose protein sequence is 78.35% identical to bafA, could complement the loss of cgnA in EVOL20 (ΔcgnAEVOL), bafB was introduced with the constitutively active gpdA promoter (ΔcgnAEVOL; bafBOE). The resulting strain reverted the N-MORPH phenotype of ΔcgnAEVOL to the H-MORPH phenotype of EVOL20 with significantly increased colony furrows and percent vegetative mycelia (FIG. 29A, 29B). As mentioned above, the majority of HAC genes are not altered in expression as a result of cgnA deletion, thus the expression of other HAC genes could still be required for bafB to generate H-MORPH. The loss of hrmA in EVOL20 (ΔhrmAEVOL) reverts the colony to N-MORPH and mRNA levels of HAC genes are significantly reduced. To determine if hrmA and subsequently the HAC cluster genes that rely on hrmA for expression are necessary to generate H-MORPH in the presence of bafB, we introduced bafB with the constitutive gpdA promoter into the ΔhrmAEVOL strain (ΔhrmAEVOL; bafBOE). Even in the absence of hrmA, bafB is sufficient to generate H-MORPH and significantly increase colony furrows and percent vegetative mycelia (FIG. 29A, 29B). In addition to H-MORPH, EVOL20 has elevated hypoxic fitness (H/N) and reduced surface adherence relative to AF293 that is dependent on both hrmA and cgnA/bafA (FIG. 28C, 28D). The over expression of bafB significantly increases hypoxic fitness of ΔhrmAEVOL and ΔcgnAEVOL (FIG. 29C); and significantly reduces adherence of these strains to a plastic surface (FIG. 29D). Importantly, bafB is sufficient to complement these phenotypes in EVOL20 without increasing HAC gene mRNA levels (FIG. 29E). In fact, the mRNA levels of hrmA are slightly, but significantly, reduced as a result of constitutive bafB expression (FIG. 29E).

To test whether bafB expression alters biofilm architecture, a HAC-dependent phenotype of EVOL20, we cultured submerged biofilms for 24 hours and imaged the bottom ˜300 m of the biofilm. As a metric for biofilm architecture, we measured the angle of hyphal deviation from the vertical axis. As has been described for the N-MORPH strains AF293, ΔcgnAEVOL, and ΔhrmAEVOL, at 24 hours the bottom ˜50 of the biofilm features filaments that grow along the surface and have a high deviation from the vertical. At depths above 50 μm for these N-MORPH strain, the hyphae orient vertically and grow polarized toward the air-liquid interface with little deviation from the vertical axis. In contrast, the H-MORPH strain EVOL20 features hyphae throughout all 300 μm that are oriented with a high deviation from the vertical, in other words more hyphae are oriented horizontally above 50 μm. When bafB is overexpressed in the N-MORPH strains cgnAEVOL and ΔhrmAEVOL, the resulting H-MORPH strains (FIG. 29A) develop biofilms that also resemble the architecture of EVOL20 (FIG. 29F). There is greater hyphal deviation from the vertical axis above 50 μm in the biofilms of ΔcgnAEVOL; bafBOE and ΔhrmAEVOL; bafBOE (FIG. 29F). Thus, introduction of a constitutively expressed bafB is sufficient to complement the HAC-dependent phenotypes of EVOL20.

The BafB protein is predicted to have a signal sequence at its N-terminus (SignalP, FungiDB). To gain insight into how bafB could directly impact the biofilm architecture of the ΔcgnAEVOL strain, a c-terminal green fluorescent protein (GFP) tagged allele of bafB was generated in ΔcgnAEVOL. Introduction of the GFP-tagged allele, like the native bafB allele, is able to revert the N-MORPH colony morphotype of ΔcgnAEVOL to H-MORPH. In mature hyphae, the localization of the GFP signal is present both in the cytosol within circular structures that resemble trafficking vesicles or vacuoles previously described in A. nidulans, and concentrated toward the distal hyphal region. At the distal region, the GFP signal is present within circular structures as well as localized along the sides of the hyphae.

Whether the protein is localized on the inner surface or the outer surface remains to be determined. However, the presence of the N-terminal secretion signal peptide and the fact protein secretion occurs at the hyphal tip lends support to the hypothesis that BafB could localize extracellularly. Importantly, the hyphal tip is the region of active fungal growth, and as the colony morphology is a consequence of fungal growth this localization pattern indicates that BafB could be acting as the H-MORPH effector. The high amino acid identity shared between bafB and the HAC-resident gene bafA raise the question of whether bafA is the HAC effector and is sufficient to generate H-MORPH in the parental strain AF293.

Overexpression of bafA Generates H-MORPH and Elevated Hypoxic Growth in the Absence of HAC Induction in Two Independent Strain Backgrounds

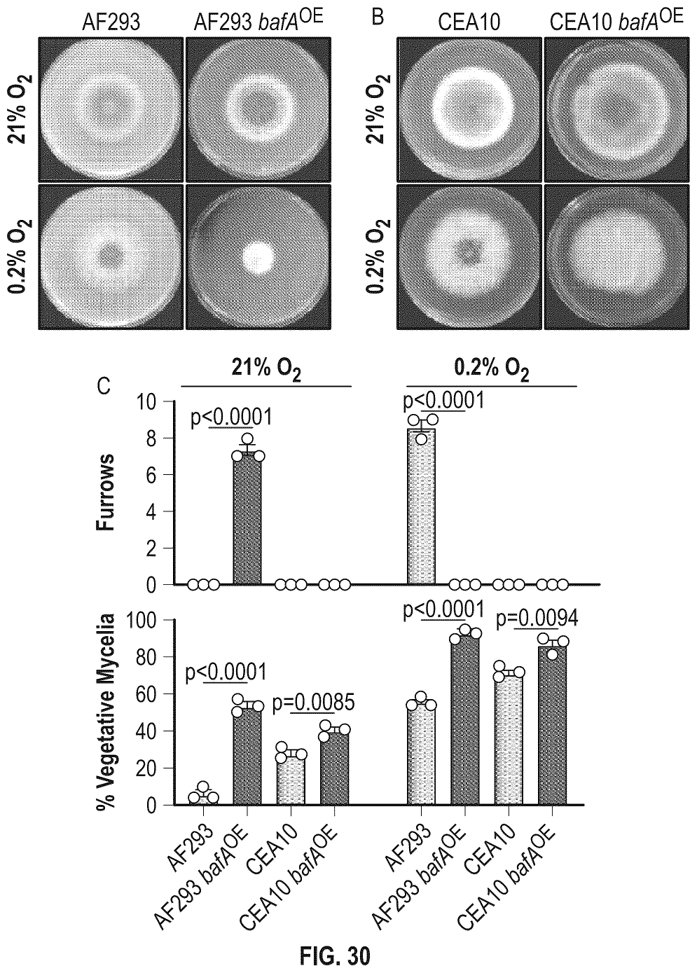

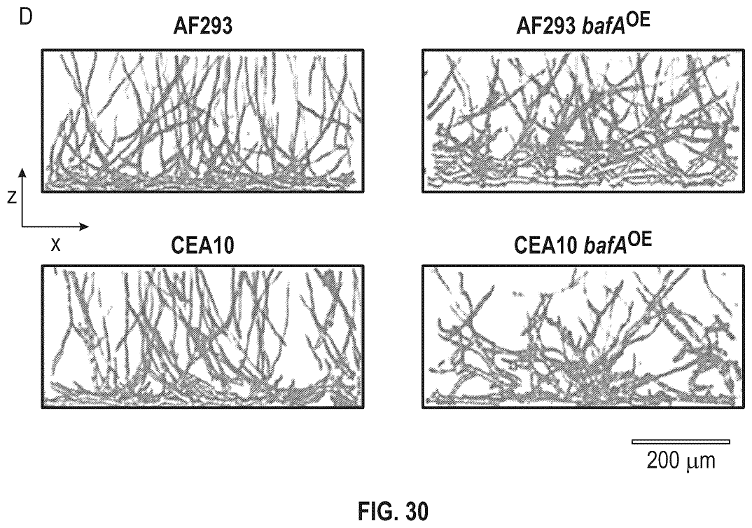

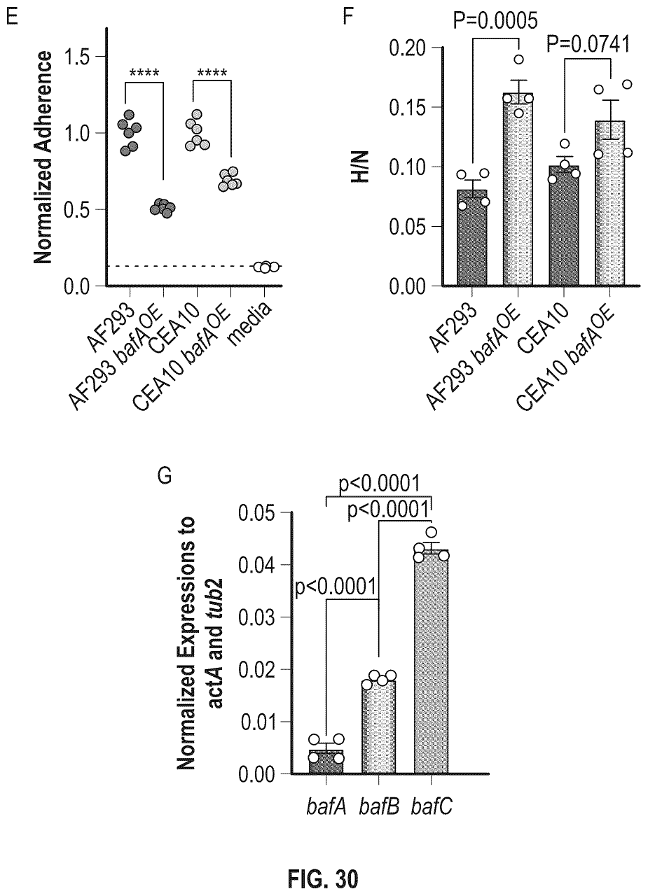

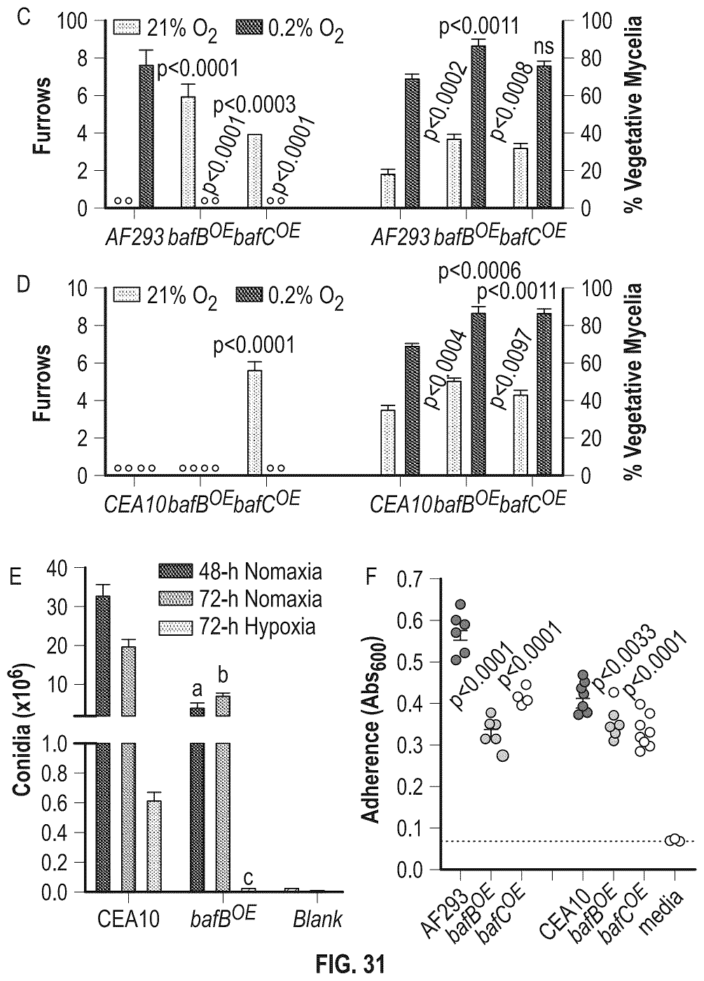

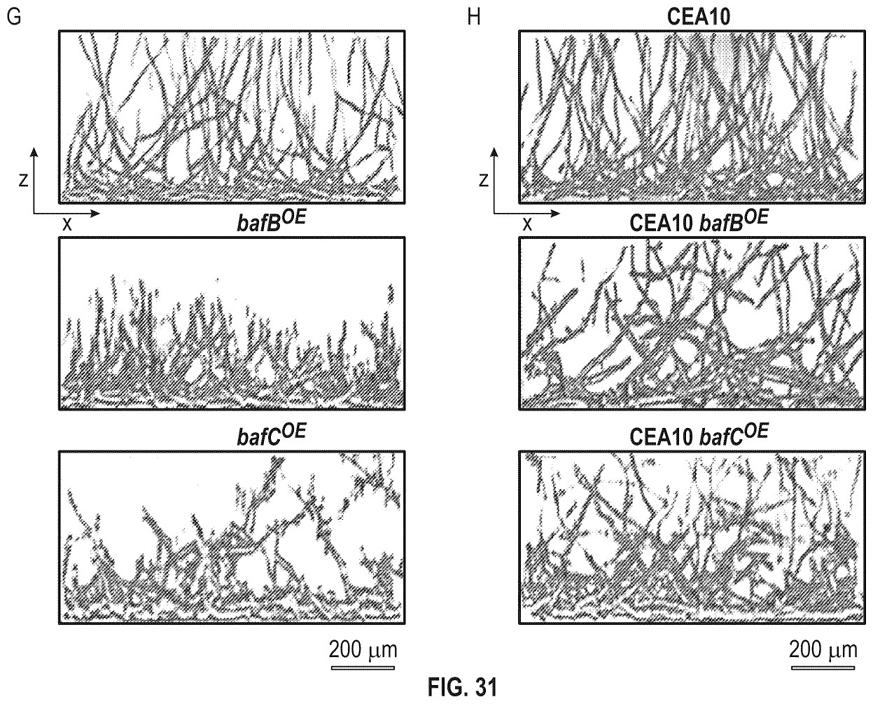

In the parental strain AF293, the basal expression of HAC is low, and previous RNA-sequencing data reveals no mapped reads to the predicted bafA ORF in AF293. In addition, qRT-PCR for bafA mRNA revealed no detection above background in AF293, but over expression of an additional bafA allele results in detectable bafA mRNA. The synthetic, elevated expression of bafA in AF293 results in H-MORPH colony morphology with significantly increased colony furrows and percent vegetative mycelia relative to AF293 (FIG. 30A, 30C). Interestingly, the colony morphology in hypoxia (0.2% O2) is also distinctly different as a result of bafA over expression. Unlike AF293, the colony in hypoxia is small, dense and lacks furrows and conidiation (FIG. 31A), resembling the previously published colony morphology resulting from constitutive hrmA expression (Example 1 above).

The strain CEA10 contains HAC, HBAC, and HCAC, but like AF293, bafA expression is below the level of detection by qRT-PCR in biofilm cultures but can be detected following introduction of a second over expressed bafA allele. Elevated expression of bafA in CEA10 qualitatively alters the colony morphology in normal (21% O2) and low oxygen (0.2% 02) and significantly increases the percent vegetative mycelia (FIG. 30B, 30C). However, no colony furrows are present as a result of bafA constitutive expression in CEA10 (FIG. 30C). Despite the absence of this macroscopic H-MORPH feature, over expression of bafA in CEA10, and in AF293, impacts biofilm architecture by increasing the deviation of hyphae from the vertical axis above the bottom 50 μm of the biofilm (FIG. 30D). Unlike AF293, even during hypoxic growth CEA10 colonies do not feature furrows, and instead abundant aerial hyphae develop generating a ‘fluffy’ colony morphotype. We speculate that perhaps there is a dichotomy among strains of A. fumigatus where some respond to low oxygen by forming aerial hyphae (i.e. CEA10) and others develop furrows (i.e. AF293).

H-MORPH in EVOL20, and other clinical isolates, coincides with reduced adherence and increased hypoxic fitness (hypoxic growth relative to normoxia growth, H/N) (Example 1 above). In both CEA10 and AF293, over expression of bafA significantly reduces hyphal adherence to plastic (FIG. 30E). Despite documented differences in hypoxic growth between AF293 and CEA10, bafA over expression also significantly increases the hypoxic fitness of both strains, though to a lesser extent in CEA10 (FIG. 30F). The inability for bafA expression to impact CEA10 colony morphology, and its apparent reduced impact on adherence and hypoxic growth relative to AF293 may be explained by the presence of the other baf genes encoded in the CEA10 genome. While bafA mRNA levels are undetectable in CEA10 during normal oxygen growth, mRNA for both bafB and bafC is detected (FIG. 30G). As the amino acid identity between these three proteins ranges from 45-78%, it was hypothesize that bafB and bafC are also sufficient to impact colony and biofilm morphology.

Overexpression of the bafA Orthologs bafB and bafC Generate H-MORPH-Like Phenotypes and Impact Hypoxic Growth

To determine if bafB and bafC are sufficient to generate H-MORPH phenotypes in the independent reference strains AF293 and CEA10, we used a constitutive promoter to drive expression of these genes and assessed colony morphology, adherence, and biofilm architecture. Introduction of either bafB or bafC in AF293 generates features of H-MORPH in normoxia with significantly increased furrows and percent vegetative mycelia (FIG. 31A, 31C). Similar to bafA over expression in CEA10, bafB overexpression did not induce H-MORPH features of colony furrows and increased percent vegetative mycelia in CEA10 (FIG. 31B, 31D). However, bafB expression significantly reduced overall conidiation in normoxia (21% O2) and hypoxia (0.2% 02), a complimentary metric to percent vegetative mycelia (FIG. 31E). Over expression of bafC in CEA10 is unique in that it does significantly increase colony furrows in normoxia relative to CEA10 (FIG. 31B, 31D). However, the percent vegetative mycelia is not significantly increased (FIG. 31D).

Despite variation in how the baf genes impact colony morphology in the two strain backgrounds, in both AF293 and CEA10 over expression of bafB or bafC results in significantly reduced adherence to plastic (FIG. 31F). CEA10 adheres less well to plastic compared to AF293, and the difference in adherence is smaller as a result of bafB or bafC over expression. As these two genes are already present and expressed in CEA10 (FIG. 30H), it is possible that this native baf expression contributes to this difference between CEA10 and AF293.

As putative biofilm architecture factors, we sought to confirm an impact of bafB and bafC on biofilm architecture, similar to that which we observe with elevated expression of bafA (FIG. 29D, 29E). In AF293, over expression of bafB visibly impacts biofilm architecture and formation in the XZ dimension (FIG. 31G) and XY dimension. The XY dimension reveals dense hyphal growth and abundant hyphal branching. The XZ dimension reveals a stunted 24 hour biofilm that reaches heights of only 200-250 μm (FIG. 31G). Similarly, regions of the 24 hour biofilms generated by the overexpression of bafC in AF293 (AF293 bafCOE) are also stunted with evidence of hyphae that are hyper branching (FIG. 31G). In regards to biofilm architecture as defined by hyphal orientation to the vertical axis, over expression of bafC but not bafB in AF293 results in increased deviation above 50 μm. In CEA10, over expression of bafB and bafC results in increased deviation from the vertical axis above 50 μm in 24 hour biofilms (FIG. 31H). There is also no evidence for hyper branching as a result of elevated bafB or bafC expression in CEA10. These data support a role for all three proposed baf genes in biofilm architecture, through multiple metrics, in two independent strain backgrounds of A. fumigatus.

Introduction of A. fumigatus bafA into Aspergillus niger Generates H-MORPH and Simultaneously Increases Hypoxic Growth

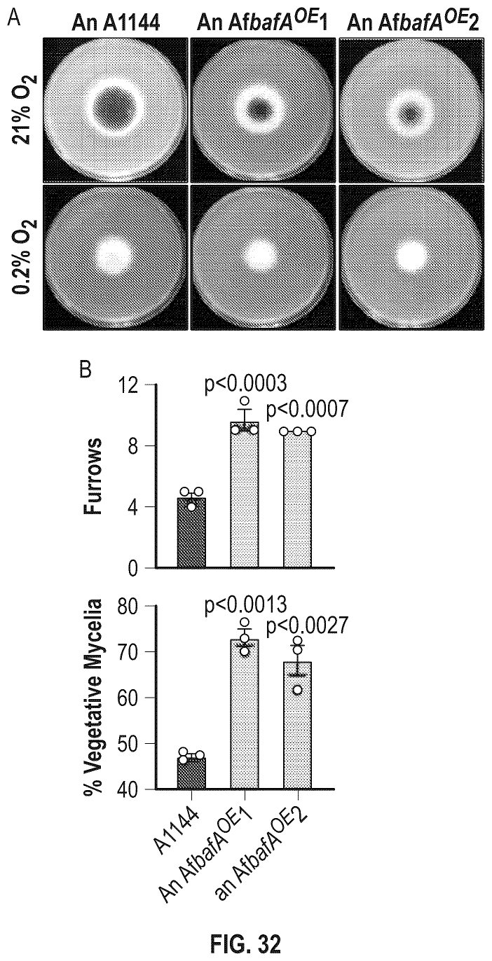

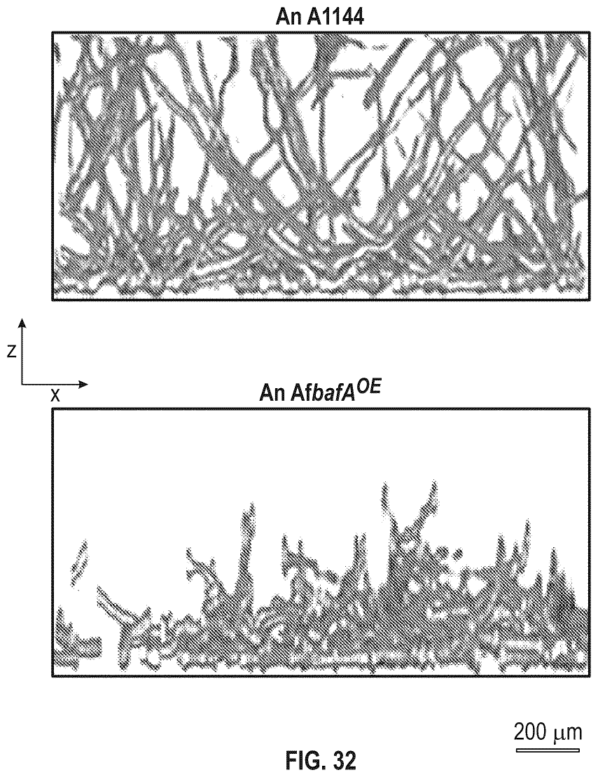

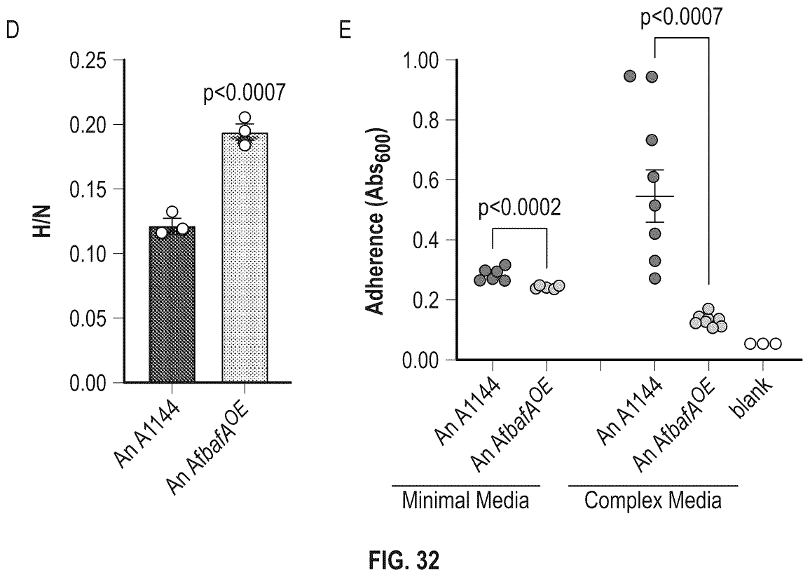

Among the Aspergilli, hrmA is absent from the notable species of A. nidulans, A. oryzae, and A. niger (Example 1 above). However, Aspergillus niger strain CBS 513.88 encodes a gene, An08g12010, with 69% nucleotide identity to A. fumigatus bafA and 41.03% amino acid identity to the predicted protein sequence of BafA. This suggests that the role of baf or baf-like genes may be conserved in other Aspergillus species. It was next determined if A. fumigatus bafA (AfbafA) could influence colony morphology, biofilm architecture, hypoxic growth, and adherence in the A. niger reference strain A1144. This strain was selected for its robust growth at 37° C. and the ease at which it is genetically manipulated. A. fumigatus bafA was overexpressed in A. niger with the constitutive gpdA promoter to generate An AfbafAOE. Over expression of bafA in A. niger generated H-MORPH colonies with significantly increased colony furrows and percent vegetative mycelia compared to the control A1144 (FIG. 32A, 32B). Intriguingly, the over expression of AfbafA in A. niger resulted in the production of a bright yellow pigment, shown here in two independent transformants (FIG. 32A). The production of yellow pigments by A. niger has been noted in the literature for decades as a result of various growth conditions and genetic manipulations.

The reference strain A1144 forms a submerged biofilm with dense filaments within the first 50 μm that are oriented perpendicular to the vertical axis (FIG. 32C). Above the ˜50 μm at the base of the biofilm, filaments become oriented more closely along the vertical axis, similar to what has been observed with N-MORPH strains of A. fumigatus (i.e. AF293) (FIG. 32C). Introduction of the constitutively expressed AfbafA alters the biofilm of A1144. At 24 hours, the hyphae are stunted reaching heights of only 200-250 μm in height (FIG. 32C). These stunted filaments highly deviate from the vertical axis throughout the height of the biofilm indicating that AfbafA is capable of impacting biofilm architecture across species. Not only does AfbafA impact the colony morphology to generate H-MORPH and modulate the biofilm architecture, but it also generates other H-MORPH and EVOL20 associated phenotypes including increased hypoxia fitness and reduced adherence. In AF293 and CEA10 expression of bafA results in increased hypoxia fitness (hypoxic growth normalized to normoxic growth); similarly, the hypoxia fitness of A1144 significantly increases with constitutive expression of AfbafA (FIG. 32D). Adherence of A. fumigatus is quantified in minimal media, however, the adherence of the reference A. niger strain A1144 is low in minimal media. Thus, the impact of AfbafA on A. niger adherence in both minimal and complex media where A1144 adherence is more robust was quantified. In both conditions, adherence was significantly reduced with expression of AfbafA compared to A1144 (FIG. 32E). Not only do reduced adherence and increased hypoxia fitness track with H-MORPH on the macro scale and microscale as has been observed previously, but they do so as a result of bafA expression across different Aspergillus species. The ability of bafA alone to generate these phenotypes in the two independent species of Aspergillus supports its role as the effector protein of HAC, and supports its application to modify biofilm architecture and function in Aspergillus species.

What is claimed:

1-26. (canceled)

27. A filamentous fungal host cell, comprising a nucleotide sequence encoding an Aspergillus niger biofilm architecture factor (baf) protein, or a homolog or ortholog thereof.

28. The filamentous fungal host cell of claim 27, wherein the filamentous fungal host cell is not Aspergillus niger.

29. The filamentous fungal host cell of claim 27, wherein the baf protein comprises bafA, or a homolog or ortholog thereof.

30. The filamentous fungal host cell of claim 27, wherein the bafA protein comprises at least 90% identity to the amino acid sequence of SEQ ID NO: 16.

31. The filamentous fungal host cell of claim 27, wherein the bafA protein comprises the amino acid sequence of SEQ ID NO: 16.

32. The filamentous fungal host cell of claim 27, wherein the fungal host cell is less adherent to plastic and glass surfaces relative to a fungal host cell that does not comprise the nucleotide sequence encoding the Aspergillus niger baf protein, or a homolog or ortholog thereof.

33. (canceled)

34. The filamentous fungal host cell of claim 27, wherein the filamentous fungal host cell produces one or more products of interest at a higher level than a filamentous fungal host cell that does not comprise a nucleotide sequence encoding an Aspergillus niger baf protein, or homologs or orthologs thereof.

35. The filamentous fungal host cell of claim 27, wherein the filamentous fungal host cell secretes one or more products of interest at a higher level than a filamentous fungal host cell that does not comprise a nucleotide sequence encoding an Aspergillus niger baf protein, or homologs or orthologs thereof.

36. The filamentous fungal host cell of claim 34, wherein the one or more products of interest comprise citric acid, gluconic acid, fumaric acid, kojic acid, lactic acid, itaconic acid, proteins, and secondary metabolites.

37. The filamentous fungal host cell of claim 36, wherein the secondary metabolites are selected from the group consisting of: β-lactams, compactin, cyclosporines, gibberellins, griseofulvin, lovastatin, mycophenolic acid, pigments, siderophores, and taxol.

38. The filamentous fungal host cell of claim 37, wherein the filamentous fungal host cell grows at a higher level in the presence of reduced oxygen than a filamentous fungal host cell that does not comprise a nucleotide sequence encoding an Aspergillus niger baf protein, or homologs or orthologs thereof.

39. The filamentous fungal host cell of claim 37, wherein filamentous fungal host cell oxygen consumption is reduced compared to a filamentous fungal host cell that does not comprise a nucleotide sequence encoding an Aspergillus niger baf protein, or homologs or orthologs thereof.

40. The filamentous fungal host cell of claim 39, wherein the oxygen consumption is reduced by about 10% to about 90%.

41. The filamentous fungal host cell of claim 39, wherein the oxygen consumption is reduced by about 10%, about 20%, about 30%, about 40%, or about 50%.

42-77. (canceled)


SEQUENCE LISTING (44 sequences)

SEQ ID NO: 1 - DNA, 1142 bp, synthetic construct
  source 1..1142: mol_type = other DNA
atggcatccacaaagcccgcttcgagtctcatttaccaggcatggaacaaactcagtatcaaccaaaccatccctagtgactcccttgaattacttggggagcgtttggctattgccttcgcacccaaactcaaggagcaacgaaggaatggccggcgtcggaatctggaatatgtggcacaacatcgacggaagattgctcgaaaaatctacttggagattctggagaaagacccaaatatctttcttccttttatcctggctgtttcccctagagcatgcttatcctttgatatctcgagctttcttgaacagcaccaaagccaaggaagacatttcctccgcaacaatgccgaagcgatcctctggggtctcgcaaagaaacatgacattgatggctccctccatttcaggaagctgatgcgtgagattttccaactgtctcctccagcgacagaagccgaaggcaaggagcattattcattgcatttaagcactctccccgcaatccgcaatgccttcggtgatgttatctttgacgcaattgaacgttcccctacacaggtgacagcgagagctaaaggttatttctctgagaaaaccgaaagtgtttggacaaaagttccctacagaagttctcaagacgcaatcatatctcttgaagtagggtcggcaatcgagcttgcgaatgtgttgttcccaatcgcaacccaaaaaattgtctctatcctttccgcatgttctcccactgtgcgccagaagaacttttctgaggctattctcggcccagaccctcaggatacaccggcaacatcatcagaaatcggtatgaagtttaaggtacacatgactgcagttgctaattccaccctgtgctagatgtggcgtattttactctgcgaggagcaacggtctcggcaattgaatcagtctttcgcgctgatatttgcgaaggtattaagggcagcgaactgagaaactgggaaaaggagcagctgctcatcgacacgacagattgtgtcacgatgcagatatggcgggcacaacctcaacatggaaccatcaagttgcgtattggattctatgcagcggtgaatttggcaaatcggctgtatgcagaaacaccccaagatcacatatag

SEQ ID NO: 2 - DNA, 8855 bp, synthetic construct
  source 1..8855: mol_type = other DNA
cgccgtaacgtaacaaagcggggttggtagtgtttgcaaatgcattcacatggaccgatcacttttctttccagtctgtccattctgtccaatctgtccgatctgacctgcccagtctgtccagtctgtcccttgtgtcgtccgatccaagctggttatcatggcatccacaaagcccgcttcgagtctcatttaccaggcatggaacaaactcagtatcaaccaaaccatccctagtgactcccttgaattacttggggagcgtttggctattgccttcgcacccaaactcaaggagcaacgaaggaatggccggcgtcggaatctggaatatgtggcacaacatcgacggaagattgctcgaaaaatctacttggagattctggagaaagacccaaatatctttcttccttttatcctggctgtttcccctagagcatgcttatcctttgatatctcgagctttcttgaacagcaccaaagccaaggaagacatttcctccgcaacaatgccgaagcgatcctctggggtctcgcaaagaaacatgacattgatggctccctccatttcaggaagctgatgcgtgagattttccaactgtctcctccagcgacagaagccgaaggcaaggagcattattcattgcatttaagcactctccccgcaatccgcaatgccttcggtgatgttatctttgacgcaattgaacgttcccctacacaggtgacagcgagagctaaaggttatttctctgagaaaaccgaaagtgtttggacaaaagttccctacagaagttctcaagacgcaatcatatctcttgaagtagggtcggcaatcgagcttgcgaatgtgttgttcccaatcgcaacccaaaaaattgtctctatcctttccgcatgttctcccactgtgcgccagaagaacttttctgaggctattctcggcccagaccctcaggatacaccggcaacatcatcagaaatcggtatgaagtttaaggtacacatgactgcagttgctaattccaccctgtgctagatgtggcgtattttactctgcgaggagcaacggtctcggcaattgaatcagtctttcgcgctgatatttgcgaaggtattaagggcagcgaactgagaaactgggaaaaggagcagctgctcatcgacacgacagattgtgtcacgatgcagatatggcgggcacaacctcaacatggaaccatcaagttgcgtattggattctatgcagcggtgaatttggcaaatcggctgtatgcagaaacaccccaagatcacatatagtaacctttcatcttttcggccttcttaaatcattgcctttctgtgagtcgcgactttccaccctttatgaatacaccaataccagggggaagaacgatttcaccgcttcccttggcaatccatatagttcccttctcattctggaacctgatttcatcgcagttgaagcaatataaattcctttcgaggttttctgcattgtagggatggaatgggtgcgtgaaatatttgtcgaaaatagaagggtccgaaagttctttccaagctgcgcgctgaaagttgttagcccatctttcgagcatatggtttggcccacatacgagagattcttttgtgatcggttgagattcttccgtgatcggttctgtcattttcattaagtcagagagccctcttgtatgccggcttttgctgtcggatccggcgagataatcgctcctaagccagtcagtcagggaaaagcaaagataaataaaatataggcgaggagtacaaccaggcacgcgtcgtagactatttttctgaagagtttgtcacgtaacctacctcatatggatgggtagttcgaatacttgattgacttgacccgaggttctgaaggcggcggaggaaattgcccaaccccaccattgcattttcaggtatcaatctctgccacactgtggctaaattcgtctttatcgacacgtgatcacgttccctcttccagccctggtatcagagaatcatcgagttatcgcttgtttcaatttcgtcttgcaattagcttagggaataagcatgtggtcacatcaacctacagagcgctaccggtctttgcgctgagactctcagtgatccgcccaacagacaactagactttgaggttgtcgatataaccacaacaaatggcctgtatatcaacgatgtccacgcaattgtctcaagcctcttcacccgacttccagcactagcatccaagcggcctctcctcttctcccatgtttctcgtagcgcgcctgcatatacttatatctggagatatgttaaaggagctggaagcctggagcatacgctggaagcctggagcatacgcttcaagtgctgccatattcagatagctgagtaggcacaattaggtctaagttcagggaattgcacctctcgcttcattgtccgtcgattcgtatcggtctctagttctccccgtttatcactctcactcggtggacagtccgtccagtccgtccagtccgtcgagcctgtccaatctgtccaatctgtccaatctgtccaatctgtccaatctgtccaatctgtccaatctgtccaatctgtccaatctgtccaatctgtccaatctgtccaatctgtccaatctgtccaatctgtccaatctgtccaatctgtccactctgtccactctgtccactctgtccaatctgtccactctgtcccctctgtccactctgtcccctctgtccactctgtcccctctgtccaatcggtccaatctgtccaatcttgatgatctcgatgatcaataccatttagcgagcgctagtgacgccttacagcgttgcggcgtcttatggcttatacatcttccgaatatcagtcgttcgatctccagatcacacctcggggtgaaacaagcgccatagttcttggtgcgcctgagcttttccctgcccggccactcagtcgtgatggcttcccagagtattccataagtcgaaaccagagataggccagcggacggcaaatctcctctgctccgttctcaaccaatactgccaaagtgagcaatggaaagtgttccaggagcaccgtggcctttaaaagcgccagccttgtcccagatcctcaccgcaattcggcacagaccaacgcagactttcattgaccattcttcatttccagatcgctgcatagtttccggtatggcttggtatagagccttactcccgtgcataccgttgtggcggaaggttctggggcgtaacagcaccgatgaggacggacggagtgaagacgaccttacttcattgagcgataaaatgcctacttttgaagaaacgacaactgtgagtactgttcgtgaaattgcctccagctgtctaatgtctccgtcggtcagatcacttctgcaaagtacatcaacggtgaaaaaatcatggagcataccgttgtggagaccaagcacattgacgaacgtggagacaccagcgtcagtaacggtgattcgaacagcactgcggtaaccagacattctgggcttagctctgtcagcctttcagatcaaagtacaattgtcgaggacgcgaatgctctggaagaacctgaactctttgctgttcactctccatatgttgacgactcaaccggcgagcagatggttagactctactacgagctcccagtaagcctcgatgatcttgagatcataggccttgaatctcgtattccagagtctgacgatgattcaattgaggcccgctttcgttatcgaggagaggatttttggctacctgttcgttattcttatgccaaagctcgaatggttttaacgggtgtatgctgaatggtctgacttcttcactgttgttattttctatttcccggctgctggccactcaatattatcgcaagcactatacaaaataaatagtcctatctctataacagagtacgtcacaaacagcgttcgtccttggcaagaatataaatagtgtcaatctgctgagaaaaggaggtatgaaatccacttcattcaagcaatggttcccttcgtatacattattatttgtatatggatgggaacttttcttgtcttagttgccctgaacgcccgctttaagaagaagcgcattgctgatcctgagtcttctgctgccttcactgatcatccccatcaagagtaacatggattctgtatgtccctttgggcatgtttatgtggcagttactaatatattagatggaaactaggaagcggcaaaacacaggggcaggaagggaaggactcattccaagaaagaaggacaaggaagaacacaagagtgtaaggggtagcaatcctcattggcgtctactagctgatgggttaaagcaagatacaggtgttcatacgctccaagacaacgatcctgttgtcgagaagccgccattaatttacaggagagctggagaagcgccgaaacatgattgggacaagaagtcacccggtttgcgccttagacggtcctgctgtagctgcgggaaggaagtaccggtaggcctggtctgtcgtatttgtcaccatgagtcttgtcctgagtgcttgaacatgcaaaagcgagattacggatgttgatcagatacacgggcccctatactaggactagttaccactaggattgtactcaaccttaacagtggcgtttggtacttcgttacagctaaggggagaggacagttcctactttcatgtgcttcaagggagaggctcctccaactactgttgctagggaggaaagacactctattcttagctgagggctgcaggaattcgcatccatgcagtcataccatccatggcatgcaatctatgttgcattagatgcaagaagtaggatagagaacccatgtacttgattcacgctagcaggacagagagagataccatacccgacgggagcccctgcatgacttcctgtctgcagcttgtcgtgcgtgtatcattcccatgcgccacgaactcataggcagtggtagttcagaacactctttttttttaaaaaaaaaaaagataggaaaataataatttaggggaagaaaagtaaaaattaaaaagaaaaagttccagatggcgcttctacttctatattcgatcgttattcaataccccagaggcacaggcattccgatctctcatcgccaacactgaaaagcagccattttcccccgtcttaaagctccaatcctccttcttctcatctacttcctcgctccttcaggaccttgagtgttccgttgagctattgggtaacttctcacctgtcaatcatcgattgtcctttctcttgacttgacttcgtgtcgccattctcatttacgatacatatccctggagcagaaaacaaagaaaagggccaattactcttgatctagttccaactctgttgctgcttggaacatccgcccatctgtgtggtgaaatcagatgccagcatccatcttgcagcttctcccacttcctgggccgatcttgaatgtttgctctcgaacctcgcttagtatttgatctccattctcatctgggtacatcctgtgagtagcatgtcgtcacttgtcacacatactacccgctctcaaatctgtttgatgggagtcaatctgcctcgaaatggctcgtctgccttcacaagcaaactacagcagatggcgggggcatggactcgagccacagtgctggctctcgcttgcatctggaccttcttattctttctcattgctgtatctttttcccccttgaggcttctggcgcgctgcacctttccaagtatcaaaccaaagctaatcaggggcgtttggcgtcctgccatggcttcactagacctggatctctgcagcctcatcaccatctcggatcacctggttctgatcaccttggaagaaagcacaaagaccttggagacaatacatattgccgccatcgcagctccctccaatctcgacagcattttcatgtgtcgggcactatctacctctcggcaattcagtaaccgtactgcctgagaaacatcaacctctcaaattacacaatggtgttcagcgcacctgctcctggtgtgggctccagtaaaaggccagcgtcatgcatgcaggacgatgttgatgagcgggataatgtcccagtaagtgataccagcattggaaaggcagatggagctgactcatctcctatatagcccatgggtctatgcgtgcgttcatcgatcaacatcccttacttccactacgccatgatctatctcgacaacatgggcagacttaaggtgatggaatctccgtctatccaggagcaaaatgagactgttttcacaaccgaagtacgtgaaagatttttggaaatccttggtgccaaggtaggatatcaaccgcccatggttcgaagtatgtaaacactccgcgcacaagtacaatattcttgctgatctcaattgaacagggttgtcagctgccggtgctacaccatacagctatgatcctcaacaaccgcttggttgcttgtcttaccgtcaaactaagcgggacagaaattccccagcccactctatgtacggtgtgccgccatccgtccagttctcagccccggttgaggaatcgccctcttgtggatcagtggacatggtcgggctcgagattggtgatactcctaatgtccttgactactatgagagatccttaaagcactttcggcaggtcaactgtcgccagatcctaaagacattcattaagttcattgagccacgaaagcaagccaagcacccctataatggaggtaaaccccctgcaggagcccctcctggtaagaagggcgacccagagaagacaaagcctgaatggtggcccgccaatgtggtccacaaggagcctgaccatcttcgaaaggatcgtacgtgtaacccttcagaaaatcttcagtgtcaagtaactttgctgacagacttagaacgcctgtctctgttaattcatatcatccgcaggcttggaagatttggtatcaccacggatcaattgcaggaaattgcccacgactgcaagcggcggctcagcgacccccacaaactccaaatcttggacgaggtcttcagagtgagaaggattgaagaacgctacgaaagaggagaagttggtaagcggcatcatctttccatgaaattcattttgacagctgttgacgagcctcagatgccaacaagatcgtatatgttgtcaaccgagagtcgaatcagaaagagaaggatggcgactccaacgtggatccggaccagaagcatgagcaagaagacgataatgcgcgggaggcacttcccattctccactccgagaagaactcaaccagcccgatgtcgaactcagccgagcacacgggcatggcggcaccaagtcgtccaatgaatatgggaggtgacagaaaccagttgtttcctttaccggagtggccgagcttcggtgagacaccccaggatgatcgaattttctttcccacgacctctaagtataccgaagattatgcatcgcagcagatgcctagaacacctgcaacaacagcacttgtcagcactaatgagacacatgcggcctttgattatatgacacaggagtccatcacctcctcctccccagagcagacttcccaccaccgccaagcacccctgcccatgcagcactcggccagcctcgacccttggacccctacgttccgacataatttcttcaacccaatggtgtatagtactgcaccccgtcacgccatgtcccaggctactatgttatctcagtttcccaggtccacgacgtctcatggccaggaaatgcctcacatggctcacggcctgccgaacctgcctcaagacagaccttcaagcatggatggcatgagcatgagaggcccttctttccgcacaggatttttgagtcatccctgtgacccatcacagcaggctcctcattctagcggatgcggccatcctgacagttggactcaaaatagaccacatgtataatcttaactgattgatccttgaccactgttttgaccctcctgcagccttgaagcttcgtttcactgatgattgttcttcgactttgtttctgtccctgactttgttgtcaatgcggacttatccatgcggcttgttccacgtcaagtgactaccaggacactccgtggttttatatggcaggtactggcgatgactttccaattcttcttcgtttagtatatatactcgtttcttgttctatgttcgatcatgtctttttccttatacatacctccaaaaatcctgttggagatggcgccagatggcatgagatgcaaatatggatgatgttcttgtgtttgttcatttcaatttctttctcttaatcatgatttgaacaattggcagcgaggtatggcggagctcgttctctttggatgccgatcagctgaataggaggtaacgaggcatgagggtgtttcattatgactctctccggtgtttgtcatttaagggtgcgagggggaagtgtccgtttcgatgtcctaggatatcgaaaatctgagtagtagccacgtgaccctatgctgacggctgggctggaagacaagcaggttgctgcttacgagaatatgttgaggtattctcgttatcttcgtgaagaatgccgtctccttggccctctagccaaagtctgggttgctgaaaggctagctggaattgagaatcgactgtctgcgtccgagtcgcctagaggtgggaaggccccctctttctcatacatatgctgactctgcagaccataccaattcgctgcccgaa

SEQ ID NO: 3 - DNA, 641 bp, unidentified
  source 1..641: mol_type = other DNA; note = Aspergillus fumigatus
atggcttggtatagagccttactcccgtgcataccgttgtggcggaaggttctggggcgtaacagcaccgatgaggacggacggagtgaagacgaccttacttcattgagcgataaaatgcctacttttgaagaaacgacaactgtgagtactgttcgtgaaattgcctccagctgtctaatgtctccgtcggtcagatcacttctgcaaagtacatcaacggtgaaaaaatcatggagcataccgttgtggagaccaagcacattgacgaacgtggagacaccagcgtcagtaacggtgattcgaacagcactgcggtaaccagacattctgggcttagctctgtcagcctttcagatcaaagtacaattgtcgaggacgcgaatgctctggaagaacctgaactctttgctgttcactctccatatgttgacgactcaaccggcgagcagatggttagactctactacgagctcccagtaagcctcgatgatcttgagatcataggccttgaatctcgtattccagagtctgacgatgattcaattgaggcccgctttcgttatcgaggagaggatttttggctacctgttcgttattcttatgccaaagctcgaatggttttaacgggtgtatgctga

SEQ ID NO: 4 - DNA, 635 bp, unidentified
  source 1..635: mol_type = other DNA; note = Aspergillus fumigatus
atggtgtggtatagggccatactcgtttgcatgccgtggtggctcatggggcgtaacagcaccaatgagggcaaacggagtgaaggcgaacgggctccaatgattgataaggtgcccactttcgaagaaatgacaattgtgagtactgttttgtggggttgcctccagctgtctaatgcttccttgcgcagaccacctctgcaaagtatgtcaacggtgaaaaaatcatggagcataccgttgtggagaccaagcaaattgacaaccgaggagacaccagcgtcagtaacaatgattcaaacagcactgcggaaaccagacattctgggcttagctccgtcagtcattcagatcaaagtaaagttgttgaggacgcgaatgccctggaaaaacctgaactctttgctgttcactctccatatgttgacgcctcaaccggcaagcagatgttaagactctactacgagctcccagtaagcctcgatgatcttgagatcacaggccttgaatctcgtattccagagtctgatgatgattcaattgaggcctgcttttgttatcggggggagaaattttggctacatgttccttattcttatgccaaagctcgaatggttttaatgggtgtatactga

SEQ ID NO: 5 - DNA, 637 bp, unidentified
  source 1..637: mol_type = other DNA; note = Aspergillus fumigatus
atggcttggtatgaagtcttcgagcaatgggtgtactggtgctggcagcgcatatggcccttcgacgacagcaggagggacggacgaaacgaagacgacctaacttcgttaaccgataaaatgcctgtttttgaagataagatcatcgtgagtactgtccatgaggaggcttccatctccctaaccttggcagaacacctctgtcagatatgtcaatggagagatcgcggcatatgtcgtccagacccagtatctcgatacccaagaggtctcctctgctagggactcttattggaaaagcgttgcggatatcaaaccgggtgacttctgctcccatagtatttcggatcagagcacaattgtcgaagaaaacgaagcgaaggcgctggaaggacctgaaccctttgctgttcgcccttcgtatattggatccactggcaaacgcacggtcgacttcttctacaaggtctctctaccactggatgatcttgagatgagagacaaggaatcgcgtgttccggagtctagcgaagatctgattgaggctctcttccactatcaaggggccgatatttgggtatatgttccttattcgtacgccaatgctcgaatggtttcaggcggtcccactgaatga

SEQ ID NO: 6 - DNA, 640 bp, unidentified
  source 1..640: mol_type = other DNA; note = Aspergillus niger
atggcttggtacagtgctttactcccgtgcatgctatggtggcggaacctcctgtggcgtaacagcaccaataggtatagacagagtacagacgaccttacttcactgactgataagatacctaatcttggagaaagggtaagtgtaaatactttttgaggtcgcttctaggagtctaatcactttggaaagacaactcccacagataacatcgatggaaagagcttcgtggcacatactgtcgtgcagaccaggcggattggtgaccgagaacgttgctgcttcaatgactcggatttgaacagcggcacagttaccaaatttattgagctttgttaatagcattccagatcagagtactactgccggaaaaattaatcacaaggccctgcgagatcctgagctctttgctatccgctcgtcatgcatcgacaaatcagccagcaagtggatggttagtctctactacgaacccccacccagccttgatgacctcgagattaagaacttcggatctcgtattccagagtcggaggatgatccaattgaggctatctttcactatgagggagagaacatttgggtatctgttccttatttgtatgctagaactagaagcctttcaagcggtctgttctga

SEQ ID NO: 7 - DNA, 582 bp, unidentified
  source 1..582: mol_type = other DNA; note = Aspergillus fumigatus
atggcttggtatagagccttactcccgtgcataccgttgtggcggaaggttctggggcgtaacagcaccgatgaggacggacggagtgaagacgaccttacttcattgagcgataaaatgcctacttttgaagaaacgacaacttctgcaaagtacatcaacggtgaaaaaatcatggagcataccgttgtggagaccaagcacattgacgaacgtggagacaccagcgtcagtaacggtgattcgaacagcactgcggtaaccagacattctgggcttagctctgtcagcctttcagatcaaagtacaattgtcgaggacgcgaatgctctggaagaacctgaactctttgctgttcactctccatatgttgacgactcaaccggcgagcagatggttagactctactacgagctcccagtaagcctcgatgatcttgagatcataggccttgaatctcgtattccagagtctgacgatgattcaattgaggcccgctttcgttatcgaggagaggatttttggctacctgttcgttattcttatgccaaagctcgaatggttttaacgggtgtatgctga

SEQ ID NO: 8 - DNA, 582 bp, unidentified
  source 1..582: mol_type = other DNA; note = Aspergillus fumigatus
atggtgtggtatagggccatactcgtttgcatgccgtggtggctcatggggcgtaacagcaccaatgagggcaaacggagtgaaggcgaacgggctccaatgattgataaggtgcccactttcgaagaaatgacaattaccacctctgcaaagtatgtcaacggtgaaaaaatcatggagcataccgttgtggagaccaagcaaattgacaaccgaggagacaccagcgtcagtaacaatgattcaaacagcactgcggaaaccagacattctgggcttagctccgtcagtcattcagatcaaagtaaagttgttgaggacgcgaatgccctggaaaaacctgaactctttgctgttcactctccatatgttgacgcctcaaccggcaagcagatgttaagactctactacgagctcccagtaagcctcgatgatcttgagatcacaggccttgaatctcgtattccagagtctgatgatgattcaattgaggcctgcttttgttatcggggggagaaattttggctacatgttccttattcttatgccaaagctcgaatggttttaatgggtgtatactga

SEQ ID NO: 9 - DNA, 591 bp, unidentified
  source 1..591: mol_type = other DNA; note = Aspergillus fumigatus
atggcttggtatgaagtcttcgagcaatgggtgtactggtgctggcagcgcatatggcccttcgacgacagcaggagggacggacgaaacgaagacgacctaacttcgttaaccgataaaatgcctgtttttgaagataagatcatcaacacctctgtcagatatgtcaatggagagatcgcggcatatgtcgtccagacccagtatctcgatacccaagaggtctcctctgctagggactcttattggaaaagcgttgcggatatcaaaccgggtgacttctgctcccatagtatttcggatcagagcacaattgtcgaagaaaacgaagcgaaggcgctggaaggacctgaaccctttgctgttcgcccttcgtatattggatccactggcaaacgcacggtcgacttcttctacaaggtctctctaccactggatgatcttgagatgagagacaaggaatcgcgtgttccggagtctagcgaagatctgattgaggctctcttccactatcaaggggccgatatttgggtatatgttccttattcgtacgccaatgctcgaatggtttcaggcggtcccactgaatga

SEQ ID NO: 10 - DNA, 465 bp, unidentified
  source 1..465: mol_type = other DNA; note = Aspergillus fumigatus
atggcttggtacagtgctttactcccgtgcatgctatggtggcggaacctcctgtggcgtaacagcaccaataggtatagacagagtacagacgaccttacttcactgactgataagatacctaatcttggagaaaggttaccaaatttattgagctttgttaatagcattccagatcagagtactactgccggaaaaattaatcacaaggccctgcgagatcctgagctctttgctatccgctcgtcatgcatcgacaaatcagccagcaagtggatggttagtctctactacgaacccccacccagccttgatgacctcgagattaagaacttcggatctcgtattccagagtcggaggatgatccaattgaggctatctttcactatgagggagagaacatttgggtatctgttccttatttgtatgctagaactagaagcctttcaagcggtctgttctga

SEQ ID NO: 11 - protein, 362 aa, unidentified
  source 1..362: note = Aspergillus fumigatus
MASTKPASSLIYQAWNKLSINQTIPSDSLELLGERLAIAFAPKLKEQRRNGRRRNLEYVAQHRRKIARKIYLEILEKDPNIFLPFILAVSPRACLSFDISSFLEQHQSQGRHFLRNNAEAILWGLAKKHDIDGSLHFRKLMREIFQLSPPATEAEGKEHYSLHLSTLPAIRNAFGDVIFDAIERSPTQVTARAKGYFSEKTESVWTKVPYRSSQDAIISLEVGSAIELANVLFPIATQKIVSILSACSPTVRQKNFSEAILGPDPQDTPATSSEIDVAYFTLRGATVSAIESVFRADICEGIKDSELRNWEKEQLLIDTTDCVTMQIWRAQPQHGTIKLRIGFYAAVNLANRLYAETPQDHI

SEQ ID NO: 12 - protein, 362 aa, synthetic construct
MASTKPASSLIYQAWNKLSINQTIPSDSLELLGERLAIAFAPKLKEQRRNGRRRNLEYVAQHRRKIARKIYLEILEKDPNIFLPFILAVSPRACLSFDISSFLEQHQSQGRHFLRNNAEAILWGLAKKHDIDGSLHFRKLMREIFQLSPPATEAEGKEHYSLHLSTLPAIRNAFGDVIFDAIERSPTQVTARAKGYFSEKTESVWTKVPYRSSQDAIISLEVGSAIELANVLFPIATQKIVSILSACSPTVRQKNFSEAILGPDPQDTPATSSEIDVAYFTLRGATVSAIESVFRADICEGIKGSELRNWEKEQLLIDTTDCVTMQIWRAQPQHGTIKLRIGFYAAVNLANRLYAETPQDHI

SEQ ID NO: 13 - protein, 192 aa, unidentified
  source 1..192: note = Aspergillus fumigatus
MAWYRALLPCIPLWRKVLGRNSTDEDGRSEDDLTSLSDKMPTFEETTTSAKYINGEKIMEHTVVETKHIDERGDTSVSNGDSNSTAVTRHSGLSSVSLSDQSTIVEDANALEEPELFAVHSPYVDDSTGEMVRLYYELPVSLDDLEIIGLESRIPESDDDSIEARFRYRGEDFWLPVRYSYAKARMVLTGVC

SEQ ID NO: 14 - protein, 192 aa, unidentified
  source 1..192: note = Aspergillus fumigatus
MVWYRAILVCMPWWLMGRNSTNEGKRSEGERAPMIDKVPTFEEMTITTSAKYVNGEKIMEHTVETKQIDNRGDTSVSNNDSNSTAETRHSGLSSVSHSDQSKVVEDANALEKPELFAVHSPYVDASTGKQMLRLYYELPVSLDDLEITGLESRIPESDDDSIEACFCYRGEKFWLHVPYSYAKARMVLMGVY

SEQ ID NO: 15 - protein, 196 aa, unidentified
  source 1..196: note = Aspergillus fumigatus
MAWYEVFEQWVYWCWQRIWPFDDSRRDGRNEDDLTSLTDKMPVFEDKIINTSVRYVNGEIAAYVVQTQYLDTQEVSSARDSYWKSVADIKPGDFCSHSISDQSTIVEENEAKALEGPEPFAVRPSYIGSTGKRTVDFFYKVSLPLDDLEMRDKESRVPESSEDLIEALFHYQGADIWVYVPYSYANARMVSGGPTE

SEQ ID NO: 16 - protein, 154 aa, unidentified
  source 1..154: note = Aspergillus niger
MAWYSALLPCMLWWRNLLWRNSTNRYRQSTDDLTSLTDKIPNLGERLPNLLSFVNSIPDQSTTAGKINHKALRDPELFAIRSSCIDKSASKWMVSLYYEPPPSLDDLEIKNFGSRIPESEDDPIEAIFHYEGENIWVSVPYLYARTRSLSSGLF

SEQ ID NO: 17-27 - skipped (11 entries)

SEQ ID NO: 28 - protein, 23 aa, synthetic construct
RRNGRRRNLEYVAQHRRKIARKI

SEQ ID NO: 29 - protein, 23 aa, synthetic construct
ARNGAAANLEYVAQHRRKIARKI

SEQ ID NO: 30 - protein, 31 aa, synthetic construct
GKEHYSLHLSTLPAIRNAFGDVIFDAIERSP

SEQ ID NO: 31 - protein, 31 aa, synthetic construct
GKEHYSLHLSTLPAIRNAAGDVIADAIERSP

SEQ ID NO: 32 - protein, 193 aa, unidentified
  source 1..193: note = Aspergillus fumigatus
MVWYRAILVCMPWWLMGRNSTNEGKRSEGERAPMIDKVPTFEEMTITTSAKYVNGEKIMEHTVVETKQIDNRGDTSVSNNDSNSTAETRHSGLSSVSHSDQSKVVEDANALEKPELFAVHSPYVDASTGKQMLRLYYELPVSLDDLEITGLESRIPESDDDSIEACFCYRGEKFWLHVPYSYAKARMVLMGVY

SEQ ID NO: 33 - protein, 193 aa, unidentified
  source 1..193: note = Aspergillus fumigatus
MAWYRALLPCIPLWRKVLGRNSTDEDGRSEDDLTSLSDKMPTFEETTTSAKYINGEKIMEHTVVETKHIDERGDTSVSNGDSNSTAVTRHSGLSSVSLSDQSTIVEDANALEEPELFAVHSPYVDDSTGEQMVRLYYELPVSLDDLEIIGLESRIPESDDDSIEARFRYRGEDFWLPVRYSYAKARMVLTGVC

SEQ ID NO: 34 - DNA, 38 bp, synthetic construct
  source 1..38: mol_type = other DNA
aaaaaaaaggcgcgccatggcatccacaaagcccgctt

SEQ ID NO: 35 - DNA, 45 bp, synthetic construct
  source 1..45: mol_type = other DNA
aaaaaaaagcggccgcctatatgtgatcttggggtgtttctgcat

SEQ ID NO: 36 - DNA, 9149 bp, synthetic construct
  source 1..9149: mol_type = other DNA
ggccgcgaagcttgagatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttggggatctgacagacgggcaattgattacgggatcccattggtaacgaaatgtaaaagctaggagatcgtccgccgatgtcaggatgatttcacttgtttcttgtccggctcaccggtcaaagctaaagaggagcaaaaggaacggatagaatcgggtgccgctgatctatacggtatagtgcccttatcacgttgactcaacccatgctatttaactcaacccctccttctgaaccccaccatcttcttccttttcctctcatcccacacaattctctatctcagatttgaattccaaaagtcctcggacgaaactgaacaagtcttcctcccttcgataaacctttggtgattggaataactgaccatcttctatagttcccaaaccaaccgacaatgtaaatacactcctcgattagccctctagtatccttgaagctgtccctgatggtcgtcatctacctgcctggacagcatggcctgcaacgcgggcatcccgatgccgccggaagcgagaagaatcataatggggaaggccatccagcctcgcgtcgagctttgaagttgctgcaagctggcttcaagccatcccatccgaatgtgatggatgcgttctttctgggccgttgcgactttggggatcgtctttcccgcgcccttggttggaggccctgtctccggtgtcccttgtcccttccaggcaagcgagcgaggtccattcagatggtgctccatcagcgttggctttccgtctccattggctcttggcaattcggtcagcggggctgactgcctcaggtggggcagtgctagtgtgtgtaccgacccgcaggattggtgctttgcccagagctctacagaatagcgcgcgcatccatatgttagttctgcaattttcttgtatcggtgctgtgactcatacttccccctttggctggccttgcggcaaccaataagaacgcacagtgaaatcttgcgggtggggagtggatccatggcgcctgcattggcttggggacgcgcactgtcgcacacttccatctgacctttcagaagggtttcgtggtgggcaaggaccaaccggttgcgcggccgtgcgtgggtgcctcgcccggcactgccagggccactgcagtggcagtttgctgcctgatacaaaatccttccctccgcccagttttccctctttgaccttcctttctcttctctgcaaccaaatccaccctatcaaaccaaaacagtatctcgaccgaggtatcaacctgaatcagcaacatcgtagccagcatttgtctccgtctctgcagaaccagcgagttgcaaacattatccaggcaacagggcaccaactcacttcttcggctttcaccaatcggtacagctcttctcagaactcgcgtccgcaacagttctacgcttcctcagcaccttcttcagcttcaatcctgaacactcagaaccgcgcacagcagcgccctcctgttcccttgtttcccaaaagtaccggtagtatttcgcacggaaagcagggcaacaagatgttctcaggtacccatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatggttgcctagtgaatgctccgtaacacccaatacgccggccgaaacttttttacaactctcctatgagtcgtttacccagaatgcacaggtacacttgtttagaggtaatccttctttctagctagaggatcctctacgccggacgcatcgtggccggcatcaccggcgccacaggtgcggttgctgactagaataattatgtgtaacaagaaagacagtataatacaaacaaagatgcaagagcggctcatcgtcaccccatgatagctagagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccttgtatctctacacacaggctcaaatcaataagaagaacggttcgtctttttcgtttatatcttgcatcgtcccaaagctattggcgggatattctgtttgcagttggctgacttgaagtaatctctgcagatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgggg

SEQ ID NO: 37 - DNA, 8747 bp, synthetic construct
  source 1..8747: mol_type = other DNA
gaattcccttgtatctctacacacaggctcaaatcaataagaagaacggttcgtctttttcgtttatatcttgcatcgtcccaaagctattggcgggatattctgtttgcagttggctgacttgaagtaatctctgcagatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatggggcgcgccaaaaaaaaggcgcgccatggcatccacaaagcccgcttcgagtctcatttaccaggcatggaacaaactcagtatcaaccaaaccatccctagtgactcccttgaattacttggggagcgtttggctattgccttcgcacccaaactcaaggagcaacgaaggaatggccggcgtcggaatctggaatatgtggcacaacatcgacggaagattgctcgaaaaatctacttggagattctggagaaagacccaaatatctttcttccttttatcctggctgtttcccctagagcatgcttatcctttgatatctcgagctttcttgaacagcaccaaagccaaggaagacatttcctccgcaacaatgccgaagcgatcctctggggtctcgcaaagaaacatgacattgatggctccctccatttcaggaagctgatgcgtgagattttccaactgtctcctccagcgacagaagccgaaggcaaggagcattattcattgcatttaagcactctccccgcaatccgcaatgccttcggtgatgttatctttgacgcaattgaacgttcccctacacaggtgacagcgagagctaaaggttatttctctgagaaaaccgaaagtgtttggacaaaagttccctacagaagttctcaagacgcaatcatatctcttgaagtagggtcggcaatcgagcttgcgaatgtgttgttcccaatcgcaacccaaaaaattgtctctatcctttccgcatgttctcccactgtgcgccagaagaacttttctgaggctattctcggcccagaccctcaggatacaccggcaacatcatcagaaatcggtatgaagtttaaggtacacatgactgcagttgctaattccaccctgtgctagatgtggcgtattttactctgcgaggagcaacggtctcggcaattgaatcagtctttcgcgctgatatttgcgaaggtattaagggcagcgaactgagaaactgggaaaaggagcagctgctcatcgacacgacagattgtgtcacgatgcagatatggcgggcacaacctcaacatggaaccatcaagttgcgtattggattctatgcagcggtgaatttggcaaatcggctgtatgcagaaacaccccaagatcacatataggcggccgcgaagcttgagatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaggcctcaaacaatgctcttcaccctcttcgcgggtctgaaataccctcacctggcaacagcaattggcgcttcatggctgtttttccgatctctctacttgtacggctatgtgtactcgggtaagccacaaggcaagggcagattgctgggaggtttcttctggttttctcaaggcgctctgtgggctctgagtgtgtttggtgttgccaaagacatgatctcttactgagagttattctgtgtctgacgaaatatgttgtgtatatatatatatgtacgttaaaagttccgtggagttaccagtgattgaccaatgttttatcttctacagttctgcctgtctaccccattctagctgtacctgactacagaatagtttaattgtggttgaccccacagtcggaggcggaggaatacagcaccgatgtggcctgtctccatccagattggcacgcaatttttacacgcggaaaagatcgagatagagtacgactttaaatttagtccccggcggcttctattttagaatatttgagatttgattctcaagcaattgatttggttgggtcaccctcaattggataatatacctcattgctcggctacttcaactcatcaatcaccgtcataccccgcatataaccctccattcccacgatgtcgtccaagtcgcaattgacttacggtgctcgagccagcaagcaccccaatcctctggcaaagagactttttgagattgccgaagcaaagaagacaaacgttaccgtctctgctgatgtgacgacaacccgagaactcctggacctcgctgaccgtacggaagctgttggatccaatacatatgccgtctagcaatggactaatcaacttttgatgatacaggtctcggtccctacatcgccgtcatcaagacacacatcgacatcctcaccgatttcagcgtcgacactatcaatggcctgaatgtgctggctcaaaagcacaactttttgatcttcgaggaccgcaaattcatcgacatcggcaataccgtccagaagcaataccacggcggtgctctgaggatctccgaatgggcccacattatcaactgcagcgttctccctggcgagggcatcgtcgaggctctggcccagaccgcatctgcgcaagacttcccctatggtcctgagagaggactgttggtcctggcagagatgacctccaaaggatcgctggctacgggcgagtataccaaggcatcggttgactacgctcgcaaatacaagaacttcgttatgggtttcgtgtcgacgcgggccctgacggaagtgcagtcggatgtgtcttcagcctcggaggatgaagatttcgtggtcttcacgacgggtgtgaacctctcttccaaaggagataagcttggacagcaataccagactcctgcatcggctattggacgcggtgccgactttatcatcgccggtcgaggcatctacgctgctcccgacccggttgaagctgcacagcggtaccagaaagaaggctgggaagcttatatggccagagtatgcggcaagtcatgatttcctcttggagcaaaagtgtagtgccagtacgagtgttgtggaggaaggctgcatacattgtgcctgtcattaaacgatgagctcgtccgtattggcccctgtaatgccatgttttccgcccccaatcgtcaaggttttccctttgttagattcctaccagtcatctagcaagtgaggtaagctttgccagaaacgccaaggctttatctatgtagtcgataagcaaagtggactgatagcttaatatggaaggtccctcagggacaagtcgacctgtgcagaagagataacagcttggcatcacgcatcagtgcctcctctcagacagaatctagagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 38 - DNA, 36 bp, synthetic construct
  source 1..36: mol_type = other DNA
aaaaggcgcgccatggcttggtacagtgctttactc

SEQ ID NO: 39 - DNA, 35 bp, synthetic construct
  source 1..35: mol_type = other DNA
aaaagcggccgctcagaacagaccgcttgaaaggc

SEQ ID NO: 40 - DNA, 9797 bp, unidentified
  source 1..9797: mol_type = other DNA; note = Aspergillus niger
gaattcccttgtatctctacacacaggctcaaatcaataagaagaacggttcgtctttttcgtttatatcttgcatcgtcccaaagctattggcgggatattctgtttgcagttggctgacttgaagtaatctctgcagatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatggggcgcgccatggcttggtacagtgctttactcccgtgcatgctatggtggcggaacctcctgtggcgtaacagcaccaataggtatagacagagtacagacgaccttacttcactgactgataagatacctaatcttggagaaagggtaagtgtaaatactttttgaggtcgcttctaggagtctaatcactttggaaagacaactcccacagataacatcgatggaaagagcttcgtggcacatactgtcgtgcagaccaggcggattggtgaccgagaacgttgctgcttcaatgactcggatttgaacagcggcacagttaccaaatttattgagctttgttaatagcattccagatcagagtactactgccggaaaaattaatcacaaggccctgcgagatcctgagctctttgctatccgctcgtcatgcatcgacaaatcagccagcaagtggatggttagtctctactacgaacccccacccagccttgatgacctcgagattaagaacttcggatctcgtattccagagtcggaggatgatccaattgaggctatctttcactatgagggagagaacatttgggtatctgttccttatttgtatgctagaactagaagcctttcaagcggtctgttctgagcggccgcgaagcttgagatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttggggatctgacagacgggcaattgattacgggatcccattggtaacgaaatgtaaaagctaggagatcgtccgccgatgtcaggatgatttcacttgtttcttgtccggctcaccggtcaaagctaaagaggagcaaaaggaacggatagaatcgggtgccgctgatctatacggtatagtgcccttatcacgttgactcaacccatgctatttaactcaacccctccttctgaaccccaccatcttcttccttttcctctcatcccacacaattctctatctcagatttgaattccaaaagtcctcggacgaaactgaacaagtcttcctcccttcgataaacctttggtgattggaataactgaccatcttctatagttcccaaaccaaccgacaatgtaaatacactcctcgattagccctctagtatccttgaagctgtccctgatggtcgtcatctacctgcctggacagcatggcctgcaacgcgggcatcccgatgccgccggaagcgagaagaatcataatggggaaggccatccagcctcgcgtcgagctttgaagttgctgcaagctggcttcaagccatcccatccgaatgtgatggatgcgttctttctgggccgttgcgactttggggatcgtctttcccgcgcccttggttggaggccctgtctccggtgtcccttgtcccttccaggcaagcgagcgaggtccattcagatggtgctccatcagcgttggctttccgtctccattggctcttggcaattcggtcagcggggctgactgcctcaggtggggcagtgctagtgtgtgtaccgacccgcaggattggtgctttgcccagagctctacagaatagcgcgcgcatccatatgttagttctgcaattttcttgtatcggtgctgtgactcatacttccccctttggctggccttgcggcaaccaataagaacgcacagtgaaatcttgcgggtggggagtggatccatggcgcctgcattggcttggggacgcgcactgtcgcacacttccatctgacctttcagaagggtttcgtggtgggcaaggaccaaccggttgcgcggccgtgcgtgggtgcctcgcccggcactgccagggccactgcagtggcagtttgctgcctgatacaaaatccttccctccgcccagttttccctctttgaccttcctttctcttctctgcaaccaaatccaccctatcaaaccaaaacagtatctcgaccgaggtatcaacctgaatcagcaacatcgtagccagcatttgtctccgtctctgcagaaccagcgagttgcaaacattatccaggcaacagggcaccaactcacttcttcggctttcaccaatcggtacagctcttctcagaactcgcgtccgcaacagttctacgcttcctcagcaccttcttcagcttcaatcctgaacactcagaaccgcgcacagcagcgccctcctgttcccttgtttcccaaaagtaccggtagtatttcgcacggaaagcagggcaacaagatgttctcaggtacccatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatggttgcctagtgaatgctccgtaacacccaatacgccggccgaaacttttttacaactctcctatgagtcgtttacccagaatgcacaggtacacttgtttagaggtaatccttctttctagctagaggatcctctacgccggacgcatcgtggccggcatcaccggcgccacaggtgcggttgctgactagaataattatgtgtaacaagaaagacagtataatacaaacaaagatgcaagagcggctcatcgtcaccccatgatagctagagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 41 - DNA, 31 bp, synthetic construct
  source 1..31: mol_type = other DNA
aaaaagatctgccgtaacgtaacaaagcggg

SEQ ID NO: 42 - DNA, 36 bp, synthetic construct
  source 1..36: mol_type = other DNA
aaaagcggccgcttcgggcagcgaattggtatggtc

SEQ ID NO: 43 - DNA, 5068 bp, synthetic construct
  source 1..5068: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcggcgcgccagatctacgcgtttaattaaccgcggtggcggccgctctagaactagtggatcccccgggctgcaggaattcgatatcaagcttatcgataccgtcgaggtctgagaggaggcactgatgcgctatcatggggtgacgatgagccgctcttgcatctttgtttgtattatactgtctttcttgttacacataattattctagaatgccccaccgttacatacgggacacagccatttacatatgcatgtggattacgagctaacgagttcattcaaatctcagaactatcacataatcatcattcccctatcgtcaaagaccgtaagacaaatccggttcatgcactgaacccattcgggtagtgagtcatttactcagcacactcgcgctgacgctcgtcgaacaccttcaatgcctcctcggcagccttgacaccactgagaaccatggcaccgaaggtagggcccatgcggttaaagccatcaatttcagacagctccataccgccgattatcaagcccttagtaacctcgcgggtgttcttgacgatggcatcctcggccgagttcatgtcgagaccacgcatgccacctagcttgtcgacgctgcccatggacaccaagcgcttcgcacagaaggcgccgaatggcccatcgtgaccagtggtactgatgatgacaggagcgttgatagtgttggggtccatgcaggagtgatcatcgtggtgaagggtgaccagcgtccagttgacgacaacaccagcaatctgggggttgccgttctcggtcggacgggtgatcaagtcctcaacagcggtagcattgaagagcttgacattggggaaggagagaaccttcgacatgagtgtcgaggtaaacagggaggcgtgcttgacgacaacgtagttggggtttgcgtcctcttcgtaaggaacacccagctcgttcaggaagacttccgcgggacggcgcatgaccatagcagaaaagagttggccacccaaccaggcaccgccacctattgcacgttagttccggaaagctgagtgcaaggcaatccatcatggactactgaccaggagagacgctggcctcgacgatagcaatcttcaggtccggacgagccttggccaagacgtacgcagtgctcagaccgcaggaaccagcaccaacaatgacaacgtcactttcagcgtacttgtccaggtcctcaaagtaacgtctggtcatggcacgagagacctggctttcgcggataggggcgaacttgaactcgtcccacttgccaccgaaatggtccaacagcttggtctgagaagctccctcaacggggacggtctcagaaaccacgaccttacccttgaggccggtagcggccacagtgggttcgtagatggcagctggaggagacatgtttcaagttgcaatgactatcatctgttagccattccatcaacaggaagaacgagagaaggcatgacccttttcgctggtattatccagatcaagttttagccgtataatctcagaacgaacccagtccatcgatgccatgtccttctagactaggatcctagagtctagggcccagcttagggagggcatgtgaatgcatcgatgactgggaacgaacaccggcccacgccaaagacgttacctaagataccttgatcattgtgagagtccagccaaaagtattccatgacttccatcgtatgccctctagagggctaatcgaggagtgtatttacattgtcggttggtttgggaactatagaagatggtcagttattccaatcaccaaaggtttatcgaagggaggaagacttgttcagtttcgtccgaggacttttggaattcaaatctgagatagagaattgtgtgggatgagaggaaaaggaagaagatggtggggttcagaaggaggggttgagttaaatagcatgggttgagtcaacgtgataagggcactataccgtatagatcagcggcacccgattctatccgttccttttgctcctctttagctttgaccggtgagccggacaagaaacaagtgaaatcatcctgacatcggcggacgatctcctagcttttacatttcgttaccaatgggatcccgtaatcaattgcccgtctgtcagatccccagagcattgtttgaggcgaccggtctcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 44 - DNA, 13900 bp, synthetic construct
  source 1..13900: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcggcgcgccagatctgccgtaacgtaacaaagcggggttggtagtgtttgcaaatgcattcacatggaccgatcacttttctttccagtctgtccattctgtccaatctgtccgatctgacctgcccagtctgtccagtctgtcccttgtgtcgtccgatccaagctggttatcatggcatccacaaagcccgcttcgagtctcatttaccaggcatggaacaaactcagtatcaaccaaaccatccctagtgactcccttgaattacttggggagcgtttggctattgccttcgcacccaaactcaaggagcaacgaaggaatggccggcgtcggaatctggaatatgtggcacaacatcgacggaagattgctcgaaaaatctacttggagattctggagaaagacccaaatatctttcttccttttatcctggctgtttcccctagagcatgcttatcctttgatatctcgagctttcttgaacagcaccaaagccaaggaagacatttcctccgcaacaatgccgaagcgatcctctggggtctcgcaaagaaacatgacattgatggctccctccatttcaggaagctgatgcgtgagattttccaactgtctcctccagcgacagaagccgaaggcaaggagcattattcattgcatttaagcactctccccgcaatccgcaatgccttcggtgatgttatctttgacgcaattgaacgttcccctacacaggtgacagcgagagctaaaggttatttctctgagaaaaccgaaagtgtttggacaaaagttccctacagaagttctcaagacgcaatcatatctcttgaagtagggtcggcaatcgagcttgcgaatgtgttgttcccaatcgcaacccaaaaaattgtctctatcctttccgcatgttctcccactgtgcgccagaagaacttttctgaggctattctcggcccagaccctcaggatacaccggcaacatcatcagaaatcggtatgaagtttaaggtacacatgactgcagttgctaattccaccctgtgctagatgtggcgtattttactctgcgaggagcaacggtctcggcaattgaatcagtctttcgcgctgatatttgcgaaggtattaagggcagcgaactgagaaactgggaaaaggagcagctgctcatcgacacgacagattgtgtcacgatgcagatatggcgggcacaacctcaacatggaaccatcaagttgcgtattggattctatgcagcggtgaatttggcaaatcggctgtatgcagaaacaccccaagatcacatatagtaacctttcatcttttcggccttcttaaatcattgcctttctgtgagtcgcgactttccaccctttatgaatacaccaataccagggggaagaacgatttcaccgcttcccttggcaatccatatagttcccttctcattctggaacctgatttcatcgcagttgaagcaatataaattcctttcgaggttttctgcattgtagggatggaatgggtgcgtgaaatatttgtcgaaaatagaagggtccgaaagttctttccaagctgcgcgctgaaagttgttagcccatctttcgagcatatggtttggcccacatacgagagattcttttgtgatcggttgagattcttccgtgatcggttctgtcattttcattaagtcagagagccctcttgtatgccggcttttgctgtcggatccggcgagataatcgctcctaagccagtcagtcagggaaaagcaaagataaataaaatataggcgaggagtacaaccaggcacgcgtcgtagactatttttctgaagagtttgtcacgtaacctacctcatatggatgggtagttcgaatacttgattgacttgacccgaggttctgaaggcggcggaggaaattgcccaaccccaccattgcattttcaggtatcaatctctgccacactgtggctaaattcgtctttatcgacacgtgatcacgttccctcttccagccctggtatcagagaatcatcgagttatcgcttgtttcaatttcgtcttgcaattagcttagggaataagcatgtggtcacatcaacctacagagcgctaccggtctttgcgctgagactctcagtgatccgcccaacagacaactagactttgaggttgtcgatataaccacaacaaatggcctgtatatcaacgatgtccacgcaattgtctcaagcctcttcacccgacttccagcactagcatccaagcggcctctcctcttctcccatgtttctcgtagcgcgcctgcatatacttatatctggagatatgttaaaggagctggaagcctggagcatacgctggaagcctggagcatacgcttcaagtgctgccatattcagatagctgagtaggcacaattaggtctaagttcagggaattgcacctctcgcttcattgtccgtcgattcgtatcggtctctagttctccccgtttatcactctcactcggtggacagtccgtccagtccgtccagtccgtcgagcctgtccaatctgtccaatctgtccaatctgtccaatctgtccaatctgtccaatctgtccaatctgtccaatctgtccaatctgtccaatctgtccaatctgtccaatctgtccaatctgtccaatctgtccaatctgtccaatctgtccactctgtccactctgtccactctgtccaatctgtccactctgtcccctctgtccactctgtcccctctgtccactctgtcccctctgtccaatcggtccaatctgtccaatcttgatgatctcgatgatcaataccatttagcgagcgctagtgacgccttacagcgttgcggcgtcttatggcttatacatcttccgaatatcagtcgttcgatctccagatcacacctcggggtgaaacaagcgccatagttcttggtgcgcctgagcttttccctgcccggccactcagtcgtgatggcttcccagagtattccataagtcgaaaccagagataggccagcggacggcaaatctcctctgctccgttctcaaccaatactgccaaagtgagcaatggaaagtgttccaggagcaccgtggcctttaaaagcgccagccttgtcccagatcctcaccgcaattcggcacagaccaacgcagactttcattgaccattcttcatttccagatcgctgcatagtttccggtatggcttggtatagagccttactcccgtgcataccgttgtggcggaaggttctggggcgtaacagcaccgatgaggacggacggagtgaagacgaccttacttcattgagcgataaaatgcctacttttgaagaaacgacaactgtgagtactgttcgtgaaattgcctccagctgtctaatgtctccgtcggtcagatcacttctgcaaagtacatcaacggtgaaaaaatcatggagcataccgttgtggagaccaagcacattgacgaacgtggagacaccagcgtcagtaacggtgattcgaacagcactgcggtaaccagacattctgggcttagctctgtcagcctttcagatcaaagtacaattgtcgaggacgcgaatgctctggaagaacctgaactctttgctgttcactctccatatgttgacgactcaaccggcgagcagatggttagactctactacgagctcccagtaagcctcgatgatcttgagatcataggccttgaatctcgtattccagagtctgacgatgattcaattgaggcccgctttcgttatcgaggagaggatttttggctacctgttcgttattcttatgccaaagctcgaatggttttaacgggtgtatgctgaatggtctgacttcttcactgttgttattttctatttcccggctgctggccactcaatattatcgcaagcactatacaaaataaatagtcctatctctataacagagtacgtcacaaacagcgttcgtccttggcaagaatataaatagtgtcaatctgctgagaaaaggaggtatgaaatccacttcattcaagcaatggttcccttcgtatacattattatttgtatatggatgggaacttttcttgtcttagttgccctgaacgcccgctttaagaagaagcgcattgctgatcctgagtcttctgctgccttcactgatcatccccatcaagagtaacatggattctgtatgtccctttgggcatgtttatgtggcagttactaatatattagatggaaactaggaagcggcaaaacacaggggcaggaagggaaggactcattccaagaaagaaggacaaggaagaacacaagagtgtaaggggtagcaatcctcattggcgtctactagctgatgggttaaagcaagatacaggtgttcatacgctccaagacaacgatcctgttgtcgagaagccgccattaatttacaggagagctggagaagcgccgaaacatgattgggacaagaagtcacccggtttgcgccttagacggtcctgctgtagctgcgggaaggaagtaccggtaggcctggtctgtcgtatttgtcaccatgagtcttgtcctgagtgcttgaacatgcaaaagcgagattacggatgttgatcagatacacgggcccctatactaggactagttaccactaggattgtactcaaccttaacagtggcgtttggtacttcgttacagctaaggggagaggacagttcctactttcatgtgcttcaagggagaggctcctccaactactgttgctagggaggaaagacactctattcttagctgagggctgcaggaattcgcatccatgcagtcataccatccatggcatgcaatctatgttgcattagatgcaagaagtaggatagagaacccatgtacttgattcacgctagcaggacagagagagataccatacccgacgggagcccctgcatgacttcctgtctgcagcttgtcgtgcgtgtatcattcccatgcgccacgaactcataggcagtggtagttcagaacactctttttttttaaaaaaaaaaaagataggaaaataataatttaggggaagaaaagtaaaaattaaaaagaaaaagttccagatggcgcttctacttctatattcgatcgttattcaataccccagaggcacaggcattccgatctctcatcgccaacactgaaaagcagccattttcccccgtcttaaagctccaatcctccttcttctcatctacttcctcgctccttcaggaccttgagtgttccgttgagctattgggtaacttctcacctgtcaatcatcgattgtcctttctcttgacttgacttcgtgtcgccattctcatttacgatacatatccctggagcagaaaacaaagaaaagggccaattactcttgatctagttccaactctgttgctgcttggaacatccgcccatctgtgtggtgaaatcagatgccagcatccatcttgcagcttctcccacttcctgggccgatcttgaatgtttgctctcgaacctcgcttagtatttgatctccattctcatctgggtacatcctgtgagtagcatgtcgtcacttgtcacacatactacccgctctcaaatctgtttgatgggagtcaatctgcctcgaaatggctcgtctgccttcacaagcaaactacagcagatggcgggggcatggactcgagccacagtgctggctctcgcttgcatctggaccttcttattctttctcattgctgtatctttttcccccttgaggcttctggcgcgctgcacctttccaagtatcaaaccaaagctaatcaggggcgtttggcgtcctgccatggcttcactagacctggatctctgcagcctcatcaccatctcggatcacctggttctgatcaccttggaagaaagcacaaagaccttggagacaatacatattgccgccatcgcagctccctccaatctcgacagcattttcatgtgtcgggcactatctacctctcggcaattcagtaaccgtactgcctgagaaacatcaacctctcaaattacacaatggtgttcagcgcacctgctcctggtgtgggctccagtaaaaggccagcgtcatgcatgcaggacgatgttgatgagcgggataatgtcccagtaagtgataccagcattggaaaggcagatggagctgactcatctcctatatagcccatgggtctatgcgtgcgttcatcgatcaacatcccttacttccactacgccatgatctatctcgacaacatgggcagacttaaggtgatggaatctccgtctatccaggagcaaaatgagactgttttcacaaccgaagtacgtgaaagatttttggaaatccttggtgccaaggtaggatatcaaccgcccatggttcgaagtatgtaaacactccgcgcacaagtacaatattcttgctgatctcaattgaacagggttgtcagctgccggtgctacaccatacagctatgatcctcaacaaccgcttggttgcttgtcttaccgtcaaactaagcgggacagaaattccccagcccactctatgtacggtgtgccgccatccgtccagttctcagccccggttgaggaatcgccctcttgtggatcagtggacatggtcgggctcgagattggtgatactcctaatgtccttgactactatgagagatccttaaagcactttcggcaggtcaactgtcgccagatcctaaagacattcattaagttcattgagccacgaaagcaagccaagcacccctataatggaggtaaaccccctgcaggagcccctcctggtaagaagggcgacccagagaagacaaagcctgaatggtggcccgccaatgtggtccacaaggagcctgaccatcttcgaaaggatcgtacgtgtaacccttcagaaaatcttcagtgtcaagtaactttgctgacagacttagaacgcctgtctctgttaattcatatcatccgcaggcttggaagatttggtatcaccacggatcaattgcaggaaattgcccacgactgcaagcggcggctcagcgacccccacaaactccaaatcttggacgaggtcttcagagtgagaaggattgaagaacgctacgaaagaggagaagttggtaagcggcatcatctttccatgaaattcattttgacagctgttgacgagcctcagatgccaacaagatcgtatatgttgtcaaccgagagtcgaatcagaaagagaaggatggcgactccaacgtggatccggaccagaagcatgagcaagaagacgataatgcgcgggaggcacttcccattctccactccgagaagaactcaaccagcccgatgtcgaactcagccgagcacacgggcatggcggcaccaagtcgtccaatgaatatgggaggtgacagaaaccagttgtttcctttaccggagtggccgagcttcggtgagacaccccaggatgatcgaattttctttcccacgacctctaagtataccgaagattatgcatcgcagcagatgcctagaacacctgcaacaacagcacttgtcagcactaatgagacacatgcggcctttgattatatgacacaggagtccatcacctcctcctccccagagcagacttcccaccaccgccaagcacccctgcccatgcagcactcggccagcctcgacccttggacccctacgttccgacataatttcttcaacccaatggtgtatagtactgcaccccgtcacgccatgtcccaggctactatgttatctcagtttcccaggtccacgacgtctcatggccaggaaatgcctcacatggctcacggcctgccgaacctgcctcaagacagaccttcaagcatggatggcatgagcatgagaggcccttctttccgcacaggatttttgagtcatccctgtgacccatcacagcaggctcctcattctagcggatgcggccatcctgacagttggactcaaaatagaccacatgtataatcttaactgattgatccttgaccactgttttgaccctcctgcagccttgaagcttcgtttcactgatgattgttcttcgactttgtttctgtccctgactttgttgtcaatgcggacttatccatgcggcttgttccacgtcaagtgactaccaggacactccgtggttttatatggcaggtactggcgatgactttccaattcttcttcgtttagtatatatactcgtttcttgttctatgttcgatcatgtctttttccttatacatacctccaaaaatcctgttggagatggcgccagatggcatgagatgcaaatatggatgatgttcttgtgtttgttcatttcaatttctttctcttaatcatgatttgaacaattggcagcgaggtatggcggagctcgttctctttggatgccgatcagctgaataggaggtaacgaggcatgagggtgtttcattatgactctctccggtgtttgtcatttaagggtgcgagggggaagtgtccgtttcgatgtcctaggatatcgaaaatctgagtagtagccacgtgaccctatgctgacggctgggctggaagacaagcaggttgctgcttacgagaatatgttgaggtattctcgttatcttcgtgaagaatgccgtctccttggccctctagccaaagtctgggttgctgaaaggctagctggaattgagaatcgactgtctgcgtccgagtcgcctagaggtgggaaggccccctctttctcatacatatgctgactctgcagaccataccaattcgctgcccgaagcggccgctctagaactagtggatcccccgggctgcaggaattcgatatcaagcttatcgataccgtcgaggtctgagaggaggcactgatgcgctatcatggggtgacgatgagccgctcttgcatctttgtttgtattatactgtctttcttgttacacataattattctagaatgccccaccgttacatacgggacacagccatttacatatgcatgtggattacgagctaacgagttcattcaaatctcagaactatcacataatcatcattcccctatcgtcaaagaccgtaagacaaatccggttcatgcactgaacccattcgggtagtgagtcatttactcagcacactcgcgctgacgctcgtcgaacaccttcaatgcctcctcggcagccttgacaccactgagaaccatggcaccgaaggtagggcccatgcggttaaagccatcaatttcagacagctccataccgccgattatcaagcccttagtaacctcgcgggtgttcttgacgatggcatcctcggccgagttcatgtcgagaccacgcatgccacctagcttgtcgacgctgcccatggacaccaagcgcttcgcacagaaggcgccgaatggcccatcgtgaccagtggtactgatgatgacaggagcgttgatagtgttggggtccatgcaggagtgatcatcgtggtgaagggtgaccagcgtccagttgacgacaacaccagcaatctgggggttgccgttctcggtcggacgggtgatcaagtcctcaacagcggtagcattgaagagcttgacattggggaaggagagaaccttcgacatgagtgtcgaggtaaacagggaggcgtgcttgacgacaacgtagttggggtttgcgtcctcttcgtaaggaacacccagctcgttcaggaagacttccgcgggacggcgcatgaccatagcagaaaagagttggccacccaaccaggcaccgccacctattgcacgttagttccggaaagctgagtgcaaggcaatccatcatggactactgaccaggagagacgctggcctcgacgatagcaatcttcaggtccggacgagccttggccaagacgtacgcagtgctcagaccgcaggaaccagcaccaacaatgacaacgtcactttcagcgtacttgtccaggtcctcaaagtaacgtctggtcatggcacgagagacctggctttcgcggataggggcgaacttgaactcgtcccacttgccaccgaaatggtccaacagcttggtctgagaagctccctcaacggggacggtctcagaaaccacgaccttacccttgaggccggtagcggccacagtgggttcgtagatggcagctggaggagacatgtttcaagttgcaatgactatcatctgttagccattccatcaacaggaagaacgagagaaggcatgacccttttcgctggtattatccagatcaagttttagccgtataatctcagaacgaacccagtccatcgatgccatgtccttctagactaggatcctagagtctagggcccagcttagggagggcatgtgaatgcatcgatgactgggaacgaacaccggcccacgccaaagacgttacctaagataccttgatcattgtgagagtccagccaaaagtattccatgacttccatcgtatgccctctagagggctaatcgaggagtgtatttacattgtcggttggtttgggaactatagaagatggtcagttattccaatcaccaaaggtttatcgaagggaggaagacttgttcagtttcgtccgaggacttttggaattcaaatctgagatagagaattgtgtgggatgagaggaaaaggaagaagatggtggggttcagaaggaggggttgagttaaatagcatgggttgagtcaacgtgataagggcactataccgtatagatcagcggcacccgattctatccgttccttttgctcctctttagctttgaccggtgagccggacaagaaacaagtgaaatcatcctgacatcggcggacgatctcctagcttttacatttcgttaccaatgggatcccgtaatcaattgcccgtctgtcagatccccagagcattgtttgaggcgaccggtctcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac